SEQUENCE LISTING (494 sequences)

SEQ ID NO: 1 - protein, 64 aa, synthetic construct
  REGION 1..64: note = Synthetic peptide
GSPKEEARELIRKQKELIKEQKKLIKEAKQKSDSRDAERIWKRSREINRESKKINKRIKELIKS

SEQ ID NO: 2 - protein, 62 aa, synthetic construct
  REGION 1..62: note = Synthetic peptide
PKKEAEELAEESEELHDRSEKLHERAEQSSNSEEARKILEDIERISERIEEISDRIERLLRS

SEQ ID NO: 3 - protein, 77 aa, synthetic construct
  REGION 1..77: note = Synthetic peptide
GTKEDILERQRKIIERAQEIHRRQQEILEELERIIRKPGSSEEAMKRMLKLLEESLRLLKELLELSEESAQLLYEQR

SEQ ID NO: 4 - protein, 83 aa, synthetic construct
  REGION 1..83: note = Synthetic peptide
GTEKRLLEEAERAHREQKEIIKKAQELHRRLEEIVRQSGSSEEAKKEAKKILEEIRELSKRSLELLREILYLSQEQKGSLVPR

SEQ ID NO: 5 - protein, 76 aa, synthetic construct
  REGION 1..76: note = Synthetic peptide
TKEDILERQRKIIERAQEIIRRQQEILEELERIIRKPGSSEEAMKRMLKLLEESLRLLKELLELLEESAQLLYEQR

SEQ ID NO: 6 - protein, 78 aa, synthetic construct
  REGION 1..78: note = Synthetic peptide
GSTEKRLLEEAERAHREAKEIIKKAQELHRRLEEIVRQSGSSEEAKKEAKKILEEIRELSKRLLELLREILYLSQEQK

SEQ ID NO: 7 - protein, 76 aa, synthetic construct
  REGION 1..76: note = Synthetic peptide
TKEDILERARKIIERAQEIHRRQQEILEELERIIRKPGSSEEAMKRMLKLLEESLRLLKELLELSEELAQLLYEQR

SEQ ID NO: 8 - protein, 78 aa, synthetic construct
  REGION 1..78: note = Synthetic peptide
GSTEKRLLEEAERAIREQKEIIKKAQELHRRLEEIVRQSGSSEEAKKEAKKILEEIRELSKRSLELLREILYLLQEQK

SEQ ID NO: 9 - protein, 35 aa, synthetic construct
  REGION 1..35: note = Synthetic peptide
TKEDILERQRKIIERAQEIHRRQQEILEELEYIIR

SEQ ID NO: 10 - protein, 121 aa, synthetic construct
  REGION 1..121: note = Synthetic peptide
MSEEAMKRMLKLLEESLRLLKELLELSEESAQLLYEQRKANNGSETEKRLLEEAERAHREQKEIIKKAQELHRRLEEIVRQSGSSEEAKKEAKKILEEIRELSKRSLELLREILYLSQEQK

SEQ ID NO: 11 - protein, 77 aa, synthetic construct
  REGION 1..77: note = Synthetic peptide
MTKEDILERQRKIIERAQEIHRRQQEILKEQEKIIRKPGSSEEAMKRSLKLIEESLRLLKELLELSEESAQLLYEQR

SEQ ID NO: 12 - protein, 77 aa, synthetic construct
  REGION 1..77: note = Synthetic peptide
GTEKRLLEEAERAHREQKEIIKKAQELHKELTKIHQQSGSSEEAKKRALKISQEIRELSKRSLELLREILYLSQEQK

SEQ ID NO: 13 - protein, 76 aa, synthetic construct
  REGION 1..76: note = Synthetic peptide
TKEDILERQRKIIERAQEIHRRQQEILKRSEEIIRKPGSSEEALETLRELQEESLRLLKELLELSEESAQLLYEQR

SEQ ID NO: 14 - protein, 78 aa, synthetic construct
  REGION 1..78: note = Synthetic peptide
GSTEKRLLEEAERAHREQKEIIKKAQELHRRTEEIIRQSGSSEEAKDELRRIQEEIRELSKRSLELLREILYLSQEQK

SEQ ID NO: 15 - protein, 36 aa, synthetic construct
  REGION 1..36: note = Synthetic peptide
TTKRYLEEAERAHREQKEIIKKAQELHRRLEEIVRQ

SEQ ID NO: 16 - protein, 116 aa, synthetic construct
  REGION 1..116: note = Synthetic peptide
GSSEEAKKEAKKILEEIRELSKRSLELLREILYLSQQVNDVDEKALERQRKIIERAQEIHRRQQEILEELERIIRKPGSSEEAMKRMLKLLEESLRLLKELLELSEESAQLLYEAR

SEQ ID NO: 17 - protein, 35 aa, synthetic construct
  REGION 1..35: note = Synthetic peptide
EAMKRMLKLLEESLRLLKELLELSEESAQLLYEAR

SEQ ID NO: 18 - protein, 116 aa, synthetic construct
  REGION 1..116: note = Synthetic peptide
TTKRYLEEAERAHREQKEIIKKAQELHRRLEEIVRQSGSSEEAKKEAKKILEEIRELSKRSLELLREILYLSQQVNDVDEKALERQRKIIERAQEIHRRQQEILEELERIIRKPGS

SEQ ID NO: 19 - protein, 77 aa, synthetic construct
  REGION 1..77: note = Synthetic peptide
TREELLRENIELAKEHIEIMREILELLQKMEELLEKARGADEDVAKTIKELLRRLKEIIERNQRIAKEHEYIARERS

SEQ ID NO: 20 - protein, 78 aa, synthetic construct
  REGION 1..78: note = Synthetic peptide
GTERKLLERSRRLQEESKRLLDEMAEIMRRIKKLLKKARGADEKVLDELRKIIERIRELLDRSRKIHERSEEIAYKEE

SEQ ID NO: 21 - protein, 76 aa, synthetic construct
  REGION 1..76: note = Synthetic peptide
GDRQELIRRNIELLKEHIKILEEISQLIEELSELLDKSSSEEVVKRYKKILERYKQLLRKSQEIHKESSEIAKKES

SEQ ID NO: 22 - protein, 82 aa, synthetic construct
  REGION 1..82: note = Synthetic peptide
GDEQKLIERSQRMQKESLELLKEIIKILDTIEKLLDKPDSEELLDTIKKLHDTLKKIHDRNKKLLKEHEEILRQRSGSLVPR

SEQ ID NO: 23 - protein, 73 aa, synthetic construct
  REGION 1..73: note = Synthetic peptide
DKEEEYKRLLDEIKEILKESKEVLKDSKRVLEDIKRKVPDDDLVKLLEKHVRLLEEHVKLLEQLIREAEKSSK

SEQ ID NO: 24 - protein, 72 aa, synthetic construct
  REGION 1..72: note = Synthetic peptide
QGSSAEELLKKIKESEKKIRDSLRKIKEIIKKSRKEGVDDKQLDLIRKVVESHRDLLRLHRDLLRLLREETS

SEQ ID NO: 25 - protein, 77 aa, synthetic construct
  REGION 1..77: note = Synthetic peptide
DIDESIKEVEKLLEEVEQSLQKLDDSLKKLLEKVNQDPDVDDSVRKIVKRHVEILKRHEEVLKRLIEVVKEHTKTVK

SEQ ID NO: 26 - protein, 72 aa, synthetic construct
  REGION 1..72: note = Synthetic peptide
GSDREEVHKEIVKLIREIIKIHKKILKIHEKIKNGEIDPSEILKLSEEIKKLTDTIIKIIEDLEQLTRDLRR

SEQ ID NO: 27 - protein, 71 aa, synthetic construct
  REGION 1..71: note = Synthetic peptide
DRKEIVKRHQKVVELLKESSKLLRESSKLLQRLLDKTGDENLQKAVDDQDKAIKRQETAIRKSQEASKKLD

SEQ ID NO: 28 - protein, 77 aa, synthetic construct
  REGION 1..77: note = Synthetic peptide
DNSEEIKKVAKTSREVAEYSERVAKENDKVVKTLEEGKIDESELLRLLEESIKIFDTALKLHEEAYKLHQDLVRKVS

SEQ ID NO: 29 - protein, 72 aa, synthetic construct
  REGION 1..72: note = Synthetic peptide
DESEAASVAIESVQILVESVKLLEESVRILLDAVKKNGVEDLLRVAQRWEKLVDEWLKVVKRWLDNVRDIQR

SEQ ID NO: 30 - protein, 76 aa, synthetic construct
  REGION 1..76: note = Synthetic peptide
GSDKAEEVEKSVRKIEESIKKIRKSIKKAEDAVQLLKEGKIDAKDFLRIVREDLEVVKEDVEIVKEDVENVREFSS

SEQ ID NO: 31 - protein, 69 aa, synthetic construct
  REGION 1..69: note = Synthetic peptide
SDKEVSDKLLKASKKLLKVSEELLEVVRRLLKALKDDELIKKIADLLRKIIDKDKKFIRTSEEIVKESR

SEQ ID NO: 32 - protein, 78 aa, synthetic construct
  REGION 1..78: note = Synthetic peptide
GSDLKEVLKTVEEAVKEIIKSSEELLQISRKILEISRVGVDEHEYISAIREYLKALEKHIQILKKFIEILKELIRAVS

SEQ ID NO: 33 - protein, 81 aa, synthetic construct
  REGION 1..81: note = Synthetic peptide
SKEEIDKIVKKHKKKIEEHKKKVDELKKLVEEHDKRVSQDKDDKVKKLSEEVKKIIKRLEEVSKRLEEVSKKLLKVISDKR

SEQ ID NO: 34 - protein, 78 aa, synthetic construct
  REGION 1..78: note = Synthetic peptide
GSNDEELKKILETLDRILKKLDKILTRLIEVLKKSEDPNLDDKDYTELVKQFIELIKKYEEVVKEYEEVVRQLIRLFS

SEQ ID NO: 35 - protein, 81 aa, synthetic construct
  REGION 1..81: note = Synthetic peptide
SKEEIDKIVKKHKKKIEELKKLVDELKKLVEEHDKRVSQDKDDKVKKLSEEVKKIIKRVEEVAKRLEEVSKKLLKVISDKR

SEQ ID NO: 36 - protein, 78 aa, synthetic construct
  REGION 1..78: note = Synthetic peptide
GSNDEELKKILETLDRILKKLEKILTRLIEVLKKSEDPNLDDKDYTELVKQFIELIKKFEEVIKEYEEVVRQLIRLFS

SEQ ID NO: 37 - protein, 81 aa, synthetic construct
  REGION 1..81: note = Synthetic peptide
SKEEIDKIVKKHKKKIEEHKKKVDEHKKLVEEHDKRVSQDKDDKVKKLSEELKKISKRLEEVSKRLEEVSKKLLKVISDKR

SEQ ID NO: 38 - protein, 78 aa, synthetic construct
  REGION 1..78: note = Synthetic peptide
GSNDEELKKILETLDRILKKLDKILTRLDEVLKKSEDPNLDDKDYTELVKQYIELVKKYEEVVKEYEEVVRQLIRLFS

SEQ ID NO: 39 - protein, 69 aa, synthetic construct
  REGION 1..69: note = Synthetic peptide
DHSRKLKEILDRLRKHVKRLKEHLDELRDLVRQVPEDKLLEHVVKLSDKILQISERAVREFTKSVDKDS

SEQ ID NO: 40 - protein, 76 aa, synthetic construct
  REGION 1..76: note = Synthetic peptide
GSDKKDELERILDEIRRLIERLDEILSRLNKLLELLKHGVPNAKEVVKDYIRLLKEYLELVKEFLKLVKRHADLVS

SEQ ID NO: 41 - protein, 72 aa, synthetic construct
  REGION 1..72: note = Synthetic peptide
DSDEHLKKLKTFLENLRRHLDRLDKHIKQLRDILSENPEDERVKDVIDLSERSVRIVKTVIKIFEDSVRKKE

SEQ ID NO: 42 - protein, 75 aa, synthetic construct
  REGION 1..75: note = Synthetic peptide
GSDDKELDKLLDTLEKILQTATKIIDDANKLLEKLRRSERKDPKVVETYVELLKRHEKAVKELLEIAKTHAKKVE

SEQ ID NO: 43 - protein, 73 aa, synthetic construct
  REGION 1..73: note = Synthetic peptide
MDEEDHLKKLKTHLEKLERHLKLLEDHAKKLEDILKERPEDSAVKESIDELRRSIELVRESIEIFRQSVEEEE

SEQ ID NO: 44 - protein, 74 aa, synthetic construct
  REGION 1..74: note = Synthetic peptide
GDVKELTKILDTLTKILETATKVIKDATKLLEEHRKSDKPDPRLIETHKKLVEEHETLVRQHKELAEEHLKRTR

SEQ ID NO: 45 - protein, 72 aa, synthetic construct
  REGION 1..72: note = Synthetic peptide
DSDEHLKKLKTFLENLRRHLDRLDKLLKELRDILSENPEDERVKDVIDELERVIRIVKTVIKIFEDSVRKKE

SEQ ID NO: 46 - protein, 75 aa, synthetic construct
  REGION 1..75: note = Synthetic peptide
GSDDKELDKLLDTLEKILQTATKIIDDLNKVLEKLRRSERKDPKVIETVVELLKRHEKAVKELLEIAKTHAKKVE

SEQ ID NO: 47 - protein, 72 aa, synthetic construct
  REGION 1..72: note = Synthetic peptide
DSDEHLKKLKTFLENLRRLEDLLDKHIKQLRDILSENPEDERVKDVIDLSERVVRTVKTVIKIFEDSVRKKE

SEQ ID NO: 48 - protein, 75 aa, synthetic construct
  REGION 1..75: note = Synthetic peptide
GSDDKELDKLLDTLEKILQTATKVVDDANKLLEKLRRSERKDPKVVETYVELLKRLEKLIKELLEIAKTHAKKVE

SEQ ID NO: 49 - protein, 37 aa, synthetic construct
  REGION 1..37: note = Synthetic peptide
DSDEHLKKLKTFLENLRRHLDRLDKHIKQLRDILSEN

SEQ ID NO: 50 - protein, 114 aa, synthetic construct
  REGION 1..114: note = Synthetic peptide
EDERVKDVIDLSERSVRIVKTVIKIFEDSVRKLEKTKPDSKTAKELDKLLDTLEKILQTATKIIDDANKLLEKLRRSERKDPKVVETYVELLKRHEKAVKELLEIAKTHAKKVE

SEQ ID NO: 51 - protein, 114 aa, synthetic construct
  REGION 1..114: note = Synthetic peptide
DSDEHLYKLKTFLENLRRHLDRLDKHIKQLRDILSENPEDERVKDAIDLSERSVRIVKTVIKIFEDSVRKKEKRPIDKRDDKELDKLLDTLEKILQTATKIIDDANKLLEYLRR

SEQ ID NO: 52 - protein, 35 aa, synthetic construct
  REGION 1..35: note = Synthetic peptide
GDPKVVETYVELLKRHEKAVKELLEIAKTHAKKVE

SEQ ID NO: 53 - protein, 72 aa, synthetic construct
  REGION 1..72: note = Synthetic peptide
DSDEHLDRLDKHLKKLKTFLENLRRHIKQLRDILSENPEDERVKDVIDLSKTVIKIFEDSVRKKERSVRIVE

SEQ ID NO: 54 - protein, 75 aa, synthetic construct
  REGION 1..75: note = Synthetic peptide
GSDDKEATKIIDDLDKLLDTLEKILQTANKLLEKLRRSERKDPKVVETYVKAVKELLEIAKTHAELLKRHEKKVE

SEQ ID NO: 55 - protein, 72 aa, synthetic construct
  REGION 1..72: note = Synthetic peptide
DSDEHIKQLRDHLDRLDKHLKKLKTFLENLRRILSENPEDERVKTVIKIFEDSVRKKERSVRIVKDVIDLSE

SEQ ID NO: 56 - protein, 75 aa, synthetic construct
  REGION 1..75: note = Synthetic peptide
GSDDKEANKLLEKATKIIDDLDKLLDTLEKILQTLRRSERKDPKAVKELLEIAKTHAELLKRHEKVVETYVKKVE

SEQ ID NO: 57 - protein, 104 aa, synthetic construct
  REGION 1..104: note = Synthetic peptide
DHSRKLEEILDRLRKHVKRLLEHLRELLSLVKENPEDKDLVEVLELSLAILRRSLEAVEAFLKSVTKKDPDDEDLRRKADEIRKEVEEIKKSLAEVEKEIYKLK

SEQ ID NO: 58 - protein, 116 aa, synthetic construct
  REGION 1..116: note = Synthetic peptide
GSSADDVLEDILKIIRELIEILDQILSLLNQLLKLLRHGVPNAKKVVEKYKEILELYLQLVSLFLKIVKTHADAVSGKIDKKAEEEIKKEEEKIKEKLRQAKDILKKLQEEIDKTR

SEQ ID NO: 59 - protein, 107 aa, synthetic construct
  REGION 1..107: note = Synthetic peptide
DRDAHLYKLLTFLEQLVRHLDRLVKHITQLRDIVKKDPEDERAVDVIRQSVRSLEIVITVLKIFVDSVSDAARSKEAEKIVRKIRKEIDEIRQKLREIDKEVKKTTS

SEQ ID NO: 60 - protein, 114 aa, synthetic construct
  REGION 1..114: note = Synthetic peptide
GSNDKVLDKILDILDRILRLATRVIDLANKLLQVKKKSTHKDPRIVETYKELLKIHETAVRLLLELADLHRRLKSKDEEANKRVETELDRIRKKVKDIEDKVRKLEDKVRKTAS

SEQ ID NO: 61 - protein, 68 aa, synthetic construct
  REGION 1..68: note = Synthetic peptide
NDLSKEVLKKLEKSVEELLRRVQKSVKEAQKRGLLSDELVDRHLKILNQLVKRHLELLQEVIKRSDKK

SEQ ID NO: 62 - protein, 78 aa, synthetic construct
  REGION 1..78: note = Synthetic peptide
GSDEAVKRVVEKSLKILDEVIKKSLDILRELIELQIRHAKDDESVIRASKSALKDAIEALKKSLDEIKKALKRSADEG

SEQ ID NO: 63 - protein, 76 aa, synthetic construct
  REGION 1..76: note = Synthetic peptide
SSEEVVKVHEKVVKLHKEILELLKKIIKIHETAARDPDDKDSIKKLSDEIKKIVKRIEDISDQAKRESSDAQRKQS

SEQ ID NO: 64 - protein, 74 aa, synthetic construct
  REGION 1..74: note = Synthetic peptide
DKEEESKELLKKLKEILKRSEELLEESKELLKLAKNGEIDESELADADRKLNKKHEKLVQDIQDLLREHERQDR

SEQ ID NO: 65 - protein, 73 aa, synthetic construct
  REGION 1..73: note = Synthetic peptide
DEKKKIDKIVKETEDLLQKSEKLLQQSKEAVKRIRSQVKENEIVDRLLRISEELLKISRRLVEISRRIASTLS

SEQ ID NO: 66 - protein, 78 aa, synthetic construct
  REGION 1..78: note = Synthetic peptide
GSSKEEVIRLLKENVRLIKENLELLTRNLKLITDLVRGSNGSEEKIKTLKELLKEYRELLKRYRKLVEDYKRLVDKHD

SEQ ID NO: 67 - protein, 81 aa, synthetic construct
  REGION 1..81: note = Synthetic peptide
EIQELIKSSRRIIEESKELIKESEEVLRRIKEILDRIRNGVDNQEDLLREILKLLTKNLKIIQRNLKLLQDNAEILKRLVS

SEQ ID NO: 68 - protein, 75 aa, synthetic construct
  REGION 1..75: note = Synthetic peptide
GSYIEDVIKKILDVSRELIKLSRTIIKISEEINKQLQQGRDTKDLVKKYDEIIKKYTRIVQHYTELIKELQKLLS

SEQ ID NO: 69 - protein, 75 aa, synthetic construct
  REGION 1..75: note = Synthetic peptide
SPTEEAIQLSQRVIELSKRVIELSKEILKLLKRVLDLLPDLDKNEEKRLDDYDKELKEYDKELKKYEKRLKDLAS

SEQ ID NO: 70 - protein, 75 aa, synthetic construct
  REGION 1..75: note = Synthetic peptide
GSEEEEILKIQKELLRIQSEILDKQKKILDTLRSNGAVTEEVRSILEKVERLSEEAKELSKEAKELTKEVSKLIS

SEQ ID NO: 71 - protein, 77 aa, synthetic construct
  REGION 1..77: note = Synthetic peptide
SPLKELNNQLLRLLRELVKVSKKIVDLSKTIIEVLKHTDLDPRLLDSLEKSQQELDKSQKELDKVVKELTKVNKKLQ

SEQ ID NO: 72 - protein, 78 aa, synthetic construct
  REGION 1..78: note = Synthetic peptide
GSPLEDLVRKYDELVKTYEKLVEEFKKAVDKYDKAVKKAPVSKEATDSLDLIRKVLELLDRNLKLIKENAKLIKELLK

SEQ ID NO: 73 - protein, 69 aa, synthetic construct
  REGION 1..69: note = Synthetic peptide
SPTRENEKVIKENEKVISDNERVLEEVVKVVETATDRKEIQDAVDEVRKSVDKLRDSVRKLEESVRTLD

SEQ ID NO: 74 - protein, 72 aa, synthetic construct
  REGION 1..72: note = Synthetic peptide
GSPIKDISKRLLEISKRLVEISDRIVELLQRIADSKDPNKDLQKEVKDVLEEYKRLVREYREVVKEYEKVVS

SEQ ID NO: 75 - protein, 75 aa, synthetic construct
  REGION 1..75: note = Synthetic peptide
DEDEHVKQLIKNADLLRKHAELLKELVKLFQEIASQIPDDRVAKKVTDVVDRIDKILKQTEKLVRRTKQILDYSR

SEQ ID NO: 76 - protein, 71 aa, synthetic construct
  REGION 1..71: note = Synthetic peptide
GSNLEELVKLLKEVLEMHERLLRIHEDLVEAHKSNASDKESERKLKKSDKDIKESLKKIKSIIDQVRYIQS

SEQ ID NO: 77 - protein, 70 aa, synthetic construct
  REGION 1..70: note = Synthetic peptide
PVEDIIEESLRLLEESLKLLNRILKLLEDSLRKLPRSEEWRQRLDEFRKKLEDWKEELERWIEDVRYKKT

SEQ ID NO: 78 - protein, 78 aa, synthetic construct
  REGION 1..78: note = Synthetic peptide
GSDEDYESREIIDEIRKLLDRSKKIVHRSQRLVERVKSTPLSEDQEDLIRRHEETINRHRELVKELEKVLEDHERHIR

SEQ ID NO: 79 - protein, 78 aa, synthetic construct
  REGION 1..78: note = Synthetic peptide
PEEDSRRVLERFVRVSREVLKVLEEFLRVSEELLREADRDRDRRLEEYERQVDELREEIRRYKEEVDKFDKEVKYYKK

SEQ ID NO: 80 - protein, 79 aa, synthetic construct
  REGION 1..79: note = Synthetic peptide
GSPEKDENRKLLDKVRKLVEKSRRLVEELRKLVDQSTKNGLIDEKALRKQQEVLRKVEEVLEKQERVLRELEEISYRVI

SEQ ID NO: 81 - protein, 39 aa, synthetic construct
  REGION 1..39: note = Synthetic peptide
GSPERDENRKLLDKVRKLVEKSRRLVEELRKLVDQSTKN

SEQ ID NO: 82 - protein, 124 aa, synthetic construct
  REGION 1..124: note = Synthetic peptide
GSDEKALRKQQEVLRKVEEVLEKQERVLRELEEISYRVITRGEDHKAEEDSRRVLERFVRVSREVLKVLEEFLRVSEELLREADRDRDRRLEEYERQVDELREEIRRYKEEVDKFDKEVKYYKK

SEQ ID NO: 83 - protein, 39 aa, synthetic construct
  REGION 1..39: note = Synthetic peptide
GSDRRLEEYERQVDELREEIRRYKEEVDKFDKEVKYYKK

SEQ ID NO: 84 - protein, 125 aa, synthetic construct
  REGION 1..125: note = Synthetic peptide
GSPERDENRKLLDKVRKLVEKSRRLVEELRKLVDQSTKNGLIDEKALRKQQEVLRKVEEVLEKQERVLRELEEISYRVITRGEDHKAEEDSRRVLERFVRVSREVLKVLEEFLRVSEELLREADR

SEQ ID NO: 85 - protein, 75 aa, synthetic construct
  REGION 1..75: note = Synthetic peptide
DLSEESKKFVEKVKKLEKESRELEKQVKKIEEDSRSVENDVQKEFLELLKRLLDIQKKVVEVLREVVKVQQYVDS

SEQ ID NO: 86 - protein, 80 aa, synthetic construct
  REGION 1..80: note = Synthetic peptide
GSDSEYESRQVLRELDTVLKDSHTVLEALRQVIRDSQDVVSKSDEESRRVIDDLEKVIQDSKKVLDDIKRLIDKSKSIKS

SEQ ID NO: 87 - protein, 75 aa, synthetic construct
  REGION 1..75: note = Synthetic peptide
NEDELLKLLTENLKLLDENLKLLRENLSLLRQANNITDKNRIREIVKQSKEIVKQSREILKQSKEIVERIKYIVS

SEQ ID NO: 88 - protein, 78 aa, synthetic construct
  REGION 1..78: note = Synthetic peptide
GSSLYELTQRYEKLVQQYEELVKDYRRLVKKLEKLKRDNKPDKRLLKEIVDVIKKSVEIIDRSLKLLEESIKILEETD

SEQ ID NO: 89 - protein, 73 aa, synthetic construct
  REGION 1..73: note = Synthetic peptide
SQERSLEILKRILDVLKESLEILKESLSILRQLASRIKNPNRKIEEILKESDKIIKESDKVLKEIEEVIRYSS

SEQ ID NO: 90 - protein, 78 aa, synthetic construct
  REGION 1..78: note = Synthetic peptide
GSDIEYESKEILELIKELLKLSRELLKESRRALELVRKSRDDSIVEEVIQVHKKVLDIHKEVLKIVRKVVEVHRRVKS

SEQ ID NO: 91 - protein, 76 aa, synthetic construct
  REGION 1..76: note = Synthetic peptide
SKKDESTKLERLAEKIDEITKRIEELVKDVKRKSSEGVDKDQQQKIDEVFQKLLDLQREILEILDRILKVQQYILD

SEQ ID NO: 92 - protein, 78 aa, synthetic construct
  REGION 1..78: note = Synthetic peptide
GSDLEYLNRRLLQLIKTLIDLNRHLLKLIDKLKKLNSREGDEEKIKEESKQIQEQFKEIVERSKEIIKQIKEIIKRSQ

SEQ ID NO: 93 - protein, 79 aa, synthetic construct
  REGION 1..79: note = Synthetic peptide
DFERSSRRLEKVVEDLRRSSDRLREVIDELRKSADEKDEDEDLRRARKEHRDLIEELKRALEKQEEIIKHLQELVYRQL

SEQ ID NO: 94 - protein, 78 aa, synthetic construct
  REGION 1..78: note = Synthetic peptide
GSEESEEVRKVVERIKKISRELEEVVKELDRVSKEFDRHGETDEIVREHERIVEKLEEIVKKHTKIVEELAEIVYKQQ

SEQ ID NO: 95 - protein, 71 aa, synthetic construct
  REGION 1..71: note = Synthetic peptide
SDDDSVRVLDEIVKILDESVKLLKESLKLLDDFLRTKPDDHLKEVVKESKKVVEQSKKVLDRIKKIIYESK

SEQ ID NO: 96 - protein, 72 aa, synthetic construct
  REGION 1..72: note = Synthetic peptide
GSDLLYLSKELLKLVRELLKLSRELVELSRRLVNSTHKSPELVKKYDKLVKKYQDLLKKLADVADEYLRQRS

SEQ ID NO: 97 - protein, 76 aa, synthetic construct
  REGION 1..76: note = Synthetic peptide
DEKDYHRRLIEHLEDLVRRHEELIKRQKKVVEELERRGLDERLRRVVDRFRRSSERWEEVIERFRQVVDKLRKSVE

SEQ ID NO: 98 - protein, 78 aa, synthetic construct
  REGION 1..78: note = Synthetic peptide
GSDAYDLDRIVKEHRRLVEEQRELVEELEKLVRRQEDHRVDKKESHEILERLERIIRRSTRILTELEKLTDEFERRTR

SEQ ID NO: 99 - protein, 77 aa, synthetic construct
  REGION 1..77: note = Synthetic peptide
DERYRAREHIRRVEEHTKRLRHILKRLREHEEKLRRELKPGDEITESVDRFKKIVDQFEESIKKFETVSEELRKSDS

SEQ ID NO: 100 - protein, 71 aa, synthetic construct
  REGION 1..71: note = Synthetic peptide
GSDRQRILDRLDKILEKLDDILKKLKDILETLSKDDVSDRRHKDLVEKFRELVDTHHKLVERYRELVYQNR

SEQ ID NO: 101 - protein, 38 aa, synthetic construct
  REGION 1..38: note = Synthetic peptide
GSDEITESVDRFKKIVDQFEESIKKFETVSEELRKSIS

SEQ ID NO: 102 - protein, 107 aa, synthetic construct
  REGION 1..107: note = Synthetic peptide
GSDPQRAADRLDKILEKLDDILKKLKDILETLSKDDVKDRRAKDLVEKFRELVDTHHKLVERYRELVYTATAGSDLARELIRRVEEHTKRLRHILKRLREHEEKLRR

SEQ ID NO: 103 - protein, 73 aa, synthetic construct
  REGION 1..73: note = Synthetic peptide
NADDQLATSIKKLEDSIDQLIKIVRKFEESVKKLQKHGVDQHHVEILRKIVEIFRQHIEKLKKHLEKLRYTSS

SEQ ID NO: 104 - protein, 70 aa, synthetic construct
  REGION 1..70: note = Synthetic peptide
GSDKEYLVTEHEKLVREHEKIVSEIEKLVKKHEAGVDESELEEILKKVEKLLRKLDEILEQLTQLLRKTE

SEQ ID NO: 105 - protein, 36 aa, synthetic construct
  REGION 1..36: note = Synthetic peptide
GSDQHVVEILRKIVEIFRQHIEKLKKHLEKLRYTSS

SEQ ID NO: 106 - protein, 114 aa, synthetic construct
  REGION 1..114: note = Synthetic peptide
GSDAEYLVTEHEKLVREHEKIVSEIEKLVKKHEKGVDESELEEILKKVEKLLRKLDEILEQLTQLLRKAEKHIDKHSKAADQLATSIKKLEDSIDQLIKIVRKFEESVKKLQKH

SEQ ID NO: 107 - protein, 74 aa, synthetic construct
  REGION 1..74: note = Synthetic peptide
DEDDDIRRVLDESRRVLEHSRRVLKRSEEVLEKASRKKEKDTEEIEKHLKRLREHAKKLEKHRRELDDFLYKEI

SEQ ID NO: 108 - protein, 74 aa, synthetic construct
  REGION 1..74: note = Synthetic peptide
GSRDKYLLERLNDILKKLDEIVDKLSDILKRLKDVRHDDRLQELVERYKEIVKEYKRIVEEYEKLVREFEEQQR

SEQ ID NO: 109 - protein, 73 aa, synthetic construct
  REGION 1..73: note = Synthetic peptide
DRDYEDKEFKKIIKELEDVQEELKKLQEKIKRFSSELEEPNELLKEQLKVNEEQLEVNKKILKILRDQLKQNE

SEQ ID NO: 110 - protein, 76 aa, synthetic construct
  REGION 1..76: note = Synthetic peptide
GSDAEYKVRESVKRSKESVKHSEDVVDKLNKSVKLSESGHSDAEKASRELVKLVREVVELSREVIKLSEKVLRVIS

SEQ ID NO: 111 - protein, 79 aa, synthetic construct
  REGION 1..79: note = Synthetic peptide
DLQYKQEKLIRHFDRVVREWDKLVRKFSKVLEKQKHESKDKELEEASRRVDELIKRLREQLKRSKEILRRLKELSRKSS

SEQ ID NO: 112 - protein, 79 aa, synthetic construct
  REGION 1..79: note = Synthetic peptide
GSDWEELLRRLEKVLQEYEEIVKELIDLIERLIKVSEDKSKDASEYKKLVTELEKLISKLEEISKKLEELVKEYEYKTE

SEQ ID NO: 113 - protein, 76 aa, synthetic construct
  REGION 1..76: note = Synthetic peptide
DAKDELEKSLQEIEESLKELKKLLEELDKSLRELTSQGRNKKLEEHIKKVQKFIELVKKYIKAVQDYLKEVRYDNS

SEQ ID NO: 114 - protein, 75 aa, synthetic construct
  REGION 1..75: note = Synthetic peptide
GSDKERAARATEEMVKLTKKLLKAVEDLVRDVRRLLKEGLISEKHARIAETILEVFKKHAKIIKKHVDIVKYDES

SEQ ID NO: 115 - protein, 78 aa, synthetic construct
  REGION 1..78: note = Synthetic peptide
GSPLKERLLEIQRDLDRVLEEVVERLLRIQERLDSVVERKPPDVHEEYKYIVDEIREIVERVVREYEEIVKRIDEEVR

SEQ ID NO: 116 - protein, 83 aa, synthetic construct
  REGION 1..83: note = Synthetic peptide
GSEEDERIRYDLDRIRKDVRRKLEEIRQRVRELEKKLRDAGHRRDEKELLRELIETSKDILRLVEELLKKIIDKSEDLLRKTE

SEQ ID NO: 117 - protein, 73 aa, synthetic construct
  REGION 1..73: note = Synthetic peptide
GSDEEDYINENVEKDVRDIEDDVRRINERIRELLEKIRTEEVLQRVLEEHHELVERVLRKLVEILRKHEEENR

SEQ ID NO: 118 - protein, 77 aa, synthetic construct
  REGION 1..77: note = Synthetic peptide
GSDEEEYYKEKLHKLLREIEELLKHYRELVRRLEELVKRGELDKDTAAHILERLSELLERIIRRVAHTLRRLSEERR

SEQ ID NO: 119 - protein, 84 aa, synthetic construct
  REGION 1..84: note = Synthetic peptide
GSDEDEISYDSKRRVEEIVRQAREKSEKSRKDIEDVAEVLRKGDVSEKEVVDELVKVLEEQVKVLREAVERLREVLKKQVDDVR

SEQ ID NO: 120 - protein, 82 aa, synthetic construct
  REGION 1..82: note = Synthetic peptide
GSDIVELVDHLLKRSLKLLEELAELVRRLLEKSTELLKRRTEEHKEEVVEESEYMVRELEERLRRVVDESEKLVRDADKHIR

SEQ ID NO: 121 - protein, 83 aa, synthetic construct
  REGION 1..83: note = Synthetic peptide
GSKEKDIVKTLVDLLRENLETLERLIEEVVRLLKENVDVRDEGRDDKDSERILRDIKRRIDEAAKESREIIERIEKEVEYRSR

SEQ ID NO: 122 - protein, 80 aa, synthetic construct
  REGION 1..80: note = Synthetic peptide
GSPEVDVLRRIVREILKASEELLRLLRKLIDEALKLSERKRDSQEYREVVDRVKKELERLLDEYRKLVEELKEKLRYDTR

SEQ ID NO: 123 - protein, 84 aa, synthetic construct
  REGION 1..84: note = Synthetic peptide
GSDKRYESEKLKRRLDEAVEKVREVVERVERESDRVLEEVRRRRESKEVVDKVIEDNDKALEDVLRVVDEVAKVVRDVVRENTR

SEQ ID NO: 124 - protein, 77 aa, synthetic construct
  REGION 1..77: note = Synthetic peptide
GSPREYHSKDILRKVDEILERIRRHADRVKKKSERLKRENVDVNEHSKDVKRVIRELLELVKELLRLAKKHSDDQQE

SEQ ID NO: 125 - protein, 79 aa, synthetic construct
  REGION 1..79: note = Synthetic peptide
GSDEDEILYHSERLLQKLKKELDDLKEKSRELLEELKKEDPDDRLIERIIRLHDEVLKDLDEVLKNILEVHREVLERLR

SEQ ID NO: 126 - protein, 75 aa, synthetic construct
  REGION 1..75: note = Synthetic peptide
DKLDRLLKIHEEALRRAEELIKRLLDIHRRALDLARRGELDDYLLKESERELREIIRRAREELKESRDRLEEISR

SEQ ID NO: 127 - protein, 80 aa, synthetic construct
  REGION 1..80: note = Synthetic peptide
GSPKEELIRRVLEEVKRLNEKLLEIIRRAAELVKRANDELPETEKLREIDRELEKKLKEIEDELRRIDKELDDALYEIED

SEQ ID NO: 128 - protein, 80 aa, synthetic construct
  REGION 1..80: note = Synthetic peptide
GSPKLDKLRELLERNLEKLREILEEVLKILRTNLERVREDIRDEDVLQEYERLIRKAEEDLRRVLKEYDDLLKKLVYELR

SEQ ID NO: 129 - protein, 75 aa, synthetic construct
  REGION 1..75: note = Synthetic peptide
GSKEDESVKRAEEIVRTLLKLLEDSLREAERSLRDIKNGEDEHNLRRISEKLEELSKRITETIERLLRELQYTSR

SEQ ID NO: 130 - protein, 84 aa, synthetic construct
  REGION 1..84: note = Synthetic peptide
GSPNQELLDRVRKILEDLLRLNEELVRLNKELLKRALEMRRKNRDSEEVLERLAEEYRKRLEEYRRELEKLLEELEETIYRYKR

SEQ ID NO: 131 - protein, 84 aa, synthetic construct
  REGION 1..84: note = Synthetic peptide
GSDESEEAQHEVEKVLDDIRRLSEHLQKRLEEVLEEVYELRREGSDRTEVVELLKEVIREIVRVNREALERLLRVVEEAVKRNE

SEQ ID NO: 132 - protein, 83 aa, synthetic construct
  REGION 1..83: note = Synthetic peptide
GSDEEELVETVKRIQKEILDRLTELAKLLVEIQREIKKLKDEGEDDKELKRLSDELEEKVRQVVEEIKRLSDELEETVEYVSR

SEQ ID NO: 133 - protein, 80 aa, synthetic construct
  REGION 1..80: note = Synthetic peptide
GSDEEEEVVRRAEELVKEHEELIERVIRTHEELVYKLEDQGADKKLVDVLKRVVEESERVAREIVKVSRELIRLLEEASR

SEQ ID NO: 134 - protein, 80 aa, synthetic construct
  REGION 1..80: note = Synthetic peptide
GSSKEEILKELEDLQRRLIEELKKLQERVVELLEELIKRLRDRGRDDKHLKRLVKEVRRLSEEVLRSIKEVSDRVRYQLR

SEQ ID NO: 135 - protein, 81 aa, synthetic construct
  REGION 1..81: note = Synthetic peptide
GSDKEEESEYLLRDLVRLLEKVKEKIEEVNREVEKLLKKVKDGRLDRREVLREILRLNRELAEIIKEVVDRIRHVVERSER

SEQ ID NO: 136 - protein, 76 aa, synthetic construct
  REGION 1..76: note = Synthetic peptide
GSDLHEVVYETKELLKRIEEVVEELRKKSEDIIRKAERGEISEDELKRLQEEIAREAKKLLDEIKRVLERHLEQTL

SEQ ID NO: 137 - protein, 81 aa, synthetic construct
  REGION 1..81: note = Synthetic peptide
GSPVEEIIKEVVKRVIEVQEKVLRIISHAVKRVVEVQKKYDPGSEESNRVVEEVKKTIEDAIRESDEVVDEVVKRIQYTVR

SEQ ID NO: 138 - protein, 80 aa, synthetic construct
  REGION 1..80: note = Synthetic peptide
GSPEQEIADRILTEIRESQKELERLARKILKLLDESQEKAKRGRLSEEESDELLERIKKELDELLERSKELLKKIEYELR

SEQ ID NO: 139 - protein, 80 aa, synthetic construct
  REGION 1..80: note = Synthetic peptide
GSDEDKEANRVLDEVLKTVRDLLETANEVLKEVLYRLKRTDDQEKVVRTLTEVLKEHLKLVEEIVRILDKVLKEHLETEK

SEQ ID NO: 140 - protein, 76 aa, synthetic construct
  REGION 1..76: note = Synthetic peptide
GSPEDDVLRRLEEVSEKILRVAEDVARQLREVSEKITQGKVDRKEWEEDIKRLKRELEELLREWKEEIERLTYELR

SEQ ID NO: 141 - protein, 78 aa, synthetic construct
  REGION 1..78: note = Synthetic peptide
GSRREEVVKRIRELLKRNKELIDRIRELLEENEYLDKDARDKDVLRRSVELLEELVRILEESVELAKEIIKLLREVVE

SEQ ID NO: 142 - protein, 75 aa, synthetic construct
  REGION 1..75: note = Synthetic peptide
GSDEKEDNRRLQHKIERILEKNEDLQRKLEEILELLERGEADEEKIDRLRKAVEDYRRVVEEIKEDVKRHKYTVR

SEQ ID NO: 143 - protein, 77 aa, synthetic construct
  REGION 1..77: note = Synthetic peptide
GSDEKEEAKKASEESVRTVERILEELLKASEESVELLRRGEDAKDVVERSKEALKRVKELLDEVVKRSDEILKYIHN

SEQ ID NO: 144 - protein, 84 aa, synthetic construct
  REGION 1..84: note = Synthetic peptide
GSDEKKLINEVVETQKRLIKEAAKRLSEVVRHQTELIRELREKNVDDKDVEKLLKESLDLAEEIVRRIKELLDESKKLVEYVSN

SEQ ID NO: 145 - protein, 76 aa, synthetic construct
  REGION 1..76: note = Synthetic peptide
GSPDMDEVKRVLDELIEIQEEILREIKRVLEKLIKIQEDNGSEYESREVVREIVEIARKLVERSRRVVKKITETLQ

SEQ ID NO: 146 - protein, 80 aa, synthetic construct
  REGION 1..80: note = Synthetic peptide
GSDERYATREIVERIERIAREILKRTEEIVREVREVLSRDVDQEEVVRRLADLLRESVELVQHLVRRVEELLQESVERKK

SEQ ID NO: 147 - protein, 79 aa, synthetic construct
  REGION 1..79: note = Synthetic peptide
GSPEREALREVLEDLKRVTDRLRELVERVLEELKKVTDHVDSERILRESRRVLKELKDIIEEILRESEKVLEKLKYTED

SEQ ID NO: 148 - protein, 80 aa, synthetic construct
  REGION 1..80: note = Synthetic peptide
GSPAREILEEVVKKHLEVVEDAARILEEIIREHEKAVREDRDKKELEEISRDLLRKAREALKKVKDISDDLSREIEYVAS

SEQ ID NO: 149 - protein, 75 aa, synthetic construct
  REGION 1..75: note = Synthetic peptide
GSPVEEAIKKVIDDLRDVQRKIRELVEELIRLLEEVQRDNDKRESEYVVERVEEILRRITETSREVVRKAVEDLS

SEQ ID NO: 150 - protein, 81 aa, synthetic construct
  REGION 1..81: note = Synthetic peptide
GSDSDEKAEYLLKEMERVVRESDEVVKKILRDLEEVLERLRRGEISEDDVTEILKELAERHIRAIEELVRRLRELLERHKR

SEQ ID NO: 151 - protein, 79 aa, synthetic construct
  REGION 1..79: note = Synthetic peptide
GSPVEEVLKELSEVNERVRDIAREIIERLSEVNEEVKETDDEDELKKISKKVVDEVEDLLRKILEVSEEVVRRVEYHDR

SEQ ID NO: 152 - protein, 76 aa, synthetic construct
  REGION 1..76: note = Synthetic peptide
GSPKEDILREVLRRHKEIVREIVRLVREAVETHLELVKRNSDDRDAQDVIRKLEEDLERLVRHAQEVIEEIFYRLH

SEQ ID NO: 153 - protein, 75 aa, synthetic construct
  REGION 1..75: note = Synthetic peptide
GSPRSYLLKELADLSQHLVRLLERLVRESERVVEVLERGEVDEEELKRLEDLHRELEKAVREVRETHREIRERSR

SEQ ID NO: 154 - protein, 76 aa, synthetic construct
  REGION 1..76: note = Synthetic peptide
GSDREYIIKDILDSQEHLLRLIEELLETQKELLEILKRRPDSVERVRELVRRSKEIADEIRRQSDRNVRLLEEVSK

SEQ ID NO: 155 - protein, 75 aa, synthetic construct
  REGION 1..75: note = Synthetic peptide
GSDEKDEIRHVIESVERLIEDIKRLLKTLRELAHDDSDKKTVKEVLDRVKEMIERHRRELEEHRKELERAEYEVR

SEQ ID NO: 156 - protein, 76 aa, synthetic construct
  REGION 1..76: note = Synthetic peptide
GSESEDRIKELLKRHIELVERHEELLHEIKKLIDLEEKDDKDREEAVKRIDDAIKESEEMLEESKEILEEIEYLNR

SEQ ID NO: 157 - protein, 77 aa, synthetic construct
  REGION 1..77: note = Synthetic peptide
GSSLEDSVRLNDEVVKVVERVVRLNQEVVRLIKHATDVEDEETVKYVLERVREVLDESREVLKRVHELLEESERRLE

SEQ ID NO: 158 - protein, 82 aa, synthetic construct
  REGION 1..82: note = Synthetic peptide
GSHEKDIVYKVEDLVRKSDRIAERAREIVKRSRDIMREIRKDKDNKKLSDDLLKVTRDLQRVVDELEELSRELLRVAEESRK

SEQ ID NO: 159 - protein, 76 aa, synthetic construct
  REGION 1..76: note = Synthetic peptide
GSPELDEVKKLIDELKKSVERLEESIREVKESIKKLRKGDIDAEENIKLLKENIKIVRENIKIIKEIIDVVQYVLR

SEQ ID NO: 160 - protein, 82 aa, synthetic construct
  REGION 1..82: note = Synthetic peptide
GSDEEEIEELLRELEKLLKKSEEALEESKKLIDESEELLRRDRLDKEKHVRASEEHVKLSEEHLRISREIVKILEKAVYSTR

SEQ ID NO: 161 - protein, 79 aa, synthetic construct
  REGION 1..79: note = Synthetic peptide
GSDESDRIRKIVEESDEIVKESRKLAERARELIKESEDKRVSEERNERLLEELLRILDENAELLKRNLELLKEVLYRTR

SEQ ID NO: 162 - protein, 79 aa, synthetic construct
  REGION 1..79: note = Synthetic peptide
GSDEDDELERLLREYHRVLREYEKLLEELRRLYEEYKRGEVSEEESDRILREIKEILDKSERLWDLSEEVWRTLLYQAE

SEQ ID NO: 163 - protein, 78 aa, synthetic construct
  REGION 1..78: note = Synthetic peptide
GSDKKDASRRAIRVLHEFVRVSEEVLEVLRKSVESLKRLDVDEKIKRTHDRIEEELRRWKRELEELIERLREWEYHQD

SEQ ID NO: 164 - protein, 75 aa, synthetic construct
  REGION 1..75: note = Synthetic peptide
GSDDEEEDKRLLEEVKRSLDTDERILEKLRHSLERQLEDVDKDEDSRRVLRELDEITKRSREVVKRLRKLAYESK

SEQ ID NO: 165 - protein, 79 aa, synthetic construct
  REGION 1..79: note = Synthetic peptide
GSDKEYKLDRILRRLDELIKQLSRILEEIERLVDELEREPLDDKEVQDVIERIVELIDEHLELLKEYIKLLEEYIKTTK

SEQ ID NO: 166 - protein, 79 aa, synthetic construct
  REGION 1..79: note = Synthetic peptide
GSPSKEYQEKSAERQKELLHEYEKLVRHLRELVEKLQRRELDKEEVLRRLVEILERLKDLHKKIEDAHRKNEEAHKENK

SEQ ID NO: 167 - protein, 78 aa, synthetic construct
  REGION 1..78: note = Synthetic peptide
GSRDRKISEELIKALEDHIRMLEELIRAIEEHIKLAERGVDEKELRESLEELKKIVDELEKSLEELRKLAERYKYETR

SEQ ID NO: 168 - protein, 77 aa, synthetic construct
  REGION 1..77: note = Synthetic peptide
GSPKEESVEELKRVIDKHEEILRELKRVLEEHERVSHDEDENELRRSLERLKHILDRLHESLKELHELLKKNEYTER

SEQ ID NO: 169 - protein, 77 aa, synthetic construct
  REGION 1..77: note = Synthetic peptide
GSDHEYWVKIVERILRVMEKHAEIVKKHLEIVERVVREGPSEDLRRKLKESLREIEESLRELKELLDELDELSEKTR

SEQ ID NO: 170 - protein, 72 aa, synthetic construct
  REGION 1..72: note = Synthetic peptide
GSDEEYVTRSQRRLKRLLEEYIKVVEEHARLVERNERDDKELKRSIDELDKLTKELLELVKRYKELVDKTET

SEQ ID NO: 171 - protein, 76 aa, synthetic construct
  REGION 1..76: note = Synthetic peptide
GSDKEEIVKLQDEVIKTLERHLDILRKHIDLLEKLKDHLSEELKERVDRSIKKLEESIKRLERIIEELQELAEYSL

SEQ ID NO: 172 - protein, 75 aa, synthetic construct
  REGION 1..75: note = Synthetic peptide
GSREEELKESAEELERSVRELKKEADKYKEEVDRLHYRGKVDKDWVRVVEKLIKLVEEHLELIREHLELLKEERR

SEQ ID NO: 173 - protein, 75 aa, synthetic construct
  REGION 1..75: note = Synthetic peptide
GSDMEYELKKSAEELRKSLEELKRILDELHKSLRELRRHGDDEEYVQTVEELRKELEEHAKKLEEHLKELERVAT

SEQ ID NO: 174 - protein, 70 aa, synthetic construct
  REGION 1..70: note = Synthetic peptide
PEYELKKSVDDLKRDVDRLVEEVEEVFELSKERLREDRKHLELVEEMVRLIEKHLELIKEHLKLADDHVR

SEQ ID NO: 175 - protein, 78 aa, synthetic construct
  REGION 1..78: note = Synthetic peptide
GSREKDESKELNDEYKKLLEEYERLLRRSEELVKRAKGPRDEKELKRILEENEDILRRTKEILERTKEISEEQKYRRR

SEQ ID NO: 176 - protein, 78 aa, synthetic construct
  REGION 1..78: note = Synthetic peptide
GSDKDERQERLNEESDKSNEESERSNRESEELNRRARGPNDEKELQEILDRHLELLERNQRLLDENKEILRESQYLND

SEQ ID NO: 177 - protein, 76 aa, synthetic construct
  REGION 1..76: note = Synthetic peptide
GSENKYILKEILKLLRENLKLLHDILRLLDENLEELEKHGAKDLDDYRRKIEEIRKKVEDYREKIEEIEKKVERDR

SEQ ID NO: 178 - protein, 75 aa, synthetic construct
  REGION 1..75: note = Synthetic peptide
GSESEYTQEEILELLKESIKLLREILRLLEESEELWRRENTKSERSEEIKERAKEAIKRSEEILERVKRLSDHSR

SEQ ID NO: 179 - protein, 74 aa, synthetic construct
  REGION 1..74: note = Synthetic peptide
GSDEEEANYVSDKAVKIAEDVQELLKELLELSEVVRRGEVDEDEYDRVLRKLQEVMKEYEEVLKEYEEVSRKHE

SEQ ID NO: 180 - protein, 80 aa, synthetic construct
  REGION 1..80: note = Synthetic peptide
GSPEKYLIKTQEELLRRHAEILEDLIRKVERQVDLRRKVDERDEDLKRELERSLRELERLVRESSRLVEEIRELSKEIKR

SEQ ID NO: 181 - protein, 78 aa, synthetic construct
  REGION 1..78: note = Synthetic peptide
GSDEEYELERISRESKELLERYKRLLREYQELLKELRHVKDLDRAVKIIHELMRVSKELVEISHRLLELHERLVRRRK

SEQ ID NO: 182 - protein, 79 aa, synthetic construct
  REGION 1..79: note = Synthetic peptide
GSEKEYIEKLSRKIEEDIRRSEERAKDSERLVRRLEELAKRKRLDLDDVLRVAEENLEILEDNLRILEEILKEQDKSNR

SEQ ID NO: 183 - protein, 75 aa, synthetic construct
  REGION 1..75: note = Synthetic peptide
GSPHEEVVELHERVMEISERAVELIQRIIDIIRRIREDDKDIEKLVKTIRDLVREYEELHRELEEIDEEIYKKSE

SEQ ID NO: 184 - protein, 82 aa, synthetic construct
  REGION 1..82: note = Synthetic peptide
GSDHEDVVRLHEDLVRKQEDARRVLEEIVRLAEEIVEVIKKDEKDKDRVTRLVEEIEKLVEEYKKKVDEMRKISDEIKYRSR

SEQ ID NO: 185 - protein, 79 aa, synthetic construct
  REGION 1..79: note = Synthetic peptide
GSRAREVVKRAKRIIEEWQKILEEWRRILEEWRRLLEDERVDDRDNERIIRENERVIRENEKIIRDVIRLLEELLYERR

SEQ ID NO: 186 - protein, 79 aa, synthetic construct
  REGION 1..79: note = Synthetic peptide
GSREDEELEEEIDRIRQMVEEYEELVKEYEELTEKYKQGKVDKEESKKIIEKSERLLDLSQDAVRKVKEIIRRILYTNR

SEQ ID NO: 187 - protein, 77 aa, synthetic construct
  REGION 1..77: note = Synthetic peptide
GSPKEEIVKLHDESAELHRRSVEVADEILKMHERSKDVDDERESRELSKEIERLIREVEEVSKRIKRLSEEVEYLVR

SEQ ID NO: 188 - protein, 77 aa, synthetic construct
  REGION 1..77: note = Synthetic peptide
GSPLEEILKIQRRINKIQDDINKILHEILRMQEKLNRSSDKDEVEESLRRIRELIKRIKDLSKEIEDLSREVKYRTT

SEQ ID NO: 189 - protein, 81 aa, synthetic construct
  REGION 1..81: note = Synthetic peptide
GSPEDEHVYVVREIYEVLREHAEVLEENREVIERLLEAKKRGDKSEELVKELKKSIDKLKEISRKLEEIVKELEKVSEKLK

SEQ ID NO: 190 - protein, 79 aa, synthetic construct
  REGION 1..79: note = Synthetic peptide
GSDEDETSYRILELLREIVRASRELIRLSEELLEVARRDDKDETVLETLIREYKELLDRYRRLIEELTRLVEEYEERSR

SEQ ID NO: 191 - protein, 74 aa, synthetic construct
  REGION 1..74: note = Synthetic peptide
GSTQEEINRIQHEVLRIQEEIDEILRDIVEKLKAISRGELDHEVVKDVEDKVREALEKSEELLDKSRKVEYKSE

SEQ ID NO: 192 - protein, 82 aa, synthetic construct
  REGION 1..82: note = Synthetic peptide
GSDEEELNRELLEKSKRLVDINRDIIRTAQELIEMLKDSKDGRVDEDTKRELRDKLRKLEEKLERVREELRKYEELLRYVQR

SEQ ID NO: 193 - protein, 77 aa, synthetic construct
  REGION 1..77: note = Synthetic peptide
GSDEKDRVYEILKEVQRLVKEYRDISKEIEDLVKHYEHITDDEAQEVSKELIDKSLRASEIVRELIRLIKELLDELE

SEQ ID NO: 194 - protein, 79 aa, synthetic construct
  REGION 1..79: note = Synthetic peptide
GSDEEDVLYHLRELLEELKRVSDDYERLVREIKETSERKDRDTKENKDMLDELVKAHREQEKLLERLVRLLEELFERKR

SEQ ID NO: 195 - protein, 54 aa, synthetic construct
  REGION 1..54: note = Synthetic peptide
PREQAIRISEEIIRISKKIIEILERTRSSTAREAMKWAKDSIRLAEESKYLLDK

SEQ ID NO: 196 - protein, 54 aa, synthetic construct
  REGION 1..54: note = Synthetic peptide
IEDDVKKIQDSTKKAQKETIEALERSTSSTARKQMEEQKEQIRLQKEAMYLLKK

SEQ ID NO: 197 - protein, 76 aa, synthetic construct
  REGION 1..76: note = Synthetic peptide
SREEIAKLQEEVIKLQRRVIELQKEVIELQRRAKELTSSYTKEILEIQRRIEEIQREIEEIQKRIEEIQEEIQRRT

SEQ ID NO: 198 - protein, 76 aa, synthetic construct
  REGION 1..76: note = Synthetic peptide
SDEEIKRLSEEVIQLSRRVIKMSREAIKLSREVQKLTPSYQKRIKEIADRSIELARESIEIAKRSEKIAEESQRRT

SEQ ID NO: 199 - protein, 56 aa, synthetic construct
  REGION 1..56: note = Synthetic peptide
PAKDEALKMANESLELAKKSARLIQESSSKEILERIEKIQRRIAELQDRIAYLIKK

SEQ ID NO: 200 - protein, 56 aa, synthetic construct
  REGION 1..56: note = Synthetic peptide
PAKDEALRMIDESRELIKKSNELIQRSSSKEILERILEIQRKIAELQKRIQYLLKS

SEQ ID NO: 201 - protein, 75 aa, synthetic construct
  REGION 1..75: note = Synthetic peptide
TDEARYRSERIVKEAKRLLDEARRRSEKIVREAKQRSNSEDAKRIMEENLRESEEAARRLREIIRRNLEESRETG

SEQ ID NO: 202 - protein, 75 aa, synthetic construct
  REGION 1..75: note = Synthetic peptide
TREALEYQRKMAEEIEDLLREALRRQEEMVREAKQRSLSEEFKRIMERILEEQERVMRLAKEALERILEEQKRTG

SEQ ID NO: 203 - protein, 76 aa, synthetic construct
  REGION 1..76: note = Synthetic peptide
SERTKREAKRSQEEILREAKEAMRRAKESQDHRQNRDGSNSEDLERLSQEQKRELEEVERRLKELAREQKYKLEDS

SEQ ID NO: 204 - protein, 76 aa, synthetic construct
  REGION 1..76: note = Synthetic peptide
SEDLKRILKEITERELKLMQDLMEILKKITEDENNLDSNNSEDLKRSIEKARRILDEALRKLEESARRAKYIQEDN

SEQ ID NO: 205 - protein, 76 aa, synthetic construct
  REGION 1..76: note = Synthetic peptide
TEDEIRESLKWLDEVLQELREIARESNEVLERNRQKSRSDKLREDIERYKKRMEEARKKLDDQLNKYKKRMDENRS

SEQ ID NO: 206 - protein, 76 aa, synthetic construct
  REGION 1..76: note = Synthetic peptide
TEEELKESKKFAEDLARSARRALKESKRVLEEISQASRSKKLEEIVRRYKEQVKRWQDEWDERAREYRKRMKENRS

SEQ ID NO: 207 - protein, 76 aa, synthetic construct
  REGION 1..76: note = Synthetic peptide
TKTEEIERLAREIKKLSEKVERLAQEIEELSRRVKEENSTDRELKEANREIERAIREIEKANKRMEEALRRMKYNG

SEQ ID NO: 208 - protein, 76 aa, synthetic construct
  REGION 1..76: note = Synthetic peptide
TKTEEHERLAREISKLADEHRKLAKIIEELARRIKEENLTDDELREAIRKIEDALRKNKEALKIMKEAAERNRYNT

SEQ ID NO: 209 - protein, 76 aa, synthetic construct
  REGION 1..76: note = Synthetic peptide
TKKEESRELARESEELARESEKLARKSLELARRAESSGSEEEKRRIIDENRKIIERNREIIERNKEIIEYNKELIS

SEQ ID NO: 210 - protein, 76 aa, synthetic construct
  REGION 1..76: note = Synthetic peptide
TKDEESLELNRESEELNRKSEELNRKSKELNDRAESSNSEEEEKEILREHKEILREHLEILRRHKEILRRHKYLTS

SEQ ID NO: 211 - protein, 75 aa, synthetic construct
  REGION 1..75: note = Synthetic peptide
TREELLRENIELAKEHIEIMREILELLQKMEELLERQSSEDILEELRKIIERIRELLDRSRKIHERSEEIAYKEE

SEQ ID NO: 212 - protein, 79 aa, synthetic construct
  REGION 1..79: note = Synthetic peptide
SEDIAREIKELLRRLKEIIERNQRIAKEHEYIARERKKLDPSNEKERKLLERSRRLQEESKRLLDEMAEIMRRIKKLLD

SEQ ID NO: 213 - protein, 75 aa, synthetic construct
  REGION 1..75: note = Synthetic peptide
DRQKLIEENIKLLDKHIKILEEILRLLKKDIDLLKKSSSEEVLEELKKIHRRIDKLLDESKKIHKRSSEIVKKRS

SEQ ID NO: 214 - protein, 75 aa, synthetic construct
  REGION 1..75: note = Synthetic peptide
DEQKLIETSQRLQEKSERLLEKFEQILREASDLYRKPDSEELLRRVEKLLRELEKLIRENQDLARKHEKILRDQS

SEQ ID NO: 215 - protein, 75 aa, synthetic construct
  REGION 1..75: note = Synthetic peptide
DRQELIRENIELLKKHIKIVKEIQKLIETFIELLKKSSSEEILRRLKKILKRIEKLYRESQEIHKRSEEIAKKRQ

SEQ ID NO: 216 - protein, 75 aa, synthetic construct
  REGION 1..75: note = Synthetic peptide
DEERLIDKSRELQKESEELLKELLKIFKRIEELLEKPDSEELIREIKKLLETLSEIHKRNEKLARTHEEILRQQS

SEQ ID NO: 217 - protein, 74 aa, synthetic construct
  REGION 1..74: note = Synthetic peptide
STRDVQREIAKAFKKMADVQKKLAEEIKRHVKNVEKKNKDNDEYRKIATELLKKATESQKKLKELLDRIRKSDS

SEQ ID NO: 218 - protein, 74 aa, synthetic construct
  REGION 1..74: note = Synthetic peptide
DKDDRSTSLLKRVEKLIDESDRIIDKFTTLIELSRNGKIDDDQYKKELKEILELLKKYDKHVKEVEELLKRLNS

SEQ ID NO: 219 - protein, 70 aa, synthetic construct
  REGION 1..70: note = Synthetic peptide
SKRKALEVSERVVRISEKVVRVLDESSDLLKKSYDDSDKFAELIDRHEEKIKKWKKLIKEWLEIIQRHKS

SEQ ID NO: 220 - protein, 68 aa, synthetic construct
  REGION 1..68: note = Synthetic peptide
SAEEFVKLSEEAVKRSKEILDIVRKQVKLVKAGVDKHEITDSLRKSEKLIEEHKELIKTHRDLLRREN

SEQ ID NO: 221 - protein, 74 aa, synthetic construct
  REGION 1..74: note = Synthetic peptide
SSTEILKRFKRALRESEKIVKHSRRVLKIIREVLKQKPTQAVHDLVRIIETQVKALEEQLKVLKRIVEALERQS

SEQ ID NO: 222 - protein, 73 aa, synthetic construct
  REGION 1..73: note = Synthetic peptide
DKQKEIKDILEKTRRIAEESRKIAEKFDEIIKRSTEGKIDESLTKELEELVKEVIKLSEDDARTSDDLVRKES

SEQ ID NO: 223 - protein, 74 aa, synthetic construct
  REGION 1..74: note = Synthetic peptide
DEDESIKLTRKSIEETRKSLKIIKEVVELIREVLKHIKDLDKEIFERIDKILDKYKKQVDTYDEILKEYEKKQR

SEQ ID NO: 224 - protein, 70 aa, synthetic construct
  REGION 1..70: note = Synthetic peptide
SELDEQKELIKKQEKLIEEQQRLLSKIRRMFKERVKDQELLREIQKVLKRSQEIVETSKKILDRSDKTTE

SEQ ID NO: 225 - protein, 76 aa, synthetic construct
  REGION 1..76: note = Synthetic peptide
DQKEINTRIVEKLERIFKKSKEIVRQSERVISTIEKKTEDERELDLLRRHVKIVREHLKLLEELLKIIKEVQKESE

SEQ ID NO: 226 - protein, 71 aa, synthetic construct
  REGION 1..71: note = Synthetic peptide
DTEELVKRLNELLKELSKLVKEFIKILETYRKDQTKDTSKISERVDRILKTYEDLLQKYKEILEKIEKQLS

SEQ ID NO: 227 - protein, 68 aa, synthetic construct
  REGION 1..68: note = Synthetic peptide
DYARLIDQAVEVTRKVVEVNVTVARVNDKFAKHLGDEELRRVSEHLKEVSKDLQEVAKKSKDAARQVK

SEQ ID NO: 228 - protein, 77 aa, synthetic construct
  REGION 1..77: note = Synthetic peptide
DVSKVAEEYLQISKTLVDISRTLLEISERLVRLVRTVADDRSEVKKAIEDSIEVLKTSEEVVRQIKRASDKLVKAIS

SEQ ID NO: 229 - protein, 76 aa, synthetic construct
  REGION 1..76: note = Synthetic peptide
DAKEIQRRVVEIQTEVVKLQKKAVDIIRKIIEAFNNSNIDQSLLEAAKEIVKEIDKLEKLTESLLEESKKLLKRSS

SEQ ID NO: 230 - protein, 76 aa, synthetic construct
  REGION 1..76: note = Synthetic peptide
SAEEVVKLAKIFLELLRESIKLLKRSVDLLRKSSDPSLDKSEAEKVSREIEKVSDTSLKLSKKALDVVKRALKVAS

SEQ ID NO: 231 - protein, 81 aa, synthetic construct
  REGION 1..81: note = Synthetic peptide
DEKDAARKARKVSEEAKEASKKIEKALEESKRILNTLKQKKDEQEVKVIKEHEDVLRQIEKIQKQVLEIQKEVAKLLESLD

SEQ ID NO: 232 - protein, 71 aa, synthetic construct
  REGION 1..71: note = Synthetic peptide
SADDVARASEKVLRVARESAKAADKSLEVFKEVVKRGDKEAFLQVVKINEEVVKINITVIRILIEVSKTAT

SEQ ID NO: 233 - protein, 112 aa, synthetic construct
  REGION 1..112: note = Synthetic peptide
DEYVKETLKQLREALASLREADKRITELVKEARKKPLSEAARKFAEAIVTHVKVVVEHVEVVLRHVEVLVEAKKNGVIDKSILDNALRIIENVIRLLSNVIRVVDEVLQDLD

SEQ ID NO: 234 - protein, 113 aa, synthetic construct
  REGION 1..113: note = Synthetic peptide
DASDVIRRIHELFEEVHRLIEAVHRAIEDVAKAAQKKGLDESAVEILAELSKELAKLSRRLAEISREIQKVVTDPDDKEAVERLKEIIKEIKKQLDELRDRLRKLQDLLYKLK

SEQ ID NO: 235 - protein, 77 aa, synthetic construct
  REGION 1..77: note = Synthetic peptide
SEDKAHHDIVRVLEELIKIHDELMKISEEILKATSDSTATDETKEELKRRSKEAQKKSDTLVKIVKELEKESRKAQS

SEQ ID NO: 236 - protein, 73 aa, synthetic construct
  REGION 1..73: note = Synthetic peptide
DDEEKYRQIIREAQEISKTAKRILRDAQEISKRIRHQGVDRSEHQRLVDLLRELIKEHHKLLRRQQEADTRND

SEQ ID NO: 237 - protein, 74 aa, synthetic construct
  REGION 1..74: note = Synthetic peptide
DRKDKARKASEKLEEVIQRWKTVADKWKKMVDLVSNGKLSQEEVARVTEELLKIQTELAKLLEEHAKVLQESAS

SEQ ID NO: 238 - protein, 77 aa, synthetic construct
  REGION 1..77: note = Synthetic peptide
SDEESIKTQSELIKTSEELLKDVKRIDEELQKLRDDPTLDESELKKRVKEWSDRVRKAKEISRKIQEIVKESKKRSS

SEQ ID NO: 239 - protein, 72 aa, synthetic construct
  REGION 1..72: note = Synthetic peptide
DKDEELRKVIEKYREMVKEYRKVIREYEEVIKSSKTIDKSSLISLSRKMVELSQRVIDVSDEVAKVLSRKQS

SEQ ID NO: 240 - protein, 69 aa, synthetic construct
  REGION 1..69: note = Synthetic peptide
TDEERLKKQTKELKEQTKQLEKQKDLLEKISNGEISKDEIQEIIKESKKIAKESQKALDSSRKALEEVS

SEQ ID NO: 241 - protein, 75 aa, synthetic construct
  REGION 1..75: note = Synthetic peptide
DEKEVSKEIIKVLKDIAKVQQKVIEVSQRLASVLRADDDNVVKRALEEYEKILEELRELNKEIEKLTDKYRKVTS

SEQ ID NO: 242 - protein, 77 aa, synthetic construct
  REGION 1..77: note = Synthetic peptide
DSDEQTKELEKLTELHKRHVEKLKKQTKESREVDSNKLWKSKDVKDKLSESEKELQKLSDQDKKAKDALESSRRKND

SEQ ID NO: 243 - protein, 76 aa, synthetic construct
  REGION 1..76: note = Synthetic peptide
DAEEQLKLLTKLLRHQQRLLQLIKESLKLIEKIDQSSQENQDEIRKWREVTKKLRELIKTSEKLVRELEKSYKKSS

SEQ ID NO: 244 - protein, 70 aa, synthetic construct
  REGION 1..70: note = Synthetic peptide
SLRDVVRRYQELVRRYDELIKTLTEILKKYQKKGAEDKDASTELVKAVRTSLKLSKELLKLNSELLKEDS

SEQ ID NO: 245 - protein, 69 aa, synthetic construct
  REGION 1..69: note = Synthetic peptide
SKEELKRKLDELKKRSDTLKELSKKLKEISERNPDDKSVHRTIIRIHREFVKNHKEIVRVIEEIVSDKS

SEQ ID NO: 246 - protein, 71 aa, synthetic construct
  REGION 1..71: note = Synthetic peptide
SKQDEHDRLLKIHDKLVKQHDELLKLLTKLSRAGDSVTKKKLEEILRKLQEVSKQLEESLKDADKVSKDIN

SEQ ID NO: 247 - protein, 69 aa, synthetic construct
  REGION 1..69: note = Synthetic peptide
TVQSLLEQHVKIVKRSIEILERHTQILQDIARSQGVSKELEDVERQVKEYRKEVKKLEEDLRQLSRNSK

SEQ ID NO: 248 - protein, 76 aa, synthetic construct
  REGION 1..76: note = Synthetic peptide
SDSDRIEKLIRESTELLKEQQKLAKRSRELAETVESLPLTEEYLKQQREHQKKIEKLLKDSEKHLEELKRLVKSEK

SEQ ID NO: 249 - protein, 70 aa, synthetic construct
  REGION 1..70: note = Synthetic peptide
DSEKRIEDILRTDLELAKRDAELVKEHIKLVKRIDLSEELKKQVEDVEKESKKLEDSSEKLVQKVRKRSS

SEQ ID NO: 250 - protein, 80 aa, synthetic construct
  REGION 1..80: note = Synthetic peptide
DEEERAKDLRKYLEEQTQYYRTVTEHLRNLEKVVEELERRGKPSSELQQILERSQRIYKETTEIYDTSKKLIEELDKHHR

SEQ ID NO: 251 - protein, 77 aa, synthetic construct
  REGION 1..77: note = Synthetic peptide
PLEDILKRHLDKVRELVRLSEEVNKLAKEVLDILKDKRVDEKELDKVLKELEKVVEEYERAVKESRDLLRELRETTR

SEQ ID NO: 252 - protein, 75 aa, synthetic construct
  REGION 1..75: note = Synthetic peptide
DKERLLEIHERIQKLLDRNLEIIERLLRLLREARDIKDDDKLDKVIKRLKELSEESKDILDKIKELLKESEKELT

SEQ ID NO: 253 - protein, 75 aa, synthetic construct
  REGION 1..75: note = Synthetic peptide
PEDEVIRVIEELLRIAAEVDEVHRRNVEVQEEASRVTDRERLERLNRESEELIKRSRELIEEQRKLIERLERLAT

SEQ ID NO: 254 - protein, 74 aa, synthetic construct
  REGION 1..74: note = Synthetic peptide
DLEELIKEYAEVVRRHHKAVRDLERLVRELANAKHASEEELKRIATEILRIVKELIRVQERLIKLSEDSNEESR

SEQ ID NO: 255 - protein, 75 aa, synthetic construct
  REGION 1..75: note = Synthetic peptide
PTDEVIEVLKELLRIHRENLRVNEEIVEVNERASRVTDREELERLLRRSNELIKRSRELNEESKKLIEKLERLAT

SEQ ID NO: 256 - protein, 74 aa, synthetic construct
  REGION 1..74: note = Synthetic peptide
DNEEIIKEARRVVEEYKKAVDRLEELVRRAENAKHASEKELKDIVREILRISKELNKVSERLIELWERSQERAR

SEQ ID NO: 257 - protein, 72 aa, synthetic construct
  REGION 1..72: note = Synthetic peptide
PKEDIDRVSRELVRVHKELLEVLRKSTEIVEAVARNEKDERTIEEVLEEQERAVRKLEEVSKKHKEAVKRLK

SEQ ID NO: 258 - protein, 77 aa, synthetic construct
  REGION 1..77: note = Synthetic peptide
ELERLSEEIQKLSDRLIELIRRHSKVLEEIVRLLKHKDNDEREVRRLLKLLRDLTRRYEEVLRKVEEIVKRQEDESR

SEQ ID NO: 259 - protein, 72 aa, synthetic construct
  REGION 1..72: note = Synthetic peptide
PEEDILRLLRKLVEVDKELLEVVRESTEVVRLVARNEKDVETVERVLRKQEEVVRKYERVSRELEEAVRRLK

SEQ ID NO: 260 - protein, 77 aa, synthetic construct
  REGION 1..77: note = Synthetic peptide
ELKDLVEEIVKLSKENLKLWEDHSRVLEEIVRLLKHKDNDEREVRRLLKLLEDLTRRAEETSRRIEEIVKEAEDRAR

SEQ ID NO: 261 - protein, 74 aa, synthetic construct
  REGION 1..74: note = Synthetic peptide
DEERELREVLRKHHRVVREWTKVVEELKRVVELLKRGETSEEDLLRVLKKLLEMDKRILEVNREVLRVLEKRLT

SEQ ID NO: 262 - protein, 70 aa, synthetic construct
  REGION 1..70: note = Synthetic peptide
SLEEIIEELVELVRRSVEIAKESDEVARRIVESEDKKKELIDTLRDLHREWQEVTKRAEELVREAEKEVR

SEQ ID NO: 263 - protein, 72 aa, synthetic construct
  REGION 1..72: note = Synthetic peptide
TAEELLEVHKKSDRVTKEHLRVSEEILKVVEVLTRGEVSSEVLKRVLRKLEELTDKLRRVTEEQRRVVEKLN

SEQ ID NO: 264 - protein, 76 aa, synthetic construct
  REGION 1..76: note = Synthetic peptide
DLEDLLRRLRRLVDEQRRLVEELERVSRRLEKAVRDNEDERELARLSREHSDIQDKHDKLAREILEVLKRLLERTE

SEQ ID NO: 265 - protein, 72 aa, synthetic construct
  REGION 1..72: note = Synthetic peptide
PEDDVVRIIKEDLESNREVLREQKEIHRILELVTRGEVSEEAIDRVLKRQEDLLKKQKESTDKARKVVEERR

SEQ ID NO: 266 - protein, 70 aa, synthetic construct
  REGION 1..70: note = Synthetic peptide
DEVRLITEWLKLSEESTRLLKELVELTRLLRNNVPNVEEILREHERISRELERLSRRLKDLADKLERTRR

SEQ ID NO: 267 - protein, 70 aa, synthetic construct
  REGION 1..70: note = Synthetic peptide
DEDEVVKVHEEHVKSHEEIHRSHEEVVRAAEEDKRDSRELRTLMEEHRKLLEENEKSIEEVKKIHERVKR

SEQ ID NO: 268 - protein, 80 aa, synthetic construct
  REGION 1..80: note = Synthetic peptide
KKEELIDISKEVLDLDDEINKISKEILELIKKLLRLKEEGREDKDKAREVKRRIRELHRRIQELNKRLRELHKRVQETKR

SEQ ID NO: 269 - protein, 77 aa, synthetic construct
  REGION 1..77: note = Synthetic peptide
PEEDIARRVEDLLRKSEELIKESEKILKESKRLLDRNDSDKRVLETNLRLIDKHTKLLERNLELLEELLKLAEDVAK

SEQ ID NO: 270 - protein, 74 aa, synthetic construct
  REGION 1..74: note = Synthetic peptide
RFKDLSREYIEVVKRLLELSREALEVLREIKDTDKTDKKRIKELIDRLRKLIEEYKRIIDRLRKLSKDLEEEHR

SEQ ID NO: 271 - protein, 73 aa, synthetic construct
  REGION 1..73: note = Synthetic peptide
DEEELVKILKELQRLSEESLEINKRLVEILRLLRRGEVPKEEVEKKLREIKKEQEKLDREHEKIKKRIEEITK

SEQ ID NO: 272 - protein, 74 aa, synthetic construct
  REGION 1..74: note = Synthetic peptide
SLKEKILEIIERNMKLVELSNRSVEIVARILKGEKDDEETLERLLREWDKITRDYEEIIKESRKLVKELEEEAK

SEQ ID NO: 273 - protein, 74 aa, synthetic construct
  REGION 1..74: note = Synthetic peptide
SKTEILRKALEIHKEQIDIVRKLIELSEEVLKLVEESKEKNLEKLKRIDEETDRLLERLDELHKRLTELAERLK

SEQ ID NO: 274 - protein, 80 aa, synthetic construct
  REGION 1..80: note = Synthetic peptide
SDDEARKQLEEMKRRLREVEKKSKRVEERVRELERLVRENREDEDRVLKTLEDLLRENEKLVRTIERHVREQRELSKEVK

SEQ ID NO: 275 - protein, 73 aa, synthetic construct
  REGION 1..73: note = Synthetic peptide
SEEELEKKADELRKLSEEWRKLQEEDKRLSEMVEKGELDLQEVDEHSLRVLERATEVHRTVDKVIEEILRTTN

SEQ ID NO: 276 - protein, 77 aa, synthetic construct
  REGION 1..77: note = Synthetic peptide
SEKERHRESQETQEEIRRTHEEIIRKLEEILRRAKAGELPEETLDRLRRIMERLKELSERLDDLVRKLRDDHRREQK

SEQ ID NO: 277 - protein, 76 aa, synthetic construct
  REGION 1..76: note = Synthetic peptide
SEKEILEELKRILKRVKDISDRLEELDKRTEEIARREPTKELVDELVKIHRDWLRLHEEILKLVDDALKKVEDATK

SEQ ID NO: 278 - protein, 73 aa, synthetic construct
  REGION 1..73: note = Synthetic peptide
DLRELLELQREASRLHRELVKLLTELVKKLELIAKGEDIREEDLKRIKERLEEIKKRSKRIKEESDEIDKKTK

SEQ ID NO: 279 - protein, 72 aa, synthetic construct
  REGION 1..72: note = Synthetic peptide
SERELQRELNKIVRRILEIHREVSELHQRAVKLIRENDNSEELEEISRRIEELSKELEKLVREHDEIVKTIE

SEQ ID NO: 280 - protein, 77 aa, synthetic construct
  REGION 1..77: note = Synthetic peptide
SEREKLDRNDEELKEINKRVEEIKERSDRITEAIEKNERSEEEIRRLSREQNEALQRLLELHKKLVKLHRELLEDTR

SEQ ID NO: 281 - protein, 76 aa, synthetic construct
  REGION 1..76: note = Synthetic peptide
DKEDVIRVHDEQHKLIEEQLELTRRIAELVREIAKNTASEEEIKEMLKEIKRLDDRSREIQDRLQKLLEEIRRKTK

SEQ ID NO: 282 - protein, 73 aa, synthetic construct
  REGION 1..73: note = Synthetic peptide
TEEEIVELNKDIQRKSKEHIDLQNELVKKIERAIRENNITEELLEELERLLRESEKIVEEIRRITDKIRKDAK

SEQ ID NO: 283 - protein, 72 aa, synthetic construct
  REGION 1..72: note = Synthetic peptide
SEKEILERLLRLSKEQNEISEEIHRLTERLVELKRRKDDDERLKRILDRQKRLVERAREISKEYEDLLRKLE

SEQ ID NO: 284 - protein, 69 aa, synthetic construct
  REGION 1..69: note = Synthetic peptide
SMEELLRKNARLSRKQLKIIDEHLELSTKLTRGEAGDETLEEIERRSREMLEEQRRVDEESKRIREKLK

SEQ ID NO: 285 - protein, 76 aa, synthetic construct
  REGION 1..76: note = Synthetic peptide
SEEEIRDIVEKLLRTHEEVLKEIKKLLDDSERVRRRELDKKDLDRIQKEQRDIQEENKEKAKRFDELVKELKKAAK

SEQ ID NO: 286 - protein, 70 aa, synthetic construct
  REGION 1..70: note = Synthetic peptide
SEEEHRRTMEKVEKEVRDIKRRSEEVKKKVKANTLSEEDLVRLLERLVEDHKRLQDLSQEIIERDEKATK

SEQ ID NO: 287 - protein, 72 aa, synthetic construct
  REGION 1..72: note = Synthetic peptide
DEDELAKEIEDVQRRNKESQEEHDKSVKKLEAAERGEIDEDSLLRVLEEDIKVLEKDIEVLERSIEVIEKAE

SEQ ID NO: 288 - protein, 73 aa, synthetic construct
  REGION 1..73: note = Synthetic peptide
SEKELIRRLLEQQRQHLRLSERLIELSRRLVEVVRKGKDNRDLLRELKKLSEEHKKHSKDDHEKVREIREREK

SEQ ID NO: 289 - protein, 75 aa, synthetic construct
  REGION 1..75: note = Synthetic peptide
DRKDLLKRNIKLLDRHLKILDTILKLLEKLSELLKKSSSEEVVKEYKKILDEIRKLLEESKEIHKESKEILERES

SEQ ID NO: 290 - protein, 75 aa, synthetic construct
  REGION 1..75: note = Synthetic peptide
DEEKLIERSKRLQEESEQLLEKFEQILRELTELLEKPDSEELARKIKKLHDELRKIIKRNQELIREHEEILRKRD

SEQ ID NO: 291 - protein, 234 aa, synthetic construct
  REGION 1..234: note = Synthetic peptide
PKEEARELIRKQKELIKEQKKLIKEAKQKSDSRDAERIWKRSREINRESKKINKRIKELIKSGSEGSGSEGSGSTKEDILERQRKIIERAQEIHRRQQEILEELERIIRKPGSSEEAMKRMLKLLEESLRLLKELLELSEESAQLLYEQRGSEGSGSEGSGSDSDEHLKKLKTFLENLRRHLDRLDKHIKQLRDILSENPEDERVKDVIDLSERSVRIVKTVIKIFEDSVRKKE

SEQ ID NO: 292 - protein, 76 aa, synthetic construct
  REGION 1..76: note = Synthetic peptide
TEKRLLEEAERAHREQKEIIKKAQELHRRLEEIVRQSGSSEEAKKEAKKILEEIRELSKRSLELLREILYLSQEQK

SEQ ID NO: 293 - protein, 93 aa, synthetic construct
  REGION 1..93: note = Synthetic peptide
GSSHHHHHHSSGENLYFQGSDDKELDKLLDTLEKILQTATKIIDDANKLLEKLRRSERKDPKVVETYVELLKRHEKAVKELLEIAKTHAKKVE

SEQ ID NO: 294 - protein, 150 aa, synthetic construct
  REGION 1..150: note = Synthetic peptide
PKEEARELIRKQKELIKEQKKLIKEAKQKSDSRDAERIWKRSREINRESKKINKRIKELIKSGSEGSGSEGSGSTKEDILERQRKIIERAQEIHRRQQEILEELERIIRKPGSSEEAMKRMLKLLEESLRLLKELLELSEESAQLLYEQR

SEQ ID NO: 295 - protein, 96 aa, synthetic construct
  REGION 1..96: note = Synthetic peptide
GSHHHHHHGSGSENLYFQGSTEKRLLEEAERAHREQKEIIKKAQELHRRLEEIVRQSGSSEEAKKEAKKILEEIRELSKRSLELLREILYLSQEQK

SEQ ID NO: 296 - protein, 156 aa, synthetic construct
  REGION 1..156: note = Synthetic peptide
TERKLLERSRRLQEESKRLLDEMAEIMRRIKKLLDDPDSEDIAREIKELLRRLKEIIERNQRIAKEHEYIARERSGPGSGSEGSDSDEHLKKLKTFLENLRRHLDRLDKHIKQLRDILSENPEDERVKDVIDLSERSVRIVKTVIKIFEDSVRKKE

SEQ ID NO: 297 - protein, 75 aa, synthetic construct; identical to SEQ ID NO: 211
  REGION 1..75: note = Synthetic peptide

SEQ ID NO: 298 - protein, 93 aa, synthetic construct
  REGION 1..93: note = Synthetic peptide
GSHHHHHHGSGSENLYFQGSDDKELDKLLDTLEKILQTATKIIDDANKLLEKLRRSERKDPKVVETYVELLKRHEKAVKELLEIAKTHAKKVE

SEQ ID NO: 299 - protein, 161 aa, synthetic construct
  REGION 1..161: note = Synthetic peptide
TEKRLLEEAERAHREQKEIIKKAQELHRRLEEIVRQSGSSEEAKKEAKKILEEIRELSKRSLELLREILYLSQEQKGSEGSGSEGSGSDDKELDKLLDTLEKILQTATKIIDDANKLLEKLRRSERKDPKVVETYVELLKRHEKAVKELLEIAKTHAKKVE

SEQ ID NO: 300 - protein, 76 aa, synthetic construct
  REGION 1..76: note = Synthetic peptide
TKEDILERQRKIIERAQEIHRRQQEILEELERIIRKPGSSEEAMKRMLKLLEESLRLLKELLELSEESAQLLYEQR

SEQ ID NO: 301 - protein, 92 aa, synthetic construct
  REGION 1..92: note = Synthetic peptide
GSSHHHHHHSSGENLYFQGSDSDEHLKKLKTFLENLRRHLDRLDKHIKQLRDILSENPEDERVKDVIDLSERSVRIVKTVIKIFEDSVRKKE

SEQ ID NO: 302 - protein, 149 aa, synthetic construct
  REGION 1..149: note = Synthetic peptide
TERKLLERSRRLQEESKRLLDEMAEIMRRIKKLLDDPDSEDIAREIKELLRRLKEIIERNQRIAKEHEYIARERSGSEGSGSEGSGSPKEEARELIRKQKELIKEQKKLIKEAKQKSDSRDAERIWKRSREINRESKKINKRIKELIKS

SEQ ID NO: 303 - protein, 147 aa, synthetic construct
  REGION 1..147: note = Synthetic peptide
PKKEAEELAEESEELHDRSEKLHERAEQSSNSEEARKILEDIERISERIEEISDRIERLLRSGSEGSGSEGSGSDDKELDKLLDTLEKILQTATKIIDDANKLLEKLRRSERKDPKVVETYVELLKRHEKAVKELLEIAKTHAKKVE

SEQ ID NO: 304 - protein, 150 aa, synthetic construct
  REGION 1..150: note = Synthetic peptide
TKEDILERQRKIIERAQEIHRRQQEILEELERIIRKPGSSEEAMKRMLKLLEESLRLLKELLELSEESAQLLYEQRGSEGSGSEGSGSPKEEARELIRKQKELIKEQKKLIKEAKQKSDSRDAERIWKRSREINRESKKINKRIKELIKS

SEQ ID NO: 305 - protein, 144 aa, synthetic construct
  REGION 1..144: note = Synthetic peptide
PKKEAEELAEESEELHDRSEKLHERAEQSSNSEEARKILEDIERISERIEEISDRIERLLRSGSEGSGSEGSDSDEHLKKLKTFLENLRRHLDRLDKHIKQLRDILSENPEDERVKDVIDLSERSVRIVKTVIKIFEDSVRKKE

SEQ ID NO: 306 - protein, 152 aa, synthetic construct
  REGION 1..152: note = Synthetic peptide
GSTERKLLERSRRLQEESKRLLDEMAEIMRRIKKLLDDPDSEDIAREIKELLRRLKEIIERNQRIAKEHEYIARERSAADDKELDKLLDTLEKILQTATKIIDDANKLLEKLRRSERKDPKVVETYVELLKRHEKAVKELLEIAKTHAKKVE

SEQ ID NO: 307 - protein, 156 aa, synthetic construct
  REGION 1..156: note = Synthetic peptide
GSTERKLLERSRRLQEESKRLLDEMAEIMRRIKKLLDDPDSEDIAREIKELLRRLKEIIERNQRIAKEHEYIARERSGGSGSPDDKELDKLLDTLEKILQTATKIIDDANKLLEKLRRSERKDPKVVETYVELLKRHEKAVKELLEIAKTHAKKVE

SEQ ID NO: 308 - protein, 162 aa, synthetic construct
  REGION 1..162: note = Synthetic peptide
GSTERKLLERSRRLQEESKRLLDEMAEIMRRIKKLLDDPDSEDIAREIKELLRRLKEIIERNQRIAKEHEYIARERSGGSGSPGGSGSPDDKELDKLLDTLEKILQTATKIIDDANKLLEKLRRSERKDPKVVETYVELLKRHEKAVKELLEIAKTHAKKVE

SEQ ID NO: 309 - protein, 174 aa, synthetic construct
  REGION 1..174: note = Synthetic peptide
GSTERKLLERSRRLQEESKRLLDEMAEIMRRIKKLLDDPDSEDIAREIKELLRRLKEIIERNQRIAKEHEYIARERSGGSGSPGGSGSPGGSGSPGGSGSPDDKELDKLLDTLEKILQTATKIIDDANKLLEKLRRSERKDPKVVETYVELLKRHEKAVKELLEIAKTHAKKVE

SEQ ID NO: 310 - protein, 158 aa, synthetic construct
  REGION 1..158: note = Synthetic peptide
PEDDVVRIIKEDLESNREVLREQKEIHRILELVTRGEVSEEAIDRVLKRQEDLLKKQKESTDKARKVVEERRGSEGSGSEGSDLEDLLRRLRRLVDEQRRLVEELERVSRRLEKAVRDNEDERELARLSREHSDIQDKHDKLAREILEVLKRLLERTE

SEQ ID NO: 311 - protein, 163 aa, synthetic construct
  REGION 1..163: note = Synthetic peptide
GSDAYDLDRIVKEHRRLVEEQRELVEELEKLVRRQEDHRVDKKESHEILERLERIIRRSTRILTELEKLTDEFERRTRGSEGSGSEGSGSDDKELDKLLDTLEKILQTATKIIDDANKLLEKLRRSERKDPKVVETYVELLKRHEKAVKELLEIAKTHAKKVE

SEQ ID NO: 312 - protein, 165 aa, synthetic construct
  REGION 1..165: note = Synthetic peptide
GSDEDDELERLLREYHRVLREYEKLLEELRRLYEEYKRGEVSEEESDRILREIKEILDKSERLWDLSEEVWRTLLYQAEGSEGSGSEGSDEKDYHRRLIEHLEDLVRRHEELIKRQKKVVEELERRGLDERLRRVVDRFRRSSERWEEVIERFRQVVDKLRKSVE

SEQ ID NO: 313 - protein, 168 aa, synthetic construct
  REGION 1..168: note = Synthetic peptide
GSTERKLLERSRRLQEESKRLLDEMAEIMRRIKKLLKKARGADEKVLDELRKIIERIRELLDRSRKIHERSEEIAYKEEGSEGSGSEGSGSDESDRIRKIVEESDEIVKESRKLAERARELIKESEDKRVSEERNERLLEELLRILDENAELLKRNLELLKEVLYRTR

SEQ ID NO: 314 - protein, 164 aa, synthetic construct
  REGION 1..164: note = Synthetic peptide
GSDEDDELERLLREYHRVLREYEKLLEELRRLYEEYKRGEVSEEESDRILREIKEILDKSERLWDLSEEVWRTLLYQAEGSEGSGSEGSGSDDKELDKLLDTLEKILQTATKIIDDANKLLEKLRRSERKDPKVVETYVELLKRHEKAVKELLEIAKTHAKKVE

SEQ ID NO: 315 - protein, 177 aa, synthetic construct
  REGION 1..177: note = Synthetic peptide
DSDEHLKKLKTFLENLRRHLDRLDKHIKQLRDILSENPEDERVKDVIDLSERSVRIVKTVIKIFEDSVRKKEGSEGSGSEGSGSEGSGSEGSGSEGSGSEGSPTDEVIEVLKELLRIHRENLRVNEEIVEVNERASRVTDREELERLLRRSNELIKRSRELNEESKKLIEKLERLAT

SEQ ID NO: 316 - protein, 155 aa, synthetic construct
  REGION 1..155: note = Synthetic peptide
DDKELDKLLDTLEKILQTATKIIDDANKLLEKLRRSERKDPKVVETYVELLKRHEKAVKELLEIAKTHAKKVEGSEGSGSEGSTAEELLEVHKKSDRVTKEHLRVSEEILKVVEVLTRGEVSSEVLKRVLRKLEELTDKLRRVTEEQRRVVEKLN

SEQ ID NO: 317 - protein, 156 aa, synthetic construct
  REGION 1..156: note = Synthetic peptide
DNEEIIKEARRVVEEYKKAVDRLEELVRRAENAKHASEKELKDIVREILRISKELNKVSERLIELWERSQERARGSEGSGSEGSTAEELLEVHKKSDRVTKEHLRVSEEILKVVEVLTRGEVSSEVLKRVLRKLEELTDKLRRVTEEQRRVVEKLN

SEQ ID NO: 318 - protein, 280 aa, synthetic construct
  REGION 1..280: note = Synthetic peptide
DDKELDKLLDTLEKILQTATKIIDDANKLLEKLRRSERKDPKVVETYVELLKRHEKAVKELLEIAKTHAKKVEGSEGSGSEGSGSEGSGSEGSGSEGSGSEGSPTDEVIEVLKELLRIHRENLRVNEEIVEVNERASRVTDREELERLLRRSNELIKRSRELNEESKKLIEKLERLATGSEGSGSEGSGSEGSGSEGSGSEGSGSEGSTAEELLEVHKKSDRVTKEHLRVSEEILKVVEVLTRGEVSSEVLKRVLRKLEELTDKLRRVTEEQRRVVEKLN

SEQ ID NO: 319 - protein, 154 aa, synthetic construct
  REGION 1..154: note = Synthetic peptide
DSDEHLKKLKTFLENLRRHLDRLDKHIKQLRDILSENPEDERVKDVIDLSERSVRIVKTVIKIFEDSVRKKEGSEGSGSEGSPEDDVVRIIKEDLESNREVLREQKEIHRILELVTRGEVSEEAIDRVLKRQEDLLKKQKESTDKARKVVEERR

SEQ ID NO: 320 - protein, 156 aa, synthetic construct
  REGION 1..156: note = Synthetic peptide
DNEEIIKEARRVVEEYKKAVDRLEELVRRAENAKHASEKELKDIVREILRISKELNKVSERLIELWERSQERARGSEGSGSEGSPEDDVVRIIKEDLESNREVLREQKEIHRILELVTRGEVSEEAIDRVLKRQEDLLKKQKESTDKARKVVEERR

SEQ ID NO: 321 - protein, 178 aa, synthetic construct
  REGION 1..178: note = Synthetic peptide
DLEDLLRRLRRLVDEQRRLVEELERVSRRLEKAVRDNEDERELARLSREHSDIQDKHDKLAREILEVLKRLLERTEGSEGSGSEGSGSEGSGSEGSGSEGSGSEGSPEDDVVRIIKEDLESNREVLREQKEIHRILELVTRGEVSEEAIDRVLKRQEDLLKKQKESTDKARKVVEERR

SEQ ID NO: 322 - protein, 160 aa, synthetic construct
  REGION 1..160: note = Synthetic peptide
GSDDKELDKLLDTLEKILQTATKIIDDANKLLEKLRRSERKDPKVVETYVELLKRHEKAVKELLEIAKTHAKKVEGSEGSGSEGSPTDEVIEVLKELLRIHRENLRVNEEIVEVNERASRVTDREELERLLRRSNELIKRSRELNEESKKLIEKLERLAT

SEQ ID NO: 323 - protein, 154 aa, synthetic construct
  REGION 1..154: note = Synthetic peptide
DSDEHLKKLKTFLENLRRHLDRLDKHIKQLRDILSENPEDERVKDVIDLSERSVRIVKTVIKIFEDSVRKKEGSEGSGSEGSTAEELLEVHKKSDRVTKEHLRVSEEILKVVEVLTRGEVSSEVLKRVLRKLEELTDKLRRVTEEQRRVVEKLN

SEQ ID NO: 324 - protein, 166 aa, synthetic construct
  REGION 1..166: note = Synthetic peptide
GTERKLLERSRRLQEESKRLLDEMAEIMRRIKKLLKKARGADEKVLDELRKIIERIRELLDRSRKIHERSEEIAYKEEGSEGSGSEGSGSDAYDLDRIVKEHRRLVEEQRELVEELEKLVRRQEDHRVDKKESHEILERLERIIRRSTRILTELEKLTDEFERRTR

SEQ ID NO: 325 - protein, 162 aa, synthetic construct
  REGION 1..162: note = Synthetic peptide
TREELLRENIELAKEHIEIMREILELLQKMEELLEKARGADEDVAKTIKELLRRLKEIIERNQRIAKEHEYIARERSGSEGSGSEGSGSDDKELDKLLDTLEKILQTATKIIDDANKLLEKLRRSERKDPKVVETYVELLKRHEKAVKELLEIAKTHAKKVE

SEQ ID NO: 326 - protein, 162 aa, synthetic construct
  REGION 1..162: note = Synthetic peptide
GSDDKELDKLLDTLEKILQTATKIIDDANKLLEKLRRSERKDPKVVETYVELLKRHEKAVKELLEIAKTHAKKVEGSEGSGSEGSGTKEDILERQRKIIERAQEIHRRQQEILEELERIIRKPGSSEEAMKRMLKLLEESLRLLKELLELSEESAQLLYEQR

SEQ ID NO: 327 - protein, 15 aa, synthetic construct
  REGION 1..15: note = Synthetic peptide
GAAGGAGATATCATC

SEQ ID NO: 328 - protein, 20 aa, synthetic construct
  REGION 1..20: note = Synthetic peptide
GSSHHHHHHSSGENLYFQGS

SEQ ID NO: 329 - protein, 40 aa, synthetic construct
  REGION 1..40: note = Synthetic peptide
SAWSHPQFEKGGGSGGGSGGSAWSHPQFEKSGENLYFQGS

SEQ ID NO: 330 - protein, 44 aa, synthetic construct
  REGION 1..44: note = Synthetic peptide
GSGATNFSLLKQAGDVEENPGPGDKAELIPEPPKKKRKVELGTA

SEQ ID NO: 331 - protein, 62 aa, synthetic construct
  REGION 1..62: note = Synthetic peptide
PKEEARELIRKQKELIKEQKKLIKEAKQKSDSRDAERIWKRSREINRESKKINKRIKELIKS

SEQ ID NO: 332 - protein, 76 aa, synthetic construct
  REGION 1..76: note = Synthetic peptide
TEKRLLEEAERAHREAKEIIKKAQELHRRLEEIVRQSGSSEEAKKEAKKILEEIRELSKRLLELLREILYLSQEQK

SEQ ID NO: 333 - protein, 5 aa, synthetic construct
  REGION 1..5: note = Synthetic peptide
YSWNS

SEQ ID NO: 334 - protein, 76 aa, synthetic construct
  REGION 1..76: note = Synthetic peptide
TEKRLLEEAERAHREQKEIIKKAQELHRRTEEIIRQSGSSEEAKDELRRIQEEIRELSKRSLELLREILYLSQEQK

SEQ ID NO: 335 - skipped

SEQ ID NO: 336 - protein, 76 aa, synthetic construct; identical to SEQ ID NO: 334
  REGION 1..76: note = Synthetic peptide

SEQ ID NO: 337 - protein, 37 aa, synthetic construct
  REGION 1..37: note = Synthetic peptide
PERDENRKLLDKVRKLVEKSRRLVEELRKLVDQSTKN

SEQ ID NO: 338 - protein, 114 aa, synthetic construct
  REGION 1..114: note = Synthetic peptide
SEEAKKEAKKILEEIRELSKRSLELLREILYLSQQVNDVDEKALERQRKIIERAQEIHRRQQEILEELERIIRKPGSSEEAMKRMLKLLEESLRLLKELLELSEESAQLLYEAR

SEQ ID NO: 339 - protein, 37 aa, synthetic construct
  REGION 1..37: note = Synthetic peptide
DRRLEEYERQVDELREEIRRYKEEVDKFDKEVKYYKK

SEQ ID NO: 340 - protein, 70 aa, synthetic construct
  REGION 1..70: note = Synthetic peptide
DREEVHKEIVKLIREIIKIHKKILKIHEKIKNGEIDPSEILKLSEEIKKLTDTIIKIIEDLEQLTRDLRR

SEQ ID NO: 341 - protein, 36 aa, synthetic construct
  REGION 1..36: note = Synthetic peptide
DEITESVDRFKKIVDQFEESIKKFETVSEELRKSIS

SEQ ID NO: 342 - protein, 74 aa, synthetic construct
  REGION 1..74: note = Synthetic peptide
DKAEEVEKSVRKIEESIKKIRKSIKKAEDAVQLLKEGKIDAKDFLRIVREDLEVVKEDVEIVKEDVENVREFSS

SEQ ID NO: 343 - protein, 34 aa, synthetic construct
  REGION 1..34: note = Synthetic peptide
DQHVVEILRKIVEIFRQHIEKLKKHLEKLRYTSS

SEQ ID NO: 344 - protein, 76 aa, synthetic construct
  REGION 1..76: note = Synthetic peptide
DLKEVLKTVEEAVKEIIKSSEELLQISRKILEISRVGVDEHEYISAIREYLKALEKHIQILKKFIEILKELIRAVS

SEQ ID NO: 345 - protein, 71 aa, synthetic construct
  REGION 1..71: note = Synthetic peptide
DEEDYINENVEKDVRDIEDDVRRINERIRELLEKIRTEEVLQRVLEEHHELVERVLRKLVEILRKHEEENR

SEQ ID NO: 346 - protein, 76 aa, synthetic construct
  REGION 1..76: note = Synthetic peptide
NDEELKKILETLDRILKKLDKILTRLIEVLKKSEDPNLDDKDYTELVKQFIELIKKYEEVVKEYEEVVRQLIRLFS

SEQ ID NO: 347 - protein, 82 aa, synthetic construct
  REGION 1..82: note = Synthetic peptide
DEDEISYDSKRRVEEIVRQAREKSEKSRKDIEDVAEVLRKGDVSEKEVVDELVKVLEEQVKVLREAVERLREVLKKQVDDVR

SEQ ID NO: 348 - protein, 76 aa, synthetic construct
  REGION 1..76: note = Synthetic peptide
NDEELKKILETLDRILKKLEKILTRLIEVLKKSEDPNLDDKDYTELVKQFIELIKKFEEVIKEYEEVVRQLIRLFS

SEQ ID NO: 349 - protein, 81 aa, synthetic construct
  REGION 1..81: note = Synthetic peptide
KEKDIVKTLVDLLRENLETLERLIEEVVRLLKENVDVRDEGRDDKDSERILRDIKRRIDEAAKESREIIERIEKEVEYRSR

SEQ ID NO: 350 - protein, 74 aa, synthetic construct
  REGION 1..74: note = Synthetic peptide
DKKDELERILDEIRRLIERLDEILSRLNKLLELLKHGVPNAKEVVKDYIRLLKEYLELVKEFLKLVKRHADLVS

SEQ ID NO: 351 - protein, 82 aa, synthetic construct
  REGION 1..82: note = Synthetic peptide
DKRYESEKLKRRLDEAVEKVREVVERVERESDRVLEEVRRRRESKEVVDKVIEDNDKALEDVLRVVDEVAKVVRDVVRENTR

SEQ ID NO: 352 - protein, 73 aa, synthetic construct
  REGION 1..73: note = Synthetic peptide
DDKELDKLLDTLEKILQTATKIIDDANKLLEKLRRSERKDPKVVETYVELLKRHEKAVKELLEIAKTHAKKVE

SEQ ID NO: 353 - protein, 77 aa, synthetic construct
  REGION 1..77: note = Synthetic peptide
DEDEILYHSERLLQKLKKELDDLKEKSRELLEELKKEDPDDRLIERIIRLHDEVLKDLDEVLKNILEVHREVLERLR

SEQ ID NO: 354 - protein, 73 aa, synthetic construct
  REGION 1..73: note = Synthetic peptide
DDKELDKLLDTLEKILQTATKIIDDLNKVLEKLRRSERKDPKVIETVVELLKRHEKAVKELLEIAKTHAKKVE

SEQ ID NO: 355 - protein, 78 aa, synthetic construct
  REGION 1..78: note = Synthetic peptide
PKEELIRRVLEEVKRLNEKLLEIIRRAAELVKRANDELPETEKLREIDRELEKKLKEIEDELRRIDKELDDALYEIED

SEQ ID NO: 356 - protein, 73 aa, synthetic construct
  REGION 1..73: note = Synthetic peptide
DDKELDKLLDTLEKILQTATKVVDDANKLLEKLRRSERKDPKVVETYVELLKRLEKLIKELLEIAKTHAKKVE

SEQ ID NO: 357 - protein, 73 aa, synthetic construct
  REGION 1..73: note = Synthetic peptide
KEDESVKRAEEIVRTLLKLLEDSLREAERSLRDIKNGEDEHNLRRISEKLEELSKRITETIERLLRELQYTSR

SEQ ID NO: 358 - protein, 73 aa, synthetic construct
  REGION 1..73: note = Synthetic peptide
DDKEATKIIDDLDKLLDTLEKILQTANKLLEKLRRSERKDPKVVETYVKAVKELLEIAKTHAELLKRHEKKVE

SEQ ID NO: 359 - protein, 82 aa, synthetic construct
  REGION 1..82: note = Synthetic peptide
DESEEAQHEVEKVLDDIRRLSEHLQKRLEEVLEEVYELRREGSDRTEVVELLKEVIREIVRVNREALERLLRVVEEAVKRNE

SEQ ID NO: 360 - protein, 73 aa, synthetic construct
  REGION 1..73: note = Synthetic peptide
DDKEANKLLEKATKIIDDLDKLLDTLEKILQTLRRSERKDPKAVKELLEIAKTHAELLKRHEKVVETYVKKVE

SEQ ID NO: 361 - protein, 78 aa, synthetic construct
  REGION 1..78: note = Synthetic peptide
DEEEEVVRRAEELVKEHEELIERVIRTHEELVYKLEDQGADKKLVDVLKRVVEESERVAREIVKVSRELIRLLEEASR

SEQ ID NO: 362 - protein, 114 aa, synthetic construct
  REGION 1..114: note = Synthetic peptide
SADDVLEDILKIIRELIEILDQILSLLNQLLKLLRHGVPNAKKVVEKYKEILELYLQLVSLFLKIVKTHADAVSGKIDKKAEEEIKKEEEKIKEKLRQAKDILKKLQEEIDKTR

SEQ ID NO: 363 - protein, 79 aa, synthetic construct
  REGION 1..79: note = Synthetic peptide
DKEEESEYLLRDLVRLLEKVKEKIEEVNREVEKLLKKVKDGRLDRREVLREILRLNRELAEIIKEVVDRIRHVVERSER

SEQ ID NO: 364 - protein, 112 aa, synthetic construct
  REGION 1..112: note = Synthetic peptide
NDKVLDKILDILDRILRLATRVIDLANKLLQVKKKSTHKDPRIVETYKELLKIHETAVRLLLELADLHRRLKSKDEEANKRVETELDRIRKKVKDIEDKVRKLEDKVRKTAS

SEQ ID NO: 365 - protein, 79 aa, synthetic construct
  REGION 1..79: note = Synthetic peptide
PVEEIIKEVVKRVIEVQEKVLRIISHAVKRVVEVQKKYDPGSEESNRVVEEVKKTIEDAIRESDEVVDEVVKRIQYTVR

SEQ ID NO: 366 - protein, 76 aa, synthetic construct
  REGION 1..76: note = Synthetic peptide
DEAVKRVVEKSLKILDEVIKKSLDILRELIELQIRHAKDDESVIRASKSALKDAIEALKKSLDEIKKALKRSADEG

SEQ ID NO: 367 - protein, 78 aa, synthetic construct
  REGION 1..78: note = Synthetic peptide
DEDKEANRVLDEVLKTVRDLLETANEVLKEVLYRLKRTDDQEKVVRTLTEVLKEHLKLVEEIVRILDKVLKEHLETEK

SEQ ID NO: 368 - protein, 76 aa, synthetic construct
  REGION 1..76: note = Synthetic peptide
SKEEVIRLLKENVRLIKENLELLTRNLKLITDLVRGSNGSEEKIKTLKELLKEYRELLKRYRKLVEDYKRLVDKHD

SEQ ID NO: 369 - protein, 76 aa, synthetic construct
  REGION 1..76: note = Synthetic peptide
RREEVVKRIRELLKRNKELIDRIRELLEENEYLDKDARDKDVLRRSVELLEELVRILEESVELAKEIIKLLREVVE

SEQ ID NO: 370 - protein, 73 aa, synthetic construct
  REGION 1..73: note = Synthetic peptide
YIEDVIKKILDVSRELIKLSRTIIKISEEINKQLQQGRDTKDLVKKYDEIIKKYTRIVQHYTELIKELQKLLS

SEQ ID NO: 371 - protein, 75 aa, synthetic construct
  REGION 1..75: note = Synthetic peptide
DEKEEAKKASEESVRTVERILEELLKASEESVELLRRGEDAKDVVERSKEALKRVKELLDEVVKRSDEILKYIHN

SEQ ID NO: 372 - protein, 73 aa, synthetic construct
  REGION 1..73: note = Synthetic peptide
EEEEILKIQKELLRIQSEILDKQKKILDTLRSNGAVTEEVRSILEKVERLSEEAKELSKEAKELTKEVSKLIS

SEQ ID NO: 373 - protein, 74 aa, synthetic construct
  REGION 1..74: note = Synthetic peptide
PDMDEVKRVLDELIEIQEEILREIKRVLEKLIKIQEDNGSEYESREVVREIVEIARKLVERSRRVVKKITETLQ

SEQ ID NO: 374 - protein, 76 aa, synthetic construct
  REGION 1..76: note = Synthetic peptide
PLEDLVRKYDELVKTYEKLVEEFKKAVDKYDKAVKKAPVSKEATDSLDLIRKVLELLDRNLKLIKENAKLIKELLK

SEQ ID NO: 375 - protein, 77 aa, synthetic construct
  REGION 1..77: note = Synthetic peptide
PEREALREVLEDLKRVTDRLRELVERVLEELKKVTDHVDSERILRESRRVLKELKDIIEEILRESEKVLEKLKYTED

SEQ ID NO: 376 - protein, 70 aa, synthetic construct
  REGION 1..70: note = Synthetic peptide
PIKDISKRLLEISKRLVEISDRIVELLQRIADSKDPNKDLQKEVKDVLEEYKRLVREYREVVKEYEKVVS

SEQ ID NO: 377 - protein, 73 aa, synthetic construct
  REGION 1..73: note = Synthetic peptide
PVEEAIKKVIDDLRDVQRKIRELVEELIRLLEEVQRDNDKRESEYVVERVEEILRRITETSREVVRKAVEDLS

SEQ ID NO: 378 - protein, 69 aa, synthetic construct
  REGION 1..69: note = Synthetic peptide
NLEELVKLLKEVLEMHERLLRIHEDLVEAHKSNASDKESERKLKKSDKDIKESLKKIKSIIDQVRYIQS

SEQ ID NO: 379 - protein, 77 aa, synthetic construct
  REGION 1..77: note = Synthetic peptide
PVEEVLKELSEVNERVRDIAREIIERLSEVNEEVKETDDEDELKKISKKVVDEVEDLLRKILEVSEEVVRRVEYHDR

SEQ ID NO: 380 - protein, 76 aa, synthetic construct
  REGION 1..76: note = Synthetic peptide
DEDYESREIIDEIRKLLDRSKKIVHRSQRLVERVKSTPLSEDQEDLIRRHEETINRHRELVKELEKVLEDHERHIR

SEQ ID NO: 381 - protein, 73 aa, synthetic construct
  REGION 1..73: note = Synthetic peptide
PRSYLLKELADLSQHLVRLLERLVRESERVVEVLERGEVDEEELKRLEDLHRELEKAVREVRETHREIRERSR

SEQ ID NO: 382 - protein, 77 aa, synthetic construct
  REGION 1..77: note = Synthetic peptide
PEKDENRKLLDKVRKLVEKSRRLVEELRKLVDQSTKNGLIDEKALRKQQEVLRKVEEVLEKQERVLRELEEISYRVI

SEQ ID NO: 383 - protein, 73 aa, synthetic construct
  REGION 1..73: note = Synthetic peptide
DEKDEIRHVIESVERLIEDIKRLLKTLRELAHDDSDKKTVKEVLDRVKEMIERHRRELEEHRKELERAEYEVR

SEQ ID NO: 384 - protein, 122 aa, synthetic construct
  REGION 1..122: note = Synthetic peptide
DEKALRKQQEVLRKVEEVLEKQERVLRELEEISYRVITRGEDHKAEEDSRRVLERFVRVSREVLKVLEEFLRVSEELLREADRDRDRRLEEYERQVDELREEIRRYKEEVDKFDKEVKYYKK

SEQ ID NO: 385 - protein, 75 aa, synthetic construct
  REGION 1..75: note = Synthetic peptide
SLEDSVRLNDEVVKVVERVVRLNQEVVRLIKHATDVEDEETVKYVLERVREVLDESREVLKRVHELLEESERRLE

SEQ ID NO: 386 - protein, 123 aa, synthetic construct
  REGION 1..123: note = Synthetic peptide
PERDENRKLLDKVRKLVEKSRRLVEELRKLVDQSTKNGLIDEKALRKQQEVLRKVEEVLEKQERVLRELEEISYRVITRGEDHKAEEDSRRVLERFVRVSREVLKVLEEFLRVSEELLREADR

SEQ ID NO: 387 - protein, 74 aa, synthetic construct
  REGION 1..74: note = Synthetic peptide
PELDEVKKLIDELKKSVERLEESIREVKESIKKLRKGDIDAEENIKLLKENIKIVRENIKIIKEIIDVVQYVLR

SEQ ID NO: 388 - protein, 78 aa, synthetic construct
  REGION 1..78: note = Synthetic peptide
DSEYESRQVLRELDTVLKDSHTVLEALRQVIRDSQDVVSKSDEESRRVIDDLEKVIQDSKKVLDDIKRLIDKSKSIKS

SEQ ID NO: 389 - protein, 77 aa, synthetic construct
  REGION 1..77: note = Synthetic peptide
DESDRIRKIVEESDEIVKESRKLAERARELIKESEDKRVSEERNERLLEELLRILDENAELLKRNLELLKEVLYRTR

SEQ ID NO: 390 - protein, 76 aa, synthetic construct
  REGION 1..76: note = Synthetic peptide
SLYELTQRYEKLVQQYEELVKDYRRLVKKLEKLKRDNKPDKRLLKEIVDVIKKSVEIIDRSLKLLEESIKILEETD

SEQ ID NO: 391 - protein, 76 aa, synthetic construct
  REGION 1..76: note = Synthetic peptide
DKKDASRRAIRVLHEFVRVSEEVLEVLRKSVESLKRLDVDEKIKRTHDRIEEELRRWKRELEELIERLREWEYHQD

SEQ ID NO: 392 - protein, 76 aa, synthetic construct
  REGION 1..76: note = Synthetic peptide
DIEYESKEILELIKELLKLSRELLKESRRALELVRKSRDDSIVEEVIQVHKKVLDIHKEVLKIVRKVVEVHRRVKS

SEQ ID NO: 393 - protein, 77 aa, synthetic construct
  REGION 1..77: note = Synthetic peptide
DKEYKLDRILRRLDELIKQLSRILEEIERLVDELEREPLDDKEVQDVIERIVELIDEHLELLKEYIKLLEEYIKTTK

SEQ ID NO: 394 - protein, 76 aa, synthetic construct
  REGION 1..76: note = Synthetic peptide
DLEYLNRRLLQLIKTLIDLNRHLLKLIDKLKKLNSREGDEEKIKEESKQIQEQFKEIVERSKEIIKQIKEIIKRSQ

SEQ ID NO: 395 - protein, 76 aa, synthetic construct
  REGION 1..76: note = Synthetic peptide
RDRKISEELIKALEDHIRMLEELIRAIEEHIKLAERGVDEKELRESLEELKKIVDELEKSLEELRKLAERYKYETR

SEQ ID NO: 396 - protein, 76 aa, synthetic construct
  REGION 1..76: note = Synthetic peptide
EESEEVRKVVERIKKISRELEEVVKELDRVSKEFDRHGETDEIVREHERIVEKLEEIVKKHTKIVEELAEIVYKQQ

SEQ ID NO: 397 - protein, 75 aa, synthetic construct
  REGION 1..75: note = Synthetic peptide
DHEYWVKIVERILRVMEKHAEIVKKHLEIVERVVREGPSEDLRRKLKESLREIEESLRELKELLDELDELSEKTR

SEQ ID NO: 398 - protein, 70 aa, synthetic construct
  REGION 1..70: note = Synthetic peptide
DLLYLSKELLKLVRELLKLSRELVELSRRLVNSTHKSPELVKKYDKLVKKYQDLLKKLADVADEYLRQRS

SEQ ID NO: 399 - protein, 74 aa, synthetic construct
  REGION 1..74: note = Synthetic peptide
DKEEIVKLQDEVIKTLERHLDILRKHIDLLEKLKDHLSEELKERVDRSIKKLEESIKRLERIIEELQELAEYSL

SEQ ID NO: 400 - protein, 76 aa, synthetic construct
  REGION 1..76: note = Synthetic peptide
DAYDLDRIVKEHRRLVEEQRELVEELEKLVRRQEDHRVDKKESHEILERLERIIRRSTRILTELEKLTDEFERRTR

SEQ ID NO: 401 - protein, 73 aa, synthetic construct
  REGION 1..73: note = Synthetic peptide
DMEYELKKSAEELRKSLEELKRILDELHKSLRELRRHGDDEEYVQTVEELRKELEEHAKKLEEHLKELERVAT

SEQ ID NO: 402 - protein, 69 aa, synthetic construct
  REGION 1..69: note = Synthetic peptide
DRQRILDRLDKILEKLDDILKKLKDILETLSKDDVSDRRHKDLVEKFRELVDTHHKLVERYRELVYQNR

SEQ ID NO: 403 - protein, 76 aa, synthetic construct
  REGION 1..76: note = Synthetic peptide
REKDESKELNDEYKKLLEEYERLLRRSEELVKRAKGPRDEKELKRILEENEDILRRTKEILERTKEISEEQKYRRR

SEQ ID NO: 404 - protein, 105 aa, synthetic construct
  REGION 1..105: note = Synthetic peptide
DPQRAADRLDKILEKLDDILKKLKDILETLSKDDVKDRRAKDLVEKFRELVDTHHKLVERYRELVYTATAGSDLARELIRRVEEHTKRLRHILKRLREHEEKLRR

SEQ ID NO: 405 - protein, 74 aa, synthetic construct
  REGION 1..74: note = Synthetic peptide
ENKYILKEILKLLRENLKLLHDILRLLDENLEELEKHGAKDLDDYRRKIEEIRKKVEDYREKIEEIEKKVERDR

SEQ ID NO: 406 - protein, 68 aa, synthetic construct
  REGION 1..68: note = Synthetic peptide
DKEYLVTEHEKLVREHEKIVSEIEKLVKKHEAGVDESELEEILKKVEKLLRKLDEILEQLTQLLRKTE

SEQ ID NO: 407 - protein, 72 aa, synthetic construct
  REGION 1..72: note = Synthetic peptide
DEEEANYVSDKAVKIAEDVQELLKELLELSEVVRRGEVDEDEYDRVLRKLQEVMKEYEEVLKEYEEVSRKHE

SEQ ID NO: 408 - protein, 112 aa, synthetic construct
  REGION 1..112: note = Synthetic peptide
DAEYLVTEHEKLVREHEKIVSEIEKLVKKHEKGVDESELEEILKKVEKLLRKLDEILEQLTQLLRKAEKHIDKHSKAADQLATSIKKLEDSIDQLIKIVRKFEESVKKLQKH

SEQ ID NO: 409 - protein, 76 aa, synthetic construct
  REGION 1..76: note = Synthetic peptide
DEEYELERISRESKELLERYKRLLREYQELLKELRHVKDLDRAVKIIHELMRVSKELVEISHRLLELHERLVRRRK

SEQ ID NO: 410 - protein, 72 aa, synthetic construct
  REGION 1..72: note = Synthetic peptide
RDKYLLERLNDILKKLDEIVDKLSDILKRLKDVRHDDRLQELVERYKEIVKEYKRIVEEYEKLVREFEEQQR

SEQ ID NO: 411 - protein, 73 aa, synthetic construct
  REGION 1..73: note = Synthetic peptide
PHEEVVELHERVMEISERAVELIQRIIDIIRRIREDDKDIEKLVKTIRDLVREYEELHRELEEIDEEIYKKSE

SEQ ID NO: 412 - protein, 74 aa, synthetic construct
  REGION 1..74: note = Synthetic peptide
DAEYKVRESVKRSKESVKHSEDVVDKLNKSVKLSESGHSDAEKASRELVKLVREVVELSREVIKLSEKVLRVIS

SEQ ID NO: 413 - protein, 77 aa, synthetic construct
  REGION 1..77: note = Synthetic peptide
RAREVVKRAKRIIEEWQKILEEWRRILEEWRRLLEDERVDDRDNERIIRENERVIRENEKIIRDVIRLLEELLYERR

SEQ ID NO: 414 - protein, 77 aa, synthetic construct
  REGION 1..77: note = Synthetic peptide
DWEELLRRLEKVLQEYEEIVKELIDLIERLIKVSEDKSKDASEYKKLVTELEKLISKLEEISKKLEELVKEYEYKTE

SEQ ID NO: 415 - protein, 75 aa, synthetic construct
  REGION 1..75: note = Synthetic peptide
PKEEIVKLHDESAELHRRSVEVADEILKMHERSKDVDDERESRELSKEIERLIREVEEVSKRIKRLSEEVEYLVR

SEQ ID NO: 416 - protein, 73 aa, synthetic construct
  REGION 1..73: note = Synthetic peptide
DKERAARATEEMVKLTKKLLKAVEDLVRDVRRLLKEGLISEKHARIAETILEVFKKHAKIIKKHVDIVKYDES

SEQ ID NO: 417 - protein, 79 aa, synthetic construct
  REGION 1..79: note = Synthetic peptide
PEDEHVYVVREIYEVLREHAEVLEENREVIERLLEAKKRGDKSEELVKELKKSIDKLKEISRKLEEIVKELEKVSEKLK

SEQ ID NO: 418 - protein, 76 aa, synthetic construct
  REGION 1..76: note = Synthetic peptide
NDEELKKILETLDRILKKLDKILTRLDEVLKKSEDPNLDDKDYTELVKQYIELVKKYEEVVKEYEEVVRQLIRLFS

SEQ ID NO: 419 - protein, 72 aa, synthetic construct
  REGION 1..72: note = Synthetic peptide
TQEEINRIQHEVLRIQEEIDEILRDIVEKLKAISRGELDHEVVKDVEDKVREALEKSEELLDKSRKVEYKSE

SEQ ID NO: 420 - protein, 81 aa, synthetic construct
  REGION 1..81: note = Synthetic peptide
EEDERIRYDLDRIRKDVRRKLEEIRQRVRELEKKLRDAGHRRDEKELLRELIETSKDILRLVEELLKKIIDKSEDLLRKTE

SEQ ID NO: 421 - protein, 75 aa, synthetic construct
  REGION 1..75: note = Synthetic peptide
DEKDRVYEILKEVQRLVKEYRDISKEIEDLVKHYEHITDDEAQEVSKELIDKSLRASEIVRELIRLIKELLDELE

SEQ ID NO: 422 - protein, 75 aa, synthetic construct
  REGION 1..75: note = Synthetic peptide
DEEEYYKEKLHKLLREIEELLKHYRELVRRLEELVKRGELDKDTAAHILERLSELLERIIRRVAHTLRRLSEERR

SEQ ID NO: 423 - protein, 5 aa, synthetic construct
  REGION 1..5: note = Synthetic peptide
GSEGS

SEQ ID NO: 424 - protein, 80 aa, synthetic construct
  REGION 1..80: note = Synthetic peptide
DIVELVDHLLKRSLKLLEELAELVRRLLEKSTELLKRRTEEHKEEVVEESEYMVRELEERLRRVVDESEKLVRDADKHIR

SEQ ID NO: 425 - protein, 4 aa, synthetic construct
  REGION 1..4: note = Synthetic peptide
GGGS

SEQ ID NO: 426 - protein, 78 aa, synthetic construct
  REGION 1..78: note = Synthetic peptide
PEVDVLRRIVREILKASEELLRLLRKLIDEALKLSERKRDSQEYREVVDRVKKELERLLDEYRKLVEELKEKLRYDTR

SEQ ID NO: 427 - protein, 12 aa, synthetic construct
  REGION 1..12: note = Synthetic peptide
GSEGSGSEGSGS

SEQ ID NO: 428 - protein, 75 aa, synthetic construct
  REGION 1..75: note = Synthetic peptide
PREYHSKDILRKVDEILERIRRHADRVKKKSERLKRENVDVNEHSKDVKRVIRELLELVKELLRLAKKHSDDQQE

SEQ ID NO: 429 - protein, 10 aa, synthetic construct
  REGION 1..10: note = Synthetic peptide
GSEGSGSEGS

SEQ ID NO: 430 - protein, 78 aa, synthetic construct
  REGION 1..78: note = Synthetic peptide
PKLDKLRELLERNLEKLREILEEVLKILRTNLERVREDIRDEDVLQEYERLIRKAEEDLRRVLKEYDDLLKKLVYELR

SEQ ID NO: 431 - protein, 94 aa, synthetic construct
  REGION 1..94: note = Synthetic peptide
HHHHHHGSGSENLYFQGSTEKRLLEEAERAHREQKEIIKKAQELHRRLEEIVRQSGSSEEAKKEAKKILEEIRELSKRSLELLREILYLSQEQK

SEQ ID NO: 432 - protein, 82 aa, synthetic construct
  REGION 1..82: note = Synthetic peptide
PNQELLDRVRKILEDLLRLNEELVRLNKELLKRALEMRRKNRDSEEVLERLAEEYRKRLEEYRRELEKLLEELEETIYRYKR

SEQ ID NO: 433 - protein, 91 aa, synthetic construct
  REGION 1..91: note = Synthetic peptide
HHHHHHGSGSENLYFQGSDDKELDKLLDTLEKILQTATKIIDDANKLLEKLRRSERKDPKVVETYVELLKRHEKAVKELLEIAKTHAKKVE

SEQ ID NO: 434 - protein, 81 aa, synthetic construct
  REGION 1..81: note = Synthetic peptide
DEEELVETVKRIQKEILDRLTELAKLLVEIQREIKKLKDEGEDDKELKRLSDELEEKVRQVVEEIKRLSDELEETVEYVSR

SEQ ID NO: 435 - protein, 90 aa, synthetic construct
  REGION 1..90: note = Synthetic peptide
SHHHHHHSSGENLYFQGSDSDEHLKKLKTFLENLRRHLDRLDKHIKQLRDILSENPEDERVKDVIDLSERSVRIVKTVIKIFEDSVRKKE

SEQ ID NO: 436 - protein, 78 aa, synthetic construct
  REGION 1..78: note = Synthetic peptide
SKEEILKELEDLQRRLIEELKKLQERVVELLEELIKRLRDRGRDDKHLKRLVKEVRRLSEEVLRSIKEVSDRVRYQLR

SEQ ID NO: 437 - protein, 91 aa, synthetic construct
  REGION 1..91: note = Synthetic peptide
SHHHHHHSSGENLYFQGSDDKELDKLLDTLEKILQTATKIIDDANKLLEKLRRSERKDPKVVETYVELLKRHEKAVKELLEIAKTHAKKVE

SEQ ID NO: 438 - protein, 74 aa, synthetic construct
  REGION 1..74: note = Synthetic peptide
DLHEVVYETKELLKRIEEVVEELRKKSEDIIRKAERGEISEDELKRLQEEIAREAKKLLDEIKRVLERHLEQTL

SEQ ID NO: 439 - protein, 150 aa, synthetic construct
  REGION 1..150: note = Synthetic peptide
TERKLLERSRRLQEESKRLLDEMAEIMRRIKKLLDDPDSEDIAREIKELLRRLKEIIERNQRIAKEHEYIARERSAADDKELDKLLDTLEKILQTATKIIDDANKLLEKLRRSERKDPKVVETYVELLKRHEKAVKELLEIAKTHAKKVE

SEQ ID NO: 440 - protein, 78 aa, synthetic construct
  REGION 1..78: note = Synthetic peptide
PEQEIADRILTEIRESQKELERLARKILKLLDESQEKAKRGRLSEEESDELLERIKKELDELLERSKELLKKIEYELR

SEQ ID NO: 441 - protein, 154 aa, synthetic construct
  REGION 1..154: note = Synthetic peptide
TERKLLERSRRLQEESKRLLDEMAEIMRRIKKLLDDPDSEDIAREIKELLRRLKEIIERNQRIAKEHEYIARERSGGSGSPDDKELDKLLDTLEKILQTATKIIDDANKLLEKLRRSERKDPKVVETYVELLKRHEKAVKELLEIAKTHAKKVE

SEQ ID NO: 442 - protein, 74 aa, synthetic construct
  REGION 1..74: note = Synthetic peptide
PEDDVLRRLEEVSEKILRVAEDVARQLREVSEKITQGKVDRKEWEEDIKRLKRELEELLREWKEEIERLTYELR

SEQ ID NO: 443 - protein, 160 aa, synthetic construct
  REGION 1..160: note = Synthetic peptide
TERKLLERSRRLQEESKRLLDEMAEIMRRIKKLLDDPDSEDIAREIKELLRRLKEIIERNQRIAKEHEYIARERSGGSGSPGGSGSPDDKELDKLLDTLEKILQTATKIIDDANKLLEKLRRSERKDPKVVETYVELLKRHEKAVKELLEIAKTHAKKVE

SEQ ID NO: 444 - protein, 73 aa, synthetic construct
  REGION 1..73: note = Synthetic peptide
DEKEDNRRLQHKIERILEKNEDLQRKLEEILELLERGEADEEKIDRLRKAVEDYRRVVEEIKEDVKRHKYTVR

SEQ ID NO: 445 - protein, 172 aa, synthetic construct
  REGION 1..172: note = Synthetic peptide
TERKLLERSRRLQEESKRLLDEMAEIMRRIKKLLDDPDSEDIAREIKELLRRLKEIIERNQRIAKEHEYIARERSGGSGSPGGSGSPGGSGSPGGSGSPDDKELDKLLDTLEKILQTATKIIDDANKLLEKLRRSERKDPKVVETYVELLKRHEKAVKELLEIAKTHAKKVE

SEQ ID NO: 446 - protein, 82 aa, synthetic construct
  REGION 1..82: note = Synthetic peptide
DEKKLINEVVETQKRLIKEAAKRLSEVVRHQTELIRELREKNVDDKDVEKLLKESLDLAEEIVRRIKELLDESKKLVEYVSN

SEQ ID NO: 447 - protein, 161 aa, synthetic construct
  REGION 1..161: note = Synthetic peptide
DAYDLDRIVKEHRRLVEEQRELVEELEKLVRRQEDHRVDKKESHEILERLERIIRRSTRILTELEKLTDEFERRTRGSEGSGSEGSGSDDKELDKLLDTLEKILQTATKIIDDANKLLEKLRRSERKDPKVVETYVELLKRHEKAVKELLEIAKTHAKKVE

SEQ ID NO: 448 - protein, 78 aa, synthetic construct
  REGION 1..78: note = Synthetic peptide
DERYATREIVERIERIAREILKRTEEIVREVREVLSRDVDQEEVVRRLADLLRESVELVQHLVRRVEELLQESVERKK

SEQ ID NO: 449 - protein, 163 aa, synthetic construct
  REGION 1..163: note = Synthetic peptide
DEDDELERLLREYHRVLREYEKLLEELRRLYEEYKRGEVSEEESDRILREIKEILDKSERLWDLSEEVWRTLLYQAEGSEGSGSEGSDEKDYHRRLIEHLEDLVRRHEELIKRQKKVVEELERRGLDERLRRVVDRFRRSSERWEEVIERFRQVVDKLRKSVE

SEQ ID NO: 450 - protein, 78 aa, synthetic construct
  REGION 1..78: note = Synthetic peptide
PAREILEEVVKKHLEVVEDAARILEEIIREHEKAVREDRDKKELEEISRDLLRKAREALKKVKDISDDLSREIEYVAS

SEQ ID NO: 451 - protein, 166 aa, synthetic construct
  REGION 1..166: note = Synthetic peptide
TERKLLERSRRLQEESKRLLDEMAEIMRRIKKLLKKARGADEKVLDELRKIIERIRELLDRSRKIHERSEEIAYKEEGSEGSGSEGSGSDESDRIRKIVEESDEIVKESRKLAERARELIKESEDKRVSEERNERLLEELLRILDENAELLKRNLELLKEVLYRTR

SEQ ID NO: 452 - protein, 79 aa, synthetic construct
  REGION 1..79: note = Synthetic peptide
DSDEKAEYLLKEMERVVRESDEVVKKILRDLEEVLERLRRGEISEDDVTEILKELAERHIRAIEELVRRLRELLERHKR

SEQ ID NO: 453 - protein, 162 aa, synthetic construct
  REGION 1..162: note = Synthetic peptide
DEDDELERLLREYHRVLREYEKLLEELRRLYEEYKRGEVSEEESDRILREIKEILDKSERLWDLSEEVWRTLLYQAEGSEGSGSEGSGSDDKELDKLLDTLEKILQTATKIIDDANKLLEKLRRSERKDPKVVETYVELLKRHEKAVKELLEIAKTHAKKVE

SEQ ID NO: 454 - protein, 74 aa, synthetic construct
  REGION 1..74: note = Synthetic peptide
PKEDILREVLRRHKEIVREIVRLVREAVETHLELVKRNSDDRDAQDVIRKLEEDLERLVRHAQEVIEEIFYRLH

SEQ ID NO: 455 - protein, 158 aa, synthetic construct
  REGION 1..158: note = Synthetic peptide
DDKELDKLLDTLEKILQTATKIIDDANKLLEKLRRSERKDPKVVETYVELLKRHEKAVKELLEIAKTHAKKVEGSEGSGSEGSPTDEVIEVLKELLRIHRENLRVNEEIVEVNERASRVTDREELERLLRRSNELIKRSRELNEESKKLIEKLERLAT

SEQ ID NO: 456 - protein, 74 aa, synthetic construct
  REGION 1..74: note = Synthetic peptide
DREYIIKDILDSQEHLLRLIEELLETQKELLEILKRRPDSVERVRELVRRSKEIADEIRRQSDRNVRLLEEVSK

SEQ ID NO: 457 - protein, 160 aa, synthetic construct
  REGION 1..160: note = Synthetic peptide
DDKELDKLLDTLEKILQTATKIIDDANKLLEKLRRSERKDPKVVETYVELLKRHEKAVKELLEIAKTHAKKVEGSEGSGSEGSGTKEDILERQRKIIERAQEIHRRQQEILEELERIIRKPGSSEEAMKRMLKLLEESLRLLKELLELSEESAQLLYEQR

SEQ ID NO: 458 - protein, 74 aa, synthetic construct
  REGION 1..74: note = Synthetic peptide
ESEDRIKELLKRHIELVERHEELLHEIKKLIDLEEKDDKDREEAVKRIDDAIKESEEMLEESKEILEEIEYLNR

SEQ ID NO: 459 - protein, 76 aa, synthetic construct
  REGION 1..76: note = Synthetic peptide
PLKERLLEIQRDLDRVLEEVVERLLRIQERLDSVVERKPPDVHEEYKYIVDEIREIVERVVREYEEIVKRIDEEVR

SEQ ID NO: 460 - protein, 80 aa, synthetic construct
  REGION 1..80: note = Synthetic peptide
HEKDIVYKVEDLVRKSDRIAERAREIVKRSRDIMREIRKDKDNKKLSDDLLKVTRDLQRVVDELEELSRELLRVAEESRK

SEQ ID NO: 461 - protein, 13 aa, synthetic construct
  REGION 1..13: note = Synthetic peptide
GSEGSGSEGSGGS

SEQ ID NO: 462 - protein, 80 aa, synthetic construct
  REGION 1..80: note = Synthetic peptide
DEEEIEELLRELEKLLKKSEEALEESKKLIDESEELLRRDRLDKEKHVRASEEHVKLSEEHLRISREIVKILEKAVYSTR

SEQ ID NO: 463 - skipped

SEQ ID NO: 464 - protein, 77 aa, synthetic construct
  REGION 1..77: note = Synthetic peptide
DEDDELERLLREYHRVLREYEKLLEELRRLYEEYKRGEVSEEESDRILREIKEILDKSERLWDLSEEVWRTLLYQAE

SEQ ID NO: 465 - skipped

SEQ ID NO: 466 - protein, 73 aa, synthetic construct
  REGION 1..73: note = Synthetic peptide
DDEEEDKRLLEEVKRSLDTDERILEKLRHSLERQLEDVDKDEDSRRVLRELDEITKRSREVVKRLRKLAYESK

SEQ ID NO: 467 - skipped

SEQ ID NO: 468 - protein, 77 aa, synthetic construct
  REGION 1..77: note = Synthetic peptide
PSKEYQEKSAERQKELLHEYEKLVRHLRELVEKLQRRELDKEEVLRRLVEILERLKDLHKKIEDAHRKNEEAHKENK

SEQ ID NO: 469 - skipped

SEQ ID NO: 470 - protein, 75 aa, synthetic construct
  REGION 1..75: note = Synthetic peptide
PKEESVEELKRVIDKHEEILRELKRVLEEHERVSHDEDENELRRSLERLKHILDRLHESLKELHELLKKNEYTER

SEQ ID NO: 471 - skipped

SEQ ID NO: 472 - protein, 70 aa, synthetic construct
  REGION 1..70: note = Synthetic peptide
DEEYVTRSQRRLKRLLEEYIKVVEEHARLVERNERDDKELKRSIDELDKLTKELLELVKRYKELVDKTET

SEQ ID NO: 473 - skipped

SEQ ID NO: 474 - protein, 73 aa, synthetic construct
  REGION 1..73: note = Synthetic peptide
REEELKESAEELERSVRELKKEADKYKEEVDRLHYRGKVDKDWVRVVEKLIKLVEEHLELIREHLELLKEERR

SEQ ID NO: 475 - skipped

SEQ ID NO: 476 - protein, 76 aa, synthetic construct
  REGION 1..76: note = Synthetic peptide
DKDERQERLNEESDKSNEESERSNRESEELNRRARGPNDEKELQEILDRHLELLERNQRLLDENKEILRESQYLND

SEQ ID NO: 477 - skipped

SEQ ID NO: 478 - protein, 73 aa, synthetic construct
  REGION 1..73: note = Synthetic peptide
ESEYTQEEILELLKESIKLLREILRLLEESEELWRRENTKSERSEEIKERAKEAIKRSEEILERVKRLSDHSR

SEQ ID NO: 479 - skipped

SEQ ID NO: 480 - protein, 78 aa, synthetic construct
  REGION 1..78: note = Synthetic peptide
PEKYLIKTQEELLRRHAEILEDLIRKVERQVDLRRKVDERDEDLKRELERSLRELERLVRESSRLVEEIRELSKEIKR

SEQ ID NO: 481 - skipped

SEQ ID NO: 482 - protein, 77 aa, synthetic construct
  REGION 1..77: note = Synthetic peptide
EKEYIEKLSRKIEEDIRRSEERAKDSERLVRRLEELAKRKRLDLDDVLRVAEENLEILEDNLRILEEILKEQDKSNR

SEQ ID NO: 483 - skipped

SEQ ID NO: 484 - protein, 80 aa, synthetic construct
  REGION 1..80: note = Synthetic peptide
DHEDVVRLHEDLVRKQEDARRVLEEIVRLAEEIVEVIKKDEKDKDRVTRLVEEIEKLVEEYKKKVDEMRKISDEIKYRSR

SEQ ID NO: 485 - skipped

SEQ ID NO: 486 - protein, 77 aa, synthetic construct
  REGION 1..77: note = Synthetic peptide
REDEELEEEIDRIRQMVEEYEELVKEYEELTEKYKQGKVDKEESKKIIEKSERLLDLSQDAVRKVKEIIRRILYTNR

SEQ ID NO: 487 - skipped

SEQ ID NO: 488 - protein, 75 aa, synthetic construct
  REGION 1..75: note = Synthetic peptide
PLEEILKIQRRINKIQDDINKILHEILRMQEKLNRSSDKDEVEESLRRIRELIKRIKDLSKEIEDLSREVKYRTT

SEQ ID NO: 489 - skipped

SEQ ID NO: 490 - protein, 77 aa, synthetic construct
  REGION 1..77: note = Synthetic peptide
DEDETSYRILELLREIVRASRELIRLSEELLEVARRDDKDETVLETLIREYKELLDRYRRLIEELTRLVEEYEERSR

SEQ ID NO: 491 - skipped

SEQ ID NO: 492 - protein, 80 aa, synthetic construct
  REGION 1..80: note = Synthetic peptide
DEEELNRELLEKSKRLVDINRDIIRTAQELIEMLKDSKDGRVDEDTKRELRDKLRKLEEKLERVREELRKYEELLRYVQR

SEQ ID NO: 493 - skipped

SEQ ID NO: 494 - protein, 77 aa, synthetic construct
  REGION 1..77: note = Synthetic peptide
DEEDVLYHLRELLEELKRVSDDYERLVREIKETSERKDRDTKENKDMLDELVKAHREQEKLLERLVRLLEELFERKR

(19) United States
(12) Patent Application Publication
(10) Pub. No.: US 2024/0101615 A1
(43) Pub. Date: Mar. 28, 2024

(54) ORTHOGONAL PROTEIN HETERODIMERS

(71) Applicant: UNIVERSITY OF WASHINGTON, Seattle, WA (US)

(72) Inventors: Zibo CHEN, Seattle, WA (US); Scott Boyken, Seattle, WA (US); Sherry Bermeo, Seattle, WA (US); Robert A. Langan, Seattle, WA (US); David Baker, Seattle, WA (US)

(21) Appl. No.: 18/158,384

(22) Filed: Jan. 23, 2023

Related U.S. Application Data
(63) Continuation of application No. 17/482,713, filed on Sep. 23, 2021, now abandoned.
Application No. 17/482,713 is a continuation of application No. 17/285,033, filed on Apr. 13, 2021, now Pat. No. 11,820,800.
(60) Provisional application No. 62/904,800, filed on Sep. 24, 2019.
(60) Provisional application No. 62/755,264, filed on Nov. 2, 2018.

(51) Int. Cl. C07K 14/435; B82Y 5/00; B82Y 10/00; B82Y 15/00; C07K 14/00; C07K 19/00; G01N 33/53; G06N 3/00; G16B 5/00; G16B 15/00; H03K 19/00

(52) U.S. Cl. CPC C07K 14/435; B82Y 5/00; B82Y 10/00; B82Y 15/00; C07K 14/00; C07K 19/00; G01N 33/53; G06N 3/002; G16B 5/00; G16B 15/00; H03K 19/00; C07K 2319/00; G16B 5/10

(57) ABSTRACT

Disclosed herein are designed heterodimer proteins, monomeric polypeptides capable of forming heterodimer proteins, protein scaffolds including such polypeptides, and methods for using the heterodimer proteins and subunit polypeptides for designing logic gates.

### CROSS REFERENCE

This application is a Continuation of U.S. patent application Ser. No. 17/482,713, filed Sep. 23, 2021, which is a Continuation of U.S. patent application Ser. No. 17/285,033, filed Apr. 13, 2021, which is a U.S. national phase of International Application No. PCT/US2019/059654, filed on Nov. 4, 2019, which claims priority to U.S. Provisional Application No. 62/904,800, filed on Sep. 24, 2019 and U.S. Provisional Application No. 62/755,264, filed on Nov. 2, 2018, all of which are incorporated by reference herein in their entirety.

### FEDERAL FUNDING STATEMENT

This invention was made with government support under Grant No. GM103533 awarded by the National Institutes of Health. The government has certain rights in the invention

### BACKGROUND

Heterodimeric interaction specificity between two DNA strands, and between protein and DNA, is often achieved by varying side chains or bases coming off the protein or DNA backbone—for example, the bases participating in Watson-Crick base pairing in the double helix, or the side chains of protein contacting DNA in TALEN-DNA complexes. This modularity enables the generation of an essentially unlimited number of orthogonal DNA-DNA and protein-DNA heterodimers. In contrast, protein-protein interaction specificity is often achieved through backbone shape complementarity, which is less modular and hence harder to generalize.

### SUMMARY

In one aspect, the disclosure provides designed heterodimer proteins, comprising:
(a) a monomer A polypeptide, wherein the monomer A polypeptide is a non-naturally occurring polypeptide comprising 1-5 alpha helices connected by amino acid linkers; and
(b) a monomer B polypeptide, wherein the monomer B polypeptide is a non-naturally occurring polypeptide comprising 1-5 alpha helices connected by amino acid linkers,
wherein monomer A and monomer B non-covalently interact to form the designed heterodimer protein. In one embodiment,
(i) monomer A comprises a polypeptide having at least 70%, 75%, 80%, 85%, 90%, 91%, 92%, 93%, 94%, 95%, 96%, 97%, 98%, 99%, or 100% sequence identity along the length of the amino acid sequence of an odd-numbered SEQ ID NO selected from the group consisting of selected from the group SEQ ID NOS: 1-290; and
(ii) monomer B comprises a polypeptide having at least 70%, 75%, 80%, 85%, 90%, 91%, 92%, 93%, 94%, 95%, 96%, 97%, 98%, 99%, or 100% sequence identity along the length of the amino acid sequence of an even-numbered SEQ ID NO selected from the group consisting of selected from the group SEQ ID NOS: 1-290, wherein the even-numbered SEQ ID NO is the binding partner of the odd-numbered SEQ ID NO. in step (i).

In another aspect, the disclosure provides non-naturally occurring polypeptide comprising a polypeptide having at least 70%, 75%, 80%, 85%, 90%, 91%, 92%, 93%, 94%, 95%, 96%, 97%, 98%, 99%, or 100% sequence identity along the length of the 20 amino acid sequence selected from the group consisting of SEQ ID NOS: 1-290.

In another aspect, the disclosure provides non-naturally occurring polypeptide comprising a polypeptide having at least 70%, 75%, 80%, 85%, 90%, 91%, 92%, 93%, 94%, 95%, 96%, 97%, 98%, 99%, or 100% sequence identity along the length of the amino acid sequence selected from the group consisting of SEQ ID NOS: 1-290, 331, 332, 334, 336-422, 424, 426, 428, 430, 432, 434, 436, 438, 440, 442, 444, 446, 448, 450, 452, 454, 456, 458-460, 462, 464, 466, 468, 470, 472, 474, 476, 478, 480, 482, 484, 486, 488, 490, 493, and 494.

In another aspect, the disclosure provides proteins comprising 2, 3, 4, or more non-naturally occurring polypeptides having at least 70%, 75%, 80%, 85%, 90%, 91%, 92%, 93%, 94%, 95%, 96%, 97%, 98%, 99%, or 100% sequence identity along the length of the amino acid sequence selected from the group consisting of SEQ ID NOS: 1-290, wherein the 2, 3, 4, or more naturally occurring polypeptides are covalently linked. In one embodiment, each of the 2, 3, 4, or more non-naturally occurring polypeptides are different. In another embodiment, each of the 2, 3, 4, or more non-naturally occurring polypeptides are present in a fusion protein.

In another aspect, the disclosure provides proteins comprising 2, 3, 4, or more non-naturally occurring polypeptides having at least 70%, 75%, 80%, 85%, 90%, 91%, 92%, 93%, 94%, 95%, 96%, 97%, 98%, 99%, or 100% sequence identity along the length of the amino acid sequence selected from the group consisting of SEQ ID NOS: 1-290, 1-290, 331, 332, 334, 336-422, 424, 426, 428, 430, 432, 434, 436, 438, 440, 442, 444, 446, 448, 450, 452, 454, 456, 458-460, 462, 464, 466, 468, 470, 472, 474, 476, 478, 480, 482, 484, 486, 488, 490, 493, and 494 wherein the 2, 3, 4, or more naturally occurring polypeptides are covalently linked. In one embodiment, each of the 2, 3, 4, or more non-naturally occurring polypeptides are different. In another embodiment, each of the 2, 3, 4, or more non-naturally occurring polypeptides are present in a fusion protein. In each of these aspects, amino acid changes from the reference amino acid sequence may be conservative amino acid substitutions. In another embodiment, at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% of amino acid residues at defined interface positions are invariant compared to the reference amino acid sequence.

In another aspect, the disclosure provides protein scaffolds, comprising
a) a first designed component comprised of any number of monomer A polypeptides and/or monomer B polypeptides, each from different heterodimers, connected into a single component by amino acid linkers.
b) a second designed component, comprising corresponding monomers for each monomer A and/or monomer B in the first designed component one;
wherein the first and second designed components interact to form the protein scaffold, and wherein each monomer A only interacts in the scaffold with its monomer B mate.

In another aspect, the disclosure provides methods of forming the designed heterodimer protein of any embodiment of the disclosure, comprising:
a) providing two of the monomers as unlinked monomers;
b) providing the other two monomers as linked monomers;
whereby the unlinked monomers associate with their respective monomer of the same heterodimer, and not with any of the other monomers.

In another aspect, the disclosure provides designed heterodimer proteins, comprising:
a) asymmetric buried hydrogen bond networks incorporated into regularly repeating backbone structures; and
b) helix hairpin helix monomers wherein the supercoil phases of the helices are fixed at 0, 90, 180, or 270 degrees and the supercoil twist (ω0) and helical twist (ω1) are held constant for either a two layer left handed super coil (ω0=−2.85 and ω1=102.85), or a 5 layer untwisted bundle (ω0=0 and ω1=100)

In another aspect, the disclosure provides fusion proteins comprising a polypeptide of the formula X—B—Z, wherein:
(a) the X domain is a non-naturally occurring polypeptide comprising 1, 2, or 3 alpha helices, wherein the X domain is capable of non-covalently binding to a first target;
(b) the Z domain is a non-naturally occurring polypeptide comprising 1, 2, or 3 alpha helices, wherein the Z domain is capable of non-covalently binding to either (i) a second target that differs from the first target, or (ii) a different non-naturally occurring polypeptide comprising 1, 2, or 3 alpha helices; and
(c) the B domain is an amino acid linker;
wherein a combined number of alpha helices from the X domain and the Z domain is 4, 5, or 6; and
wherein the X domain and the Z domain interact at a binding interface, wherein the binding interface comprises a hydrogen bond network in which at least one side chain in each alpha helix hydrogen of the X domain bonds with a side chain in an alpha helix in the Z domain, and wherein the binding interface comprises a plurality of hydrophobic residues.

In another aspect, the disclosure provides kits or compositions, comprising at least two fusion proteins comprising the formula X—B—Z, wherein
the B domain in each fusion protein is independently a polypeptide linker;
the X domain in each fusion protein comprises a first non-naturally occurring polypeptide comprising 1, 2, or 3 alpha helices;
the Z domain in each fusion protein comprises a second non-naturally occurring polypeptide comprising 1, 2, or 3 alpha helices, wherein a combined number of alpha helices from the X domain and the Z domain in each individual fusion protein is 4, 5, or 6; wherein the X domain and the Z domain interact at a binding interface, wherein the binding interface comprises a hydrogen bond network in which at least one side chain in each X domain alpha helix bonds with a side chain in an alpha helix in the Z domain; wherein
the X domain in a first fusion protein is capable of non-covalently binding to a first target;
the Z domain in a second fusion protein is capable of non-covalently binding to a second target; and
the X domains and Z domains in each individual fusion protein that are not capable of non-covalently binding to the first target or the second target are capable of non-covalently binding to an X or a Z domain of a different fusion protein in the plurality of fusion proteins.

In one embodiment of the fusion proteins, kits, or compositions, each X domain and each Z domain comprise a polypeptide that is 50%, 55%, 60%, 65%, 70%, 75%, 80%, 85%, 90%, 91%, 92%, 93%, 94%, 95%, 96%, 97%, 98%, 99%, or 100% identical to the full length of a polypeptide selected from the group including, but not limited to a polypeptide comprising the amino acid sequence selected from SEQ ID NO:1-290, with the proviso that the X domain and the Z domain do not do not form a heterodimer (a-b) pair. In another embodiment at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% of amino acid residues at defined interface positions of each X domain and each Z domain are invariant compared to the reference amino acid sequence.

In one aspect, the disclosure provides methods, comprising:
(i) contacting a fusion protein according to any embodiment of the disclosure with a biological sample under conditions to promote non-covalent binding of the fusion protein with first target and second target present in the sample, and
(ii) detecting non-covalent binding of the one or more fusion proteins to the first target and/or the second target in the biological sample.

In one embodiment, the method comprises detecting cooperative non-covalently binding of the one or more fusion proteins to the first target and the second target in the biological sample. In another embodiment, the method comprises detecting non-covalent binding of the one or more fusion proteins to the first target or the second target in the biological sample.

In another aspect, the disclosure provides methods for target detection, comprising
(a) contacting a biological sample with at least two fusion proteins, wherein each of the at least two fusion proteins comprises the formula X—B—Z, wherein
each B is independently a polypeptide linker;
each X domain comprises a first non-naturally occurring polypeptide comprising 1, 2, or 3 alpha helices;
each Z domain comprises a second non-naturally occurring polypeptide comprising 1, 2, or 3 alpha helices, wherein a combined number of alpha helices from the X domain and the Z domain in each individual fusion protein is 4, 5, or 6; wherein the X domain and the Z domain interact at a binding interface, wherein the binding interface comprises a hydrogen bond network in which at least one side chain in each X domain alpha helix bonds with a side chain in an alpha helix in the Z domain; wherein
the X domain in a first fusion protein is capable of non-covalently binding to a first target;
the Z domain in a second fusion protein is capable of non-covalently binding to a second target; and
the X domains and Z domains in each individual fusion protein that are not capable of non-covalently binding to the first target or the second target are capable of non-covalently binding to an X or a Z domain of a different fusion protein in the plurality of fusion proteins;
(b) detecting non-covalent binding of the two or more fusion proteins to the first target and/or the second target in the biological sample.

In one aspect, the disclosure provides compositions comprising
(a) a first polypeptide comprising 2 alpha helices, wherein the first polypeptide is capable of non-covalently binding a first target; and
(b) a second polypeptide comprising 2 alpha helices, wherein the first polypeptide is capable of non-covalently binding to the second polypeptide, and wherein the second polypeptide is capable of non-covalently binding a second target that differs from the first target; wherein:
   (i) a binding affinity of the first polypeptide for the first target is approximately equal to a binding affinity of the second polypeptide for the second target; and
   (ii) the binding affinity of the first polypeptide for the first target and the binding affinity of the second polypeptide for the second target are greater than the binding affinity of the first target and the second target for each other.

In one aspect, the disclosure provides compositions comprising
(a) a first polypeptide comprising 2 alpha helices, wherein the first polypeptide is capable of non-covalently binding a first target; and
(b) a second polypeptide comprising 2 alpha helices, wherein the first polypeptide is capable of non-covalently binding to the second polypeptide, and wherein the second polypeptide is capable of non-covalently binding a second target that differs from the first target; wherein:
   (i) a binding affinity of the first polypeptide for the second polypeptide is greater than a binding affinity of the second polypeptide for the second target;
   (ii) a binding affinity of the first polypeptide for the first target is approximately equal to a binding affinity of the second polypeptide for the second target; and
   (iii) the binding affinity of the first polypeptide for the first target and the binding affinity of the second polypeptide for the second target are greater than the binding affinity of the first target and the second target for each other.

In another aspect, the disclosure provides compositions comprising
(a) a first polypeptide comprising 4 alpha helices, wherein the first polypeptide is capable of non-covalently binding a first target; and
(b) a second polypeptide comprising 4 alpha helices, wherein the second polypeptide is capable of non-covalently binding a second target that differs from the first target; wherein:
   (i) a binding affinity of the first target for the second target is greater than a binding affinity of the first polypeptide for the first target;
   (ii) a binding affinity of the first polypeptide for the first target is approximately equal to a binding affinity of the second polypeptide for the second target; and
   (iii) the sum of the binding affinity of (A) the first polypeptide for the first target and (B) the binding affinity of the second polypeptide for the second target, is greater than the binding affinity of the first target and the second target.

In various embodiments for each composition of the disclosure, the composition may further comprise the first target and the second target, and the first target and/or the second target further may comprise one or more effector polypeptide domains. In one embodiment, the first polypeptide and/or the second polypeptide comprise a polypeptide that is 50%, 55%, 60%, 65%, 70%, 75%, 80%, 85%, 90%, 91%, 92%, 93%, 94%, 95%, 96%, 97%, 98%, 99%, or 100% identical to the full length of a polypeptide selected from the group including, but not limited to a polypeptide comprising the amino acid sequence selected from the group consisting of SEQ ID NO:1-290, or the group consisting of SEQ ID NOS:1-290, 331, 332, 334, 336-422, 424, 426, 428, 430, 432, 434, 436, 438, 440, 442, 444, 446, 448, 450, 452, 454, 456, 458-460, 462, 464, 466, 468, 470, 472, 474, 476, 478, 480, 482, 484, 486, 488, 490, 493, and 494. In another embodiment, the first target and/or the second target each comprise a polypeptide that is 50%, 55%, 60%, 65%, 70%, 75%, 80%, 85%, 90%, 91%, 92%, 93%, 94%, 95%, 96%, 97%, 98%, 99%, or 100% identical to the full length of a polypeptide selected from the group including, but not limited to a polypeptide comprising the amino acid sequence selected from the group consisting of SEQ ID NO:1-290, or the group consisting of SEQ ID NOS:1-290, 331, 332, 334, 336-422, 424, 426, 428, 430, 432, 434, 436, 438, 440, 442, 444, 446, 448, 450, 452, 454, 456, 458-460, 462, 464, 466, 468, 470, 472, 474, 476, 478, 480, 482, 484, 486, 488, 490, 493, and 494, with the proviso that the first target forms a heterodimer (a-b) pair with the first polypeptide, and the second target forms a heterodimer (a-b) pair with the second polypeptide. In another embodiment, the compositions are contacted with a biological sample and binding is detected, such as detecting an output signal caused by actions of effector polypeptides upon binding.

The disclosure also provides nucleic acids encoding the polypeptides, proteins, and fusion proteins of the disclosure; expression vectors comprising the nucleic acids operatively linked to a promoter; and host cells comprising the nucleic acids, expression vectors, and/or polypeptides, proteins, fusion proteins, scaffolds, and designed heterodimer pairs of the disclosure.

### DESCRIPTION OF THE FIGURES

FIGS. 1A to 1H shows modular heterodimer design. FIG. 1A shows individual helix generation: the helical phase (A 1), supercoil radius (R) and offset along the Z-axis (Z offset) were exhaustively sampled; a total of 11 free parameters since there is no z offset for the first helix. FIG. 1B shows top-down view of the parallel twisted backbone. FIG. 1C shows representative hydrogen bond networks identified using HBNET™. FIG. 1D shows matches of multiple HBNET™ containing heptads to a single full length backbone. FIG. 1E shows addition of loops to connect the 4 helices into two helix hairpins. FIGS. 1F, 1G, and 1H show SEC trace, CD spectra and (inset) temperature melt, and SAXS (black, experimental SAXS data; red, spectra computed from the designed backbones) profile of the design DHD37_ABXB. Experiments were performed once.

FIGS. 2A to 2F show structural characterization of designed heterodimers. FIGS. 2A-2D show crystal structures superimposed on design models with monomers; cross-sections on backbones (left) indicate locations of designed hydrogen-bond networks (middle panels). Solid and dashed boxes compare networks in design model and crystal structure. Black boxes compare overall hydrophobic packing. FIG. 2A shows DHD_131, 2.4 Å resolution with 1.0 Å Cu RMSD. FIG. 2B shows DHD37_1:234, 3.3 Å resolution with 1.4 Å RMSD. FIG. 2C shows DHD_127, 1.8 Å resolution with 1.7 Å RMSD. FIG. 2D shows DHD_15, 3.4 Å resolution with 0.9 Å RMSD; hydrogen bond networks were not well resolved. FIGS. 2E-2F show DHD_39 and DHD_120 backbones and designed hydrogen bond networks. Experimental SAXS data (black) are similar to spectra computed from the designed backbones.

FIGS. 3A to 3C shows new functionality from DHD combinations. FIG. 3A shows induced dimerizer formed from “b” component of DHD13_XAAA fused to “b” component of DHD37 ABXB with an intervening flexible linker. The “a” components of the two heterodimers are brought into close proximity by the heterodimerizer. FIG. 3B shows Y2H data on 4 induced dimerization systems. For each pair of bars: left, without heterodimerizer fusion; right, with heterodimerizer fusion. Dashed line indicates background growth with unfused AD and DBD. Data are mean±s.d. from 3 biological repeats. FIG. 3C shows 9_a, 13_XAAA_a and 37_ABXB_a were covalently linked to form a scaffold, recruiting 9_b (hexahistidine tagged), 13_XAAA_b and 37_ABXB_b.

FIGS. 4A to 4C show all-against-all orthogonality assessment. FIG. 4A shows Y2H for 21 heterodimers show heterodimer formation with little homodimer formation. First letter at bottom indicates monomer fused to AD, second letter, to DBD. FIG. 4B shows Y2H all by all testing of 9 pairs of heterodimers, colors indicate growth. Boxes indicate designed cognate heterodimer pairs, dashed black box indicates a set of 6 orthogonal heterodimers. FIG. 4C shows Off-target binding of DHD15_a and DHD13_XAAA_b, in the absence (left) or presence (right) of DHD15_b and DHD13_XAAA_a. Data are mean±s.d.. Red dashed line indicates background growth with unfused AD and DBD.

FIGS. 5A to 5B show example HBNets resulting from the systematic search. FIG. 5A shows overlay of 50 backbones with different Crick parameters for each helix. FIG. 5B shows example hydrogen bond networks from the systematic search, each involving at least 4 residues and contacting all 4 helices.

FIGS. 6A to 6C show thermal and chemical denaturation of DHDs. FIGS. 6A and 6B show CD spectra for thermal denaturation of DHD_15 and DHD_20, respectively. Top, wavelength scan at 25° C., 75° C., 95° C., and final 25° C. Designs were alpha helical and stable up to 95° C. Bottom, CD temperature melts, monitoring absorption at 222 nm as temperature was increased from 25° C. to 95° C. FIG. 6C shows GdnHCl denaturation of DHD_127 by CD monitoring absorption at 222 nm. All CD experiments were performed once.

FIG. 7A to 7B show backbone and hydrogen bond network permutations. FIG. 7A shows on a 2+2 backbone (left), two loops were designed to connect the 4 helices into a single monomer in 2 different ways (middle), after which 4 different cut points were introduced to generate 4 possible backbone permuted heterodimers of a single helix and a three helix bundle (3+1 heterodimers, right). For example, 2:134 refers to a heterodimer where the original helix 2 is a single helix, and helices 1, 3, and 4 were connected into a 3 helix bundle. FIG. 7B shows hydrogen bond network permutation. Each unique network was assigned a letter (Networks “A” and “B” in this case), and with the hydrophobic packing assigned X. The backbone on the left reads “ABXB”, with its first heptad accommodates network “A”, its second and fourth heptad accommodate network “B”, and its third heptad accommodates hydrophobic packing only.

FIGS. 8A to 8H show crystal structure of the domain swapped DHD_15 and biophysical characterization of higher order oligomers. FIG. 8A shows crystal structure of DHD_15 at pH 6.5, with 2.25 Å resolution. FIG. 8B shows superposition of design models in color onto both halves of the crystal structure in white, with backbone RMSD of 1.83 Å. FIGS. 8C to 8F show SEC traces of the induced dimerization DHD_9-13 fusion, DHD_15-37 fusion, DHD_13-37 fusion, and the scaffolding complex in FIG. 3C (the peak at around 15 mL corresponds to the fully assembled complex, followed by a peak representing excess of individual components). FIG. 8G shows CD thermal melt curves for the scaffolding complex in FIG. 3C. Wavelength scan was performed at 25° C., 75° C., 95° C., and final 25° C. Design was alpha helical and stable up to 95° C. FIG. 8H, CD chemical denaturation profile of the scaffolding complex in FIG. 3C. 2 (FIG. 8C to 8F) or 1 (FIG. 8G to 8H) biologically independent repeats were performed.

FIGS. 9A to 91 show Y2H all-against-all assay of 16 DHDs. FIG. 9A shows Y2H assay with cell growth on agar plates containing 100 mM 3-AT, lacking tryptophan, leucine and histidine. Plates were imaged at Day 5. White, no growth on agar plates; grey, weak growth forming non-circular colonies; black, strong growth. FIG. 9B shows Y2H result by growing yeast culture in liquid media containing 100 mM 3-AT, lacking tryptophan, leucine and histidine. OD 600 values were measured at Day 2 to evaluate cell growth. FIG. 9C shows an additional set of DHDs tested by Y2H showing improved orthogonality. FIG. 9D shows distribution of OD 600 values for non-cognate interactions in FIG. 9B, the majority of cells grew to OD 600 values less than 0.4, indicating weak interactions for non-cognate binding. FIG. 9E shows more buried bulky polar residues strongly correlates with design success. f, Successful designs tend to have bigger polar interface surface area. FIG. 9G shows designs with better hydrophobic packing (as reported by the ROSETTA™ filter value Average Degree on Ile, Leu and Val residues) tend to have a higher chance of being constitutive heterodimers. Two (FIGS. 9A to 9C) independent experiments were performed.

FIG. 10 shows hydrogen bond network sequence motifs of the set of 6 orthogonal pairs in Y2H experiments. Letters patches mark the location of hydrogen bond network forming residues on the backbones, and indicate residue identities.

FIGS. 11A to 11H show cooperativity of CIPHR logic gates. FIG. 11A shows backbone structure of A:A′ heterodimer building block, with hydrogen bond network detail in inset. Bottom right, condensed representation used throughout figures. FIG. 11B shows thermodynamic cycle describing the induced dimerization system. FIG. 1C shows simulation of the induced dimerization system under thermodynamic equilibrium. A and B′ monomers were held constant while titrating in various initial amounts of the A′-B dimerizer proteins. If binding is not cooperative (small c), the final amount of trimeric complexes decreases when the dimerizer protein is in excess. FIG. 11D shows equilibrium denaturation experiments monitored by CD for designs with 6- and 12-amino acid (AA) linkers. Circles represent experimental data, and lines are fits to the 3-state unimolecular unfolding model. Design models are shown on the side. FIG. 11E shows experimental SAXS profile of 1′-2′ with a 6-residue linker (in black), fitted to the calculated profile of 1:1′ heterodimer. FIG. 11F shows an induced dimerization system using a 6-residue linker. FIG. 11G shows a two-input AND gate schematic. FIG. 11H shows a three-input AND gate.

FIGS. 12A to 12G show CIPHR two input logic gates. FIG. 12A shows CIPHR gates are built from DHDs (top) with monomers or covalently connected monomers as inputs (left); some gates utilize only the designed cognate interactions (left side of middle panel), while others take advantage of observed inter and intramolecular binding affinity hierarchies (right side of middle panel). FIGS. 12B and 12C show two-input AND (12B) and OR (12C) CIPHR logic gates based on orthogonal DHD interactions. FIGS. 12D to 12G show NOT (12D), NOR (12E), XNOR (12F), and NAND (12G) CIPHR logic gates made from multispecific and competitive protein binding. For each gate, black dots represent individual Y2H growth measurement corrected over background growth, with their average values shown in bars. * indicates no yeast growth over background. 0s and 1s in the middle and right blocks represent different input states and expected outputs, respectively.

FIGS. 13A to 13E show three-input CIPHR logic gates. FIG. 13A shows schematic of a three-input AND gate. FIG. 13B shows schematic of a three-input OR gate. FIG. 13C shows Y2H results confirmed activation of the 3-input OR gate with either of the inputs. FIG. 13D shows schematic of a DNF gate. FIG. 13E shows Y2H results confirmed proper activation of the gate. For each gate, black dots represent individual measurements corrected over background growth, with their average values shown in bars.

FIG. 14A shows molecular implementation of the cooperative induced dimerization system, binding only occurs when all three components are present. FIG. 14B shows size exclusion chromatography profiles of 1′-2′ variants with 0, 2, 6, 12, and 24 amino acids in the flexible linker connecting 1′ and 2.

FIGS. 15A and 15B show binding affinity gradient from individual Y2H experiments. FIG. 15A shows the 8:8′ heterodimer binds more tightly than the homodimers of its monomers. FIG. 15B shows binding affinity gradient among the monomers of 1:1′, 9:9′, and 10:10′ pairs.

FIG. 16 shows exemplary heterodimer proteins comprising combinations of monomer A and monomer B.

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2^{nd} Ed. (R. I. Freshney. 1987. Liss, Inc. New York, NY), Gene Transfer and Expression Protocols, pp. 109-128, ed. E. J. Murray, The Humana Press Inc., Clifton, N.J.), and the Ambion 1998 Catalog (Ambion, Austin, TX).

As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

It is understood that wherever aspects are described herein with the language “comprising,” otherwise analogous aspects described in terms of “consisting of” and/or “consisting essentially of” are also provided.

The term “interface residue” or “interface position”, as used herein, means amino acid residues or positions that are interacting between at least two monomers in heterodimer, heterotrimer, heterotetramer, etc. The interaction comprises a hydrogen bond network in which at least a hydrogen from an alpha helix in the first monomer binds to a side chain in an alpha helix in the second monomer. In some aspects, the interaction comprises at least one hydrogen bond, at least two hydrogen bonds, at least three hydrogen bonds, at least four hydrogen bonds, at least five hydrogen bonds, at least six hydrogen bonds, at least seven hydrogen bonds, at least eight hydrogen bonds, at least nine hydrogen bonds, and at least ten hydrogen bonds. In some aspects, the interface residue comprises hydrophobic residues.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

In a first aspect the disclosure provide designed heterodimer proteins, comprising:
(a) a monomer A polypeptide, wherein the monomer A polypeptide is a non-naturally occurring polypeptide comprising 1-5 alpha helices connected by amino acid linkers; and
(b) a monomer B polypeptide, wherein the monomer B polypeptide is a non-naturally occurring polypeptide comprising 1-5 alpha helices connected by amino acid linkers,
wherein monomer A and monomer B non-covalently interact to form the designed heterodimer protein.

The disclosure provides designed heterodimer proteins according to this aspect formed by the non-covalent interaction of two different alpha-helix-containing polypeptides (monomer A and monomer B).

By doubling the interaction surface area of protein coiled coils with an additional helix, and incorporating modular hydrogen bond networks, a wide range of heterodimeric interaction specificities can be achieved, as described herein. Millions of helical backbones with varying degrees of supercoiling around a central axis were generated and searched for those accommodating extensive hydrogen bond networks, followed by connecting the helices with short loops and designing the remainder of the sequence. As disclosed in the examples that follow, designs expressed in E coli exclusively formed heterodimers, and crystal structures of exemplary designs fit the computational models and confirmed the designed hydrogen bond networks. Following mixing of independently expressed and purified heterodimer designs, the vast majority of the interactions observed by native mass spectrometry were between the designed cognate pairs. The large sets of orthogonal polypeptide heterodimers disclosed herein can be used, for example, to generate synthetic protein logic gates, transcriptional networks and other synthetic biology applications.

Heterodimers are generally more useful than homodimers in bioengineering because of their ability to bring together two different entities (often fusion proteins). A long standing challenge in the field has been to come up with a set of orthogonally interacting protein heterodimers—monomers that selectively form cognate pairs and in the meantime avoid binding to other non-cognate monomers. Disclosed herein include such sets of orthogonal heterodimers, which can be programmably expanded into an even bigger set. The ability to bring together two different fusion proteins via genetically fused heterodimers allowed the design of protein-based logic gates, as also disclosed herein.

In one embodiment, monomer A and monomer B have their interaction specificity determined by at least one designed hydrogen bond network at the interface between monomer A and monomer B. In some aspects, (i) monomer A comprises 1 helix, and monomer B comprises 1 helix; (ii) monomer A comprises 1 helix and monomer B comprises 2 helices; (iii) monomer A comprises 1 helix and monomer B comprises 3 helices, (iv) monomer A comprises 1 helix and monomer B comprises 4 helices; or (v) monomer A comprises 1 helix and monomer B comprises 5 helices. In some aspects, (i) monomer A comprises 2 helices, and monomer B comprises 1 helix; (ii) monomer A comprises 2 helices and monomer B comprises 2 helices; (iii) monomer A comprises 2 helices and monomer B comprises 3 helices, (iv) monomer A comprises 2 helices and monomer B comprises 4 helices; or (v) monomer A comprises 2 helices and monomer B comprises 5 helices. In some aspects, (i) monomer A comprises 3 helices, and monomer B comprises 1 helix; (ii) monomer A comprises 3 helices and monomer B comprises 2 helices; (iii) monomer A comprises 3 helices and monomer B comprises 3 helices, (iv) monomer A comprises 3 helices and monomer B comprises 4 helices; or (v) monomer A comprises 3 helices and monomer B comprises 5 helices. In some aspects, (i) monomer A comprises 4 helices, and monomer B comprises 1 helix; (ii) monomer A comprises 4 helices and monomer B comprises 2 helices; (iii) monomer A comprises 4 helices and monomer B comprises 3 helices, (iv) monomer A comprises 4 helices and monomer B comprises 4 helices; or (v) monomer A comprises 4 helices and monomer B comprises 5 helices. In some aspects, (i) monomer A comprises 5 helices, and monomer B comprises 1 helix; (ii) monomer A comprises 5 helices and monomer B comprises 2 helices; (iii) monomer A comprises 5 helices and monomer B comprises 3 helices, (iv) monomer A comprises 5 helices and monomer B comprises 4 helices; or (v) monomer A comprises 5 helices and monomer B comprises 5 helices.

Any suitable amino acid linkers can be used to separate the alpha helices in each monomer. The length and amino acid content may vary based on an intended use, and can be determined by one of skill in the art based on the teachings herein. The polypeptide monomers may include any other useful sequences, including detectable tags and purification tags. In one non-limiting embodiment, at least one of monomer A and monomer B comprises a hexahistidine tag.

In another embodiment, the disclosure provides heterodimers, comprising:
(a) a monomer A polypeptide, wherein the monomer A polypeptide is a non-naturally occurring polypeptide comprising 1-5 alpha helices, wherein adjacent alpha helices may optionally be connected by an amino acid linker; and
(b) a monomer B polypeptide, wherein the monomer B polypeptide is a non-naturally occurring polypeptide comprising 1-5 alpha helices, wherein adjacent alpha helices may optionally be connected by an amino acid linker;
wherein the monomer A polypeptide and the monomer B polypeptide non-covalently interact to form the designed heterodimer protein.

In one embodiment, the monomer A polypeptide and the monomer B polypeptide have their interaction specificity determined by at least one hydrogen bond network at the interface between the monomer A polypeptide and the monomer B polypeptide. In another embodiment,
(i) the monomer A polypeptide comprises 2 alpha helices, and the monomer B polypeptide comprises 3 alpha helices;
(ii) the monomer A polypeptide comprises 3 alpha helices and the monomer B polypeptide comprises 3 alpha helices;
(iii) the monomer A polypeptide comprises 3 alpha helices and the monomer B polypeptide comprises 4 alpha helices,
(iv) the monomer A polypeptide comprises 4 alpha helices and the monomer B polypeptide 3 alpha helices;
(v) the monomer A polypeptide comprises 4 alpha helices and the monomer B polypeptide comprises 4 alpha helices;
(vi) the monomer A polypeptide comprises 5 alpha helices and the monomer B polypeptide comprises 4 alpha helices;
(vii) the monomer A polypeptide comprises 4 alpha helices and the monomer B polypeptide comprises 5 alpha helices;
(viii) the monomer A polypeptide comprises 5 alpha helices and the monomer B polypeptide comprises 5 alpha helices;
(ix) the monomer A polypeptide comprises 2 alpha helices and the monomer B polypeptide comprises 2 alpha helices; or
(x) the monomer A polypeptide comprises 3 alpha helices and the monomer B polypeptide comprises 2 alpha helices.

In one embodiment of any of the above embodiments,
(i) monomer A comprises a polypeptide having at least 70%, 75%, 80%, 85%, 90%, 91%, 92%, 93%, 94%, 95%, 96%, 97%, 98%, 99%, or 100% sequence identity along the length of the amino acid sequence of an odd-numbered SEQ ID NO selected from the group consisting of selected from the group SEQ ID NOS: 1-290; and
(ii) monomer B comprises a polypeptide having at least 70%, 75%, 80%, 85%, 90%, 91%, 92%, 93%, 94%, 95%, 96%, 97%, 98%, 99%, or 100% sequence identity along the length of the amino acid sequence of an even-numbered SEQ ID NO selected from the group consisting of selected from the group SEQ ID NOS: 1-290, wherein the even-numbered SEQ ID NO is the binding partner of the odd-numbered SEQ ID NO. in step (i).

The amino acid sequences of SEQ ID NOS: 1-290 are provided in Table A. The “binding partners” are sequentially numbered (and similarly named) as shown in the Table. For example, SEQ ID NO:1 (DHD9 A) and SEQ ID NO:2 (DHD9 B) are binding partners, so that if monomer A comprises the polypeptide having at least 70% 75%, 80%, 85%, 90%, 91%, 92%, 93%, 94%, 95%, 96%, 97%, 98%, 99%, or 100% sequence identity along the length of the amino acid sequence of SEQ ID NO:1, then monomer B comprises the polypeptide having at least 70%, 75%, 80%, 85%, 90%, 91%, 92%, 93%, 94%, 95%, 96%, 97%, 98%, 99%, or 100% sequence identity along the length of the amino acid sequence of SEQ ID NO:2. Similarly, SEQ ID NOS:3-4 are binding partners, SEQ ID NO:5-6 are binding partners . . . SEQ ID NOS:289-290 are binding partners. Those of skill in the art will clearly understand what is meant by binding partner based on the teachings herein.

TABLE 1A

| Design | Oligomerization | | |
|---|---|---|---|
| name | State | Chain | Design sequence |
| DHD9 | Heterodimer | a | GSPKEEARELIRKQKELIKEQKKLIKEAKQKSDSRDAERIWKRSREINRES |
| | | | KKINKRIKELIKS SEQ ID NO: 1 |
| DHD9 | Heterodimer | b | PKKEAEELAEESEELHDRSEKLHERAEQSSNSEEARKILEDIERISERIEE |
| | | | ISDRIERLLRS SEQ ID NO: 2 |
| DHD13_ | Heterodimer | a | GTKEDILERQRKIIERAQEIHRRQQEILEELERIIRKPGSSEEAMKRMLKL |
| XAAA | | | LEESLRLLKELLELSEESAQLLYEQR SEQ ID NO: 3 |
| DHD13_ | Heterodimer | b | GTEKRLLEEAERAHREQKEIIKKAQELHRRLEEIVRQSGSSEEAKKEAKKI |
| XAAA | | | LEEIRELSKRSLELLREILYLSQEQKGSLVPR SEQ ID NO: 4 |
| DHD13_ | Heterodimer | a | TKEDILERQRKIIERAQEIIRRQQEILEELERIIRKPGSSEEAMKRMLKLL |
| XAXA | | | EESLRLLKELLELLEESAQLLYEQR SEQ ID NO: 5 |
| DHD13_ | Heterodimer | b | GSTEKRLLEEAERAHREAKEIIKKAQELHRRLEEIVRQSGSSEEAKKEAKK |
| XAXA | | | ILEEIRELSKRLLELLREILYLSQEQK SEQ ID NO: 6 |
| DHD13_ | Heterodimer | a | TKEDILERARKIIERAQEIHRRQQEILEELERIIRKPGSSEEAMKRMLKLL |
| XAAX | | | EESLRLLKELLELSEELAQLLYEQR SEQ ID NO: 7 |
| DHD13_ | Heterodimer | b | GSTEKRLLEEAERAIREQKEIIKKAQELHRRLEEIVRQSGSSEEAKKEAKK |
| XAAX | | | ILEEIRELSKRSLELLREILYLLQEQK SEQ ID NO: 8 |
| DHD13_ | Heterodimer | a | TKEDILERQRKIIERAQEIHRRQQEILEELEYIIR SEQ ID NO: 9 |
| 2:341 | | | |
| DHD13_ | Heterodimer | b | MSEEAMKRMLKLLEESLRLLKELLELSEESAQLLYEQRKANNGSETEKRLL |
| 2:341 | | | EEAERAHREQKEIIKKAQELHRRLEEIVRQSGSSEEAKKEAKKILEEIREL |
| | | | SKRSLELLREILYLSQEQK SEQ ID NO: 10 |
| DHD13_ | Heterodimer | a | MTKEDILERQRKIIERAQEIHRRQQELKEQEKIIRKPGSSEEAMKRSLKL |
| AAAA | | | IEESLRLLKELLELSEESAQLLYEQR SEQ ID NO: 11 |
| DHD13_ | Heterodimer | b | GTEKRLLEEAERAHREQKEIIKKAQELHKELTKIHQQSGSSEEAKKRALKI |
| AAAA | | | SQEIRELSKRSLELLREILYLSQEQK SEQ ID NO: 12 |
| DHD13_ | Heterodimer | a | TKEDILERQRKIIERAQEIHRRQQEILKRSEEIIRKPGSSEEALETLRELQ |
| BAAA | | | EESLRLLKELLELSEESAQLLYEQR SEQ ID NO: 13 |
| DHD13_ | Heterodimer | b | GSTEKRLLEEAERAHREQKEIIKKAQELHRRTEEIIRQSGSSEEAKDELRR |
| BAAA | | | IQEEIRELSKRSLELLREILYLSQEQK SEQ ID NO: 14 |
| DHD13_ | Heterodimer | a | TTKRYLEEAERAHREQKEIIKKAQELHRRLEEIVRQ SEQ ID NO: 15 |
| 4:123 | | | |
| DHD13_ | Heterodimer | b | GSSEEAKKEAKKILEEIRELSKRSLELLREILYLSQQVNDVDEKALERQRK |
| 4:123 | | | IIERAQEIHRRQQEILEELERIIRKPGSSEEAMKRMLKLLEESLRLLKELL |
| | | | ELSEESAQLLYEAR SEQ ID NO: 16 |
| DHD13_ | Heterodimer | a | EAMKRMLKLLEESLRLLKELLELSEESAQLLYEAR SEQ ID NO: 17 |
| 1:234 | | | |
| DHD13_ | Heterodimer | b | TTKRYLEEAERAHREQKEIIKKAQELHRRLEEIVRQSGSSEEAKKEAKKIL |
| 1:234 | | | EEIRELSKRSLELLREILYLSQQVNDVDEKALERQRKIIERAQEIHRRQQE |
| | | | ILEELERIIRKPGS SEQ ID NO: 18 |
| DHD15 | Heterodimer | a | TREELLRENIELAKEHIEIMREILELLQKMEELLEKARGADEDVAKTIKEL |
| | | | LRRLKEIIERNQRIAKEHEYIARERS SEQ ID NO: 19 |
| DHD15 | Heterodimer | b | GTERKLLERSRRLQEESKRLLDEMAEIMRRIKKLLKKARGADEKVLDELRK |
| | | | IIERIRELLDRSRKIHERSEEIAYKEE SEQ ID NO: 20 |
| DHD20 | Heterodimer | a | GDRQELIRRNIELLKEHIKILEEISQLIEELSELLDKSSSEEVVKRYKKIL |
| | | | ERYKQLLRKSQEIHKESSEIAKKES SEQ ID NO: 21 |
| DHD20 | Heterodimer | b | GDEQKLIERSQRMQKESLELLKEIIKILDTIEKLLDKPDSEELLDTIKKLH |
| | | | DTLKKIHDRNKKLLKEHEEILRQRSGSLVPR SEQ ID NO: 22 |
| DHD21 | Heterodimer | a | DKEEEYKRLLDEIKEILKESKEVLKDSKRVLEDIKRKVPDDDLVKLLEKHV |
| | | | RLLEEHVKLLEQLIREAEKSSK SEQ ID NO: 23 |
| DHD21 | Heterodimer | b | QGSSAEELLKKIKESEKKIRDSLRKIKEIIKKSRKEGVDDKQLDLIRKVVE |
| | | | SHRDLLRLHRDLLRLLREETS SEQ ID NO: 24 |
| DHD25 | Heterodimer | a | DIDESIKEVEKLLEEVEQSLQKLDDSLKKLLEKVNQDPDVDDSVRKIVKRH |
| | | | VEILKRHEEVLKRLIEVVKEHTKTVK SEQ ID NO: 25 |
| DHD25 | Heterodimer | b | GSDREEVHKEIVKLIREIIKIHKKILKIHEKIKNGEIDPSEILKLSEEIKK |
| | | | LTDTIIKIIEDLEQLTRDLRR SEQ ID NO: 26 |
| DHD27 | Heterodimer | a | DRKEIVKRHQKVVELLKESSKLLRESSKLLQRLLDKTGDENLQKAVDDQDK |
| | | | AIKRQETAIRKSQEASKKLD SEQ ID NO: 27 |
| DHD27 | Heterodimer | b | DNSEEIKKVAKTSREVAEYSERVAKENDKVVKTLEEGKIDESELLRLLEES |
| | | | IKIFDTALKLHEEAYKLHQDLVRKVS SEQ ID NO: 28 |
| DHD30 | Heterodimer | a | DESEAASVAIESVQILVESVKLLEESVRILLDAVKKNGVEDLLRVAQRWEK |
| | | | LVDEWLKVVKRWLDNVRDIQR SEQ ID NO: 29 |
| DHD30 | Heterodimer | b | GSDKAEEVEKSVRKIEESIKKIRKSIKKAEDAVQLLKEGKIDAKDELRIVR |
| | | | EDLEVVKEDVEIVKEDVENVREFSS SEQ ID NO: 30 |
| DHD33 | Heterodimer | a | SDKEVSDKLLKASKKLLKVSEELLEVVRRLLKALKDDELIKKIADLLRKII |
| | | | DKDKKFIRTSEEIVKESR SEQ ID NO: 31 |
| DHD33 | Heterodimer | b | GSDLKEVLKTVEEAVKEIIKSSEELLQISRKILEISRVGVDEHEYISAIRE |
| | | | YLKALEKHIQILKKFIEILKELIRAVS SEQ ID NO: 32 |
| DHD34_ | Heterodimer | a | SKEEIDKIVKKHKKKIEEHKKKVDELKKLVEEHDKRVSQDKDDKVKKLSEE |
| XAAXA | | | VKKIIKRLEEVSKRLEEVSKKLLKVISDKR SEQ ID NO: 33 |
| DHD34_ | Heterodimer | b | GSNDEELKKILETLDRILKKLDKILTRLIEVLKKSEDPNLDDKDYTELVKQ |
| XAAXA | | | FIELIKKYEEVVKEYEEVVRQLIRLES SEQ ID NO: 34 |
| DHD34_ | Heterodimer | a | SKEEIDKIVKKHKKKIEELKKLVDELKKLVEEHDKRVSQDKDDKVKKLSEE |
| XAXXA | | | VKKIIKRVEEVAKRLEEVSKKLLKVISDKR SEQ ID NO: 35 |
| DHD34_ | Heterodimer | b | GSNDEELKKILETLDRILKKLEKILTRLIEVLKKSEDPNLDDKDYTELVKQ |
| XAXXA | | | FIELIKKFEEVIKEYEEVVRQLIRLES SEQ ID NO: 36 |
| DHD34_ | Heterodimer | a | SKEEIDKIVKKHKKKIEEHKKKVDEHKKLVEEHDKRVSQDKDDKVKKLSEE |
| XAAAA | | | LKKISKRLEEVSKRLEEVSKKLLKVISDKR SEQ ID NO: 37 |
| DHD34_ | Heterodimer | b | GSNDEELKKILETLDRILKKLDKILTRLDEVLKKSEDPNLDDKDYTELVKQ |
| XAAAA | | | YIELVKKYEEVVKEYEEVVRQLIRLES SEQ ID NO: 38 |
| DHD36 | Heterodimer | a | DHSRKLKEILDRLRKHVKRLKEHLDELRDLVRQVPEDKLLEHVVKLSDKIL |
| | | | QISERAVREFTKSVDKDS SEQ ID NO: 39 |
| DHD36 | Heterodimer | b | GSDKKDELERILDEIRRLIERLDEILSRLNKLLELLKHGVPNAKEVVKDYI |
| | | | RLLKEYLELVKEFLKLVKRHADLVS SEQ ID NO: 40 |
| DHD37_ | Heterodimer | a | DSDEHLKKLKTFLENLRRHLDRLDKHIKQLRDILSENPEDERVKDVIDLSE |
| ABXB | | | RSVRIVKTVIKIFEDSVRKKE SEQ ID NO: 41 |
| DHD37_ | Heterodimer | b | GSDDKELDKLLDTLEKILQTATKIIDDANKLLEKLRRSERKDPKVVETYVE |
| ABXB | | | LLKRHEKAVKELLEIAKTHAKKVE SEQ ID NO: 42 |
| DHD37_ | Heterodimer | a | MDEEDHLKKLKTHLEKLERHLKLLEDHAKKLEDILKERPEDSAVKESIDEL |
| BBBB | | | RRSIELVRESIEIFRQSVEEEE SEQ ID NO: 43 |
| DHD37_ | Heterodimer | b | GDVKELTKILDTLTKILETATKVIKDATKLLEEHRKSDKPDPRLIETHKKL |
| BBBB | | | VEEHETLVRQHKELAEEHLKRTR SEQ ID NO: 44 |
| DHD37_ | Heterodimer | a | DSDEHLKKLKTFLENLRRHLDRLDKLLKELRDILSENPEDERVKDVIDELE |
| XBXB | | | RVIRIVKTVIKIFEDSVRKKE SEQ ID NO: 45 |
| DHD37_ | Heterodimer | b | GSDDKELDKLLDTLEKILQTATKIIDDLNKVLEKLRRSERKDPKVIETVVE |
| XBXB | | | LLKRHEKAVKELLEIAKTHAKKVE SEQ ID NO: 46 |
| DHD37_ | Heterodimer | a | DSDEHLKKLKTFLENLRRLEDLLDKHIKQLRDILSENPEDERVKDVIDLSE |
| AXXB | | | RVVRTVKTVIKIFEDSVRKKE SEQ ID NO: 47 |
| DHD37_ | Heterodimer | b | GSDDKELDKLLDTLEKILQTATKVVDDANKLLEKLRRSERKDPKVVETYVE |
| AXXB | | | LLKRLEKLIKELLEIAKTHAKKVE SEQ ID NO: 48 |
| DHD37_ | Heterodimer | a | DSDEHLKKLKTFLENLRRHLDRLDKHIKQLRDILSEN SEQ ID NO: 49 |
| 3:124 | | | |
| DHD37_ | Heterodimer | b | EDERVKDVIDLSERSVRIVKTVIKIFEDSVRKLEKTKPDSKTAKELDKLLD |
| 3:124 | | | TLEKILQTATKIIDDANKLLEKLRRSERKDPKVVETYVELLKRHEKAVKEL |
| | | | LEIAKTHAKKVE SEQ ID NO: 50 |
| DHD37_ | Heterodimer | a | DSDEHLYKLKTFLENLRRHLDRLDKHIKQLRDILSENPEDERVKDAIDLSE |
| 1:234 | | | RSVRIVKTVIKIFEDSVRKKEKRPIDKRDDKELDKLLDTLEKILQTATKII |
| | | | DDANKLLEYLRR SEQ ID NO: 51 |
| DHD37_ | Heterodimer | b | GDPKVVETYVELLKRHEKAVKELLEIAKTHAKKVE SEQ ID NO: 52 |
| 1:234 | | | |
| DHD37_ | Heterodimer | a | DSDEHLDRLDKHLKKLKTFLENLRRHIKQLRDILSENPEDERVKDVIDLSK |
| AXBB | | | TVIKIFEDSVRKKERSVRIVE SEQ ID NO: 53 |
| DHD37_ | Heterodimer | b | GSDDKEATKIIDDLDKLLDTLEKILQTANKLLEKLRRSERKDPKVVETYVK |
| AXBB | | | AVKELLEIAKTHAELLKRHEKKVE SEQ ID NO: 54 |
| DHD37_ | Heterodimer | a | DSDEHIKQLRDHLDRLDKHLKKLKTFLENLRRILSENPEDERVKTVIKIFE |
| XBBA | | | DSVRKKERSVRIVKDVIDLSE SEQ ID NO: 55 |
| DHD37_ | Heterodimer | b | GSDDKEANKLLEKATKIIDDLDKLLDTLEKILQTLRRSERKDPKAVKELLE |
| XBBA | | | IAKTHAELLKRHEKVVETYVKKVE SEQ ID NO: 56 |
| DHD39 | Heterodimer | a | DHSRKLEEILDRLRKHVKRLLEHLRELLSLVKENPEDKDLVEVLELSLAIL |
| | | | RRSLEAVEAFLKSVTKKDPDDEDLRRKADEIRKEVEEIKKSLAEVEKEIYK |
| | | | LK SEQ ID NO: 57 |
| DHD39 | Heterodimer | b | GSSADDVLEDILKIIRELIEILDQILSLLNQLLKLLRHGVPNAKKVVEKYK |
| | | | EILELYLQLVSLFLKIVKTHADAVSGKIDKKAEEEIKKEEEKIKEKLRQAK |
| | | | DILKKLQEEIDKTR SEQ ID NO: 58 |
| DHD40 | Heterodimer | a | DRDAHLYKLLTFLEQLVRHLDRLVKHITQLRDIVKKDPEDERAVDVIRQSV |
| | | | RSLEIVITVLKIFVDSVSDAARSKEAEKIVRKIRKEIDEIRQKLREIDKEV |
| | | | KKTTS SEQ ID NO: 59 |
| DHD40 | Heterodimer | b | GSNDKVLDKILDILDRILRLATRVIDLANKLLQVKKKSTHKDPRIVETYKE |
| | | | LLKIHETAVRLLLELADLHRRLKSKDEEANKRVETELDRIRKKVKDIEDKV |
| | | | RKLEDKVRKTAS SEQ ID NO: 60 |
| DHD43 | Heterodimer | a | NDLSKEVLKKLEKSVEELLRRVQKSVKEAQKRGLLSDELVDRHLKILNQLV |
| | | | KRHLELLQEVIKRSDKK SEQ ID NO: 61 |
| DHD43 | Heterodimer | b | GSDEAVKRVVEKSLKILDEVIKKSLDILRELIELQIRHAKDDESVIRASKS |
| | | | ALKDAIEALKKSLDEIKKALKRSADEG SEQ ID NO: 62 |
| DHD65 | Heterodimer | a | SSEEVVKVHEKVVKLHKEILELLKKIIKIHETAARDPDDKDSIKKLSDEIK |
| | | | KIVKRIEDISDQAKRESSDAQRKQS SEQ ID NO: 63 |
| DHD65 | Heterodimer | b | DKEEESKELLKKLKEILKRSEELLEESKELLKLAKNGEIDESELADADRKL |
| | | | NKKHEKLVQDIQDLLREHERQDR SEQ ID NO: 64 |
| DHD70 | Heterodimer | a | DEKKKIDKIVKETEDLLQKSEKLLQQSKEAVKRIRSQVKENEIVDRLLRIS |
| | | | EELLKISRRLVEISRRIASTLS SEQ ID NO: 65 |
| DHD70 | Heterodimer | b | GSSKEEVIRLLKENVRLIKENLELLTRNLKLITDLVRGSNGSEEKIKTLKE |
| | | | LLKEYRELLKRYRKLVEDYKRLVDKHD SEQ ID NO: 66 |
| DHD88 | Heterodimer | a | EIQELIKSSRRIIEESKELIKESEEVLRRIKEILDRIRNGVDNQEDLLREI |
| | | | LKLLTKNLKIIQRNLKLLQDNAEILKRLVS SEQ ID NO: 67 |
| DHD88 | Heterodimer | b | GSYIEDVIKKILDVSRELIKLSRTIIKISEEINKQLQQGRDTKDLVKKYDE |
| | | | IIKKYTRIVQHYTELIKELQKLLS SEQ ID NO: 68 |
| DHD89 | Heterodimer | a | SPTEEAIQLSQRVIELSKRVIELSKEILKLLKRVLDLLPDLDKNEEKRLDD |
| | | | YDKELKEYDKELKKYEKRLKDLAS SEQ ID NO: 69 |
| DHD89 | Heterodimer | b | GSEEEEILKIQKELLRIQSEILDKQKKILDTLRSNGAVTEEVRSILEKVER |
| | | | LSEEAKELSKEAKELTKEVSKLIS SEQ ID NO: 70 |
| DHD90 | Heterodimer | a | SPLKELNNQLLRLLRELVKVSKKIVDLSKTIIEVLKHTDLDPRLLDSLEKS |
| | | | QQELDKSQKELDKVVKELTKVNKKLQ SEQ ID NO: 71 |
| DHD90 | Heterodimer | b | GSPLEDLVRKYDELVKTYEKLVEEFKKAVDKYDKAVKKAPVSKEATDSLDL |
| | | | IRKVLELLDRNLKLIKENAKLIKELLK SEQ ID NO: 72 |
| DHD91 | Heterodimer | a | SPTRENEKVIKENEKVISDNERVLEEVVKVVETATDRKEIQDAVDEVRKSV |
| | | | DKLRDSVRKLEESVRTLD SEQ ID NO: 73 |
| DHD91 | Heterodimer | b | GSPIKDISKRLLEISKRLVEISDRIVELLQRIADSKDPNKDLQKEVKDVLE |
| | | | EYKRLVREYREVVKEYEKVVS SEQ ID NO: 74 |
| DHD92 | Heterodimer | a | DEDEHVKQLIKNADLLRKHAELLKELVKLFQEIASQIPDDRVAKKVTDVVD |
| | | | RIDKILKQTEKLVRRTKQILDYSR SEQ ID NO: 75 |
| DHD92 | Heterodimer | b | GSNLEELVKLLKEVLEMHERLLRIHEDLVEAHKSNASDKESERKLKKSDKD |
| | | | IKESLKKIKSIIDQVRYIQS SEQ ID NO: 76 |
| DHD93 | Heterodimer | a | PVEDIIEESLRLLEESLKLLNRILKLLEDSLRKLPRSEEWRQRLDEFRKKL |
| | | | EDWKEELERWIEDVRYKKT SEQ ID NO: 77 |
| DHD93 | Heterodimer | b | GSDEDYESREIIDEIRKLLDRSKKIVHRSQRLVERVKSTPLSEDQEDLIRR |
| | | | HEETINRHRELVKELEKVLEDHERHIR SEQ ID NO: 78 |
| DHD94 | Heterodimer | a | PEEDSRRVLERFVRVSREVLKVLEEFLRVSEELLREADRDRDRRLEEYERQ |
| | | | VDELREEIRRYKEEVDKEDKEVKYYKK SEQ ID NO: 79 |
| DHD94 | Heterodimer | b | GSPEKDENRKLLDKVRKLVEKSRRLVEELRKLVDQSTKNGLIDEKALRKQQ |
| | | | EVLRKVEEVLEKQERVLRELEEISYRVI SEQ ID NO: 80 |
| DHD94_ | Heterodimer | a | GSPERDENRKLLDKVRKLVEKSRRLVEELRKLVDQSTKN SEQ ID |
| 3:214 | | | NO: 81 |
| DHD94_ | Heterodimer | b | GSDEKALRKQQEVLRKVEEVLEKQERVLRELEEISYRVITRGEDHKAEEDS |
| 3:214 | | | RRVLERFVRVSREVLKVLEEFLRVSEELLREADRDRDRRLEEYERQVDELR |
| | | | EEIRRYKEEVDKEDKEVKYYKK SEQ ID NO: 82 |
| DHD94_ | Heterodimer | a | GSDRRLEEYERQVDELREEIRRYKEEVDKFDKEVKYYKK SEQ ID |
| 2:143 | | | NO: 83 |
| DHD94_ | Heterodimer | b | GSPERDENRKLLDKVRKLVEKSRRLVEELRKLVDQSTKNGLIDEKALRKQQ |
| 2:143 | | | EVLRKVEEVLEKQERVLRELEEISYRVITRGEDHKAEEDSRRVLERFVRVS |
| | | | REVLKVLEEFLRVSEELLREADR SEQ ID NO: 84 |
| DHD95 | Heterodimer | a | DLSEESKKFVEKVKKLEKESRELEKQVKKIEEDSRSVENDVQKEFLELLKR |
| | | | LLDIQKKVVEVLREVVKVQQYVDS SEQ ID NO: 85 |
| DHD95 | Heterodimer | b | GSDSEYESRQVLRELDTVLKDSHTVLEALRQVIRDSQDVVSKSDEESRRVI |
| | | | DDLEKVIQDSKKVLDDIKRLIDKSKSIKS SEQ ID NO: 86 |
| DHD96 | Heterodimer | a | NEDELLKLLTENLKLLDENLKLLRENLSLLRQANNITDKNRIREIVKQSKE |
| | | | IVKQSREILKQSKEIVERIKYIVS SEQ ID NO: 87 |
| DHD96 | Heterodimer | b | GSSLYELTQRYEKLVQQYEELVKDYRRLVKKLEKLKRDNKPDKRLLKEIVD |
| | | | VIKKSVEIIDRSLKLLEESIKILEETD SEQ ID NO: 88 |
| DHD97 | Heterodimer | a | SQERSLEILKRILDVLKESLEILKESLSILRQLASRIKNPNRKIEEILKES |
| | | | DKIIKESDKVLKEIEEVIRYSS SEQ ID NO: 89 |
| DHD97 | Heterodimer | b | GSDIEYESKEILELIKELLKLSRELLKESRRALELVRKSRDDSIVEEVIQV |
| | | | HKKVLDIHKEVLKIVRKVVEVHRRVKS SEQ ID NO: 90 |
| DHD98 | Heterodimer | a | SKKDESTKLERLAEKIDEITKRIEELVKDVKRKSSEGVDKDQQQKIDEVFQ |
| | | | KLLDLQREILEILDRILKVQQYILD SEQ ID NO: 91 |
| DHD98 | Heterodimer | b | GSDLEYLNRRLLQLIKTLIDLNRHLLKLIDKLKKLNSREGDEEKIKEESKQ |
| | | | IQEQFKEIVERSKEIIKQIKEIIKRSQ SEQ ID NO: 92 |
| DHD99 | Heterodimer | a | DFERSSRRLEKVVEDLRRSSDRLREVIDELRKSADEKDEDEDLRRARKEHR |
| | | | DLIEELKRALEKQEEIIKHLQELVYRQL SEQ ID NO: 93 |
| DHD99 | Heterodimer | b | GSEESEEVRKVVERIKKISRELEEVVKELDRVSKEFDRHGETDEIVREHER |
| | | | IVEKLEEIVKKHTKIVEELAEIVYKQQ SEQ ID NO: 94 |
| DHD100 | Heterodimer | a | SDDDSVRVLDEIVKILDESVKLLKESLKLLDDFLRTKPDDHLKEVVKESKK |
| | | | VVEQSKKVLDRIKKIIYESK SEQ ID NO: 95 |
| DHD100 | Heterodimer | b | GSDLLYLSKELLKLVRELLKLSRELVELSRRLVNSTHKSPELVKKYDKLVK |
| | | | KYQDLLKKLADVADEYLRQRS SEQ ID NO: 96 |
| DHD101 | Heterodimer | a | DEKDYHRRLIEHLEDLVRRHEELIKRQKKVVEELERRGLDERLRRVVDRER |
| | | | RSSERWEEVIERFRQVVDKLRKSVE SEQ ID NO: 97 |
| DHD101 | Heterodimer | b | GSDAYDLDRIVKEHRRLVEEQRELVEELEKLVRRQEDHRVDKKESHEILER |
| | | | LERIIRRSTRILTELEKLTDEFERRTR SEQ ID NO: 98 |
| DHD102 | Heterodimer | a | DERYRAREHIRRVEEHTKRLRHILKRLREHEEKLRRELKPGDEITESVDRF |
| | | | KKIVDQFEESIKKFETVSEELRKSDS SEQ ID NO: 99 |
| DHD102 | Heterodimer | b | GSDRQRILDRLDKILEKLDDILKKLKDILETLSKDDVSDRRHKDLVEKFRE |
| | | | LVDTHHKLVERYRELVYQNR SEQ ID NO: 100 |
| DHD102_ | Heterodimer | a | GSDEITESVDRFKKIVDQFEESIKKFETVSEELRKSIS SEQ ID |
| 1:243 | | | NO: 101 |
| DHD102_ | Heterodimer | b | GSDPQRAADRLDKILEKLDDILKKLKDILETLSKDDVKDRRAKDLVEKFRE |
| 1:243 | | | LVDTHHKLVERYRELVYTATAGSDLARELIRRVEEHTKRLRHILKRLREHE |
| | | | EKLRR SEQ ID NO: 102 |
| DHD103 | Heterodimer | a | NADDQLATSIKKLEDSIDQLIKIVRKFEESVKKLQKHGVDQHHVEILRKIV |
| | | | EIFRQHIEKLKKHLEKLRYTSS SEQ ID NO: 103 |
| DHD103 | Heterodimer | b | GSDKEYLVTEHEKLVREHEKIVSEIEKLVKKHEAGVDESELEEILKKVEKL |
| | | | LRKLDEILEQLTQLLRKTE SEQ ID NO: 104 |
| DHD103_ | Heterodimer | a | GSDQHVVEILRKIVEIFRQHIEKLKKHLEKLRYTSS SEQ ID NO: 105 |
| 1:423 | | | |
| DHD103_ | Heterodimer | b | GSDAEYLVTEHEKLVREHEKIVSEIEKLVKKHEKGVDESELEEILKKVEKL |
| 1:423 | | | LRKLDEILEQLTQLLRKAEKHIDKHSKAADQLATSIKKLEDSIDQLIKIVR |
| | | | KFEESVKKLQKH SEQ ID NO: 106 |
| DHD104 | Heterodimer | a | DEDDDIRRVLDESRRVLEHSRRVLKRSEEVLEKASRKKEKDTEEIEKHLKR |
| | | | LREHAKKLEKHRRELDDFLYKEI SEQ ID NO: 107 |
| DHD104 | Heterodimer | b | GSRDKYLLERLNDILKKLDEIVDKLSDILKRLKDVRHDDRLQELVERYKEI |
| | | | VKEYKRIVEEYEKLVREFEEQQR SEQ ID NO: 108 |
| DHD105 | Heterodimer | a | DRDYEDKEFKKIIKELEDVQEELKKLQEKIKRFSSELEEPNELLKEQLKVN |
| | | | EEQLEVNKKILKILRDQLKQNE SEQ ID NO: 109 |
| DHD105 | Heterodimer | b | GSDAEYKVRESVKRSKESVKHSEDVVDKLNKSVKLSESGHSDAEKASRELV |
| | | | KLVREVVELSREVIKLSEKVLRVIS SEQ ID NO: 110 |
| DHD106 | Heterodimer | a | DLQYKQEKLIRHFDRVVREWDKLVRKFSKVLEKQKHESKDKELEEASRRVD |
| | | | ELIKRLREQLKRSKEILRRLKELSRKSS SEQ ID NO: 111 |
| DHD106 | Heterodimer | b | GSDWEELLRRLEKVLQEYEEIVKELIDLIERLIKVSEDKSKDASEYKKLVT |
| | | | ELEKLISKLEEISKKLEELVKEYEYKTE SEQ ID NO: 112 |
| DHD107 | Heterodimer | a | DAKDELEKSLQEIEESLKELKKLLEELDKSLRELTSQGRNKKLEEHIKKVQ |
| | | | KFIELVKKYIKAVQDYLKEVRYDNS SEQ ID NO: 113 |
| DHD107 | Heterodimer | b | GSDKERAARATEEMVKLTKKLLKAVEDLVRDVRRLLKEGLISEKHARIAET |
| | | | ILEVFKKHAKIIKKHVDIVKYDES SEQ ID NO: 114 |
| DHD108 | Heterodimer | a | GSPLKERLLEIQRDLDRVLEEVVERLLRIQERLDSVVERKPPDVHEEYKYI |
| | | | VDEIREIVERVVREYEEIVKRIDEEVR SEQ ID NO: 115 |
| DHD108 | Heterodimer | b | GSEEDERIRYDLDRIRKDVRRKLEEIRQRVRELEKKLRDAGHRRDEKELLR |
| | | | ELIETSKDILRLVEELLKKIIDKSEDLLRKTE SEQ ID NO: 116 |
| DHD109 | Heterodimer | a | GSDEEDYINENVEKDVRDIEDDVRRINERIRELLEKIRTEEVLQRVLEEHH |
| | | | ELVERVLRKLVEILRKHEEENR SEQ ID NO: 117 |
| DHD109 | Heterodimer | b | GSDEEEYYKEKLHKLLREIEELLKHYRELVRRLEELVKRGELDKDTAAHIL |
| | | | ERLSELLERIIRRVAHTLRRLSEERR SEQ ID NO: 118 |
| DHD110 | Heterodimer | a | GSDEDEISYDSKRRVEEIVRQAREKSEKSRKDIEDVAEVLRKGDVSEKEVV |
| | | | DELVKVLEEQVKVLREAVERLREVLKKQVDDVR SEQ ID NO: 119 |
| DHD110 | Heterodimer | b | GSDIVELVDHLLKRSLKLLEELAELVRRLLEKSTELLKRRTEEHKEEVVEE |
| | | | SEYMVRELEERLRRVVDESEKLVRDADKHIR SEQ ID NO: 120 |
| DHD111 | Heterodimer | a | GSKEKDIVKTLVDLLRENLETLERLIEEVVRLLKENVDVRDEGRDDKDSER |
| | | | ILRDIKRRIDEAAKESREIIERIEKEVEYRSR SEQ ID NO: 121 |
| DHD111 | Heterodimer | b | GSPEVDVLRRIVREILKASEELLRLLRKLIDEALKLSERKRDSQEYREVVD |
| | | | RVKKELERLLDEYRKLVEELKEKLRYDTR SEQ ID NO: 122 |
| DHD112 | Heterodimer | a | GSDKRYESEKLKRRLDEAVEKVREVVERVERESDRVLEEVRRRRESKEVVD |
| | | | KVIEDNDKALEDVLRVVDEVAKVVRDVVRENTR SEQ ID NO: 123 |
| DHD112 | Heterodimer | b | GSPREYHSKDILRKVDEILERIRRHADRVKKKSERLKRENVDVNEHSKDVK |
| | | | RVIRELLELVKELLRLAKKHSDDQQE SEQ ID NO: 124 |
| DHD113 | Heterodimer | a | GSDEDEILYHSERLLQKLKKELDDLKEKSRELLEELKKEDPDDRLIERIIR |
| | | | LHDEVLKDLDEVLKNILEVHREVLERLR SEQ ID NO: 125 |
| DHD113 | Heterodimer | b | DKLDRLLKIHEEALRRAEELIKRLLDIHRRALDLARRGELDDYLLKESERE |
| | | | LREIIRRAREELKESRDRLEEISR SEQ ID NO: 126 |
| DHD114 | Heterodimer | a | GSPKEELIRRVLEEVKRLNEKLLEIIRRAAELVKRANDELPETEKLREIDR |
| | | | ELEKKLKEIEDELRRIDKELDDALYEIED SEQ ID NO: 127 |
| DHD114 | Heterodimer | b | GSPKLDKLRELLERNLEKLREILEEVLKILRTNLERVREDIRDEDVLQEYE |
| | | | RLIRKAEEDLRRVLKEYDDLLKKLVYELR SEQ ID NO: 128 |
| DHD115 | Heterodimer | a | GSKEDESVKRAEEIVRTLLKLLEDSLREAERSLRDIKNGEDEHNLRRISEK |
| | | | LEELSKRITETIERLLRELQYTSR SEQ ID NO: 129 |
| DHD115 | Heterodimer | b | GSPNQELLDRVRKILEDLLRLNEELVRLNKELLKRALEMRRKNRDSEEVLE |
| | | | RLAEEYRKRLEEYRRELEKLLEELEETIYRYKR SEQ ID NO: 130 |
| DHD116 | Heterodimer | a | GSDESEEAQHEVEKVLDDIRRLSEHLQKRLEEVLEEVYELRREGSDRTEVV |
| | | | ELLKEVIREIVRVNREALERLLRVVEEAVKRNE SEQ ID NO: 131 |
| DHD116 | Heterodimer | b | GSDEEELVETVKRIQKEILDRLTELAKLLVEIQREIKKLKDEGEDDKELKR |
| | | | LSDELEEKVRQVVEEIKRLSDELEETVEYVSR SEQ ID NO: 132 |
| DHD117 | Heterodimer | a | GSDEEEEVVRRAEELVKEHEELIERVIRTHEELVYKLEDQGADKKLVDVLK |
| | | | RVVEESERVAREIVKVSRELIRLLEEASR SEQ ID NO: 133 |
| DHD117 | Heterodimer | b | GSSKEEILKELEDLQRRLIEELKKLQERVVELLEELIKRLRDRGRDDKHLK |
| | | | RLVKEVRRLSEEVLRSIKEVSDRVRYQLR SEQ ID NO: 134 |
| DHD118 | Heterodimer | a | GSDKEEESEYLLRDLVRLLEKVKEKIEEVNREVEKLLKKVKDGRLDRREVL |
| | | | REILRLNRELAEIIKEVVDRIRHVVERSER SEQ ID NO: 135 |
| DHD118 | Heterodimer | b | GSDLHEVVYETKELLKRIEEVVEELRKKSEDIIRKAERGEISEDELKRLQE |
| | | | EIAREAKKLLDEIKRVLERHLEQTL SEQ ID NO: 136 |
| DHD119 | Heterodimer | a | GSPVEEIIKEVVKRVIEVQEKVLRIISHAVKRVVEVQKKYDPGSEESNRVV |
| | | | EEVKKTIEDAIRESDEVVDEVVKRIQYTVR SEQ ID NO: 137 |
| DHD119 | Heterodimer | b | GSPEQEIADRILTEIRESQKELERLARKILKLLDESQEKAKRGRLSEEESD |
| | | | ELLERIKKELDELLERSKELLKKIEYELR SEQ ID NO: 138 |
| DHD120 | Heterodimer | a | GSDEDKEANRVLDEVLKTVRDLLETANEVLKEVLYRLKRTDDQEKVVRTLT |
| | | | EVLKEHLKLVEEIVRILDKVLKEHLETEK SEQ ID NO: 139 |
| DHD120 | Heterodimer | b | GSPEDDVLRRLEEVSEKILRVAEDVARQLREVSEKITQGKVDRKEWEEDIK |
| | | | RLKRELEELLREWKEEIERLTYELR SEQ ID NO: 140 |
| DHD121 | Heterodimer | a | GSRREEVVKRIRELLKRNKELIDRIRELLEENEYLDKDARDKDVLRRSVEL |
| | | | LEELVRILEESVELAKEIIKLLREVVE SEQ ID NO: 141 |
| DHD121 | Heterodimer | b | GSDEKEDNRRLQHKIERILEKNEDLQRKLEEILELLERGEADEEKIDRLRK |
| | | | AVEDYRRVVEEIKEDVKRHKYTVR SEQ ID NO: 142 |
| DHD122 | Heterodimer | a | GSDEKEEAKKASEESVRTVERILEELLKASEESVELLRRGEDAKDVVERSK |
| | | | EALKRVKELLDEVVKRSDEILKYIHN SEQ ID NO: 143 |
| DHD122 | Heterodimer | b | GSDEKKLINEVVETQKRLIKEAAKRLSEVVRHQTELIRELREKNVDDKDVE |
| | | | KLLKESLDLAEEIVRRIKELLDESKKLVEYVSN SEQ ID NO: 144 |
| DHD123 | Heterodimer | a | GSPDMDEVKRVLDELIEIQEEILREIKRVLEKLIKIQEDNGSEYESREVVR |
| | | | EIVEIARKLVERSRRVVKKITETLQ SEQ ID NO: 145 |
| DHD123 | Heterodimer | b | GSDERYATREIVERIERIAREILKRTEEIVREVREVLSRDVDQEEVVRRLA |
| | | | DLLRESVELVQHLVRRVEELLQESVERKK SEQ ID NO: 146 |
| DHD124 | Heterodimer | a | GSPEREALREVLEDLKRVTDRLRELVERVLEELKKVTDHVDSERILRESRR |
| | | | VLKELKDIIEEILRESEKVLEKLKYTED SEQ ID NO: 147 |
| DHD124 | Heterodimer | b | GSPAREILEEVVKKHLEVVEDAARILEEIIREHEKAVREDRDKKELEEISR |
| | | | DLLRKAREALKKVKDISDDLSREIEYVAS SEQ ID NO: 148 |
| DHD125 | Heterodimer | a | GSPVEEAIKKVIDDLRDVQRKIRELVEELIRLLEEVQRDNDKRESEYVVER |
| | | | VEEILRRITETSREVVRKAVEDLS SEQ ID NO: 149 |
| DHD125 | Heterodimer | b | GSDSDEKAEYLLKEMERVVRESDEVVKKILRDLEEVLERLRRGEISEDDVT |
| | | | EILKELAERHIRAIEELVRRLRELLERHKR SEQ ID NO: 150 |
| DHD126 | Heterodimer | a | GSPVEEVLKELSEVNERVRDIAREIIERLSEVNEEVKETDDEDELKKISKK |
| | | | VVDEVEDLLRKILEVSEEVVRRVEYHDR SEQ ID NO: 151 |
| DHD126 | Heterodimer | b | GSPKEDILREVLRRHKEIVREIVRLVREAVETHLELVKRNSDDRDAQDVIR |
| | | | KLEEDLERLVRHAQEVIEEIFYRLH SEQ ID NO: 152 |
| DHD127 | Heterodimer | a | GSPRSYLLKELADLSQHLVRLLERLVRESERVVEVLERGEVDEEELKRLED |
| | | | LHRELEKAVREVRETHREIRERSR SEQ ID NO: 153 |
| DHD127 | Heterodimer | b | GSDREYIIKDILDSQEHLLRLIEELLETQKELLEILKRRPDSVERVRELVR |
| | | | RSKEIADEIRRQSDRNVRLLEEVSK SEQ ID NO: 154 |
| DHD128 | Heterodimer | a | GSDEKDEIRHVIESVERLIEDIKRLLKTLRELAHDDSDKKTVKEVLDRVKE |
| | | | MIERHRRELEEHRKELERAEYEVR SEQ ID NO: 155 |
| DHD128 | Heterodimer | b | GSESEDRIKELLKRHIELVERHEELLHEIKKLIDLEEKDDKDREEAVKRID |
| | | | DAIKESEEMLEESKEILEEIEYLNR SEQ ID NO: 156 |
| DHD129 | Heterodimer | a | GSSLEDSVRLNDEVVKVVERVVRLNQEVVRLIKHATDVEDEETVKYVLERV |
| | | | REVLDESREVLKRVHELLEESERRLE SEQ ID NO: 157 |
| DHD129 | Heterodimer | b | GSHEKDIVYKVEDLVRKSDRIAERAREIVKRSRDIMREIRKDKDNKKLSDD |
| | | | LLKVTRDLQRVVDELEELSRELLRVAEESRK SEQ ID NO: 158 |
| DHD130 | Heterodimer | a | GSPELDEVKKLIDELKKSVERLEESIREVKESIKKLRKGDIDAEENIKLLK |
| | | | ENIKIVRENIKIIKEIIDVVQYVLR SEQ ID NO: 159 |
| DHD130 | Heterodimer | b | GSDEEEIEELLRELEKLLKKSEEALEESKKLIDESEELLRRDRLDKEKHVR |
| | | | ASEEHVKLSEEHLRISREIVKILEKAVYSTR SEQ ID NO: 160 |
| DHD131 | Heterodimer | a | GSDESDRIRKIVEESDEIVKESRKLAERARELIKESEDKRVSEERNERLLE |
| | | | ELLRILDENAELLKRNLELLKEVLYRTR SEQ ID NO: 161 |
| DHD131 | Heterodimer | b | GSDEDDELERLLREYHRVLREYEKLLEELRRLYEEYKRGEVSEEESDRILR |
| | | | EIKEILDKSERLWDLSEEVWRTLLYQAE SEQ ID NO: 162 |
| DHD132 | Heterodimer | a | GSDKKDASRRAIRVLHEFVRVSEEVLEVLRKSVESLKRLDVDEKIKRTHDR |
| | | | IEEELRRWKRELEELIERLREWEYHQD SEQ ID NO: 163 |
| DHD132 | Heterodimer | b | GSDDEEEDKRLLEEVKRSLDTDERILEKLRHSLERQLEDVDKDEDSRRVLR |
| | | | ELDEITKRSREVVKRLRKLAYESK SEQ ID NO: 164 |
| DHD133 | Heterodimer | a | GSDKEYKLDRILRRLDELIKQLSRILEEIERLVDELEREPLDDKEVQDVIE |
| | | | RIVELIDEHLELLKEYIKLLEEYIKTTK SEQ ID NO: 165 |
| DHD133 | Heterodimer | b | GSPSKEYQEKSAERQKELLHEYEKLVRHLRELVEKLQRRELDKEEVLRRLV |
| | | | EILERLKDLHKKIEDAHRKNEEAHKENK SEQ ID NO: 166 |
| DHD134 | Heterodimer | a | GSRDRKISEELIKALEDHIRMLEELIRAIEEHIKLAERGVDEKELRESLEE |
| | | | LKKIVDELEKSLEELRKLAERYKYETR SEQ ID NO: 167 |
| DHD134 | Heterodimer | b | GSPKEESVEELKRVIDKHEEILRELKRVLEEHERVSHDEDENELRRSLERL |
| | | | KHILDRLHESLKELHELLKKNEYTER SEQ ID NO: 168 |
| DHD135 | Heterodimer | a | GSDHEYWVKIVERILRVMEKHAEIVKKHLEIVERVVREGPSEDLRRKLKES |
| | | | LREIEESLRELKELLDELDELSEKTR SEQ ID NO: 169 |
| DHD135 | Heterodimer | b | GSDEEYVTRSQRRLKRLLEEYIKVVEEHARLVERNERDDKELKRSIDELDK |
| | | | LTKELLELVKRYKELVDKTET SEQ ID NO: 170 |
| DHD136 | Heterodimer | a | GSDKEEIVKLQDEVIKTLERHLDILRKHIDLLEKLKDHLSEELKERVDRSI |
| | | | KKLEESIKRLERIIEELQELAEYSL SEQ ID NO: 171 |
| DHD136 | Heterodimer | b | GSREEELKESAEELERSVRELKKEADKYKEEVDRLHYRGKVDKDWVRVVEK |
| | | | LIKLVEEHLELIREHLELLKEERR SEQ ID NO: 172 |
| DHD137 | Heterodimer | a | GSDMEYELKKSAEELRKSLEELKRILDELHKSLRELRRHGDDEEYVQTVEE |
| | | | LRKELEEHAKKLEEHLKELERVAT SEQ ID NO: 173 |
| DHD137 | Heterodimer | b | PEYELKKSVDDLKRDVDRLVEEVEEVFELSKERLREDRKHLELVEEMVRLI |
| | | | EKHLELIKEHLKLADDHVR SEQ ID NO: 174 |
| DHD138 | Heterodimer | a | GSREKDESKELNDEYKKLLEEYERLLRRSEELVKRAKGPRDEKELKRILEE |
| | | | NEDILRRTKEILERTKEISEEQKYRRR SEQ ID NO: 175 |
| DHD138 | Heterodimer | b | GSDKDERQERLNEESDKSNEESERSNRESEELNRRARGPNDEKELQEILDR |
| | | | HLELLERNQRLLDENKEILRESQYLND SEQ ID NO: 176 |
| DHD139 | Heterodimer | a | GSENKYILKEILKLLRENLKLLHDILRLLDENLEELEKHGAKDLDDYRRKI |
| | | | EEIRKKVEDYREKIEEIEKKVERDR SEQ ID NO: 177 |
| DHD139 | Heterodimer | b | GSESEYTQEEILELLKESIKLLREILRLLEESEELWRRENTKSERSEEIKE |
| | | | RAKEAIKRSEEILERVKRLSDHSR SEQ ID NO: 178 |
| DHD140 | Heterodimer | a | GSDEEEANYVSDKAVKIAEDVQELLKELLELSEVVRRGEVDEDEYDRVLRK |
| | | | LQEVMKEYEEVLKEYEEVSRKHE SEQ ID NO: 179 |
| DHD140 | Heterodimer | b | GSPEKYLIKTQEELLRRHAEILEDLIRKVERQVDLRRKVDERDEDLKRELE |
| | | | RSLRELERLVRESSRLVEEIRELSKEIKR SEQ ID NO: 180 |
| DHD141 | Heterodimer | a | GSDEEYELERISRESKELLERYKRLLREYQELLKELRHVKDLDRAVKIIHE |
| | | | LMRVSKELVEISHRLLELHERLVRRRK SEQ ID NO: 181 |
| DHD141 | Heterodimer | b | GSEKEYIEKLSRKIEEDIRRSEERAKDSERLVRRLEELAKRKRLDLDDVLR |
| | | | VAEENLEILEDNLRILEEILKEQDKSNR SEQ ID NO: 182 |
| DHD142 | Heterodimer | a | GSPHEEVVELHERVMEISERAVELIQRIIDIIRRIREDDKDIEKLVKTIRD |
| | | | LVREYEELHRELEEIDEEIYKKSE SEQ ID NO: 183 |
| DHD142 | Heterodimer | b | GSDHEDVVRLHEDLVRKQEDARRVLEEIVRLAEEIVEVIKKDEKDKDRVTR |
| | | | LVEEIEKLVEEYKKKVDEMRKISDEIKYRSR SEQ ID NO: 184 |
| DHD143 | Heterodimer | a | GSRAREVVKRAKRIIEEWQKILEEWRRILEEWRRLLEDERVDDRDNERIIR |
| | | | ENERVIRENEKIIRDVIRLLEELLYERR SEQ ID NO: 185 |
| DHD143 | Heterodimer | b | GSREDEELEEEIDRIRQMVEEYEELVKEYEELTEKYKQGKVDKEESKKIIE |
| | | | KSERLLDLSQDAVRKVKEIIRRILYTNR SEQ ID NO: 186 |
| DHD144 | Heterodimer | a | GSPKEEIVKLHDESAELHRRSVEVADEILKMHERSKDVDDERESRELSKEI |
| | | | ERLIREVEEVSKRIKRLSEEVEYLVR SEQ ID NO: 187 |
| DHD144 | Heterodimer | b | GSPLEEILKIQRRINKIQDDINKILHEILRMQEKLNRSSDKDEVEESLRRI |
| | | | RELIKRIKDLSKEIEDLSREVKYRTT SEQ ID NO: 188 |
| DHD145 | Heterodimer | a | GSPEDEHVYVVREIYEVLREHAEVLEENREVIERLLEAKKRGDKSEELVKE |
| | | | LKKSIDKLKEISRKLEEIVKELEKVSEKLK SEQ ID NO: 189 |
| DHD145 | Heterodimer | b | GSDEDETSYRILELLREIVRASRELIRLSEELLEVARRDDKDETVLETLIR |
| | | | EYKELLDRYRRLIEELTRLVEEYEERSR SEQ ID NO: 190 |
| DHD146 | Heterodimer | a | GSTQEEINRIQHEVLRIQEEIDEILRDIVEKLKAISRGELDHEVVKDVEDK |
| | | | VREALEKSEELLDKSRKVEYKSE SEQ ID NO: 191 |
| DHD146 | Heterodimer | b | GSDEEELNRELLEKSKRLVDINRDIIRTAQELIEMLKDSKDGRVDEDTKRE |
| | | | LRDKLRKLEEKLERVREELRKYEELLRYVQR SEQ ID NO: 192 |
| DHD147 | Heterodimer | a | GSDEKDRVYEILKEVQRLVKEYRDISKEIEDLVKHYEHITDDEAQEVSKEL |
| | | | IDKSLRASEIVRELIRLIKELLDELE SEQ ID NO: 193 |
| DHD147 | Heterodimer | b | GSDEEDVLYHLRELLEELKRVSDDYERLVREIKETSERKDRDTKENKDMLD |
| | | | ELVKAHREQEKLLERLVRLLEELFERKR SEQ ID NO: 194 |
| DHD1 | Heterodimer | a | PREQAIRISEEIIRISKKIIEILERTRSSTAREAMKWAKDSIRLAEESKYL |
| | | | LDK SEQ ID NO: 195 |
| DHD1 | Heterodimer | b | IEDDVKKIQDSTKKAQKETIEALERSTSSTARKQMEEQKEQIRLQKEAMYL |
| | | | LKK SEQ ID NO: 196 |
| DHD2 | Heterodimer | a | SREEIAKLQEEVIKLQRRVIELQKEVIELQRRAKELTSSYTKEILEIQRRI |
| | | | EEIQREIEEIQKRIEEIQEEIQRRT SEQ ID NO: 197 |
| DHD2 | Heterodimer | b | SDEEIKRLSEEVIQLSRRVIKMSREAIKLSREVQKLTPSYQKRIKEIADRS |
| | | | IELARESIEIAKRSEKIAEESQRRT SEQ ID NO: 198 |
| DHD3 | Heterodimer | a | PAKDEALKMANESLELAKKSARLIQESSSKEILERIEKIQRRIAELQDRIA |
| | | | YLIKK SEQ ID NO: 199 |
| DHD3 | Heterodimer | b | PAKDEALRMIDESRELIKKSNELIQRSSSKEILERILEIQRKIAELQKRIQ |
| | | | YLLKS SEQ ID NO: 200 |
| DHD4 | Heterodimer | a | TDEARYRSERIVKEAKRLLDEARRRSEKIVREAKQRSNSEDAKRIMEENLR |
| | | | ESEEAARRLREIIRRNLEESRETG SEQ ID NO: 201 |
| DHD4 | Heterodimer | b | TREALEYQRKMAEEIEDLLREALRRQEEMVREAKQRSLSEEFKRIMERILE |
| | | | EQERVMRLAKEALERILEEQKRTG SEQ ID NO: 202 |
| DHD5 | Heterodimer | a | SERTKREAKRSQEEILREAKEAMRRAKESQDHRQNRDGSNSEDLERLSQEQ |
| | | | KRELEEVERRLKELAREQKYKLEDS SEQ ID NO: 203 |
| DHD5 | Heterodimer | b | SEDLKRILKEITERELKLMQDLMEILKKITEDENNLDSNNSEDLKRSIEKA |
| | | | RRILDEALRKLEESARRAKYIQEDN SEQ ID NO: 204 |
| DHD6 | Heterodimer | a | TEDEIRESLKWLDEVLQELREIARESNEVLERNRQKSRSDKLREDIERYKK |
| | | | RMEEARKKLDDQLNKYKKRMDENRS SEQ ID NO: 205 |
| DHD6 | Heterodimer | b | TEEELKESKKFAEDLARSARRALKESKRVLEEISQASRSKKLEEIVRRYKE |
| | | | QVKRWQDEWDERAREYRKRMKENRS SEQ ID NO: 206 |
| DHD7 | Heterodimer | a | TKTEEIERLAREIKKLSEKVERLAQEIEELSRRVKEENSTDRELKEANREI |
| | | | ERAIREIEKANKRMEEALRRMKYNG SEQ ID NO: 207 |
| DHD7 | Heterodimer | b | TKTEEHERLAREISKLADEHRKLAKIIEELARRIKEENLTDDELREAIRKI |
| | | | EDALRKNKEALKIMKEAAERNRYNT SEQ ID NO: 208 |
| DHD8 | Heterodimer | a | TKKEESRELARESEELARESEKLARKSLELARRAESSGSEEEKRRIIDENR |
| | | | KIIERNREIIERNKEIIEYNKELIS SEQ ID NO: 209 |
| DHD8 | Heterodimer | b | TKDEESLELNRESEELNRKSEELNRKSKELNDRAESSNSEEEEKEILREHK |
| | | | EILREHLEILRRHKEILRRHKYLTS SEQ ID NO: 210 |
| DHD16 | Heterodimer | a | TREELLRENIELAKEHIEIMREILELLQKMEELLERQSSEDILEELRKIIE |
| | | | RIRELLDRSRKIHERSEEIAYKEE SEQ ID NO: 211 |
| DHD16 | Heterodimer | b | SEDIAREIKELLRRLKEIIERNQRIAKEHEYIARERKKLDPSNEKERKLLE |
| | | | RSRRLQEESKRLLDEMAEIMRRIKKLLD SEQ ID NO: 212 |
| DHD18 | Heterodimer | a | DRQKLIEENIKLLDKHIKILEEILRLLKKDIDLLKKSSSEEVLEELKKIHR |
| | | | RIDKLLDESKKIHKRSSEIVKKRS SEQ ID NO: 213 |
| DHD18 | Heterodimer | b | DEQKLIETSQRLQEKSERLLEKFEQILREASDLYRKPDSEELLRRVEKLLR |
| | | | ELEKLIRENQDLARKHEKILRDQS SEQ ID NO: 214 |
| DHD19 | Heterodimer | a | DRQELIRENIELLKKHIKIVKEIQKLIETFIELLKKSSSEEILRRLKKILK |
| | | | RIEKLYRESQEIHKRSEEIAKKRQ SEQ ID NO: 215 |
| DHD19 | Heterodimer | b | DEERLIDKSRELQKESEELLKELLKIFKRIEELLEKPDSEELIREIKKLLE |
| | | | TLSEIHKRNEKLARTHEEILRQQS SEQ ID NO: 216 |
| DHD22 | Heterodimer | a | STRDVQREIAKAFKKMADVQKKLAEEIKRHVKNVEKKNKDNDEYRKIATEL |
| | | | LKKATESQKKLKELLDRIRKSDS SEQ ID NO: 217 |
| DHD22 | Heterodimer | b | DKDDRSTSLLKRVEKLIDESDRIIDKFTTLIELSRNGKIDDDQYKKELKEI |
| | | | LELLKKYDKHVKEVEELLKRLNS SEQ ID NO: 218 |
| DHD23 | Heterodimer | a | SKRKALEVSERVVRISEKVVRVLDESSDLLKKSYDDSDKFAELIDRHEEKI |
| | | | KKWKKLIKEWLEIIQRHKS SEQ ID NO: 219 |
| DHD23 | Heterodimer | b | SAEEFVKLSEEAVKRSKEILDIVRKQVKLVKAGVDKHEITDSLRKSEKLIE |
| | | | EHKELIKTHRDLLRREN SEQ ID NO: 220 |
| DHD24 | Heterodimer | a | SSTEILKRFKRALRESEKIVKHSRRVLKIIREVLKQKPTQAVHDLVRIIET |
| | | | QVKALEEQLKVLKRIVEALERQS SEQ ID NO: 221 |
| DHD24 | Heterodimer | b | DKQKEIKDILEKTRRIAEESRKIAEKFDEIIKRSTEGKIDESLTKELEELV |
| | | | KEVIKLSEDDARTSDDLVRKES SEQ ID NO: 222 |
| DHD26 | Heterodimer | a | DEDESIKLTRKSIEETRKSLKIIKEVVELIREVLKHIKDLDKEIFERIDKI |
| | | | LDKYKKQVDTYDEILKEYEKKQR SEQ ID NO: 223 |
| DHD26 | Heterodimer | b | SELDEQKELIKKQEKLIEEQQRLLSKIRRMFKERVKDQELLREIQKVLKRS |
| | | | QEIVETSKKILDRSDKTTE SEQ ID NO: 224 |
| DHD28 | Heterodimer | a | DQKEINTRIVEKLERIFKKSKEIVRQSERVISTIEKKTEDERELDLLRRHV |
| | | | KIVREHLKLLEELLKIIKEVQKESE SEQ ID NO: 225 |
| DHD28 | Heterodimer | b | DTEELVKRLNELLKELSKLVKEFIKILETYRKDQTKDTSKISERVDRILKT |
| | | | YEDLLQKYKEILEKIEKQLS SEQ ID NO: 226 |
| DHD29 | Heterodimer | a | DYARLIDQAVEVTRKVVEVNVTVARVNDKFAKHLGDEELRRVSEHLKEVSK |
| | | | DLQEVAKKSKDAARQVK SEQ ID NO: 227 |
| DHD29 | Heterodimer | b | DVSKVAEEYLQISKTLVDISRTLLEISERLVRLVRTVADDRSEVKKAIEDS |
| | | | IEVLKTSEEVVRQIKRASDKLVKAIS SEQ ID NO: 228 |
| DHD31 | Heterodimer | a | DAKEIQRRVVEIQTEVVKLQKKAVDIIRKIIEAFNNSNIDQSLLEAAKEIV |
| | | | KEIDKLEKLTESLLEESKKLLKRSS SEQ ID NO: 229 |
| DHD31 | Heterodimer | b | SAEEVVKLAKIFLELLRESIKLLKRSVDLLRKSSDPSLDKSEAEKVSREIE |
| | | | KVSDTSLKLSKKALDVVKRALKVAS SEQ ID NO: 230 |
| DHD32 | Heterodimer | a | DEKDAARKARKVSEEAKEASKKIEKALEESKRILNTLKQKKDEQEVKVIKE |
| | | | HEDVLRQIEKIQKQVLEIQKEVAKLLESLD SEQ ID NO: 231 |
| DHD32 | Heterodimer | b | SADDVARASEKVLRVARESAKAADKSLEVFKEVVKRGDKEAFLQVVKINEE |
| | | | VVKINITVIRILIEVSKTAT SEQ ID NO: 232 |
| DHD38 | Heterodimer | a | DEYVKETLKQLREALASLREADKRITELVKEARKKPLSEAARKFAEAIVTH |
| | | | VKVVVEHVEVVLRHVEVLVEAKKNGVIDKSILDNALRIIENVIRLLSNVIR |
| | | | VVDEVLQDLD SEQ ID NO: 233 |
| DHD38 | Heterodimer | b | DASDVIRRIHELFEEVHRLIEAVHRAIEDVAKAAQKKGLDESAVEILAELS |
| | | | KELAKLSRRLAEISREIQKVVTDPDDKEAVERLKEIIKEIKKQLDELRDRL |
| | | | RKLQDLLYKLK SEQ ID NO: 234 |
| DHD60 | Heterodimer | a | SEDKAHHDIVRVLEELIKIHDELMKISEEILKATSDSTATDETKEELKRRS |
| | | | KEAQKKSDTLVKIVKELEKESRKAQS SEQ ID NO: 235 |
| DHD60 | Heterodimer | b | DDEEKYRQIIREAQEISKTAKRILRDAQEISKRIRHQGVDRSEHQRLVDLL |
| | | | RELIKEHHKLLRRQQEADTRND SEQ ID NO: 236 |
| DHD63 | Heterodimer | a | DRKDKARKASEKLEEVIQRWKTVADKWKKMVDLVSNGKLSQEEVARVTEEL |
| | | | LKIQTELAKLLEEHAKVLQESAS SEQ ID NO: 237 |
| DHD63 | Heterodimer | b | SDEESIKTQSELIKTSEELLKDVKRIDEELQKLRDDPTLDESELKKRVKEW |
| | | | SDRVRKAKEISRKIQEIVKESKKRSS SEQ ID NO: 238 |
| DHD66 | Heterodimer | a | DKDEELRKVIEKYREMVKEYRKVIREYEEVIKSSKTIDKSSLISLSRKMVE |
| | | | LSQRVIDVSDEVAKVLSRKQS SEQ ID NO: 239 |
| DHD66 | Heterodimer | b | TDEERLKKQTKELKEQTKQLEKQKDLLEKISNGEISKDEIQEIIKESKKIA |
| | | | KESQKALDSSRKALEEVS SEQ ID NO: 240 |
| DHD67 | Heterodimer | a | DEKEVSKEIIKVLKDIAKVQQKVIEVSQRLASVLRADDDNVVKRALEEYEK |
| | | | ILEELRELNKEIEKLTDKYRKVTS SEQ ID NO: 241 |
| DHD67 | Heterodimer | b | DSDEQTKELEKLTELHKRHVEKLKKQTKESREVDSNKLWKSKDVKDKLSES |
| | | | EKELQKLSDQDKKAKDALESSRRKND SEQ ID NO: 242 |
| DHD69 | Heterodimer | a | DAEEQLKLLTKLLRHQQRLLQLIKESLKLIEKIDQSSQENQDEIRKWREVT |
| | | | KKLRELIKTSEKLVRELEKSYKKSS SEQ ID NO: 243 |
| DHD69 | Heterodimer | b | SLRDVVRRYQELVRRYDELIKTLTEILKKYQKKGAEDKDASTELVKAVRTS |
| | | | LKLSKELLKINSELLKEDS SEQ ID NO: 244 |
| DHD71 | Heterodimer | a | SKEELKRKLDELKKRSDTLKELSKKLKEISERNPDDKSVHRTIIRIHREFV |
| | | | KNHKEIVRVIEEIVSDKS SEQ ID NO: 245 |
| DHD71 | Heterodimer | b | SKQDEHDRLLKIHDKLVKQHDELLKLLTKLSRAGDSVTKKKLEEILRKLQE |
| | | | VSKQLEESLKDADKVSKDIN SEQ ID NO: 246 |
| DHD72 | Heterodimer | a | TVQSLLEQHVKIVKRSIEILERHTQILQDIARSQGVSKELEDVERQVKEYR |
| | | | KEVKKLEEDLRQLSRNSK SEQ ID NO: 247 |
| DHD72 | Heterodimer | b | SDSDRIEKLIRESTELLKEQQKLAKRSRELAETVESLPLTEEYLKQQREHQ |
| | | | KKIEKLLKDSEKHLEELKRLVKSEK SEQ ID NO: 248 |
| DHD73 | Heterodimer | a | DSEKRIEDILRTDLELAKRDAELVKEHIKLVKRIDLSEELKKQVEDVEKES |
| | | | KKLEDSSEKLVQKVRKRSS SEQ ID NO: 249 |
| DHD73 | Heterodimer | b | DEEERAKDLRKYLEEQTQYYRTVTEHLRNLEKVVEELERRGKPSSELQQIL |
| | | | ERSQRIYKETTEIYDTSKKLIEELDKHHR SEQ ID NO: 250 |
| DHD148 | Heterodimer | a | PLEDILKRHLDKVRELVRLSEEVNKLAKEVLDILKDKRVDEKELDKVLKEL |
| | | | EKVVEEYERAVKESRDLLRELRETTR SEQ ID NO: 251 |
| DHD148 | Heterodimer | b | DKERLLEIHERIQKLLDRNLEIIERLLRLLREARDIKDDDKLDKVIKRLKE |
| | | | LSEESKDILDKIKELLKESEKELT SEQ ID NO: 252 |
| DHD149 | Heterodimer | a | PEDEVIRVIEELLRIAAEVDEVHRRNVEVQEEASRVTDRERLERLNRESEE |
| | | | LIKRSRELIEEQRKLIERLERLAT SEQ ID NO: 253 |
| DHD149 | Heterodimer | b | DLEELIKEYAEVVRRHHKAVRDLERLVRELANAKHASEEELKRIATEILRI |
| | | | VKELIRVQERLIKLSEDSNEESR SEQ ID NO: 254 |
| DHD150 | Heterodimer | a | PTDEVIEVLKELLRIHRENLRVNEEIVEVNERASRVTDREELERLLRRSNE |
| | | | LIKRSRELNEESKKLIEKLERLAT SEQ ID NO: 255 |
| DHD150 | Heterodimer | b | DNEEIIKEARRVVEEYKKAVDRLEELVRRAENAKHASEKELKDIVREILRI |
| | | | SKELNKVSERLIELWERSQERAR SEQ ID NO: 256 |
| DHD151 | Heterodimer | a | PKEDIDRVSRELVRVHKELLEVLRKSTEIVEAVARNEKDERTIEEVLEEQE |
| | | | RAVRKLEEVSKKHKEAVKRLK SEQ ID NO: 257 |
| DHD151 | Heterodimer | b | ELERLSEEIQKLSDRLIELIRRHSKVLEEIVRLLKHKDNDEREVRRLLKLL |
| | | | RDLTRRYEEVLRKVEEIVKRQEDESR SEQ ID NO: 258 |
| DHD152 | Heterodimer | a | PEEDILRLLRKLVEVDKELLEVVRESTEVVRLVARNEKDVETVERVLRKQE |
| | | | EVVRKYERVSRELEEAVRRLK SEQ ID NO: 259 |
| DHD152 | Heterodimer | b | ELKDLVEEIVKLSKENLKLWEDHSRVLEEIVRLLKHKDNDEREVRRLLKLL |
| | | | EDLTRRAEETSRRIEEIVKEAEDRAR SEQ ID NO: 260 |
| DHD153 | Heterodimer | a | DEERELREVLRKHHRVVREWTKVVEELKRVVELLKRGETSEEDLLRVLKKL |
| | | | LEMDKRILEVNREVLRVLEKRLT SEQ ID NO: 261 |
| DHD153 | Heterodimer | b | SLEEIIEELVELVRRSVEIAKESDEVARRIVESEDKKKELIDTLRDLHREW |
| | | | QEVTKRAEELVREAEKEVR SEQ ID NO: 262 |
| DHD154 | Heterodimer | a | TAEELLEVHKKSDRVTKEHLRVSEEILKVVEVLTRGEVSSEVLKRVLRKLE |
| | | | ELTDKLRRVTEEQRRVVEKLN SEQ ID NO: 263 |
| DHD154 | Heterodimer | b | DLEDLLRRLRRLVDEQRRLVEELERVSRRLEKAVRDNEDERELARLSREHS |
| | | | DIQDKHDKLAREILEVLKRLLERTE SEQ ID NO: 264 |
| DHD155 | Heterodimer | a | PEDDVVRIIKEDLESNREVLREQKEIHRILELVTRGEVSEEAIDRVLKRQE |
| | | | DLLKKQKESTDKARKVVEERR SEQ ID NO: 265 |
| DHD155 | Heterodimer | b | DEVRLITEWLKLSEESTRLLKELVELTRLLRNNVPNVEEILREHERISREL |
| | | | ERLSRRLKDLADKLERTRR SEQ ID NO: 266 |
| DHD156 | Heterodimer | a | DEDEVVKVHEEHVKSHEEIHRSHEEVVRAAEEDKRDSRELRTLMEEHRKLL |
| | | | EENEKSIEEVKKIHERVKR SEQ ID NO: 267 |
| DHD156 | Heterodimer | b | KKEELIDISKEVLDLDDEINKISKEILELIKKLLRLKEEGREDKDKAREVK |
| | | | RRIRELHRRIQELNKRLRELHKRVQETKR SEQ ID NO: 268 |
| DHD157 | Heterodimer | a | PEEDIARRVEDLLRKSEELIKESEKILKESKRLLDRNDSDKRVLETNLRLI |
| | | | DKHTKLLERNLELLEELLKLAEDVAK SEQ ID NO: 269 |
| DHD157 | Heterodimer | b | RFKDLSREYIEVVKRLLELSREALEVLREIKDTDKTDKKRIKELIDRLRKL |
| | | | IEEYKRIIDRLRKLSKDLEEEHR SEQ ID NO: 270 |
| DHD158 | Heterodimer | a | DEEELVKILKELQRLSEESLEINKRLVEILRLLRRGEVPKEEVEKKLREIK |
| | | | KEQEKLDREHEKIKKRIEEITK SEQ ID NO: 271 |
| DHD158 | Heterodimer | b | SLKEKILEIIERNMKLVELSNRSVEIVARILKGEKDDEETLERLLREWDKI |
| | | | TRDYEEIIKESRKLVKELEEEAK SEQ ID NO: 272 |
| DHD159 | Heterodimer | a | SKTEILRKALEIHKEQIDIVRKLIELSEEVLKLVEESKEKNLEKLKRIDEE |
| | | | TDRLLERLDELHKRLTELAERLK SEQ ID NO: 273 |
| DHD159 | Heterodimer | b | SDDEARKQLEEMKRRLREVEKKSKRVEERVRELERLVRENREDEDRVLKTL |
| | | | EDLLRENEKLVRTIERHVREQRELSKEVK SEQ ID NO: 274 |
| DHD160 | Heterodimer | a | SEEELEKKADELRKLSEEWRKLQEEDKRLSEMVEKGELDLQEVDEHSLRVL |
| | | | ERATEVHRTVDKVIEEILRTTN SEQ ID NO: 275 |
| DHD160 | Heterodimer | b | SEKERHRESQETQEEIRRTHEEIIRKLEEILRRAKAGELPEETLDRLRRIM |
| | | | ERLKELSERLDDLVRKLRDDHRREQK SEQ ID NO: 276 |
| DHD1 61 | Heterodimer | a | SEKEILEELKRILKRVKDISDRLEELDKRTEEIARREPTKELVDELVKIHR |
| | | | DWLRLHEEILKLVDDALKKVEDATK SEQ ID NO: 277 |
| DHD61 | Heterodimer | b | DLRELLELQREASRLHRELVKLLTELVKKLELIAKGEDIREEDLKRIKERL |
| | | | EEIKKRSKRIKEESDEIDKKTK SEQ ID NO: 278 |
| DHD162 | Heterodimer | a | SERELQRELNKIVRRILEIHREVSELHQRAVKLIRENDNSEELEEISRRIE |
| | | | ELSKELEKLVREHDEIVKTIE SEQ ID NO: 279 |
| DHD162 | Heterodimer | b | SEREKLDRNDEELKEINKRVEEIKERSDRITEAIEKNERSEEEIRRLSREQ |
| | | | NEALQRLLELHKKLVKLHRELLEDTR SEQ ID NO: 280 |
| DHD163 | Heterodimer | a | DKEDVIRVHDEQHKLIEEQLELTRRIAELVREIAKNTASEEEIKEMLKEIK |
| | | | RLDDRSREIQDRLQKLLEEIRRKTK SEQ ID NO: 281 |
| DHD163 | Heterodimer | b | TEEEIVELNKDIQRKSKEHIDLQNELVKKIERAIRENNITEELLEELERLL |
| | | | RESEKIVEEIRRITDKIRKDAK SEQ ID NO: 282 |
| DHD164 | Heterodimer | a | SEKEILERLLRLSKEQNEISEEIHRLTERLVELKRRKDDDERLKRILDRQK |
| | | | RLVERAREISKEYEDLLRKLE SEQ ID NO: 283 |
| DHD164 | Heterodimer | b | SMEELLRKNARLSRKQLKIIDEHLELSTKLTRGEAGDETLEEIERRSREML |
| | | | EEQRRVDEESKRIREKLK SEQ ID NO: 284 |
| DHD165 | Heterodimer | a | SEEEIRDIVEKLLRTHEEVLKEIKKLLDDSERVRRRELDKKDLDRIQKEQR |
| | | | DIQEENKEKAKRFDELVKELKKAAK SEQ ID NO: 285 |
| DHD165 | Heterodimer | b | SEEEHRRTMEKVEKEVRDIKRRSEEVKKKVKANTLSEEDLVRLLERLVEDH |
| | | | KRLQDLSQEIIERDEKATK SEQ ID NO: 286 |
| DHD166 | Heterodimer | a | DEDELAKEIEDVQRRNKESQEEHDKSVKKLEAAERGEIDEDSLLRVLEEDI |
| | | | KVLEKDIEVLERSIEVIEKAE SEQ ID NO: 287 |
| DHD166 | Heterodimer | b | SEKELIRRLLEQQRQHLRLSERLIELSRRLVEVVRKGKDNRDLLRELKKLS |
| | | | EEHKKHSKDDHEKVREIREREK SEQ ID NO: 288 |
| DHS17 | Heterodimer | a | DRKDLLKRNIKLLDRHLKILDTILKLLEKLSELLKKSSSEEVVKEYKKILD |
| | | | EIRKLLEESKEIHKESKEILERES SEQ ID NO: 289 |
| DHD17 | Heterodimer | b | DEEKLIERSKRLQEESEQLLEKFEQILRELTELLEKPDSEELARKIKKLHD |
| | | | ELRKIIKRNQELIREHEEILRKRD SEQ ID NO: 290 |

In one aspect, the monomer A polypeptide comprises a polypeptide having at least 70%, 75%, 80%, 85%, 90%, 91%, 92%, 93%, 94%, 95%, 96%, 97%, 98%, 99%, or 100% sequence identity along the length of the amino acid sequence of an odd-numbered SEQ ID NO selected from the group consisting of selected from the group SEQ ID NOS: 1-290; wherein GlySer at amino acids 1 and 2 of SEQ ID NO: 1, 55, 81, 83, 101, 105, 115, 117, 119, 121, 123, 125, 127, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, or 193 are optional, e.g., GlySer at amino acids 1 and 2 of SEQ ID NO: 1, 55, 81, 83, 101, 105, 115, 117, 119, 121, 123, 125, 127, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, or 193 are not present, and wherein the odd-numbered SEQ ID NO (“chain a”) is the binding partner of the SEQ ID NO. (“chain b”) in Tables 1A.

In another aspect, the monomer B polypeptide comprises a polypeptide having at least 70%, 75%, 80%, 85%, 90%, 91%, 92%, 93%, 94%, 95%, 96%, 97%, 98%, 99%, or 100% sequence identity along the length of the amino acid sequence of an even-numbered SEQ ID NO selected from the group consisting of selected from the group SEQ ID NOS: 1-290, wherein GlySer at amino acids 1 and 2 of SEQ ID NO: 6, 8, 14, 16, 26, 30, 32, 34, 36, 38, 40, 42, 46, 48, 54, 56, 58, 60, 62, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 128, 130, 132, 134, 136, 138, 140, 142, 144, 146, 148, 150, 152, 154, 156, 158, 160, 162, 164, 166, 168, 170, 172, 176, 178, 180, 182, 184, 186, 188, 190, 192, or 194 are optional, e.g., GlySer at amino acids 1 and 2 of SEQ ID NO: 6, 8, 14, 16, 26, 30, 32, 34, 36, 38, 40, 42, 46, 48, 54, 56, 58, 60, 62, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 128, 130, 132, 134, 136, 138, 140, 142, 144, 146, 148, 150, 152, 154, 156, 158, 160, 162, 164, 166, 168, 170, 172, 176, 178, 180, 182, 184, 186, 188, 190, 192, or 194 are not present, wherein the even-numbered SEQ ID NO (“chain b”) is the binding partner of the SEQ ID NO. (“chain a”) in Table 1A.

In another embodiment of any of the above embodiments,
(i) monomer A comprises a polypeptide having at least 70%, 75%, 80%, 85%, 90%, 91%, 92%, 93%, 94%, 95%, 96%, 97%, 98%, 99%, or 100% sequence identity along the length of the amino acid sequence of an odd-numbered SEQ ID NO selected from the group consisting of selected from the group SEQ ID NOS: 1-290, 331, 336-422, 424, 426, 428, 430, 432, 434, 436, 438, 440, 442, 444, 446, 448, 450, 452, 454, 456, 458-460, 462, 464, 466, 468, 470, 472, 474, 476, 478, 480, 482, 484, 486, 488, 490, 493, and 494; and
(ii) monomer B comprises a polypeptide having at least 70%, 75%, 80%, 85%, 90%, 91%, 92%, 93%, 94%, 95%, 96%, 97%, 98%, 99%, or 100% sequence identity along the length of the amino acid sequence of an even-numbered SEQ ID NO selected from the group consisting of selected from the group SEQ ID NOS: 1-290, 331, 336-422, 424, 426, 428, 430, 432, 434, 436, 438, 440, 442, 444, 446, 448, 450, 452, 454, 456, 458-460, 462, 464, 466, 468, 470, 472, 474, 476, 478, 480, 482, 484, 486, 488, 490, 493, and 494, wherein the even-numbered SEQ ID NO is the binding partner of the odd-numbered SEQ ID NO. in step (i).

The amino acid sequences of SEQ ID NOS:1-290, 331, 336-422, 424, 426, 428, 430, 432, 434, 436, 438, 440, 442, 444, 446, 448, 450, 452, 454, 456, 458-460, 462, 464, 466, 468, 470, 472, 474, 476, 478, 480, 482, 484, 486, 488, 490, 493, and 494 are provided in Table 1B. The “binding partners” have similar design names as shown in Table 1B. For example, SEQ ID NO:1 (DHD9 A) and SEQ ID NO:2 (DHD9 B) are binding partners, and For example, SEQ ID NO:331 (DHD9 A) and SEQ ID NO:2 (DHD9 B) are binding partners, so that if monomer A comprises the polypeptide having at least 70%, 75%, 80%, 85%, 90%, 91%, 92%, 93%, 94%, 95%, 96%, 97%, 98%, 99%, or 100% sequence identity along the length of the amino acid sequence of SEQ ID NO:1 or SEQ ID NO:331, then monomer B comprises the polypeptide having at least 70%, 75%, 80%, 85%, 90%, 91%, 92%, 93%, 94%, 95%, 96%, 97%, 98%, 99%, or 100% sequence identity along the length of the amino acid sequence of SEQ ID NO:2. Similarly, SEQ ID NOS:3-4 are binding partners, SEQ ID NO:5-6 and 5-332 are binding partners, etc. Those of skill in the art will clearly understand what is meant by binding partner based on the teachings herein.

TABLE 1B

| Design | Oligomerization | | |
|---|---|---|---|
| name | State | Chain | Design sequence |
| DHD9 | Heterodimer | a | GSPKEEARELIRKQKELIKEQKKLIKEAKOKSDSRDAERIWKRSREINRES |
| | | | KKINKRIKELIKS SEQ ID NO: 1 |
| | | | PKEEARELIRKQKELIKEQKKLIKEAKQKSDSRDAERIWKRSREINRESKK |
| | | | INKRIKELIKS SEQ ID NO: 331 |
| DHD9 | Heterodimer | b | PKKEAEELAEESEELHDRSEKLHERAEQSSNSEEARKILEDIERISERIEE |
| | | | ISDRIERLLRS SEQ ID NO: 2 |
| DHD13_ | Heterodimer | a | GTKEDILERQRKIIERAQEIHRRQQEILEELERIIRKPGSSEEAMKRMLKL |
| XAAA | | | LEESLRLLKELLELSEESAQLLYEQR SEQ ID NO: 3 |
| DHD13_ | Heterodimer | b | GTEKRLLEEAERAHREQKEIIKKAQELHRRLEEIVRQSGSSEEAKKEAKKI |
| XAAA | | | LEEIRELSKRSLELLREILYLSQEQKGSLVPR SEQ ID NO: 4 |
| DHD13_ | Heterodimer | a | TKEDILERQRKIIERAQEIIRRQQEILEELERIIRKPGSSEEAMKRMLKLL |
| XAXA | | | EESLRLLKELLELLEESAQLLYEQR SEQ ID NO: 5 |
| DHD13_ | Heterodimer | b | GSTEKRLLEEAERAHREAKEIIKKAQELHRRLEEIVRQSGSSEEAKKEAKK |
| XAXA | | | ILEEIRELSKRLLELLREILYLSQEQK SEQ ID NO: 6 |
| | | | TEKRLLEEAERAHREAKEIIKKAQELHRRLEEIVRQSGSSEEAKKEAKKIL |
| | | | EEIRELSKRLLELLREILYLSQEQK SEQ ID NO: 332 |
| DHD13_ | Heterodimer | a | TKEDILERARKIIERAQEIHRRQQEILEELERIIRKPGSSEEAMKRMLKLL |
| XAAX | | | EESLRLLKELLELSEELAQLLYEQR SEQ ID NO: 7 |
| DHD13_ | Heterodimer | b | GSTEKRLLEEAERAIREQKEIIKKAQELHRRLEEIVRQSGSSEEAKKEAKK |
| XAAX | | | ILEEIRELSKRSLELLREILYLLQEQK SEQ ID NO: 8 |
| | | | TEKRLLEEAERAIREQKEIIKKAQELHRRLEEIVRQSGSSEEAKKEAKKIL |
| | | | EEIRELSKRSLELLREILYLLQEQK SEQ ID NO: 334 |
| DHD13_ | Heterodimer | a | TKEDILERQRKIIERAQEIHRRQQEILEELEYIIR SEQ ID NO: 9 |
| 2:341 | | | |
| DHD13_ | Heterodimer | b | MSEEAMKRMLKLLEESLRLLKELLELSEESAQLLYEQRKANNGSETEKRLL |
| 2:341 | | | EEAERAHREQKEIIKKAQELHRRLEEIVRQSGSSEEAKKEAKKILEEIREL |
| | | | SKRSLELLREILYLSQEQK SEQ ID NO: 10 |
| DHD13_ | Heterodimer | a | MTKEDILERQRKIIERAQEIHRRQQEILKEQEKIIRKPGSSEEAMKRSLKL |
| AAAA | | | IEESLRLLKELLELSEESAQLLYEQR SEQ ID NO: 11 |
| DHD13_ | Heterodimer | b | GTEKRLLEEAERAHREQKEIIKKAQELHKELTKIHQQSGSSEEAKKRALKI |
| AAAA | | | SQEIRELSKRSLELLREILYLSQEQK SEQ ID NO: 12 |
| DHD13_ | Heterodimer | a | TKEDILERQRKIIERAQEIHRRQQEILKRSEEIIRKPGSSEEALETLRELQ |
| BAAA | | | EESLRLLKELLELSEESAQLLYEQR SEQ ID NO: 13 |
| DHD13_ | Heterodimer | b | GSTEKRLLEEAERAHREQKEIIKKAQELHRRTEEIIRQSGSSEEAKDELRR |
| BAAA | | | IQEEIRELSKRSLELLREILYLSQEQK SEQ ID NO: 14 |
| | | | TEKRLLEEAERAHREQKEIIKKAQELHRRTEEIIRQSGSSEEAKDELRRIQ |
| | | | EEIRELSKRSLELLREILYLSQEQK SEQ ID NO: 336 |
| DHD13_ | Heterodimer | a | TTKRYLEEAERAHREQKEIIKKAQELHRRLEEIVRQ SEQ ID NO: 15 |
| 4:123 | | | |
| DHD13_ | Heterodimer | b | GSSEEAKKEAKKILEEIRELSKRSLELLREILYLSQQVNDVDEKALERQRK |
| 4:123 | | | IIERAQEIHRRQQEILEELERIIRKPGSSEEAMKRMLKLLEESLRLLKELL |
| | | | ELSEESAQLLYEAR SEQ ID NO: 16 |
| | | | SEEAKKEAKKILEEIRELSKRSLELLREILYLSQQVNDVDEKALERQRKII |
| | | | ERAQEIHRRQQEILEELERIIRKPGSSEEAMKRMLKLLEESLRLLKELLEL |
| | | | SEESAQLLYEAR SEQ ID NO: 338 |
| DHD13_ | Heterodimer | a | EAMKRMLKLLEESLRLLKELLELSEESAQLLYEAR SEQ ID NO: 17 |
| 1:234 | | | |
| DHD13_ | Heterodimer | b | TTKRYLEEAERAHREQKEIIKKAQELHRRLEEIVRQSGSSEEAKKEAKKIL |
| 7:234 | | | EEIRELSKRSLELLREILYLSQQVNDVDEKALERQRKIIERAQEIHRRQQE |
| | | | ILEELERIIRKPGS SEQ ID NO: 18 |
| DHD15 | Heterodimer | a | TREELLRENIELAKEHIEIMREILELLQKMEELLEKARGADEDVAKTIKEL |
| | | | LRRLKEIIERNQRIAKEHEYIARERS SEQ ID NO: 19 |
| DHD15 | Heterodimer | b | GTERKLLERSRRLQEESKRLLDEMAEIMRRIKKLLKKARGADEKVLDELRK |
| | | | IIERIRELLDRSRKIHERSEEIAYKEE SEQ ID NO: 20 |
| DHD20 | Heterodimer | a | GDRQELIRRNIELLKEHIKILEEISQLIEELSELLDKSSSEEVVKRYKKIL |
| | | | ERYKQLLRKSQEIHKESSEIAKKES SEQ ID NO: 21 |
| DHD20 | Heterodimer | b | GDEQKLIERSQRMQKESLELLKEIIKILDTIEKLLDKPDSEELLDTIKKLH |
| | | | DTLKKIHDRNKKLLKEHEEILRQRSGSLVPR SEQ ID NO: 22 |
| DHD21 | Heterodimer | a | DKEEEYKRLLDEIKEILKESKEVLKDSKRVLEDIKRKVPDDDLVKLLEKHV |
| | | | RLLEEHVKLLEQLIREAEKSSK SEQ ID NO: 23 |
| DHD21 | Heterodimer | b | QGSSAEELLKKIKESEKKIRDSLRKIKEIIKKSRKEGVDDKQLDLIRKVVE |
| | | | SHRDLLRLHRDLIRLLREETS SEQ ID NO: 24 |
| DHD25 | Heterodimer | a | DIDESIKEVEKLLEEVEQSLOKLDDSLKKLLEKVNQDPDVDDSVRKIVKRH |
| | | | VEILKRHEEVLKRLIEVVKEHTKTVK SEQ ID NO: 25 |
| DHD25 | Heterodimer | b | GSDREEVHKEIVKLIREIIKIHKKILKIHEKIKNGEIDPSEILKLSEEIKK |
| | | | LTDTIIKIIEDLEQLTRDLRR SEQ ID NO: 26 |
| | | | DREEVHKEIVKLIREIIKIHKKILKIHEKIKNGEIDPSEILKLSEEIKKLT |
| | | | DTIIKIIEDLEQLTRDLRR SEQ ID NO: 340 |
| DHD27 | Heterodimer | a | DRKEIVKRHQKVVELLKESSKLLRESSKLLQRLLDKTGDENLQKAVDDQDK |
| | | | AIKRQETAIRKSQEASKKLD SEQ ID NO: 27 |
| DHD27 | Heterodimer | b | DNSEEIKKVAKTSREVAEYSERVAKENDKVVKTLEEGKIDESELLRLLEES |
| | | | IKIFDTALKLHEEAYKLHQDLVRKVS SEQ ID NO: 28 |
| DHD30 | Heterodimer | a | DESEAASVAIESVQILVESVKLLEESVRILLDAVKKNGVEDLLRVAQRWEK |
| | | | LVDEWLKVVKRWLDNVRDIQR SEQ ID NO: 29 |
| DHD30 | Heterodimer | b | GSDKAEEVEKSVRKIEESIKKIRKSIKKAEDAVQLLKEGKIDAKDELRIVR |
| | | | EDLEVVKEDVEIVKEDVENVREFSS SEQ ID NO: 30 |
| | | | DKAEEVEKSVRKIEESIKKIRKSIKKAEDAVQLLKEGKIDAKDFLRIVRED |
| | | | LEVVKEDVEIVKEDVENVREFSS SEQ ID NO: 342 |
| DHD33 | Heterodimer | a | SDKEVSDKLLKASKKLLKVSEELLEVVRRLLKALKDDELIKKIADLLRKII |
| | | | DKDKKFIRTSEEIVKESR SEQ ID NO: 31 |
| DHD33 | Heterodimer | b | GSDLKEVLKTVEEAVKEIIKSSEELLQISRKILEISRVGVDEHEYISAIRE |
| | | | YLKALEKHIQILKKFIEILKELIRAVS SEQ ID NO: 32 |
| | | | DLKEVLKTVEEAVKEIIKSSEELLQISRKILEISRVGVDEHEYISAIREYL |
| | | | KALEKHIQILKKFIEILKELIRAVS SEQ ID NO: 344 |
| DHD34_ | Heterodimer | a | SKEEIDKIVKKHKKKIEEHKKKVDELKKLVEEHDKRVSQDKDDKVKKLSEE |
| XAAXA | | | VKKIIKRLEEVSKRLEEVSKKLLKVISDKR SEQ ID NO: 33 |
| DHD34_ | Heterodimer | b | GSNDEELKKILETLDRILKKLDKILTRLIEVLKKSEDPNLDDKDYTELVKQ |
| XAAXA | | | FIELIKKYEEVVKEYEEVVROLIRLES SEQ ID NO: 34 |
| | | | NDEELKKILETLDRILKKLDKILTRLIEVLKKSEDPNLDDKDYTELVKQFI |
| | | | ELIKKYEEVVKEYEEVVRQLIRLES SEQ ID NO: 346 |
| DHD34_ | Heterodimer | a | SKEEIDKIVKKHKKKIEELKKLVDELKKLVEEHDKRVSQDKDDKVKKLSEE |
| XAXXA | | | VKKIIKRVEEVAKRLEEVSKKLLKVISDKR SEQ ID NO: 35 |
| DHD34_ | Heterodimer | b | GSNDEELKKILETLDRILKKLEKILTRLIEVLKKSEDPNLDDKDYTELVKQ |
| XAXXA | | | FIELIKKFEEVIKEYEEVVRQLIRLES SEQ ID NO: 36 |
| | | | NDEELKKILETLDRILKKLEKILTRLIEVLKKSEDPNLDDKDYTELVKQFI |
| | | | ELIKKFEEVIKEYEEVVROLIRLES SEQ ID NO: 348 |
| DHD34_ | Heterodimer | a | SKEEIDKIVKKHKKKIEEHKKKVDEHKKLVEEHDKRVSQDKDDKVKKLSEE |
| XAAAA | | | LKKISKRLEEVSKRLEEVSKKLLKVISDKR SEQ ID NO: 37 |
| DHD34_ | Heterodimer | b | GSNDEELKKILETLDRILKKLDKILTRLDEVLKKSEDPNLDDKDYTELVKQ |
| XAAAA | | | YIELVKKYEEVVKEYEEVVRQLIRLES SEQ ID NO: 38 |
| | | | NDEELKKILETLDRILKKLDKILTRLDEVLKKSEDPNLDDKDYTELVKQYI |
| | | | ELVKKYEEVVKEYEEVVRQLIRLES SEQ ID NO: 418 |
| DHD36 | Heterodimer | a | DHSRKLKEILDRLRKHVKRLKEHLDELRDLVRQVPEDKLLEHVVKLSDKIL |
| | | | QISERAVREFTKSVDKDS SEQ ID NO: 39 |
| DHD36 | Heterodimer | b | GSDKKDELERILDEIRRLIERLDEILSRLNKLLELLKHGVPNAKEVVKDYI |
| | | | RLLKEYLELVKEFLKLVKRHADLVS SEQ ID NO: 40 |
| | | | DKKDELERILDEIRRLIERLDEILSRLNKLLELLKHGVPNAKEVVKDYIRL |
| | | | LKEYLELVKEFLKLVKRHADLVS SEQ ID NO: 350 |
| DHD37_ | Heterodimer | a | DSDEHLKKLKTFLENLRRHLDRLDKHIKQLRDILSENPEDERVKDVIDLSE |
| ABXB | | | RSVRIVKTVIKIFEDSVRKKE SEQ ID NO: 41 |
| DHD37_ | Heterodimer | b | GSDDKELDKLLDTLEKILQTATKIIDDANKLLEKLRRSERKDPKVVETYVE |
| ABXB | | | LLKRHEKAVKELLEIAKTHAKKVE SEQ ID NO: 42 |
| | | | DDKELDKLLDTLEKILQTATKIIDDANKLLEKLRRSERKDPKVVETYVELL |
| | | | KRHEKAVKELLEIAKTHAKKVE SEQ ID NO: 352 |
| DHD37_ | Heterodimer | a | MDEEDHLKKLKTHLEKLERHLKLLEDHAKKLEDILKERPEDSAVKESIDEL |
| BBBB | | | RRSIELVRESIEIFRQSVEEEE SEQ ID NO: 43 |
| DHD37_ | Heterodimer | b | GDVKELTKILDTLTKILETATKVIKDATKLLEEHRKSDKPDPRLIETHKKL |
| BBBB | | | VEEHETLVRQHKELAEEHLKRTR SEQ ID NO: 44 |
| DHD37_ | Heterodimer | a | DSDEHLKKLKTFLENLRRHLDRLDKLLKELRDILSENPEDERVKDVIDELE |
| XBXB | | | RVIRIVKTVIKIFEDSVRKKE SEQ ID NO: 45 |
| DHD37_ | Heterodimer | b | GSDDKELDKLLDTLEKILQTATKIIDDINKVLEKLRRSERKDPKVIETVVE |
| XBXB | | | LLKRHEKAVKELLEIAKTHAKKVE SEQ ID NO: 46 |
| | | | DDKELDKLLDTLEKILQTATKIIDDINKVLEKLRRSERKDPKVIETVVELL |
| | | | KRHEKAVKELLEIAKTHAKKVE SEQ ID NO: 354 |
| DHD37_ | Heterodimer | a | DSDEHLKKLKTFLENLRRLEDLLDKHIKQLRDILSENPEDERVKDVIDLSE |
| AXXB | | | RVVRTVKTVIKIFEDSVRKKE SEQ ID NO: 47 |
| DHD37_ | Heterodimer | b | GSDDKELDKLLDTLEKILQTATKVVDDANKLLEKLRRSERKDPKVVETYVE |
| AXXB | | | LLKRLEKLIKELLEIAKTHAKKVE SEQ ID NO: 48 |
| | | | DDKELDKLLDTLEKILQTATKVVDDANKLLEKLRRSERKDPKVVETYVELL |
| | | | KRLEKLIKELLEIAKTHAKKVE SEQ ID NO: 356 |
| DHD37_ | Heterodimer | a | DSDEHLKKLKTFLENLRRHLDRLDKHIKQLRDILSEN SEQ ID NO: 49 |
| 3:124 | | | |
| DHD37_ | Heterodimer | b | EDERVKDVIDLSERSVRIVKTVIKIFEDSVRKLEKTKPDSKTAKELDKLLD |
| 3:124 | | | TLEKILQTATKIIDDANKLLEKLRRSERKDPKVVETYVELLKRHEKAVKEL |
| | | | LEIAKTHAKKVE SEQ ID NO: 50 |
| DHD37_ | Heterodimer | a | DSDEHLYKLKTFLENLRRHLDRLDKHIKQLRDILSENPEDERVKDAIDLSE |
| 1:234 | | | RSVRIVKTVIKIFEDSVRKKEKRPIDKRDDKELDKLLDTLEKILQTATKII |
| | | | DDANKLLEYLRR SEQ ID NO: 51 |
| DHD37_ | Heterodimer | b | GDPKVVETYVELLKRHEKAVKELLEIAKTHAKKVE SEQ ID NO: 52 |
| 1:234 | | | |
| DHD37_ | Heterodimer | a | DSDEHLDRLDKHLKKLKTFLENLRRHIKQLRDILSENPEDERVKDVIDLSK |
| AXBB | | | TVIKIFEDSVRKKERSVRIVE SEQ ID NO: 53 |
| DHD37_ | Heterodimer | b | GSDDKEATKIIDDLDKLLDTLEKILQTANKLLEKLRRSERKDPKVVETYVK |
| AXBB | | | AVKELLEIAKTHAELLKRHEKKVE SEQ ID NO: 54 |
| | | | DDKEATKIIDDLDKLLDTLEKILQTANKLLEKLRRSERKDPKVVETYVKAV |
| | | | KELLEIAKTHAELLKRHEKKVE SEQ ID NO: 358 |
| DHD37_ | Heterodimer | a | DSDEHIKQLRDHLDRLDKHLKKLKTFLENLRRILSENPEDERVKTVIKIFE |
| XBBA | | | DSVRKKERSVRIVKDVIDLSE SEQ ID NO: 55 |
| DHD37_ | Heterodimer | b | GSDDKEANKLLEKATKIIDDLDKLLDTLEKILQTLRRSERKDPKAVKELLE |
| XBBA | | | IAKTHAELLKRHEKVVETYVKKVE SEQ ID NO: 56 |
| | | | DDKEANKLLEKATKIIDDLDKLLDTLEKILQTLRRSERKDPKAVKELLEIA |
| | | | KTHAELLKRHEKVVETYVKKVE SEQ ID NO: 360 |
| DHD39 | Heterodimer | a | DHSRKLEEILDRLRKHVKRLLEHLRELLSLVKENPEDKDLVEVLELSLAIL |
| | | | RRSLEAVEAFLKSVTKKDPDDEDLRRKADEIRKEVEEIKKSLAEVEKEIYK |
| | | | LK SEQ ID NO: 57 |
| DHD39 | Heterodimer | b | GSSADDVLEDILKIIRELIEILDQILSLLNQLLKLLRHGVPNAKKVVEKYK |
| | | | EILELYLQLVSLFLKIVKTHADAVSGKIDKKAEEEIKKEEEKIKEKLRQAK |
| | | | DILKKLQEEIDKTR SEQ ID NO: 58 |
| | | | SADDVLEDILKIIRELIEILDQILSLLNQLLKLLRHGVPNAKKVVEKYKEI |
| | | | LELYLQLVSLELKIVKTHADAVSGKIDKKAEEEIKKEEEKIKEKLRQAKDI |
| | | | LKKLQEEIDKTR SEQ ID NO: 362 |
| DHD40 | Heterodimer | a | DRDAHLYKLLTFLEQLVRHLDRLVKHITQLRDIVKKDPEDERAVDVIRQSV |
| | | | RSLEIVITVLKIFVDSVSDAARSKEAEKIVRKIRKEIDEIRQKLREIDKEV |
| | | | KKTTS SEQ ID NO: 59 |
| DHD40 | Heterodimer | b | GSNDKVLDKILDILDRILRLATRVIDLANKLLQVKKKSTHKDPRIVETYKE |
| | | | LLKIHETAVRLLLELADLHRRLKSKDEEANKRVETELDRIRKKVKDIEDKV |
| | | | RKLEDKVRKTAS SEQ ID NO: 60 |
| | | | NDKVLDKILDILDRILRLATRVIDLANKLLQVKKKSTHKDPRIVETYKELL |
| | | | KIHETAVRLLLELADLHRRLKSKDEEANKRVETELDRIRKKVKDIEDKVRK |
| | | | LEDKVRKTAS SEQ ID NO: 364 |
| DHD43 | Heterodimer | a | NDLSKEVLKKLEKSVEELLRRVQKSVKEAQKRGLLSDELVDRHLKILNQLV |
| | | | KRHLELLQEVIKRSDKK SEQ ID NO: 61 |
| DHD43 | Heterodimer | b | GSDEAVKRVVEKSLKILDEVIKKSLDILRELIELQIRHAKDDESVIRASKS |
| | | | ALKDAIEALKKSLDEIKKALKRSADEG SEQ ID NO: 62 |
| | | | DEAVKRVVEKSLKILDEVIKKSLDILRELIELQIRHAKDDESVIRASKSAL |
| | | | KDAIEALKKSLDEIKKALKRSADEG SEQ ID NO: 366 |
| DHD65 | Heterodimer | a | SSEEVVKVHEKVVKLHKEILELLKKIIKIHETAARDPDDKDSIKKLSDEIK |
| | | | KIVKRIEDISDQAKRESSDAQRKQS SEQ ID NO: 63 |
| DHD65 | Heterodimer | b | DKEEESKELLKKLKEILKRSEELLEESKELLKLAKNGEIDESELADADRKL |
| | | | NKKHEKLVQDIQDLLREHERQDR SEQ ID NO: 64 |
| DHD70 | Heterodimer | a | DEKKKIDKIVKETEDLLQKSEKLLQQSKEAVKRIRSQVKENEIVDRLLRIS |
| | | | EELLKISRRLVEISRRIASTLS SEQ ID NO: 65 |
| DHD70 | Heterodimer | b | GSSKEEVIRLLKENVRLIKENLELLTRNLKLITDLVRGSNGSEEKIKTLKE |
| | | | LLKEYRELLKRYRKLVEDYKRLVDKHD SEQ ID NO: 66 |
| | | | SKEEVIRLLKENVRLIKENLELLTRNLKLITDLVRGSNGSEEKIKTLKELL |
| | | | KEYRELLKRYRKLVEDYKRLVDKHD SEQ ID NO: 368 |
| DHD88 | Heterodimer | a | EIQELIKSSRRIIEESKELIKESEEVLRRIKEILDRIRNGVDNQEDLLREI |
| | | | LKLLTKNLKIIQRNLKLLQDNAEILKRLVS SEQ ID NO: 67 |
| DHD88 | Heterodimer | b | GSYIEDVIKKILDVSRELIKLSRTIIKISEEINKQLQQGRDTKDLVKKYDE |
| | | | IIKKYTRIVQHYTELIKELQKLLS SEQ ID NO: 68 |
| | | | YIEDVIKKILDVSRELIKLSRTIIKISEEINKQLQQGRDTKDLVKKYDEII |
| | | | KKYTRIVQHYTELIKELQKLLS SEQ ID NO: 370 |
| DHD89 | Heterodimer | a | SPTEEAIQLSQRVIELSKRVIELSKEILKLLKRVLDLLPDLDKNEEKRLDD |
| | | | YDKELKEYDKELKKYEKRLKDLAS SEQ ID NO: 69 |
| DHD89 | Heterodimer | b | GSEEEEILKIQKELLRIQSEILDKQKKILDTLRSNGAVTEEVRSILEKVER |
| | | | LSEEAKELSKEAKELTKEVSKLIS SEQ ID NO: 70 |
| | | | EEEEILKIQKELLRIQSEILDKQKKILDTLRSNGAVTEEVRSILEKVERLS |
| | | | EEAKELSKEAKELTKEVSKLIS SEQ ID NO: 372 |
| DHD90 | Heterodimer | a | SPLKELNNQLLRLLRELVKVSKKIVDLSKTIIEVLKHTDLDPRLLDSLEKS |
| | | | QQELDKSQKELDKVVKELTKVNKKLQ SEQ ID NO: 71 |
| DHD90 | Heterodimer | b | GSPLEDLVRKYDELVKTYEKLVEEFKKAVDKYDKAVKKAPVSKEATDSLDL |
| | | | IRKVLELLDRNLKLIKENAKLIKELLK SEQ ID NO: 72 |
| | | | PLEDLVRKYDELVKTYEKLVEEFKKAVDKYDKAVKKAPVSKEATDSLDLIR |
| | | | KVLELLDRNLKLIKENAKLIKELLK SEQ ID NO: 374 |
| DHD91 | Heterodimer | a | SPTRENEKVIKENEKVISDNERVLEEVVKVVETATDRKEIQDAVDEVRKSV |
| | | | DKLRDSVRKLEESVRTLD SEQ ID NO: 73 |
| DHD91 | Heterodimer | b | GSPIKDISKRLLEISKRLVEISDRIVELLQRIADSKDPNKDLQKEVKDVLE |
| | | | EYKRLVREYREVVKEYEKVVS SEQ ID NO: 74 |
| | | | PIKDISKRLLEISKRLVEISDRIVELLQRIADSKDPNKDLQKEVKDVLEEY |
| | | | KRLVREYREVVKEYEKVVS SEQ ID NO: 376 |
| DHD92 | Heterodimer | a | DEDEHVKQLIKNADLLRKHAELLKELVKLFQEIASQIPDDRVAKKVTDVVD |
| | | | RIDKILKQTEKLVRRTKQILDYSR SEQ ID NO: 75 |
| DHD92 | Heterodimer | b | GSNLEELVKLLKEVLEMHERLLRIHEDLVEAHKSNASDKESERKLKKSDKD |
| | | | IKESLKKIKSIIDQVRYIQS SEQ ID NO: 76 |
| | | | NLEELVKLLKEVLEMHERLLRIHEDLVEAHKSNASDKESERKLKKSDKDIK |
| | | | ESLKKIKSIIDQVRYIQS SEQ ID NO: 378 |
| DHD93 | Heterodimer | a | PVEDIIEESLRLLEESLKLLNRILKLLEDSLRKLPRSEEWRQRLDEFRKKL |
| | | | EDWKEELERWIEDVRYKKT SEQ ID NO: 77 |
| DHD93 | Heterodimer | b | GSDEDYESREIIDEIRKLLDRSKKIVHRSQRLVERVKSTPLSEDQEDLIRR |
| | | | HEETINRHRELVKELEKVLEDHERHIR SEQ ID NO: 78 |
| | | | DEDYESREIIDEIRKLLDRSKKIVHRSQRLVERVKSTPLSEDQEDLIRRHE |
| | | | ETINRHRELVKELEKVLEDHERHIR SEQ ID NO: 380 |
| DHD94 | Heterodimer | a | PEEDSRRVLERFVRVSREVLKVLEEFLRVSEELLREADRDRDRRLEEYERQ |
| | | | VDELREEIRRYKEEVDKEDKEVKYYKK SEQ ID NO: 79 |
| DHD94 | Heterodimer | b | GSPEKDENRKLLDKVRKLVEKSRRLVEELRKLVDQSTKNGLIDEKALRKQQ |
| | | | EVLRKVEEVLEKQERVLRELEEISYRVI SEQ ID NO: 80 |
| | | | PEKDENRKLLDKVRKLVEKSRRLVEELRKLVDQSTKNGLIDEKALRKQQEV |
| | | | LRKVEEVLEKQERVLRELEEISYRVI SEQ ID NO: 382 |
| DHD94_ | Heterodimer | a | GSPERDENRKLLDKVRKLVEKSRRLVEELRKLVDQSTKN SEQ ID |
| 3:214 | | | NO: 81 |
| | | | PERDENRKLLDKVRKLVEKSRRLVEELRKLVDQSTKN SEQ ID NO: 337 |
| DHD94_ | Heterodimer | b | GSDEKALRKQQEVLRKVEEVLEKQERVLRELEEISYRVITRGEDHKAEEDS |
| 3:214 | | | RRVLERFVRVSREVLKVLEEFLRVSEELLREADRDRDRRLEEYERQVDELR |
| | | | EEIRRYKEEVDKEDKEVKYYKK SEQ ID NO: 82 |
| | | | DEKALRKQQEVLRKVEEVLEKQERVLRELEEISYRVITRGEDHKAEEDSRR |
| | | | VLERFVRVSREVLKVLEEFLRVSEELLREADRDRDRRLEEYERQVDELREE |
| | | | IRRYKEEVDKEDKEVKYYKK SEQ ID NO: 384 |
| DHD94_ | Heterodimer | a | GSDRRLEEYERQVDELREEIRRYKEEVDKFDKEVKYYKK SEQ ID |
| 2:143 | | | NO: 83 |
| | | | DRRLEEYERQVDELREEIRRYKEEVDKFDKEVKYYKK SEQ ID NO: 339 |
| DHD94_ | Heterodimer | b | GSPERDENRKLLDKVRKLVEKSRRLVEELRKLVDQSTKNGLIDEKALRKQQ |
| 2:143 | | | EVLRKVEEVLEKQERVLRELEEISYRVITRGEDHKAEEDSRRVLERFVRVS |
| | | | REVLKVLEEFLRVSEELLREADR SEQ ID NO: 84 |
| | | | PERDENRKLLDKVRKLVEKSRRLVEELRKLVDQSTKNGLIDEKALRKQQEV |
| | | | LRKVEEVLEKQERVLRELEEISYRVITRGEDHKAEEDSRRVLERFVRVSRE |
| | | | VLKVLEEFLRVSEELLREADR SEQ ID NO: 386 |
| DHD95 | Heterodimer | a | DLSEESKKFVEKVKKLEKESRELEKQVKKIEEDSRSVENDVQKEFLELLKR |
| | | | LLDIQKKVVEVLREVVKVQQYVDS SEQ ID NO: 85 |
| DHD95 | Heterodimer | b | GSDSEYESRQVLRELDTVLKDSHTVLEALRQVIRDSQDVVSKSDEESRRVI |
| | | | DDLEKVIQDSKKVLDDIKRLIDKSKSIKS SEQ ID NO: 86 |
| | | | DSEYESRQVLRELDTVLKDSHTVLEALRQVIRDSQDVVSKSDEESRRVIDD |
| | | | LEKVIQDSKKVLDDIKRLIDKSKSIKS SEQ ID NO: 388 |
| DHD96 | Heterodimer | a | NEDELLKLLTENLKLLDENLKLLRENLSLLRQANNITDKNRIREIVKQSKE |
| | | | IVKQSREILKQSKEIVERIKYIVS SEQ ID NO: 87 |
| DHD96 | Heterodimer | b | GSSLYELTQRYEKLVQQYEELVKDYRRLVKKLEKLKRDNKPDKRLLKEIVD |
| | | | VIKKSVEIIDRSLKLLEESIKILEETD SEQ ID NO: 88 |
| | | | SLYELTQRYEKLVQQYEELVKDYRRLVKKLEKLKRDNKPDKRLLKEIVDVI |
| | | | KKSVEIIDRSLKLLEESIKILEETD SEQ ID NO: 390 |
| DHD97 | Heterodimer | a | SQERSLEILKRILDVLKESLEILKESLSILRQLASRIKNPNRKIEEILKES |
| | | | DKIIKESDKVLKEIEEVIRYSS SEQ ID NO: 89 |
| DHD97 | Heterodimer | b | GSDIEYESKEILELIKELLKLSRELLKESRRALELVRKSRDDSIVEEVIQV |
| | | | HKKVLDIHKEVLKIVRKVVEVHRRVKS SEQ ID NO: 90 |
| | | | DIEYESKEILELIKELLKLSRELLKESRRALELVRKSRDDSIVEEVIQVHK |
| | | | KVLDIHKEVLKIVRKVVEVHRRVKS SEQ ID NO: 392 |
| DHD98 | Heterodimer | a | SKKDESTKLERLAEKIDEITKRIEELVKDVKRKSSEGVDKDQQQKIDEVFQ |
| | | | KLLDLQREILEILDRILKVQQYILD SEQ ID NO: 91 |
| DHD98 | Heterodimer | b | GSDLEYLNRRLLQLIKTLIDLNRHLLKLIDKLKKLNSREGDEEKIKEESKQ |
| | | | IQEQFKEIVERSKEIIKQIKEIIKRSQ SEQ ID NO: 92 |
| | | | DLEYLNRRLLQLIKTLIDLNRHLLKLIDKLKKLNSREGDEEKIKEESKQIQ |
| | | | EQFKEIVERSKEIIKQIKEIIKRSQ SEQ ID NO: 394 |
| DHD99 | Heterodimer | a | DFERSSRRLEKVVEDLRRSSDRLREVIDELRKSADEKDEDEDLRRARKEHR |
| | | | DLIEELKRALEKQEEIIKHLQELVYRQL SEQ ID NO: 93 |
| DHD99 | Heterodimer | b | GSEESEEVRKVVERIKKISRELEEVVKELDRVSKEFDRHGETDEIVREHER |
| | | | IVEKLEEIVKKHTKIVEELAEIVYKQQ SEQ ID NO: 94 |
| | | | EESEEVRKVVERIKKISRELEEVVKELDRVSKEFDRHGETDEIVREHERIV |
| | | | EKLEEIVKKHTKIVEELAEIVYKQQ SEQ ID NO: 396 |
| DHD100 | Heterodimer | a | SDDDSVRVLDEIVKILDESVKLLKESLKLLDDFLRTKPDDHLKEVVKESKK |
| | | | VVEQSKKVLDRIKKIIYESK SEQ ID NO: 95 |
| DHD100 | Heterodimer | b | GSDLLYLSKELLKLVRELLKLSRELVELSRRLVNSTHKSPELVKKYDKLVK |
| | | | KYQDLLKKLADVADEYLRORS SEQ ID NO: 96 |
| | | | DLLYLSKELLKLVRELLKLSRELVELSRRLVNSTHKSPELVKKYDKLVKKY |
| | | | QDLLKKLADVADEYLRORS SEQ ID NO: 398 |
| DHD101 | Heterodimer | a | DEKDYHRRLIEHLEDLVRRHEELIKRQKKVVEELERRGLDERLRRVVDRER |
| | | | RSSERWEEVIERFRQVVDKLRKSVE SEQ ID NO: 97 |
| DHD101 | Heterodimer | b | GSDAYDLDRIVKEHRRLVEEQRELVEELEKLVRRQEDHRVDKKESHEILER |
| | | | LERIIRRSTRILTELEKLTDEFERRTR SEQ ID NO: 98 |
| | | | DAYDLDRIVKEHRRLVEEQRELVEELEKLVRRQEDHRVDKKESHEILERLE |
| | | | RIIRRSTRILTELEKLTDEFERRTR SEQ ID NO: 400 |
| DHD102 | Heterodimer | a | DERYRAREHIRRVEEHTKRLRHILKRLREHEEKLRRELKPGDEITESVDRF |
| | | | KKIVDQFEESIKKFETVSEELRKSDS SEQ ID NO: 99 |
| DHD102 | Heterodimer | b | GSDRQRILDRLDKILEKLDDILKKLKDILETLSKDDVSDRRHKDLVEKFRE |
| | | | LVDTHHKLVERYRELVYQNR SEQ ID NO: 100 |
| | | | DRQRILDRLDKILEKLDDILKKLKDILETLSKDDVSDRRHKDLVEKFRELV |
| | | | DTHHKLVERYRELVYQNR SEQ ID NO: 402 |
| DHD102_ | Heterodimer | a | GSDEITESVDRFKKIVDQFEESIKKFETVSEELRKSIS SEQ ID |
| 1:243 | | | NO: 101 |
| | | | DEITESVDRFKKIVDQFEESIKKFETVSEELRKSIS SEQ ID NO: 341 |
| DHD102_ | Heterodimer | b | GSDPQRAADRLDKILEKLDDILKKLKDILETLSKDDVKDRRAKDLVEKFRE |
| 1:243 | | | LVDTHHKLVERYRELVYTATAGSDLARELIRRVEEHTKRLRHILKRLREHE |
| | | | EKLRR SEQ ID NO: 102 |
| | | | DPQRAADRLDKILEKLDDILKKLKDILETLSKDDVKDRRAKDLVEKFRELV |
| | | | DTHHKLVERYRELVYTATAGSDLARELIRRVEEHTKRLRHILKRLREHEEK |
| | | | LRR SEQ ID NO: 404 |
| DHD103 | Heterodimer | a | NADDQLATSIKKLEDSIDQLIKIVRKFEESVKKLOKHGVDQHHVEILRKIV |
| | | | EIFRQHIEKLKKHLEKLRYTSS SEQ ID NO: 103 |
| DHD103 | Heterodimer | b | GSDKEYLVTEHEKLVREHEKIVSEIEKLVKKHEAGVDESELEEILKKVEKL |
| | | | LRKLDEILEQLTOLLRKTE SEQ ID NO: 104 |
| | | | DKEYLVTEHEKLVREHEKIVSEIEKLVKKHEAGVDESELEEILKKVEKLLR |
| | | | KLDEILEQLTOLLRKTE SEQ ID NO: 406 |
| DHD103_ | Heterodimer | a | GSDQHVVEILRKIVEIFRQHIEKLKKHLEKLRYTSS SEQ ID NO: 105 |
| 1:423 | | | DQHVVEILRKIVEIFRQHIEKLKKHLEKLRYTSS SEQ ID NO: 343 |
| DHD103_ | Heterodimer | b | GSDAEYLVTEHEKLVREHEKIVSEIEKLVKKHEKGVDESELEEILKKVEKL |
| 1:423 | | | LRKLDEILEQLTQLLRKAEKHIDKHSKAADQLATSIKKLEDSIDQLIKIVR |
| | | | KFEESVKKLQKH SEQ ID NO: 106 |
| | | | DAEYLVTEHEKLVREHEKIVSEIEKLVKKHEKGVDESELEEILKKVEKLLR |
| | | | KLDEILEQLTQLLRKAEKHIDKHSKAADQLATSIKKLEDSIDQLIKIVRKF |
| | | | EESVKKLQKH SEQ ID NO: 408 |
| DHD104 | Heterodimer | a | DEDDDIRRVLDESRRVLEHSRRVLKRSEEVLEKASRKKEKDTEEIEKHLKR |
| | | | LREHAKKLEKHRRELDDFLYKEI SEQ ID NO: 107 |
| DHD104 | Heterodimer | b | GSRDKYLLERLNDILKKLDEIVDKLSDILKRLKDVRHDDRLQELVERYKEI |
| | | | VKEYKRIVEEYEKLVREFEEQQR SEQ ID NO: 108 |
| | | | RDKYLLERLNDILKKLDEIVDKLSDILKRLKDVRHDDRLQELVERYKEIVK |
| | | | EYKRIVEEYEKLVREFEEQQR SEQ ID NO: 410 |
| DHD105 | Heterodimer | a | DRDYEDKEFKKIIKELEDVQEELKKLQEKIKRFSSELEEPNELLKEQLKVN |
| | | | EEQLEVNKKILKILRDQLKQNE SEQ ID NO: 109 |
| DHD105 | Heterodimer | b | GSDAEYKVRESVKRSKESVKHSEDVVDKLNKSVKLSESGHSDAEKASRELV |
| | | | KLVREVVELSREVIKLSEKVLRVIS SEQ ID NO: 110 |
| | | | DAEYKVRESVKRSKESVKHSEDVVDKLNKSVKLSESGHSDAEKASRELVKL |
| | | | VREVVELSREVIKLSEKVLRVIS SEQ ID NO: 412 |
| DHD106 | Heterodimer | a | DLQYKQEKLIRHFDRVVREWDKLVRKFSKVLEKQKHESKDKELEEASRRVD |
| | | | ELIKRLREQLKRSKEILRRLKELSRKSS SEQ ID NO: 111 |
| DHD106 | Heterodimer | b | GSDWEELLRRLEKVLQEYEEIVKELIDLIERLIKVSEDKSKDASEYKKLVT |
| | | | ELEKLISKLEEISKKLEELVKEYEYKTE SEQ ID NO: 112 |
| | | | DWEELLRRLEKVLQEYEEIVKELIDLIERLIKVSEDKSKDASEYKKLVTEL |
| | | | EKLISKLEEISKKLEELVKEYEYKTE SEQ ID NO: 414 |
| DHD107 | Heterodimer | a | DAKDELEKSLQEIEESLKELKKLLEELDKSLRELTSQGRNKKLEEHIKKVQ |
| | | | KFIELVKKYIKAVODYLKEVRYDNS SEQ ID NO:113 |
| DHD107 | Heterodimer | b | GSDKERAARATEEMVKLTKKLLKAVEDLVRDVRRLLKEGLISEKHARIAET |
| | | | ILEVFKKHAKIIKKHVDIVKYDES SEQ ID NO: 114 |
| | | | DKERAARATEEMVKLTKKLLKAVEDLVRDVRRLLKEGLISEKHARIAETIL |
| | | | EVFKKHAKIIKKHVDIVKYDES SEQ ID NO: 416 |
| DHD108 | Heterodimer | a | GSPLKERLLEIQRDLDRVLEEVVERLLRIQERLDSVVERKPPDVHEEYKYI |
| | | | VDEIREIVERVVREYEEIVKRIDEEVR SEQ ID NO: 115 |
| | | | PLKERLLEIQRDLDRVLEEVVERLLRIQERLDSVVERKPPDVHEEYKYIVD |
| | | | EIREIVERVVREYEEIVKRIDEEVR SEQ ID NO: 459 |
| DHD108 | Heterodimer | b | GSEEDERIRYDLDRIRKDVRRKLEEIRQRVRELEKKLRDAGHRRDEKELLR |
| | | | ELIETSKDILRLVEELLKKIIDKSEDLLRKTE SEQ ID NO: 116 |
| | | | EEDERIRYDLDRIRKDVRRKLEEIRQRVRELEKKLRDAGHRRDEKELLREL |
| | | | IETSKDILRLVEELLKKIIDKSEDLLRKTE SEQ ID NO: 420 |
| DHD109 | Heterodimer | a | GSDEEDYINENVEKDVRDIEDDVRRINERIRELLEKIRTEEVLORVLEEHH |
| | | | ELVERVLRKLVEILRKHEEENR SEQ ID NO: 117 |
| | | | DEEDYINENVEKDVRDIEDDVRRINERIRELLEKIRTEEVLQRVLEEHHEL |
| | | | VERVLRKLVEILRKHEEENR SEQ ID NO: 345 |
| DHD109 | Heterodimer | b | GSDEEEYYKEKLHKLLREIEELLKHYRELVRRLEELVKRGELDKDTAAHIL |
| | | | ERLSELLERIIRRVAHTLRRLSEERR SEQ ID NO: 118 |
| | | | DEEEYYKEKLHKLLREIEELLKHYRELVRRLEELVKRGELDKDTAAHILER |
| | | | LSELLERIIRRVAHTLRRLSEERR SEQ ID NO: 422 |
| DHD110 | Heterodimer | a | GSDEDEISYDSKRRVEEIVRQAREKSEKSRKDIEDVAEVLRKGDVSEKEVV |
| | | | DELVKVLEEQVKVLREAVERLREVLKKQVDDVR SEQ ID NO: 119 |
| | | | DEDEISYDSKRRVEEIVRQAREKSEKSRKDIEDVAEVLRKGDVSEKEVVDE |
| | | | LVKVLEEQVKVLREAVERLREVLKKQVDDVR SEQ ID NO: 347 |
| DHD110 | Heterodimer | b | GSDIVELVDHLLKRSLKLLEELAELVRRLLEKSTELLKRRTEEHKEEVVEE |
| | | | SEYMVRELEERLRRVVDESEKLVRDADKHIR SEQ ID NO: 120 |
| | | | DIVELVDHLLKRSLKLLEELAELVRRLLEKSTELLKRRTEEHKEEVVEESE |
| | | | YMVRELEERLRRVVDESEKLVRDADKHIR SEQ ID NO: 424 |
| DHD111 | Heterodimer | a | GSKEKDIVKTLVDLLRENLETLERLIEEVVRLLKENVDVRDEGRDDKDSER |
| | | | ILRDIKRRIDEAAKESREIIERIEKEVEYRSR SEQ ID NO: 121 |
| | | | KEKDIVKTLVDLLRENLETLERLIEEVVRLLKENVDVRDEGRDDKDSERIL |
| | | | RDIKRRIDEAAKESREIIERIEKEVEYRSR SEQ ID NO: 349 |
| DHD111 | Heterodimer | b | GSPEVDVLRRIVREILKASEELLRLLRKLIDEALKLSERKRDSQEYREVVD |
| | | | RVKKELERLLDEYRKLVEELKEKLRYDTR SEQ ID NO: 122 |
| | | | PEVDVLRRIVREILKASEELLRLLRKLIDEALKLSERKRDSQEYREVVDRV |
| | | | KKELERLLDEYRKLVEELKEKLRYDTR SEQ ID NO: 426 |
| DHD112 | Heterodimer | a | GSDKRYESEKLKRRLDEAVEKVREVVERVERESDRVLEEVRRRRESKEVVD |
| | | | KVIEDNDKALEDVLRVVDEVAKVVRDVVRENTR SEQ ID NO: 123 |
| | | | DKRYESEKLKRRLDEAVEKVREVVERVERESDRVLEEVRRRRESKEVVDKV |
| | | | IEDNDKALEDVLRVVDEVAKVVRDVVRENTR SEQ ID NO: 351 |
| DHD112 | Heterodimer | b | GSPREYHSKDILRKVDEILERIRRHADRVKKKSERLKRENVDVNEHSKDVK |
| | | | RVIRELLELVKELLRLAKKHSDDQQE SEQ ID NO: 124 |
| | | | PREYHSKDILRKVDEILERIRRHADRVKKKSERLKRENVDVNEHSKDVKRV |
| | | | IRELLELVKELLRLAKKHSDDQQE SEQ ID NO: 428 |
| DHD113 | Heterodimer | a | GSDEDEILYHSERLLQKLKKELDDLKEKSRELLEELKKEDPDDRLIERIIR |
| | | | LHDEVLKDLDEVLKNILEVHREVLERLR SEQ ID NO: 125 |
| | | | DEDEILYHSERLLQKLKKELDDLKEKSRELLEELKKEDPDDRLIERIIRLH |
| | | | DEVLKDLDEVLKNILEVHREVLERLR SEQ ID NO: 353 |
| DHD113 | Heterodimer | b | DKLDRLLKIHEEALRRAEELIKRLLDIHRRALDLARRGELDDYLLKESERE |
| | | | LREIIRRAREELKESRDRLEEISR SEQ ID NO: 126 |
| DHD114 | Heterodimer | a | GSPKEELIRRVLEEVKRLNEKLLEIIRRAAELVKRANDELPETEKLREIDR |
| | | | ELEKKLKEIEDELRRIDKELDDALYEIED SEQ ID NO: 127 |
| | | | PKEELIRRVLEEVKRLNEKLLEIIRRAAELVKRANDELPETEKLREIDREL |
| | | | EKKLKEIEDELRRIDKELDDALYEIED SEQ ID NO: 355 |
| DHD114 | Heterodimer | b | GSPKLDKLRELLERNLEKLREILEEVLKILRTNLERVREDIRDEDVLQEYE |
| | | | RLIRKAEEDLRRVLKEYDDLLKKLVYELR SEQ ID NO: 128 |
| | | | PKLDKLRELLERNLEKLREILEEVLKILRTNLERVREDIRDEDVLQEYERL |
| | | | IRKAEEDLRRVLKEYDDLLKKLVYELR SEQ ID NO: 430 |
| DHD115 | Heterodimer | a | GSKEDESVKRAEEIVRTLLKLLEDSLREAERSLRDIKNGEDEHNLRRISEK |
| | | | LEELSKRITETIERLLRELQYTSR SEQ ID NO: 129 |
| | | | KEDESVKRAEEIVRTLLKLLEDSLREAERSLRDIKNGEDEHNLRRISEKLE |
| | | | ELSKRITETIERLLRELQYTSR SEQ ID NO: 357 |
| DHD115 | Heterodimer | b | GSPNQELLDRVRKILEDLLRLNEELVRLNKELLKRALEMRRKNRDSEEVLE |
| | | | RLAEEYRKRLEEYRRELEKLLEELEETIYRYKR SEQ ID NO: 130 |
| | | | PNQELLDRVRKILEDLLRLNEELVRINKELLKRALEMRRKNRDSEEVLERL |
| | | | AEEYRKRLEEYRRELEKLLEELEETIYRYKR SEQ ID NO: 432 |
| DHD116 | Heterodimer | a | GSDESEEAQHEVEKVLDDIRRLSEHLQKRLEEVLEEVYELRREGSDRTEVV |
| | | | ELLKEVIREIVRVNREALERLLRVVEEAVKRNE SEQ ID NO: 131 |
| | | | DESEEAQHEVEKVLDDIRRLSEHLQKRLEEVLEEVYELRREGSDRTEVVEL |
| | | | LKEVIREIVRVNREALERLLRVVEEAVKRNE SEQ ID NO: 359 |
| DHD116 | Heterodimer | b | GSDEEELVETVKRIQKEILDRLTELAKLLVEIQREIKKLKDEGEDDKELKR |
| | | | LSDELEEKVRQVVEEIKRLSDELEETVEYVSR SEQ ID NO: 132 |
| | | | DEEELVETVKRIQKEILDRLTELAKLLVEIQREIKKLKDEGEDDKELKRLS |
| | | | DELEEKVRQVVEEIKRLSDELEETVEYVSR SEQ ID NO: 434 |
| DHD117 | Heterodimer | a | GSDEEEEVVRRAEELVKEHEELIERVIRTHEELVYKLEDQGADKKLVDVLK |
| | | | RVVEESERVAREIVKVSRELIRLLEEASR SEQ ID NO: 133 |
| | | | DEEEEVVRRAEELVKEHEELIERVIRTHEELVYKLEDQGADKKLVDVLKRV |
| | | | VEESERVAREIVKVSRELIRLLEEASR SEQ ID NO: 361 |
| DHD117 | Heterodimer | b | GSSKEEILKELEDLQRRLIEELKKLQERVVELLEELIKRLRDRGRDDKHLK |
| | | | RLVKEVRRLSEEVLRSIKEVSDRVRYQLR SEQ ID NO: 134 |
| | | | SKEEILKELEDLQRRLIEELKKLQERVVELLEELIKRLRDRGRDDKHLKRL |
| | | | VKEVRRLSEEVLRSIKEVSDRVRYQLR SEQ ID NO: 436 |
| DHD118 | Heterodimer | a | GSDKEEESEYLLRDLVRLLEKVKEKIEEVNREVEKLLKKVKDGRLDRREVL |
| | | | REILRLNRELAEIIKEVVDRIRHVVERSER SEQ ID NO: 135 |
| | | | DKEEESEYLLRDLVRLLEKVKEKIEEVNREVEKLLKKVKDGRLDRREVLRE |
| | | | ILRLNRELAEIIKEVVDRIRHVVERSER SEQ ID NO: 363 |
| DHD118 | Heterodimer | b | GSDLHEVVYETKELLKRIEEVVEELRKKSEDIIRKAERGEISEDELKRLQE |
| | | | EIAREAKKLLDEIKRVLERHLEQTL SEQ ID NO: 136 |
| | | | DLHEVVYETKELLKRIEEVVEELRKKSEDIIRKAERGEISEDELKRLQEEI |
| | | | AREAKKLLDEIKRVLERHLEQTL SEQ ID NO: 438 |
| DHD119 | Heterodimer | a | GSPVEEIIKEVVKRVIEVQEKVLRIISHAVKRVVEVQKKYDPGSEESNRVV |
| | | | EEVKKTIEDAIRESDEVVDEVVKRIQYTVR SEQ ID NO: 137 |
| | | | PVEEIIKEVVKRVIEVQEKVLRIISHAVKRVVEVQKKYDPGSEESNRVVEE |
| | | | VKKTIEDAIRESDEVVDEVVKRIQYTVR SEQ ID NO: 365 |
| DHD119 | Heterodimer | b | GSPEQEIADRILTEIRESQKELERLARKILKLLDESQEKAKRGRLSEEESD |
| | | | ELLERIKKELDELLERSKELLKKIEYELR SEQ ID NO: 138 |
| | | | PEQEIADRILTEIRESQKELERLARKILKLLDESQEKAKRGRLSEEESDEL |
| | | | LERIKKELDELLERSKELLKKIEYELR SEQ ID NO: 440 |
| DHD120 | Heterodimer | a | GSDEDKEANRVLDEVLKTVRDLLETANEVLKEVLYRLKRTDDQEKVVRTLT |
| | | | EVLKEHLKLVEEIVRILDKVLKEHLETEK SEQ ID NO: 139 |
| | | | DEDKEANRVLDEVLKTVRDLLETANEVLKEVLYRLKRTDDQEKVVRTLTEV |
| | | | LKEHLKLVEEIVRILDKVLKEHLETEK SEQ ID NO: 367 |
| DHD120 | Heterodimer | b | GSPEDDVLRRLEEVSEKILRVAEDVARQLREVSEKITQGKVDRKEWEEDIK |
| | | | RLKRELEELLREWKEEIERLTYELR SEQ ID NO: 140 |
| | | | PEDDVLRRLEEVSEKILRVAEDVARQLREVSEKITQGKVDRKEWEEDIKRL |
| | | | KRELEELLREWKEEIERLTYELR SEQ ID NO: 442 |
| DHD121 | Heterodimer | a | GSRREEVVKRIRELLKRNKELIDRIRELLEENEYLDKDARDKDVLRRSVEL |
| | | | LEELVRILEESVELAKEIIKLLREVVE SEQ ID NO: 141 |
| | | | RREEVVKRIRELLKRNKELIDRIRELLEENEYLDKDARDKDVLRRSVELLE |
| | | | ELVRILEESVELAKEIIKLLREVVE SEQ ID NO: 369 |
| DHD121 | Heterodimer | b | GSDEKEDNRRLQHKIERILEKNEDLQRKLEEILELLERGEADEEKIDRLRK |
| | | | AVEDYRRVVEEIKEDVKRHKYTVR SEQ ID NO: 142 |
| | | | DEKEDNRRLQHKIERILEKNEDLQRKLEEILELLERGEADEEKIDRLRKAV |
| | | | EDYRRVVEEIKEDVKRHKYTVR SEQ ID NO: 444 |
| DHD122 | Heterodimer | a | GSDEKEEAKKASEESVRTVERILEELLKASEESVELLRRGEDAKDVVERSK |
| | | | EALKRVKELLDEVVKRSDEILKYIHN SEQ ID NO: 143 |
| | | | DEKEEAKKASEESVRTVERILEELLKASEESVELLRRGEDAKDVVERSKEA |
| | | | LKRVKELLDEVVKRSDEILKYIHN SEQ ID NO: 371 |
| DHD122 | Heterodimer | b | GSDEKKLINEVVETQKRLIKEAAKRLSEVVRHQTELIRELREKNVDDKDVE |
| | | | KLLKESLDLAEEIVRRIKELLDESKKLVEYVSN SEQ ID NO: 144 |
| | | | DEKKLINEVVETQKRLIKEAAKRLSEVVRHQTELIRELREKNVDDKDVEKL |
| | | | LKESLDLAEEIVRRIKELLDESKKLVEYVSN SEQ ID NO: 446 |
| DHD123 | Heterodimer | a | GSPDMDEVKRVLDELIEIQEEILREIKRVLEKLIKIQEDNGSEYESREVVR |
| | | | EIVEIARKLVERSRRVVKKITETLQ SEQ ID NO: 145 |
| | | | PDMDEVKRVLDELIEIQEEILREIKRVLEKLIKIQEDNGSEYESREVVREI |
| | | | VEIARKLVERSRRVVKKITETLQ SEQ ID NO: 373 |
| DHD123 | Heterodimer | b | GSDERYATREIVERIERIAREILKRTEEIVREVREVLSRDVDQEEVVRRLA |
| | | | DLLRESVELVQHLVRRVEELLQESVERKK SEQ ID NO: 146 |
| | | | DERYATREIVERIERIAREILKRTEEIVREVREVLSRDVDQEEVVRRLADL |
| | | | LRESVELVQHLVRRVEELLQESVERKK SEQ ID NO: 448 |
| DHD124 | Heterodimer | a | GSPEREALREVLEDLKRVTDRLRELVERVLEELKKVTDHVDSERILRESRR |
| | | | VLKELKDIIEEILRESEKVLEKLKYTED SEQ ID NO: 147 |
| | | | PEREALREVLEDLKRVTDRLRELVERVLEELKKVTDHVDSERILRESRRVL |
| | | | KELKDIIEEILRESEKVLEKLKYTED SEQ ID NO: 375 |
| DHD124 | Heterodimer | b | GSPAREILEEVVKKHLEVVEDAARILEEIIREHEKAVREDRDKKELEEISR |
| | | | DLLRKAREALKKVKDISDDLSREIEYVAS SEQ ID NO: 148 |
| | | | PAREILEEVVKKHLEVVEDAARILEEIIREHEKAVREDRDKKELEEISRDL |
| | | | LRKAREALKKVKDISDDLSREIEYVAS SEQ ID NO: 450 |
| DHD125 | Heterodimer | a | GSPVEEAIKKVIDDLRDVQRKIRELVEELIRLLEEVQRDNDKRESEYVVER |
| | | | VEEILRRITETSREVVRKAVEDLS SEQ ID NO: 149 |
| | | | PVEEAIKKVIDDLRDVQRKIRELVEELIRLLEEVQRDNDKRESEYVVERVE |
| | | | EILRRITETSREVVRKAVEDLS SEQ ID NO: 377 |
| DHD125 | Heterodimer | b | GSDSDEKAEYLLKEMERVVRESDEVVKKILRDLEEVLERLRRGEISEDDVT |
| | | | EILKELAERHIRAIEELVRRLRELLERHKR SEQ ID NO: 150 |
| | | | DSDEKAEYLLKEMERVVRESDEVVKKILRDLEEVLERLRRGEISEDDVTEI |
| | | | LKELAERHIRAIEELVRRLRELLERHKR SEQ ID NO: 452 |
| DHD126 | Heterodimer | a | GSPVEEVLKELSEVNERVRDIAREIIERLSEVNEEVKETDDEDELKKISKK |
| | | | VVDEVEDLLRKILEVSEEVVRRVEYHDR SEQ ID NO: 151 |
| | | | PVEEVLKELSEVNERVRDIAREIIERLSEVNEEVKETDDEDELKKISKKVV |
| | | | DEVEDLLRKILEVSEEVVRRVEYHDR SEQ ID NO: 379 |
| DHD126 | Heterodimer | b | GSPKEDILREVLRRHKEIVREIVRLVREAVETHLELVKRNSDDRDAQDVIR |
| | | | KLEEDLERLVRHAQEVIEEIFYRLH SEQ ID NO: 152 |
| | | | PKEDILREVLRRHKEIVREIVRLVREAVETHLELVKRNSDDRDAQDVIRKL |
| | | | EEDLERLVRHAQEVIEEIFYRLH SEQ ID NO: 454 |
| DHD127 | Heterodimer | a | GSPRSYLLKELADLSQHLVRLLERLVRESERVVEVLERGEVDEEELKRLED |
| | | | LHRELEKAVREVRETHREIRERSR SEQ ID NO: 153 |
| | | | PRSYLLKELADLSQHLVRLLERLVRESERVVEVLERGEVDEEELKRLEDLH |
| | | | RELEKAVREVRETHREIRERSR SEQ ID NO: 381 |
| DHD127 | Heterodimer | b | GSDREYIIKDILDSQEHLLRLIEELLETQKELLEILKRRPDSVERVRELVR |
| | | | RSKEIADEIRRQSDRNVRLLEEVSK SEQ ID NO: 154 |
| | | | DREYIIKDILDSQEHLLRLIEELLETQKELLEILKRRPDSVERVRELVRRS |
| | | | KEIADEIRRQSDRNVRLLEEVSK SEQ ID NO: 456 |
| DHD128 | Heterodimer | a | GSDEKDEIRHVIESVERLIEDIKRLLKTLRELAHDDSDKKTVKEVLDRVKE |
| | | | MIERHRRELEEHRKELERAEYEVR SEQ ID NO: 155 |
| | | | DEKDEIRHVIESVERLIEDIKRLLKTLRELAHDDSDKKTVKEVLDRVKEMI |
| | | | ERHRRELEEHRKELERAEYEVR SEQ ID NO: 383 |
| DHD128 | Heterodimer | b | GSESEDRIKELLKRHIELVERHEELLHEIKKLIDLEEKDDKDREEAVKRID |
| | | | DAIKESEEMLEESKEILEEIEYLNR SEQ ID NO: 156 |
| | | | ESEDRIKELLKRHIELVERHEELLHEIKKLIDLEEKDDKDREEAVKRIDDA |
| | | | IKESEEMLEESKEILEEIEYLNR SEQ ID NO: 458 |
| DHD129 | Heterodimer | a | GSSLEDSVRLNDEVVKVVERVVRLNQEVVRLIKHATDVEDEETVKYVLERV |
| | | | REVLDESREVLKRVHELLEESERRLE SEQ ID NO: 157 |
| | | | SLEDSVRLNDEVVKVVERVVRLNQEVVRLIKHATDVEDEETVKYVLERVRE |
| | | | VLDESREVLKRVHELLEESERRLE SEQ ID NO: 385 |
| DHD129 | Heterodimer | b | GSHEKDIVYKVEDLVRKSDRIAERAREIVKRSRDIMREIRKDKDNKKLSDD |
| | | | LLKVTRDLQRVVDELEELSRELLRVAEESRK SEQ ID NO: 158 |
| | | | HEKDIVYKVEDLVRKSDRIAERAREIVKRSRDIMREIRKDKDNKKLSDDLL |
| | | | KVTRDLORVVDELEELSRELLRVAEESRK SEQ ID NO: 460 |
| DHD130 | Heterodimer | a | GSPELDEVKKLIDELKKSVERLEESIREVKESIKKLRKGDIDAEENIKLLK |
| | | | ENIKIVRENIKIIKEIIDVVQYVLR SEQ ID NO: 159 |
| | | | PELDEVKKLIDELKKSVERLEESIREVKESIKKLRKGDIDAEENIKLLKEN |
| | | | IKIVRENIKIIKEIIDVVQYVLR SEQ ID NO: 387 |
| DHD130 | Heterodimer | b | GSDEEEIEELLRELEKLLKKSEEALEESKKLIDESEELLRRDRLDKEKHVR |
| | | | ASEEHVKLSEEHLRISREIVKILEKAVYSTR SEQ ID NO: 160 |
| | | | DEEEIEELLRELEKLLKKSEEALEESKKLIDESEELLRRDRLDKEKHVRAS |
| | | | EEHVKLSEEHLRISREIVKILEKAVYSTR SEQ ID NO: 462 |
| DHD131 | Heterodimer | a | GSDESDRIRKIVEESDEIVKESRKLAERARELIKESEDKRVSEERNERLLE |
| | | | ELLRILDENAELLKRNLELLKEVLYRTR SEQ ID NO: 161 |
| | | | DESDRIRKIVEESDEIVKESRKLAERARELIKESEDKRVSEERNERLLEEL |
| | | | LRILDENAELLKRNLELLKEVLYRTR SEQ ID NO: 389 |
| DHD131 | Heterodimer | b | GSDEDDELERLLREYHRVLREYEKLLEELRRLYEEYKRGEVSEEESDRILR |
| | | | EIKEILDKSERLWDLSEEVWRTLLYQAE SEQ ID NO: 162 |
| | | | DEDDELERLLREYHRVLREYEKLLEELRRLYEEYKRGEVSEEESDRILREI |
| | | | KEILDKSERLWDLSEEVWRTLLYQAE SEQ ID NO: 464 |
| DHD132 | Heterodimer | a | GSDKKDASRRAIRVLHEFVRVSEEVLEVLRKSVESLKRLDVDEKIKRTHDR |
| | | | IEEELRRWKRELEELIERLREWEYHQD SEQ ID NO: 163 |
| | | | DKKDASRRAIRVLHEFVRVSEEVLEVLRKSVESLKRLDVDEKIKRTHDRIE |
| | | | EELRRWKRELEELIERLREWEYHQD SEQ ID NO: 391 |
| DHD132 | Heterodimer | b | GSDDEEEDKRLLEEVKRSLDTDERILEKLRHSLERQLEDVDKDEDSRRVLR |
| | | | ELDEITKRSREVVKRLRKLAYESK SEQ ID NO: 164 |
| | | | DDEEEDKRLLEEVKRSLDTDERILEKLRHSLERQLEDVDKDEDSRRVLREL |
| | | | DEITKRSREVVKRLRKLAYESK SEQ ID NO: 466 |
| DHD133 | Heterodimer | a | GSDKEYKLDRILRRLDELIKQLSRILEEIERLVDELEREPLDDKEVQDVIE |
| | | | RIVELIDEHLELLKEYIKLLEEYIKTTK SEQ ID NO: 165 |
| | | | DKEYKLDRILRRLDELIKQLSRILEEIERLVDELEREPLDDKEVQDVIERI |
| | | | VELIDEHLELLKEYIKLLEEYIKTTK SEQ ID NO: 393 |
| DHD133 | Heterodimer | b | GSPSKEYQEKSAERQKELLHEYEKLVRHLRELVEKLORRELDKEEVLRRLV |
| | | | EILERLKDLHKKIEDAHRKNEEAHKENK SEQ ID NO: 166 |
| | | | PSKEYQEKSAERQKELLHEYEKLVRHLRELVEKLORRELDKEEVLRRLVEI |
| | | | LERLKDLHKKIEDAHRKNEEAHKENK SEQ ID NO: 468 |
| DHD134 | Heterodimer | a | GSRDRKISEELIKALEDHIRMLEELIRAIEEHIKLAERGVDEKELRESLEE |
| | | | LKKIVDELEKSLEELRKLAERYKYETR SEQ ID NO: 167 |
| | | | RDRKISEELIKALEDHIRMLEELIRAIEEHIKLAERGVDEKELRESLEELK |
| | | | KIVDELEKSLEELRKLAERYKYETR SEQ ID NO: 395 |
| DHD134 | Heterodimer | b | GSPKEESVEELKRVIDKHEEILRELKRVLEEHERVSHDEDENELRRSLERL |
| | | | KHILDRLHESLKELHELLKKNEYTER SEQ ID NO: 168 |
| | | | PKEESVEELKRVIDKHEEILRELKRVLEEHERVSHDEDENELRRSLERLKH |
| | | | ILDRLHESLKELHELLKKNEYTER SEQ ID NO: 470 |
| DHD135 | Heterodimer | a | GSDHEYWVKIVERILRVMEKHAEIVKKHLEIVERVVREGPSEDLRRKLKES |
| | | | LREIEESLRELKELLDELDELSEKTR SEQ ID NO: 169 |
| | | | DHEYWVKIVERILRVMEKHAEIVKKHLEIVERVVREGPSEDLRRKLKESLR |
| | | | EIEESLRELKELLDELDELSEKTR SEQ ID NO: 397 |
| DHD135 | Heterodimer | b | GSDEEYVTRSQRRLKRLLEEYIKVVEEHARLVERNERDDKELKRSIDELDK |
| | | | LTKELLELVKRYKELVDKTET SEQ ID NO: 170 |
| | | | DEEYVTRSQRRLKRLLEEYIKVVEEHARLVERNERDDKELKRSIDELDKLT |
| | | | KELLELVKRYKELVDKTET SEQ ID NO: 472 |
| DHD136 | Heterodimer | a | GSDKEEIVKLQDEVIKTLERHLDILRKHIDLLEKLKDHLSEELKERVDRSI |
| | | | KKLEESIKRLERIIEELQELAEYSL SEQ ID NO: 171 |
| | | | DKEEIVKLQDEVIKTLERHLDILRKHIDLLEKLKDHLSEELKERVDRSIKK |
| | | | LEESIKRLERIIEELQELAEYSL SEQ ID NO: 399 |
| DHD136 | Heterodimer | b | GSREEELKESAEELERSVRELKKEADKYKEEVDRLHYRGKVDKDWVRVVEK |
| | | | LIKLVEEHLELIREHLELLKEERR SEQ ID NO: 172 |
| | | | REEELKESAEELERSVRELKKEADKYKEEVDRLHYRGKVDKDWVRVVEKLI |
| | | | KLVEEHLELIREHLELLKEERR SEQ ID NO: 474 |
| DHD137 | Heterodimer | a | GSDMEYELKKSAEELRKSLEELKRILDELHKSLRELRRHGDDEEYVQTVEE |
| | | | LRKELEEHAKKLEEHLKELERVAT SEQ ID NO: 173 |
| | | | DMEYELKKSAEELRKSLEELKRILDELHKSLRELRRHGDDEEYVQTVEELR |
| | | | KELEEHAKKLEEHLKELERVAT SEQ ID NO: 401 |
| DHD137 | Heterodimer | b | PEYELKKSVDDLKRDVDRLVEEVEEVFELSKERLREDRKHLELVEEMVRLI |
| | | | EKHLELIKEHLKLADDHVR SEQ ID NO: 174 |
| DHD138 | Heterodimer | a | GSREKDESKELNDEYKKLLEEYERLLRRSEELVKRAKGPRDEKELKRILEE |
| | | | NEDILRRTKEILERTKEISEEQKYRRR SEQ ID NO: 175 |
| | | | REKDESKELNDEYKKLLEEYERLLRRSEELVKRAKGPRDEKELKRILEENE |
| | | | DILRRTKEILERTKEISEEQKYRRR SEQ ID NO: 403 |
| DHD138 | Heterodimer | b | GSDKDERQERLNEESDKSNEESERSNRESEELNRRARGPNDEKELQEILDR |
| | | | HLELLERNQRLLDENKEILRESQYLND SEQ ID NO: 176 |
| | | | DKDERQERLNEESDKSNEESERSNRESEELNRRARGPNDEKELQEILDRHL |
| | | | ELLERNQRLLDENKEILRESQYIND SEQ ID NO: 476 |
| DHD139 | Heterodimer | a | GSENKYILKEILKLLRENLKLLHDILRLLDENLEELEKHGAKDLDDYRRKI |
| | | | EEIRKKVEDYREKIEEIEKKVERDR SEQ ID NO: 177 |
| | | | ENKYILKEILKLLRENLKLLHDILRLLDENLEELEKHGAKDLDDYRRKIEE |
| | | | IRKKVEDYREKIEEIEKKVERDR SEQ ID NO: 405 |
| DHD139 | Heterodimer | b | GSESEYTQEEILELLKESIKLLREILRLLEESEELWRRENTKSERSEEIKE |
| | | | RAKEAIKRSEEILERVKRLSDHSR SEQ ID NO: 178 |
| | | | ESEYTQEEILELLKESIKLLREILRLLEESEELWRRENTKSERSEEIKERA |
| | | | KEAIKRSEEILERVKRLSDHSR SEQ ID NO: 478 |
| DHD140 | Heterodimer | a | GSDEEEANYVSDKAVKIAEDVQELLKELLELSEVVRRGEVDEDEYDRVLRK |
| | | | LQEVMKEYEEVLKEYEEVSRKHE SEQ ID NO: 179 |
| | | | DEEEANYVSDKAVKIAEDVQELLKELLELSEVVRRGEVDEDEYDRVLRKLQ |
| | | | EVMKEYEEVLKEYEEVSRKHE SEQ ID NO: 407 |
| DHD140 | Heterodimer | b | GSPEKYLIKTQEELLRRHAEILEDLIRKVERQVDLRRKVDERDEDLKRELE |
| | | | RSLRELERLVRESSRLVEEIRELSKEIKR SEQ ID NO: 180 |
| | | | PEKYLIKTQEELLRRHAEILEDLIRKVERQVDLRRKVDERDEDLKRELERS |
| | | | LRELERLVRESSRLVEEIRELSKEIKR SEQ ID NO: 480 |
| DHD141 | Heterodimer | a | GSDEEYELERISRESKELLERYKRLLREYQELLKELRHVKDLDRAVKIIHE |
| | | | LMRVSKELVEISHRLLELHERLVRRRK SEQ ID NO: 181 |
| | | | DEEYELERISRESKELLERYKRLLREYQELLKELRHVKDLDRAVKIIHELM |
| | | | RVSKELVEISHRLLELHERLVRRRK SEQ ID NO: 409 |
| DHD141 | Heterodimer | b | GSEKEYIEKLSRKIEEDIRRSEERAKDSERLVRRLEELAKRKRLDLDDVLR |
| | | | VAEENLEILEDNLRILEEILKEQDKSNR SEQ ID NO: 182 |
| | | | EKEYIEKLSRKIEEDIRRSEERAKDSERLVRRLEELAKRKRLDLDDVLRVA |
| | | | EENLEILEDNLRILEEILKEQDKSNR SEQ ID NO: 482 |
| DHD142 | Heterodimer | a | GSPHEEVVELHERVMEISERAVELIQRIIDIIRRIREDDKDIEKLVKTIRD |
| | | | LVREYEELHRELEEIDEEIYKKSE SEQ ID NO: 183 |
| | | | PHEEVVELHERVMEISERAVELIQRIIDIIRRIREDDKDIEKLVKTIRDLV |
| | | | REYEELHRELEEIDEEIYKKSE SEQ ID NO: 411 |
| DHD142 | Heterodimer | b | GSDHEDVVRLHEDLVRKQEDARRVLEEIVRLAEEIVEVIKKDEKDKDRVTR |
| | | | LVEEIEKLVEEYKKKVDEMRKISDEIKYRSR SEQ ID NO: 184 |
| | | | DHEDVVRLHEDLVRKQEDARRVLEEIVRLAEEIVEVIKKDEKDKDRVTRLV |
| | | | EEIEKLVEEYKKKVDEMRKISDEIKYRSR SEQ ID NO: 484 |
| DHD143 | Heterodimer | a | GSRAREVVKRAKRIIEEWQKILEEWRRILEEWRRLLEDERVDDRDNERIIR |
| | | | ENERVIRENEKIIRDVIRLLEELLYERR SEQ ID NO: 185 |
| | | | RAREVVKRAKRIIEEWQKILEEWRRILEEWRRLLEDERVDDRDNERIIREN |
| | | | ERVIRENEKIIRDVIRLLEELLYERR SEQ ID NO: 413 |
| DHD143 | Heterodimer | b | GSREDEELEEEIDRIRQMVEEYEELVKEYEELTEKYKQGKVDKEESKKIIE |
| | | | KSERLLDLSQDAVRKVKEIIRRILYTNR SEQ ID NO: 186 |
| | | | REDEELEEEIDRIRQMVEEYEELVKEYEELTEKYKQGKVDKEESKKIIEKS |
| | | | ERLLDLSQDAVRKVKEIIRRILYTNR SEQ ID NO: 486 |
| DHD144 | Heterodimer | a | GSPKEEIVKLHDESAELHRRSVEVADEILKMHERSKDVDDERESRELSKEI |
| | | | ERLIREVEEVSKRIKRLSEEVEYLVR SEQ ID NO: 187 |
| | | | PKEEIVKLHDESAELHRRSVEVADEILKMHERSKDVDDERESRELSKEIER |
| | | | LIREVEEVSKRIKRLSEEVEYLVR SEQ ID NO: 415 |
| DHD144 | Heterodimer | b | GSPLEEILKIQRRINKIQDDINKILHEILRMQEKLNRSSDKDEVEESLRRI |
| | | | RELIKRIKDLSKEIEDLSREVKYRTT SEQ ID NO: 188 |
| | | | PLEEILKIQRRINKIQDDINKILHEILRMQEKLNRSSDKDEVEESLRRIRE |
| | | | LIKRIKDLSKEIEDLSREVKYRTT SEQ ID NO: 488 |
| DHD145 | Heterodimer | a | GSPEDEHVYVVREIYEVLREHAEVLEENREVIERLLEAKKRGDKSEELVKE |
| | | | LKKSIDKLKEISRKLEEIVKELEKVSEKLK SEQ ID NO: 189 |
| | | | PEDEHVYVVREIYEVLREHAEVLEENREVIERLLEAKKRGDKSEELVKELK |
| | | | KSIDKLKEISRKLEEIVKELEKVSEKLK SEQ ID NO: 417 |
| DHD145 | Heterodimer | b | GSDEDETSYRILELLREIVRASRELIRLSEELLEVARRDDKDETVLETLIR |
| | | | EYKELLDRYRRLIEELTRLVEEYEERSR SEQ ID NO: 190 |
| | | | DEDETSYRILELLREIVRASRELIRLSEELLEVARRDDKDETVLETLIREY |
| | | | KELLDRYRRLIEELTRLVEEYEERSR SEQ ID NO: 490 |
| DHD146 | Heterodimer | a | GSTQEEINRIQHEVLRIQEEIDEILRDIVEKLKAISRGELDHEVVKDVEDK |
| | | | VREALEKSEELLDKSRKVEYKSE SEQ ID NO: 191 |
| | | | TQEEINRIQHEVLRIQEEIDEILRDIVEKLKAISRGELDHEVVKDVEDKVR |
| | | | EALEKSEELLDKSRKVEYKSE SEQ ID NO: 419 |
| DHD146 | Heterodimer | b | GSDEEELNRELLEKSKRLVDINRDIIRTAQELIEMLKDSKDGRVDEDTKRE |
| | | | LRDKLRKLEEKLERVREELRKYEELLRYVQR SEQ ID NO: 192 |
| | | | DEEELNRELLEKSKRLVDINRDIIRTAQELIEMLKDSKDGRVDEDTKRELR |
| | | | DKLRKLEEKLERVREELRKYEELLRYVQR SEQ ID NO: 492 |
| DHD147 | Heterodimer | a | GSDEKDRVYEILKEVQRLVKEYRDISKEIEDLVKHYEHITDDEAQEVSKEL |
| | | | IDKSLRASEIVRELIRLIKELLDELE SEQ ID NO:193 |
| | | | DEKDRVYEILKEVQRLVKEYRDISKEIEDLVKHYEHITDDEAQEVSKELID |
| | | | KSLRASEIVRELIRLIKELLDELE SEQ ID NO: 421 |
| DHD147 | Heterodimer | b | GSDEEDVLYHLRELLEELKRVSDDYERLVREIKETSERKDRDTKENKDMLD |
| | | | ELVKAHREQEKLLERLVRLLEELFERKR SEQ ID NO: 194 |
| | | | DEEDVLYHLRELLEELKRVSDDYERLVREIKETSERKDRDTKENKDMLDEL |
| | | | VKAHREQEKLLERLVRLLEELFERKR SEQ ID NO: 494 |
| DHD1 | Heterodimer | a | PREQAIRISEEIIRISKKIIEILERTRSSTAREAMKWAKDSIRLAEESKYL |
| | | | LDK SEQ ID NO: 195 |
| DHD1 | Heterodimer | b | IEDDVKKIQDSTKKAQKETIEALERSTSSTARKQMEEQKEQIRLQKEAMYL |
| | | | LKK SEQ ID NO: 196 |
| DHD2 | Heterodimer | a | SREEIAKLQEEVIKLQRRVIELQKEVIELQRRAKELTSSYTKEILEIQRRI |
| | | | EEIQREIEEIQKRIEEIQEEIQRRT SEQ ID NO: 197 |
| DHD2 | Heterodimer | b | SDEEIKRLSEEVIQLSRRVIKMSREAIKLSREVQKLTPSYQKRIKEIADRS |
| | | | IELARESIEIAKRSEKIAEESQRRT SEQ ID NO: 198 |
| DHD3 | Heterodimer | a | PAKDEALKMANESLELAKKSARLIQESSSKEILERIEKIQRRIAELQDRIA |
| | | | YLIKK SEQ ID NO: 199 |
| DHD3 | Heterodimer | b | PAKDEALRMIDESRELIKKSNELIQRSSSKEILERILEIQRKIAELQKRIQ |
| | | | YLLKS SEQ ID NO: 200 |
| DHD4 | Heterodimer | a | TDEARYRSERIVKEAKRLLDEARRRSEKIVREAKORSNSEDAKRIMEENLR |
| | | | ESEEAARRLREIIRRNLEESRETG SEQ ID NO: 201 |
| DHD4 | Heterodimer | b | TREALEYQRKMAEEIEDLLREALRRQEEMVREAKORSLSEEFKRIMERILE |
| | | | EQERVMRLAKEALERILEEQKRTG SEQ ID NO: 202 |
| DHD5 | Heterodimer | a | SERTKREAKRSQEEILREAKEAMRRAKESQDHRQNRDGSNSEDLERLSQEQ |
| | | | KRELEEVERRLKELAREQKYKLEDS SEQ ID NO: 203 |
| DHD5 | Heterodimer | b | SEDLKRILKEITERELKLMQDLMEILKKITEDENNLDSNNSEDLKRSIEKA |
| | | | RRILDEALRKLEESARRAKYIQEDN SEQ ID NO: 204 |
| DHD6 | Heterodimer | a | TEDEIRESLKWLDEVLQELREIARESNEVLERNRQKSRSDKLREDIERYKK |
| | | | RMEEARKKLDDQLNKYKKRMDENRS SEQ ID NO: 205 |
| DHD6 | Heterodimer | b | TEEELKESKKFAEDLARSARRALKESKRVLEEISQASRSKKLEEIVRRYKE |
| | | | QVKRWQDEWDERAREYRKRMKENRS SEQ ID NO: 206 |
| DHD7 | Heterodimer | a | TKTEEIERLAREIKKLSEKVERLAQEIEELSRRVKEENSTDRELKEANREI |
| | | | ERAIREIEKANKRMEEALRRMKYNG SEQ ID NO: 207 |
| DHD7 | Heterodimer | b | TKTEEHERLAREISKLADEHRKLAKIIEELARRIKEENLTDDELREAIRKI |
| | | | EDALRKNKEALKIMKEAAERNRYNT SEQ ID NO: 208 |
| DHD8 | Heterodimer | a | TKKEESRELARESEELARESEKLARKSLELARRAESSGSEEEKRRIIDENR |
| | | | KIIERNREIIERNKEIIEYNKELIS SEQ ID NO: 209 |
| DHD8 | Heterodimer | b | TKDEESLELNRESEELNRKSEELNRKSKELNDRAESSNSEEEEKEILREHK |
| | | | EILREHLEILRRHKEILRRHKYLTS SEQ ID NO: 210 |
| DHD16 | Heterodimer | a | TREELLRENIELAKEHIEIMREILELLQKMEELLERQSSEDILEELRKIIE |
| | | | RIRELLDRSRKIHERSEEIAYKEE SEQ ID NO: 211 |
| DHD16 | Heterodimer | b | SEDIAREIKELLRRLKEIIERNQRIAKEHEYIARERKKLDPSNEKERKLLE |
| | | | RSRRLQEESKRLLDEMAEIMRRIKKLLD SEQ ID NO: 212 |
| DHD18 | Heterodimer | a | DRQKLIEENIKLLDKHIKILEEILRLLKKDIDLLKKSSSEEVLEELKKIHR |
| | | | RIDKLLDESKKIHKRSSEIVKKRS SEQ ID NO: 213 |
| DHD18 | Heterodimer | b | DEQKLIETSQRLQEKSERLLEKFEQILREASDLYRKPDSEELLRRVEKLLR |
| | | | ELEKLIRENQDLARKHEKILRDQS SEQ ID NO: 214 |
| DHD19 | Heterodimer | a | DRQELIRENIELLKKHIKIVKEIQKLIETFIELLKKSSSEEILRRLKKILK |
| | | | RIEKLYRESQEIHKRSEEIAKKRQ SEQ ID NO: 215 |
| DHD19 | Heterodimer | b | DEERLIDKSRELQKESEELLKELLKIFKRIEELLEKPDSEELIREIKKLLE |
| | | | TLSEIHKRNEKLARTHEEILRQQS SEQ ID NO: 216 |
| DHD22 | Heterodimer | a | STRDVQREIAKAFKKMADVQKKLAEEIKRHVKNVEKKNKDNDEYRKIATEL |
| | | | LKKATESQKKLKELLDRIRKSDS SEQ ID NO: 217 |
| DHD22 | Heterodimer | b | DKDDRSTSLLKRVEKLIDESDRIIDKFTTLIELSRNGKIDDDQYKKELKEI |
| | | | LELLKKYDKHVKEVEELLKRLNS SEQ ID NO: 218 |
| DHD23 | Heterodimer | a | SKRKALEVSERVVRISEKVVRVLDESSDLLKKSYDDSDKFAELIDRHEEKI |
| | | | KKWKKLIKEWLEIIQRHKS SEQ ID NO: 219 |
| DHD23 | Heterodimer | b | SAEEFVKLSEEAVKRSKEILDIVRKQVKLVKAGVDKHEITDSLRKSEKLIE |
| | | | EHKELIKTHRDLLRREN SEQ ID NO: 220 |
| DHD24 | Heterodimer | a | SSTEILKRFKRALRESEKIVKHSRRVLKIIREVLKQKPTQAVHDLVRIIET |
| | | | QVKALEEQLKVLKRIVEALERQS SEQ ID NO: 221 |
| DHD24 | Heterodimer | b | DKQKEIKDILEKTRRIAEESRKIAEKFDEIIKRSTEGKIDESLTKELEELV |
| | | | KEVIKLSEDDARTSDDLVRKES SEQ ID NO: 222 |
| DHD26 | Heterodimer | a | DEDESIKLTRKSIEETRKSLKIIKEVVELIREVLKHIKDLDKEIFERIDKI |
| | | | LDKYKKQVDTYDEILKEYEKKQR SEQ ID NO: 223 |
| DHD26 | Heterodimer | b | SELDEQKELIKKQEKLIEEQQRLLSKIRRMFKERVKDQELLREIQKVLKRS |
| | | | QEIVETSKKILDRSDKTTE SEQ ID NO: 224 |
| DHD28 | Heterodimer | a | DQKEINTRIVEKLERIFKKSKEIVRQSERVISTIEKKTEDERELDLLRRHV |
| | | | KIVREHLKLLEELLKIIKEVQKESE SEQ ID NO: 225 |
| DHD28 | Heterodimer | b | DTEELVKRLNELLKELSKLVKEFIKILETYRKDQTKDTSKISERVDRILKT |
| | | | YEDLLQKYKEILEKIEKQLS SEQ ID NO: 226 |
| DHD29 | Heterodimer | a | DYARLIDQAVEVTRKVVEVNVTVARVNDKFAKHLGDEELRRVSEHLKEVSK |
| | | | DLQEVAKKSKDAARQVK SEQ ID NO: 227 |
| DHD29 | Heterodimer | b | DVSKVAEEYLQISKTLVDISRTLLEISERLVRLVRTVADDRSEVKKAIEDS |
| | | | IEVLKTSEEVVRQIKRASDKLVKAIS SEQ ID NO: 228 |
| DHD31 | Heterodimer | a | DAKEIQRRVVEIQTEVVKLOKKAVDIIRKIIEAFNNSNIDQSLLEAAKEIV |
| | | | KEIDKLEKLTESLLEESKKLLKRSS SEQ ID NO: 229 |
| DHD31 | Heterodimer | b | SAEEVVKLAKIFLELLRESIKLLKRSVDLLRKSSDPSLDKSEAEKVSREIE |
| | | | KVSDTSLKLSKKALDVVKRALKVAS SEQ ID NO: 230 |
| DHD32 | Heterodimer | a | DEKDAARKARKVSEEAKEASKKIEKALEESKRILNTLKQKKDEQEVKVIKE |
| | | | HEDVLRQIEKIQKQVLEIQKEVAKLLESLD SEQ ID NO: 231 |
| DHD32 | Heterodimer | b | SADDVARASEKVLRVARESAKAADKSLEVFKEVVKRGDKEAFLQVVKINEE |
| | | | VVKINITVIRILIEVSKTAT SEQ ID NO: 232 |
| DHD38 | Heterodimer | a | DEYVKETLKQLREALASLREADKRITELVKEARKKPLSEAARKFAEAIVTH |
| | | | VKVVVEHVEVVLRHVEVLVEAKKNGVIDKSILDNALRIIENVIRLLSNVIR |
| | | | VVDEVLQDLD SEQ ID NO: 233 |
| DHD38 | Heterodimer | b | DASDVIRRIHELFEEVHRLIEAVHRAIEDVAKAAQKKGLDESAVEILAELS |
| | | | KELAKLSRRLAEISREIQKVVTDPDDKEAVERLKEIIKEIKKQLDELRDRL |
| | | | RKLQDLLYKLK SEQ ID NO: 234 |
| DHD60 | Heterodimer | a | SEDKAHHDIVRVLEELIKIHDELMKISEEILKATSDSTATDETKEELKRRS |
| | | | KEAQKKSDTLVKIVKELEKESRKAQS SEQ ID NO: 235 |
| DHD60 | Heterodimer | b | DDEEKYRQIIREAQEISKTAKRILRDAQEISKRIRHQGVDRSEHQRLVDLL |
| | | | RELIKEHHKLLRRQQEADTRND SEQ ID NO: 236 |
| DHD63 | Heterodimer | a | DRKDKARKASEKLEEVIQRWKTVADKWKKMVDLVSNGKLSQEEVARVTEEL |
| | | | LKIQTELAKLLEEHAKVLQESAS SEQ ID NO: 237 |
| DHD63 | Heterodimer | b | SDEESIKTQSELIKTSEELLKDVKRIDEELQKLRDDPTLDESELKKRVKEW |
| | | | SDRVRKAKEISRKIQEIVKESKKRSS SEQ ID NO: 238 |
| DHD66 | Heterodimer | a | DKDEELRKVIEKYREMVKEYRKVIREYEEVIKSSKTIDKSSLISLSRKMVE |
| | | | LSQRVIDVSDEVAKVLSRKQS SEQ ID NO: 239 |
| DHD66 | Heterodimer | b | TDEERLKKQTKELKEQTKQLEKQKDLLEKISNGEISKDEIQEIIKESKKIA |
| | | | KESQKALDSSRKALEEVS SEQ ID NO: 240 |
| DHD67 | Heterodimer | a | DEKEVSKEIIKVLKDIAKVQQKVIEVSQRLASVLRADDDNVVKRALEEYEK |
| | | | ILEELRELNKEIEKLTDKYRKVTS SEQ ID NO: 241 |
| DHD67 | Heterodimer | b | DSDEQTKELEKLTELHKRHVEKLKKQTKESREVDSNKLWKSKDVKDKLSES |
| | | | EKELQKLSDQDKKAKDALESSRRKND SEQ ID NO: 242 |
| DHD69 | Heterodimer | a | DAEEQLKLLTKLLRHQQRLLQLIKESLKLIEKIDQSSQENQDEIRKWREVT |
| | | | KKLRELIKTSEKLVRELEKSYKKSS SEQ ID NO: 243 |
| DHD69 | Heterodimer | b | SLRDVVRRYQELVRRYDELIKTLTEILKKYQKKGAEDKDASTELVKAVRTS |
| | | | LKLSKELLKINSELLKEDS SEQ ID NO: 244 |
| DHD71 | Heterodimer | a | SKEELKRKLDELKKRSDTLKELSKKLKEISERNPDDKSVHRTIIRIHREFV |
| | | | KNHKEIVRVIEEIVSDKS SEQ ID NO: 245 |
| DHD71 | Heterodimer | b | SKQDEHDRLLKIHDKLVKQHDELLKLLTKLSRAGDSVTKKKLEEILRKLQE |
| | | | VSKQLEESLKDADKVSKDIN SEQ ID NO: 246 |
| DHD72 | Heterodimer | a | TVQSLLEQHVKIVKRSIEILERHTQILQDIARSQGVSKELEDVERQVKEYR |
| | | | KEVKKLEEDLRQLSRNSK SEQ ID NO: 247 |
| DHD72 | Heterodimer | b | SDSDRIEKLIRESTELLKEQQKLAKRSRELAETVESLPLTEEYLKQQREHQ |
| | | | KKIEKLLKDSEKHLEELKRLVKSEK SEQ ID NO: 248 |
| DHD73 | Heterodimer | a | DSEKRIEDILRTDLELAKRDAELVKEHIKLVKRIDLSEELKKQVEDVEKES |
| | | | KKLEDSSEKLVQKVRKRSS SEQ ID NO: 249 |
| DHD73 | Heterodimer | b | DEEERAKDLRKYLEEQTQYYRTVTEHLRNLEKVVEELERRGKPSSELQQIL |
| | | | ERSQRIYKETTEIYDTSKKLIEELDKHHR SEQ ID NO: 250 |
| DHD148 | Heterodimer | a | PLEDILKRHLDKVRELVRLSEEVNKLAKEVLDILKDKRVDEKELDKVLKEL |
| | | | EKVVEEYERAVKESRDLLRELRETTR SEQ ID NO: 251 |
| DHD148 | Heterodimer | b | DKERLLEIHERIQKLLDRNLEIIERLLRLLREARDIKDDDKLDKVIKRLKE |
| | | | LSEESKDILDKIKELLKESEKELT SEQ ID NO: 252 |
| DHD149 | Heterodimer | a | PEDEVIRVIEELLRIAAEVDEVHRRNVEVQEEASRVTDRERLERLNRESEE |
| | | | LIKRSRELIEEQRKLIERLERLAT SEQ ID NO: 253 |
| DHD149 | Heterodimer | b | DLEELIKEYAEVVRRHHKAVRDLERLVRELANAKHASEEELKRIATEILRI |
| | | | VKELIRVQERLIKLSEDSNEESR SEQ ID NO: 254 |
| DHD150 | Heterodimer | a | PTDEVIEVLKELLRIHRENLRVNEEIVEVNERASRVTDREELERLLRRSNE |
| | | | LIKRSRELNEESKKLIEKLERLAT SEQ ID NO: 255 |
| DHD150 | Heterodimer | b | DNEEIIKEARRVVEEYKKAVDRLEELVRRAENAKHASEKELKDIVREILRI |
| | | | SKELNKVSERLIELWERSQERAR SEQ ID NO: 256 |
| DHD151 | Heterodimer | a | PKEDIDRVSRELVRVHKELLEVLRKSTEIVEAVARNEKDERTIEEVLEEQE |
| | | | RAVRKLEEVSKKHKEAVKRLK SEQ ID NO: 257 |
| DHD151 | Heterodimer | b | ELERLSEEIQKLSDRLIELIRRHSKVLEEIVRLLKHKDNDEREVRRLLKLL |
| | | | RDLTRRYEEVLRKVEEIVKRQEDESR SEQ ID NO: 258 |
| DHD152 | Heterodimer | a | PEEDILRLLRKLVEVDKELLEVVRESTEVVRLVARNEKDVETVERVLRKQE |
| | | | EVVRKYERVSRELEEAVRRLK SEQ ID NO: 259 |
| DHD152 | Heterodimer | b | ELKDLVEEIVKLSKENLKLWEDHSRVLEEIVRLLKHKDNDEREVRRLLKLL |
| | | | EDLTRRAEETSRRIEEIVKEAEDRAR SEQ ID NO: 260 |
| DHD153 | Heterodimer | a | DEERELREVLRKHHRVVREWTKVVEELKRVVELLKRGETSEEDLLRVLKKL |
| | | | LEMDKRILEVNREVLRVLEKRLT SEQ ID NO: 261 |
| DHD153 | Heterodimer | b | SLEEIIEELVELVRRSVEIAKESDEVARRIVESEDKKKELIDTLRDLHREW |
| | | | QEVTKRAEELVREAEKEVR SEQ ID NO: 262 |
| DHD154 | Heterodimer | a | TAEELLEVHKKSDRVTKEHLRVSEEILKVVEVLTRGEVSSEVLKRVLRKLE |
| | | | ELTDKLRRVTEEQRRVVEKLN SEQ ID NO: 263 |
| DHD154 | Heterodimer | b | DLEDLLRRLRRLVDEQRRLVEELERVSRRLEKAVRDNEDERELARLSREHS |
| | | | DIQDKHDKLAREILEVLKRLLERTE SEQ ID NO: 264 |
| DHD155 | Heterodimer | a | PEDDVVRIIKEDLESNREVLREQKEIHRILELVTRGEVSEEAIDRVLKRQE |
| | | | DLLKKQKESTDKARKVVEERR SEQ ID NO: 265 |
| DHD155 | Heterodimer | b | DEVRLITEWLKLSEESTRLLKELVELTRLLRNNVPNVEEILREHERISREL |
| | | | ERLSRRLKDLADKLERTRR SEQ ID NO: 266 |
| DHD156 | Heterodimer | a | DEDEVVKVHEEHVKSHEEIHRSHEEVVRAAEEDKRDSRELRTLMEEHRKLL |
| | | | EENEKSIEEVKKIHERVKR SEQ ID NO: 267 |
| DHD156 | Heterodimer | b | KKEELIDISKEVLDLDDEINKISKEILELIKKLLRLKEEGREDKDKAREVK |
| | | | RRIRELHRRIQELNKRLRELHKRVQETKR SEQ ID NO: 268 |
| DHD157 | Heterodimer | a | PEEDIARRVEDLLRKSEELIKESEKILKESKRLLDRNDSDKRVLETNLRLI |
| | | | DKHTKLLERNLELLEELLKLAEDVAK SEQ ID NO: 269 |
| DHD157 | Heterodimer | b | RFKDLSREYIEVVKRLLELSREALEVLREIKDTDKTDKKRIKELIDRLRKL |
| | | | IEEYKRIIDRLRKLSKDLEEEHR SEQ ID NO: 270 |
| DHD158 | Heterodimer | a | DEEELVKILKELQRLSEESLEINKRLVEILRLLRRGEVPKEEVEKKLREIK |
| | | | KEQEKLDREHEKIKKRIEEITK SEQ ID NO: 271 |
| DHD158 | Heterodimer | b | SLKEKILEIIERNMKLVELSNRSVEIVARILKGEKDDEETLERLLREWDKI |
| | | | TRDYEEIIKESRKLVKELEEEAK SEQ ID NO: 272 |
| DHD159 | Heterodimer | a | SKTEILRKALEIHKEQIDIVRKLIELSEEVLKLVEESKEKNLEKLKRIDEE |
| | | | TDRLLERLDELHKRLTELAERLK SEQ ID NO: 273 |
| DHD159 | Heterodimer | b | SDDEARKQLEEMKRRLREVEKKSKRVEERVRELERLVRENREDEDRVLKTL |
| | | | EDLLRENEKLVRTIERHVREQRELSKEVK SEQ ID NO: 274 |
| DHD160 | Heterodimer | a | SEEELEKKADELRKLSEEWRKLQEEDKRLSEMVEKGELDLQEVDEHSLRVL |
| | | | ERATEVHRTVDKVIEEILRTTN SEQ ID NO: 275 |
| DHD160 | Heterodimer | b | SEKERHRESQETQEEIRRTHEEIIRKLEEILRRAKAGELPEETLDRLRRIM |
| | | | ERLKELSERLDDLVRKLRDDHRREQK SEQ ID NO: 276 |
| DHD161 | Heterodimer | a | SEKEILEELKRILKRVKDISDRLEELDKRTEEIARREPTKELVDELVKIHR |
| | | | DWLRLHEEILKLVDDALKKVEDATK SEQ ID NO: 277 |
| DHD161 | Heterodimer | b | DLRELLELQREASRLHRELVKLLTELVKKLELIAKGEDIREEDLKRIKERL |
| | | | EEIKKRSKRIKEESDEIDKKTK SEQ ID NO: 278 |
| DHD162 | Heterodimer | a | SERELQRELNKIVRRILEIHREVSELHQRAVKLIRENDNSEELEEISRRIE |
| | | | ELSKELEKLVREHDEIVKTIE SEQ ID NO: 279 |
| DHD162 | Heterodimer | b | SEREKLDRNDEELKEINKRVEEIKERSDRITEAIEKNERSEEEIRRLSREQ |
| | | | NEALQRLLELHKKLVKLHRELLEDTR SEQ ID NO: 280 |
| DHD163 | Heterodimer | a | DKEDVIRVHDEQHKLIEEQLELTRRIAELVREIAKNTASEEEIKEMLKEIK |
| | | | RLDDRSREIQDRLQKLLEEIRRKTK SEQ ID NO: 281 |
| DHD163 | Heterodimer | b | TEEEIVELNKDIQRKSKEHIDLQNELVKKIERAIRENNITEELLEELERLL |
| | | | RESEKIVEEIRRITDKIRKDAK SEQ ID NO: 282 |
| DHD164 | Heterodimer | a | SEKEILERLLRLSKEQNEISEEIHRLTERLVELKRRKDDDERLKRILDRQK |
| | | | RLVERAREISKEYEDLLRKLE SEQ ID NO:283 |
| DHD164 | Heterodimer | b | SMEELLRKNARLSRKOLKIIDEHLELSTKLTRGEAGDETLEEIERRSREML |
| | | | EEQRRVDEESKRIREKLK SEQ ID NO: 284 |
| DHD165 | Heterodimer | a | SEEEIRDIVEKLLRTHEEVLKEIKKLLDDSERVRRRELDKKDLDRIQKEQR |
| | | | DIQEENKEKAKRFDELVKELKKAAK SEQ ID NO: 285 |
| DHD165 | Heterodimer | b | SEEEHRRTMEKVEKEVRDIKRRSEEVKKKVKANTLSEEDLVRLLERLVEDH |
| | | | KRLQDLSQEIIERDEKATK SEQ ID NO: 286 |
| DHD166 | Heterodimer | a | DEDELAKEIEDVQRRNKESQEEHDKSVKKLEAAERGEIDEDSLLRVLEEDI |
| | | | KVLEKDIEVLERSIEVIEKAE SEQ ID NO: 287 |
| DHD166 | Heterodimer | b | SEKELIRRLLEQQRQHLRLSERLIELSRRLVEVVRKGKDNRDLLRELKKLS |
| | | | EEHKKHSKDDHEKVREIREREK SEQ ID NO: 288 |
| DHS17 | Heterodimer | a | DRKDLLKRNIKLLDRHLKILDTILKLLEKLSELLKKSSSEEVVKEYKKILD |
| | | | EIRKLLEESKEIHKESKEILERES SEQ ID NO: 289 |
| DHD17 | Heterodimer | b | DEEKLIERSKRLQEESEQLLEKFEQILRELTELLEKPDSEELARKIKKLHD |
| | | | ELRKIIKRNQELIREHEEILRKRD SEQ ID NO: 290 |

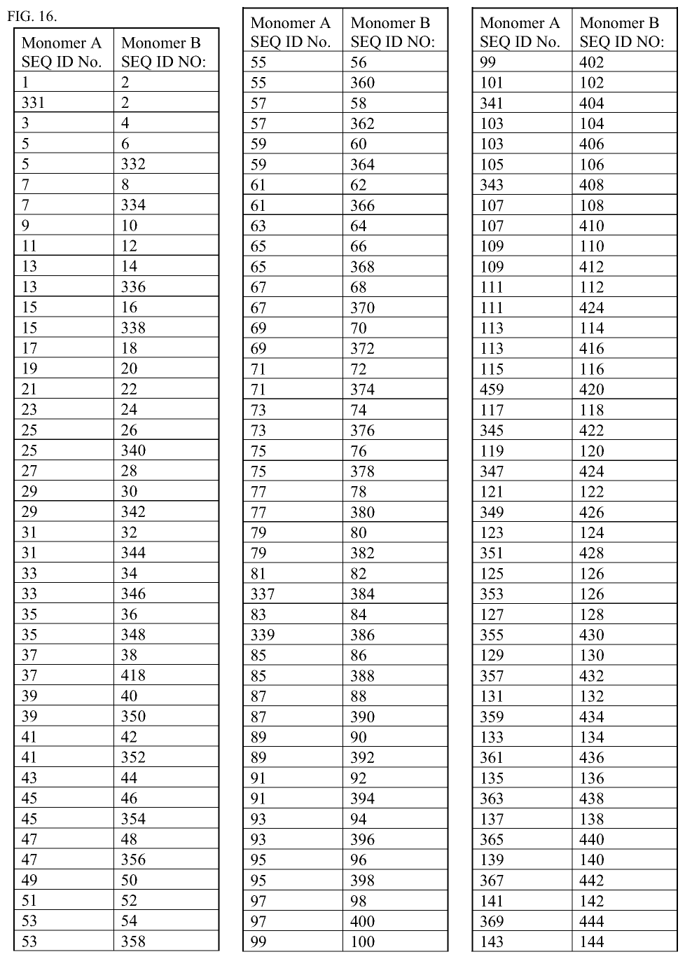

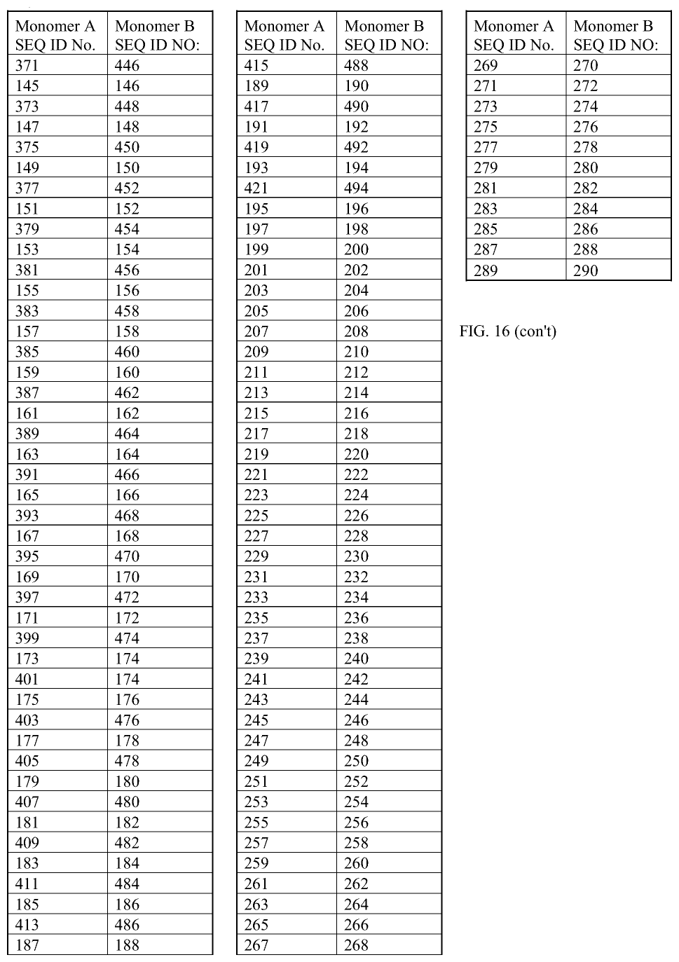

In some aspects, non-limiting examples of the monomer A polypeptide and monomer B polypeptide pairs are shown in FIG. 16.

In some aspects, the amino acid sequence of SEQ ID NOs: 331, 332, 334, 336-422, 424, 426, 428, 430, 432, 434, 436, 438, 440, 442, 444, 446, 448, 450, 452, 454, 456, 458-460, 462, 464, 466, 468, 470, 472, 474, 476, 478, 480, 482, 484, 486, 488, 490, 493, and 494 useful for the monomer A polypeptide or the monomer B polypeptide is not linked to GlySer at the N terminus of the sequence or does not comprise GlySer at the N terminus. In some aspects, the monomer A polypeptide and/or the monomer B polypeptide comprises at least one amino acid, at least two amino acids, at least three amino acids, at least four amino acids, at least five amino acids, at least six amino acids, at least seven amino acids, at least eight amino acids, at least nine amino acids, or at least ten amino acids at the N terminus or the C terminus of the amino acid sequence. In some aspects, the additional amino acids are not GlySer at the N terminus.

In some aspects, the protein of the present disclosure comprises a heterodimer comprising a monomer A polypeptide and a monomer B polypeptide, wherein the monomer A polypeptide comprises an amino acid sequence at least 70%, 75%, 80%, 85%, 90%, 91%, 92%, 93%, 94%, 95%, 96%, 97%, 98%, 99%, or 100% sequence identity along the length of the amino acid sequence as set forth in SEQ ID NO: 331, 5, 7, 13, 15, 25, 29, 31, 33, 35, 37, 39, 41, 45, 47, 53, 55, 57, 59, 61, 65, 67, 69, 71, 73, 75, 77, 79, 337, 339, 85, 87, 89, 91, 93, 95, 97, 99, 341, 103, 343, 107, 109, 111, 113, 459, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, or 421 and the monomer B polypeptide comprises an amino acid sequence at least 70%, 75%, 80%, 85%, 90%, 91%, 92%, 93%, 94%, 95%, 96%, 97%, 98%, 99%, or 100% sequence identity along the length of the amino acid sequence as set forth in SEQ ID NO: 2, 332, 334, 336, 338, 340, 342, 344, 346, 348, 418, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 420, 422, 424, 426, 428, 126, 430, 432, 434, 436, 438, 440, 442, 444, 446, 448, 450, 452, 454, 456, 458, 460, 462, 464, 466, 468, 470, 472, 474, 174, 476, 478, 480, 482, 484, 486, 488, 490, 492, or 494, respectively.

In some aspects, the protein of the present disclosure comprises a heterodimer comprising a monomer A polypeptide and a monomer B polypeptide, wherein the monomer A polypeptide comprises an amino acid sequence at least 70%, 75%, 80%, 85%, 90%, 91%, 92%, 93%, 94%, 95%, 96%, 97%, 98%, 99%, or 100% sequence identity along the length of SEQ ID NO: 331, 5, 7, 13, 15, 25, 29, 31, 33, 35, 37, 39, 41, 45, 47, 53, 55, 57, 59, 61, 65, 67, 69, 71, 73, 75, 77, 79, 337, 339, 85, 87, 89, 91, 93, 95, 97, 99, 341, 103, 343, 107, 109, 111, 113, 459, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, or 421, wherein the amino acid sequence of the monomer A polypeptide does not comprise GlySer at the N terminus, and the monomer B polypeptide comprises an amino acid sequence at least 70%, 75%, 80%, 85%, 90%, 91%, 92%, 93%, 94%, 95%, 96%, 97%, 98%, 99%, or 100% sequence identity along the length of SEQ ID NO: 2, 332, 334, 336, 338, 340, 342, 344, 346, 348, 418, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 420, 422, 424, 426, 428, 126, 430, 432, 434, 436, 438, 440, 442, 444, 446, 448, 450, 452, 454, 456, 458, 460, 462, 464, 466, 468, 470, 472, 474, 174, 476, 478, 480, 482, 484, 486, 488, 490, 492, or 494, respectively, wherein the amino acid sequence of the monomer B polypeptide does not comprise GlySer at the N terminus.

In some aspects, the protein of the present disclosure comprises a heterodimer comprising a monomer A polypeptide and a monomer B polypeptide, wherein the monomer A polypeptide consists of an amino acid sequence at least 70%, 75%, 80%, 85%, 90%, 91%, 92%, 93%, 94%, 95%, 96%, 97%, 98%, 99%, or 100% sequence identity along the length of the amino acid sequence as set forth in SEQ ID NO: 331, 5, 7, 13, 15, 25, 29, 31, 33, 35, 37, 39, 41, 45, 47, 53, 55, 57, 59, 61, 65, 67, 69, 71, 73, 75, 77, 79, 337, 339, 85, 87, 89, 91, 93, 95, 97, 99, 341, 103, 343, 107, 109, 111, 113, 459, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, or 421 and the monomer B polypeptide consists of an amino acid sequence at least 70%, 75%, 80%, 85%, 90%, 91%, 92%, 93%, 94%, 95%, 96%, 97%, 98%, 99%, or 100% sequence identity along the length of the amino acid sequence as set forth in SEQ ID NO: 2, 332, 334, 336, 338, 340, 342, 344, 346, 348, 418, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 420, 422, 424, 426, 428, 126, 430, 432, 434, 436, 438, 440, 442, 444, 446, 448, 450, 452, 454, 456, 458, 460, 462, 464, 466, 468, 470, 472, 474, 174, 476, 478, 480, 482, 484, 486, 488, 490, 492, or 494, respectively.

In one embodiment of any of the above embodiments, amino acid changes from the reference amino acid sequence are conservative amino acid substitutions. As used herein, “conservative amino acid substitution” means an amino acid substitution that does not alter or substantially alter polypeptide function or other characteristics. A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. antigen-binding activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into H is; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In another embodiment of any of the above embodiments, amino acid residues at 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% of defined interface positions are invariant compared to the reference amino acid sequence. Table 2 below provides the residue numbers within each A and B monomer that are present at the interface in the heterodimer. The position of interface residues are the same for A-B binding partners. Table 2 is organized by heterodimer design name (see the left-hand column in Tables 1A and 1B). Note that for purpose of defining the position of interface residues for each polypeptide in Table 1A and 1B, the “GS” residues at the amino terminus, if present, are not included.

TABLE 2
Interface residues by position number across both chains ‘a’ and ‘b’

DHD_1
[5, 6, 8, 9, 12, 13, 16, 19, 20, 22, 23, 31, 34, 35, 38, 41, 42, 45, 48, 52, 55, 59, 63, 66, 70, 73, 74, 77, 80, 81, 85,
88, 89, 92, 95, 96, 99, 102, 103, 106]
DHD_2
[5, 6, 9, 12, 13, 16, 19, 20, 23, 26, 27, 30, 33, 36, 37, 38, 41, 44, 45, 48, 51, 55, 58, 62, 65, 69, 72, 73, 76, 81, 85,
88, 89, 92, 95, 96, 99, 102, 103, 106, 109, 110, 112, 114, 117, 120, 123, 124, 127, 128, 131, 134, 135, 137, 138,
141, 144, 145, 148, 149, 152]
DHD_3
[6, 7, 10, 11, 13, 14, 17, 20, 21, 24, 25, 28, 33, 36, 40, 43, 44, 47, 50, 51, 54, 62, 63, 66, 69, 73, 76, 77, 80, 81, 8
4, 89, 92, 93, 96, 99, 100, 103, 106, 107, 110, 112]
DHD_4
[1, 8, 11, 12, 15, 19, 22, 26, 29, 30, 33, 38, 39, 42, 45, 46, 49, 50, 53, 56, 57, 60, 63, 64, 67, 68, 71, 75, 76, 80, 8
3, 87, 90, 94, 98, 101, 105, 108, 113, 114, 117, 121, 124, 125, 128, 132, 135, 139, 142, 143, 146, 150]
DHD_5
[4, 8, 12, 15, 16, 19, 22, 23, 26, 30, 34, 38, 39, 44, 48, 51, 55, 58, 62, 66, 69, 73, 76, 80, 84, 87, 88, 92, 95, 96, 9
8, 99, 102, 105, 106, 110, 114, 115, 117, 120, 124, 127, 131, 135, 138, 142, 145, 149, 152]
DHD_6
[1, 5, 8, 9, 11, 12, 15, 16, 19, 22, 23, 26, 27, 30, 33, 35, 37, 42, 46, 49, 53, 56, 60, 64, 67, 71, 74, 77, 81, 84, 87,
88, 91, 92, 95, 99, 102, 105, 106, 109, 110, 111, 112, 113, 118, 122, 125, 129, 132, 133, 136, 140, 143, 147, 150
]
DHD_7
[3, 6, 10, 13, 17, 20, 24, 27, 31, 34, 44, 47, 48, 51, 55, 58, 61, 62, 65, 69, 72, 75, 76, 79, 82, 86, 89, 90, 93, 96, 1
00, 103, 107, 110, 115, 120, 124, 127, 130, 131, 134, 138, 141, 145, 148, 151, 152]
DHD_8
[6, 10, 13, 17, 20, 24, 27, 28, 31, 34, 47, 50, 54, 57, 61, 64, 68, 71, 75, 76, 82, 83, 86, 89, 93, 96, 100, 103, 107,
110, 123, 126, 130, 133, 134, 137, 140, 144, 147, 150, 151, 152]
DHD_9
[12, 16, 19, 23, 26, 30, 33, 38, 39, 42, 46, 49, 52, 53, 56, 59, 60, 63, 67, 69, 88, 92, 95, 99, 102, 106, 109, 115, 1
22, 125, 129, 132, 136, 139, 143, 145]
DHD_16
[6, 9, 10, 13, 16, 17, 20, 23, 24, 27, 28, 30, 34, 43, 46, 50, 53, 57, 60, 64, 67, 71, 72, 5, 8, 12, 15, 19, 22, 23, 26,
29, 33, 41, 50, 57, 60, 64, 67, 68, 71, 74, 78]
DHD_18
[3, 6, 9, 10, 13, 16, 17, 20, 23, 24, 27, 31, 34, 38, 43, 46, 50, 53, 57, 60, 64, 67, 68, 71, 75, 78, 81, 85, 88, 91, 95,
98, 102, 105, 106, 109, 118, 121, 125, 128, 132, 135, 136, 139, 142, 146, 149, 150]
DHD_19
[3, 6, 9, 10, 13, 16, 17, 20, 23, 24, 27, 30, 31, 34, 38, 43, 46, 50, 53, 57, 60, 61, 64, 67, 71, 75, 81, 88, 91, 95, 98,
99, 102, 105, 109, 118, 121, 125, 128, 129, 132, 135, 139, 142, 146, 149, 150]
DHD_22
[1, 2, 5, 6, 9, 10, 12, 13, 16, 19, 20, 23, 27, 30, 31, 33, 34, 38, 41, 44, 47, 48, 51, 55, 58, 62, 65, 69, 72, 80, 81, 8
4, 87, 91, 94, 97, 98, 101, 102, 105, 108, 118, 122, 125, 126, 129, 132, 136, 139, 143, 146, 147, 148]
DHD_23
[1, 5, 6, 8, 9, 12, 15, 16, 19, 22, 23, 26, 29, 30, 33, 34, 40, 43, 47, 51, 54, 57, 58, 61, 64, 65, 68, 72, 75, 76, 79, 8
2, 83, 86, 89, 90, 93, 96, 97, 100, 104, 109, 110, 113, 116, 120, 123, 126, 127, 130, 134, 138]
DHD_24
[1, 5, 9, 12, 16, 19, 22, 26, 29, 30, 33, 40, 41, 42, 45, 48, 49, 51, 52, 55, 56, 59, 62, 63, 66, 69, 70, 73, 77, 80, 83,
84, 87, 91, 94, 98, 101, 105, 108, 109, 113, 118, 121, 125, 128, 129, 132, 136, 139, 142, 143, 147]
DHD_26
[5, 8, 9, 12, 16, 19, 22, 23, 26, 29, 30, 33, 37, 40, 44, 45, 48, 51, 52, 55, 58, 59, 62, 65, 66, 69, 73, 77, 80, 84, 87,
91, 94, 95, 98, 101, 105, 109, 112, 114, 115, 118, 119, 122, 125, 126, 129, 132, 136, 139, 143]
DHD_28
[2, 5, 6, 9, 13, 16, 20, 23, 26, 27, 30, 31, 33, 34, 46, 47, 50, 53, 54, 57, 60, 61, 64, 67, 68, 71, 75, 78, 82, 85, 86,
89, 92, 93, 96, 99, 100, 103, 106, 110, 114, 115, 118, 121, 125, 128, 132, 135, 139, 142, 146, 147]
DHD_29
[2, 5, 6, 8, 9, 10, 12, 13, 16, 19, 20, 22, 23, 26, 27, 30, 33, 34, 39, 42, 45, 46, 49, 53, 56, 60, 63, 66, 67, 70, 71, 7
4, 77, 78, 81, 84, 85, 88, 91, 92, 95, 98, 99, 102, 106, 112, 115, 116, 119, 120, 122, 123, 126, 129, 130, 133, 137
, 140, 141, 144, 145]
DHD_31
[2, 5, 6, 9, 10, 12, 13, 16, 17, 19, 20, 23, 24, 26, 27, 30, 31, 33, 34, 37, 38, 39, 43, 46, 50, 54, 57, 60, 61, 64, 65,
68, 71, 75, 78, 81, 82, 85, 88, 89, 92, 95, 96, 99, 102, 103, 106, 109, 110, 119, 123, 126, 130, 133, 134, 137, 140
, 141, 144, 147, 148, 151, 152]
DHD_32
[5, 6, 9, 12, 16, 19, 23, 26, 27, 30, 33, 34, 37, 44, 48, 49, 52, 55, 56, 58, 59, 62, 63, 65, 66, 69, 70, 73, 76, 77, 79,
80, 82, 83, 86, 87, 90, 93, 94, 97, 100, 101, 104, 107, 108, 111, 114, 115, 118, 123, 124, 127, 130, 133, 134, 13
7, 138, 140, 141, 144, 145, 147, 148, 151, 152]
DHD_38
[42, 43, 46, 49, 50, 52, 53, 56, 57, 60, 63, 64, 67, 70, 71, 74, 77, 79, 80, 84, 87, 88, 91, 94, 95, 98, 101, 102, 105,
109, 116, 117, 120, 123, 124, 127, 131, 134, 137, 138, 141, 144, 145, 149, 153, 158, 161, 162, 165, 168, 169, 1
72, 175, 176, 179, 182, 183, 185, 186, 187, 189]
DHD_39
[2, 6, 9, 13, 16, 20, 23, 27, 29, 30, 34, 40, 43, 46, 47, 50, 51, 54, 57, 58, 60, 61, 64, 110, 114, 117, 121, 124, 128,
131, 132, 135, 138, 145, 149, 152, 156, 159, 160, 163, 164, 166, 167, 170, 173, 174, 176, 177, 178]
DHD_40
[5, 9, 11, 12, 15, 16, 19, 23, 26, 29, 30, 33, 43, 46, 49, 50, 53, 56, 57, 59, 60, 63, 64, 67, 71, 108, 112, 116, 119,
123, 127, 130, 133, 134, 137, 138, 148, 151, 154, 158, 161, 165, 168, 169, 172, 175]
DHD_43
[3, 4, 7, 11, 14, 18, 22, 25, 29, 34, 35, 39, 43, 46, 47, 50, 51, 54, 57, 58, 61, 62, 65, 71, 72, 75, 76, 79, 82, 83, 86,
87, 90, 93, 94, 97, 98, 100, 101, 104, 111, 114, 115, 117, 118, 122, 125, 126, 129, 133, 136, 137, 140, 144]
DHD_60
[6, 7, 9, 10, 13, 16, 17, 20, 23, 24, 27, 30, 31, 34, 35, 51, 54, 55, 58, 61, 62, 65, 68, 72, 75, 76, 77, 83, 87, 90, 91,
93, 94, 97, 100, 101, 104, 105, 108, 111, 115, 116, 121, 122, 124, 125, 128, 131, 132, 135, 136, 138, 139, 142,
143, 149]
DHD_63
[6, 10, 13, 17, 20, 24, 27, 30, 31, 34, 41, 44, 45, 48, 51, 52, 55, 56, 58, 59, 62, 65, 66, 68, 69, 70, 72, 73, 74, 80,
83, 84, 87, 90, 93, 94, 97, 104, 105, 118, 122, 125, 126, 129, 132, 136, 139, 140, 143, 146, 150]
DHD_65
[2, 5, 6, 8, 9, 12, 13, 16, 19, 20, 23, 26, 27, 29, 30, 33, 34, 37, 43, 47, 50, 54, 57, 61, 64, 68, 71, 72, 75, 82, 85, 8
6, 89, 92, 93, 96, 99, 100, 103, 106, 107, 110, 118, 120, 121, 123, 127, 128, 131, 134, 135, 138, 139, 141, 142, 1
45, 148]
DHD_66
[6, 10, 13, 16, 17, 20, 24, 27, 31, 34, 42, 43, 46, 49, 50, 53, 54, 56, 57, 60, 63, 64, 67, 68, 71, 78, 81, 82, 85, 88,
89, 92, 95, 99, 102, 103, 107, 112, 113, 115, 116, 119, 123, 126, 127, 130, 133, 137, 140, 141]
DHD_67
[6, 9, 10, 13, 16, 17, 20, 21, 23, 24, 27, 28, 30, 31, 34, 36, 42, 46, 49, 52, 53, 56, 60, 63, 67, 70, 74, 77, 80, 81, 8
4, 87, 88, 91, 94, 95, 98, 101, 102, 105, 110, 114, 123, 130, 131, 133, 134, 140, 144, 147, 151]
DHD_69
[2, 5, 6, 9, 10, 12, 13, 16, 17, 19, 20, 23, 26, 27, 30, 33, 35, 44, 47, 51, 54, 58, 61, 65, 68, 72, 75, 76, 78, 82, 85,
86, 89, 92, 96, 99, 100, 102, 103, 106, 107, 111, 117, 118, 120, 121, 124, 127, 128, 131, 134, 135, 138, 139, 141
, 142, 146]
DHD_70
[6, 9, 10, 13, 16, 17, 20, 23, 24, 26, 27, 30, 31, 34, 37, 38, 41, 43, 44, 47, 50, 51, 54, 57, 58, 61, 64, 65, 68, 71, 7
2, 73, 74, 78, 81, 82, 85, 88, 89, 92, 95, 96, 99, 102, 103, 106, 107, 109, 110, 111, 119, 120, 123, 124, 127, 130,
131, 134, 137, 138, 141, 144, 145, 148]
DHD_71
[9, 16, 19, 23, 30, 34, 40, 43, 44, 46, 47, 50, 51, 54, 57, 58, 61, 64, 65, 69, 72, 75, 78, 79, 82, 85, 86, 89, 92, 93,
96, 97, 99, 100, 103, 106, 111, 115, 118, 119, 121, 122, 125, 128, 129, 132, 136, 139, 140]
DHD_72
[1, 2, 5, 6, 8, 9, 12, 16, 19, 23, 26, 27, 30, 33, 34, 40, 43, 46, 47, 50, 54, 57, 63, 64, 67, 68, 75, 78, 79, 82, 85, 86,
89, 92, 93, 96, 99, 102, 103, 106, 108, 112, 115, 116, 119, 120, 123, 126, 130, 133, 137, 140, 143]
DHD_73
[2, 6, 9, 10, 12, 16, 17, 23, 24, 27, 30, 31, 34, 36, 37, 40, 43, 44, 47, 51, 54, 57, 58, 61, 65, 69, 79, 82, 86, 89, 92,
93, 96, 99, 100, 103, 104, 107, 117, 120, 124, 127, 131, 134, 137, 138, 141, 142, 145, 148, 149]
DHD_88
[2, 3, 6, 9, 13, 16, 20, 23, 27, 30, 34, 37, 48, 51, 52, 55, 56, 58, 59, 62, 63, 65, 66, 69, 70, 72, 73, 76, 79, 80, 81,
83, 87, 90, 91, 94, 97, 98, 101, 104, 105, 108, 111, 112, 115, 116, 121, 125, 128, 132, 135, 136, 139, 140, 142, 1
43, 146, 149, 150, 153, 154]
DHD_89
[1, 2, 5, 8, 9, 12, 15, 16, 19, 22, 23, 26, 29, 30, 33, 34, 36, 37, 38, 48, 51, 55, 58, 62, 65, 69, 72, 79, 82, 83, 86, 8
7, 89, 90, 93, 94, 97, 100, 101, 103, 104, 107, 108, 109, 110, 111, 114, 117, 118, 121, 124, 128, 131, 135, 138, 1
39, 142, 145, 146]
DHD_90
[1, 2, 5, 6, 8, 9, 12, 13, 16, 19, 20, 23, 26, 27, 29, 30, 33, 34, 37, 39, 43, 46, 47, 50, 54, 57, 61, 64, 68, 71, 75, 77,
78, 81, 82, 85, 88, 89, 91, 92, 95, 96, 99, 102, 103, 106, 109, 110, 113, 116, 119, 122, 125, 126, 129, 132, 133,
136, 139, 140, 143, 146, 147, 150, 151]
DHD_91
[2, 5, 9, 12, 16, 17, 19, 23, 26, 27, 30, 33, 39, 40, 43, 46, 50, 53, 57, 60, 64, 67, 70, 74, 77, 78, 81, 84, 85, 88, 91,
92, 95, 96, 98, 99, 101, 109, 112, 116, 119, 123, 126, 130, 133, 137, 138]
DHD_92
[5, 6, 8, 9, 12, 13, 15, 16, 19, 20, 22, 23, 26, 27, 29, 30, 33, 36, 37, 38, 42, 46, 49, 50, 53, 56, 57, 59, 60, 63, 67,
70, 71, 74, 77, 80, 81, 84, 87, 88, 91, 94, 95, 98, 101, 102, 105, 109, 110, 114, 118, 121, 125, 128, 129, 132, 135
, 136, 139, 142, 143]
DHD_93
[2, 5, 6, 9, 10, 12, 13, 16, 19, 20, 23, 26, 27, 30, 34, 35, 37, 40, 44, 47, 51, 54, 58, 61, 65, 76, 79, 83, 86, 87, 90,
93, 94, 97, 100, 104, 107, 109, 113, 116, 117, 120, 123, 124, 127, 130, 131, 134, 137, 138, 141, 144, 145]
DHD_94
[1, 5, 8, 9, 12, 15, 16, 19, 22, 23, 26, 29, 30, 33, 37, 45, 48, 51, 52, 55, 59, 62, 66, 69, 73, 75, 76, 84, 87, 88, 91,
94, 98, 101, 102, 105, 108, 109, 111, 112, 115, 117, 118, 122, 126, 129, 130, 133, 136, 140, 143, 147, 150, 151,
154]
DHD_95
[2, 6, 9, 13, 16, 20, 23, 26, 27, 30, 34, 37, 39, 41, 42, 45, 48, 49, 52, 55, 56, 59, 62, 63, 66, 69, 70, 72, 73, 75, 81,
85, 88, 91, 92, 95, 96, 98, 99, 102, 105, 106, 109, 110, 113, 114, 120, 123, 124, 127, 130, 131, 134, 137, 138, 1
41, 145, 148, 151]
DHD_96
[1, 5, 6, 9, 10, 12, 13, 16, 19, 20, 23, 26, 27, 30, 33, 34, 42, 45, 46, 49, 52, 53, 56, 59, 60, 63, 66, 67, 70, 74, 75,
77, 81, 84, 88, 91, 95, 98, 102, 105, 114, 118, 119, 122, 123, 126, 129, 130, 132, 133, 136, 137, 139, 140, 143, 1
44, 146, 147, 150]
DHD_97
[1, 2, 5, 6, 8, 9, 12, 15, 16, 19, 20, 22, 23, 26, 27, 29, 30, 32, 33, 37, 39, 41, 44, 47, 51, 54, 58, 61, 65, 68, 71, 72,
79, 82, 83, 86, 89, 90, 93, 96, 97, 100, 103, 104, 107, 110, 115, 116, 119, 120, 121, 123, 126, 127, 130, 133, 13
4, 136, 137, 140, 141, 144, 147]
DHD_98
[6, 7, 9, 13, 16, 20, 23, 27, 30, 34, 35, 38, 43, 44, 50, 51, 53, 54, 57, 60, 61, 64, 67, 68, 71, 72, 74, 75, 78, 82, 85,
86, 89, 92, 93, 96, 99, 100, 103, 106, 110, 119, 123, 127, 130, 133, 134, 137, 141, 144, 148, 151, 152]
DHD_99
[2, 5, 6, 9, 12, 16, 19, 20, 23, 26, 30, 33, 34, 46, 50, 53, 57, 60, 64, 67, 70, 71, 74, 78, 82, 85, 89, 92, 96, 99, 103,
106, 110, 113, 123, 126, 130, 133, 137, 140, 141, 144, 147, 148, 151, 154, 155]
DHD_100
[1, 5, 8, 9, 12, 15, 16, 19, 22, 23, 26, 29, 30, 33, 38, 41, 42, 45, 49, 52, 55, 56, 59, 63, 66, 70, 73, 74, 77, 80, 81,
84, 87, 88, 91, 94, 95, 98, 101, 102, 104, 105, 106, 108, 109, 112, 115, 119, 122, 123, 126, 129, 130, 133, 136, 1
37]
DHD_101
[5, 6, 9, 12, 13, 16, 20, 23, 27, 30, 34, 39, 43, 46, 47, 50, 53, 54, 57, 60, 64, 67, 68, 71, 74, 75, 78, 79, 81, 84, 85,
88, 92, 95, 99, 102, 106, 109, 119, 120, 123, 126, 130, 133, 134, 137, 138, 140, 144, 147, 151]
DHD_102
[6, 9, 13, 16, 20, 22, 23, 27, 30, 34, 38, 44, 47, 51, 54, 55, 57, 58, 61, 65, 68, 69, 72, 75, 80, 83, 86, 90, 93, 97, 1
00, 104, 107, 108, 112, 113, 117, 121, 124, 128, 131, 132, 135, 138, 142, 145]
DHD_103
[1, 2, 5, 6, 8, 9, 13, 16, 17, 19, 20, 23, 27, 30, 34, 39, 43, 46, 47, 50, 53, 54, 57, 61, 64, 68, 70, 71, 79, 80, 82, 86,
89, 93, 94, 96, 100, 103, 106, 112, 116, 119, 123, 126, 130, 133, 134, 137, 140]
DHD_104
[6, 9, 10, 13, 16, 17, 19, 20, 23, 24, 27, 30, 31, 34, 45, 48, 52, 55, 59, 62, 66, 69, 80, 83, 84, 87, 90, 94, 97, 98, 1
01, 104, 113, 114, 117, 120, 124, 127, 131, 134, 138, 141, 144, 145]
DHD_105
[9, 12, 16, 19, 23, 26, 30, 33, 37, 41, 43, 44, 47, 50, 51, 54, 57, 58, 61, 64, 65, 68, 71, 72, 79, 82, 83, 86, 89, 90,
93, 97, 100, 101, 103, 104, 107, 118, 121, 122, 125, 128, 129, 132, 135, 136, 139, 142, 143, 146, 147]
DHD_106
[2, 3, 6, 9, 10, 12, 13, 16, 17, 20, 23, 24, 27, 30, 34, 43, 46, 50, 53, 57, 60, 64, 67, 71, 74, 78, 81, 85, 88, 92, 93,
95, 99, 102, 103, 106, 109, 110, 113, 120, 121, 123, 127, 128, 130, 134, 135, 137, 141, 144, 148, 151, 155]
DHD_107
[2, 6, 9, 10, 13, 16, 20, 23, 24, 27, 30, 31, 34, 40, 43, 46, 50, 53, 56, 57, 60, 63, 64, 67, 71, 73, 75, 81, 84, 85, 88,
91, 92, 95, 98, 99, 102, 106, 109, 114, 115, 116, 119, 122, 123, 125, 126, 129, 130, 133, 136, 137, 140, 143, 14
4, 146]
DHD_108
[2, 6, 7, 10, 17, 20, 21, 24, 25, 28, 35, 42, 43, 46, 50, 53, 57, 60, 61, 64, 68, 71, 75, 82, 86, 89, 93, 97, 100, 104,
111, 115, 116, 123, 124, 127, 128, 130, 131, 134, 135, 138, 141, 142, 145, 146, 149, 152, 153, 156]
DHD_109
[6, 7, 10, 14, 17, 21, 25, 28, 32, 35, 41, 42, 44, 45, 48, 49, 52, 55, 56, 59, 60, 62, 63, 66, 70, 77, 81, 84, 85, 88, 9
2, 95, 99, 102, 106, 109, 111, 116, 117, 120, 123, 124, 127, 130, 131, 134, 135, 138, 141, 142]
DHD_110
[5, 6, 9, 13, 17, 20, 24, 27, 31, 35, 38, 48, 49, 52, 53, 56, 59, 60, 63, 66, 67, 70, 74, 77, 78, 81, 84, 85, 88, 91, 92,
95, 96, 99, 102, 103, 106, 109, 110, 113, 114, 117, 128, 129, 132, 136, 139, 143, 147, 150, 154, 157, 161]
DHD_111
[5, 6, 9, 10, 13, 16, 17, 20, 23, 24, 27, 28, 31, 34, 35, 47, 51, 54, 58, 62, 65, 69, 72, 76, 80, 84, 87, 90, 91, 94, 95,
98, 101, 102, 105, 109, 112, 113, 116, 122, 125, 129, 132, 136, 140, 143, 147, 154, 158]
DHD_112
[6, 13, 16, 17, 20, 24, 27, 31, 35, 38, 48, 51, 52, 55, 58, 59, 62, 63, 66, 69, 70, 73, 76, 77, 80, 81, 88, 91, 92, 95,
99, 102, 106, 109, 113, 120, 121, 123, 124, 126, 127, 130, 133, 134, 137, 138, 141, 144, 145, 148, 151, 152, 155
, 156]
DHD_113
[6, 9, 13, 14, 16, 20, 23, 27, 30, 31, 34, 39, 43, 44, 47, 48, 51, 54, 55, 58, 61, 62, 65, 66, 68, 69, 72, 73, 76, 80, 8
3, 84, 87, 90, 91, 94, 97, 98, 101, 102, 105, 108, 109, 112, 117, 122, 125, 129, 133, 136, 140, 143, 147, 151]
DHD_114
[5, 6, 9, 10, 13, 16, 17, 20, 23, 24, 27, 30, 31, 34, 35, 38, 39, 41, 44, 47, 51, 55, 58, 62, 65, 69, 72, 76, 81, 84, 87,
88, 91, 95, 98, 99, 102, 105, 106, 108, 109, 113, 117, 122, 126, 129, 130, 133, 137, 140, 144, 147, 151, 155]
DHD_115
[5, 6, 9, 12, 13, 16, 17, 20, 23, 24, 27, 30, 31, 34, 37, 43, 47, 50, 54, 57, 58, 61, 65, 68, 69, 72, 74, 75, 76, 78, 79,
82, 85, 86, 89, 90, 93, 96, 97, 100, 103, 104, 107, 108, 121, 125, 128, 132, 135, 139, 143, 146, 150, 153]
DHD_116
[3, 6, 7, 10, 14, 17, 21, 24, 25, 28, 32, 35, 36, 48, 49, 52, 56, 59, 60, 63, 66, 67, 70, 71, 74, 77, 78, 81, 87, 88, 91,
95, 98, 99, 102, 103, 105, 106, 109, 110, 113, 116, 129, 133, 136, 140, 144, 147, 151, 154, 158, 161, 162]
DHD_117
[6, 10, 13, 17, 20, 21, 24, 27, 28, 31, 35, 38, 40, 44, 47, 48, 51, 55, 58, 59, 62, 65, 66, 69, 72, 73, 76, 77, 79, 83,
84, 87, 90, 91, 94, 98, 101, 102, 105, 108, 109, 112, 116, 125, 126, 129, 133, 136, 140, 141, 143, 144, 147, 151,
154, 155]
DHD_118
[6, 10, 13, 14, 17, 20, 24, 28, 31, 35, 38, 49, 52, 53, 56, 59, 60, 63, 66, 67, 70, 74, 77, 81, 82, 85, 88, 92, 95, 99,
102, 106, 109, 110, 113, 116, 118, 119, 123, 127, 130, 131, 134, 137, 138, 141, 145, 148, 149, 152, 153]
DHD_119
[2, 6, 10, 13, 14, 17, 20, 21, 24, 25, 28, 31, 32, 35, 40, 41, 45, 46, 49, 52, 56, 60, 63, 67, 71, 74, 75, 78, 82, 84, 8
5, 88, 89, 92, 95, 96, 99, 103, 106, 107, 110, 113, 114, 117, 120, 122, 127, 131, 134, 138, 141, 142, 145, 148, 14
9, 152, 156]
DHD_120
[7, 10, 13, 14, 17, 21, 25, 28, 31, 32, 35, 41, 44, 45, 48, 49, 51, 52, 55, 56, 59, 62, 63, 66, 69, 70, 73, 74, 79, 83,
84, 87, 91, 94, 95, 98, 101, 102, 105, 109, 112, 113, 122, 126, 129, 133, 136, 137, 140, 144, 147, 148, 151]
DHD_121
[5, 6, 9, 13, 16, 20, 23, 27, 30, 37, 43, 46, 47, 50, 53, 54, 57, 60, 61, 64, 67, 68, 71, 74, 75, 82, 85, 86, 89, 92, 93,
96, 99, 100, 103, 106, 107, 110, 113, 115, 120, 123, 127, 130, 134, 137, 141, 144, 148]
DHD_122
[6, 10, 13, 14, 17, 21, 24, 25, 28, 31, 32, 35, 41, 44, 45, 48, 51, 52, 55, 59, 62, 63, 66, 69, 70, 73, 74, 75, 81, 82,
85, 88, 92, 95, 96, 99, 100, 102, 103, 106, 107, 110, 113, 123, 127, 131, 134, 138, 141, 145, 152, 156, 157]
DHD_123
[1, 3, 6, 10, 13, 14, 17, 20, 21, 24, 28, 31, 32, 35, 38, 39, 40, 42, 44, 48, 51, 52, 55, 59, 62, 66, 69, 70, 73, 74, 80,
83, 84, 87, 91, 94, 95, 98, 102, 105, 109, 110, 115, 118, 119, 122, 123, 126, 129, 130, 133, 134, 136, 137, 140,
144, 145, 147, 148]
DHD_124
[6, 9, 10, 13, 16, 17, 20, 24, 27, 28, 31, 34, 35, 38, 43, 44, 47, 50, 51, 54, 58, 61, 62, 65, 68, 69, 72, 79, 83, 86, 8
7, 90, 91, 94, 97, 98, 101, 104, 105, 108, 112, 121, 125, 128, 129, 132, 136, 139, 143, 146, 147, 150, 154, 155]
DHD_125
[2, 6, 9, 10, 13, 16, 17, 20, 24, 27, 28, 31, 35, 38, 43, 46, 47, 50, 54, 57, 58, 61, 65, 68, 69, 72, 73, 75, 79, 82, 83,
86, 90, 93, 97, 100, 101, 104, 107, 108, 111, 121, 122, 125, 129, 132, 133, 136, 139, 140, 143, 147, 150]
DHD_126
[1, 2, 5, 6, 9, 10, 12, 13, 16, 20, 23, 24, 27, 28, 30, 31, 34, 43, 47, 50, 51, 54, 58, 61, 62, 65, 68, 69, 72, 83, 86, 8
7, 90, 94, 97, 98, 101, 105, 108, 109, 112, 115, 121, 122, 125, 128, 132, 136, 139, 140, 143, 146, 147, 150, 151]
DHD_127
[3, 6, 9, 10, 13, 14, 16, 17, 20, 23, 24, 27, 31, 34, 39, 44, 47, 51, 54, 58, 61, 65, 68, 72, 79, 83, 86, 90, 93, 96, 97,
100, 103, 104, 107, 111, 114, 117, 121, 124, 128, 131, 135, 139, 142, 145, 146]
DHD_128
[6, 9, 10, 13, 16, 17, 20, 23, 24, 27, 30, 31, 32, 40, 44, 47, 51, 54, 58, 61, 65, 68, 72, 79, 83, 86, 87, 90, 93, 97, 1
00, 104, 118, 121, 125, 128, 132, 135, 139, 142, 146]
DHD_129
[2, 5, 6, 9, 12, 13, 16, 19, 20, 23, 24, 26, 27, 30, 34, 42, 46, 49, 53, 56, 60, 63, 64, 67, 70, 74, 80, 81, 84, 88, 91,
94, 95, 98, 101, 102, 105, 109, 112, 122, 125, 126, 129, 132, 133, 135, 136, 139, 143, 146, 147, 150, 153]
DHD_130
[3, 6, 10, 13, 17, 20, 24, 27, 31, 34, 37, 41, 44, 45, 48, 51, 52, 55, 58, 59, 62, 65, 66, 69, 70, 72, 73, 79, 83, 86, 9
0, 93, 97, 100, 104, 107, 111, 116, 121, 122, 125, 128, 129, 132, 135, 136, 139, 142, 143, 146, 149, 150, 153]
DHD_131
[3, 6, 9, 10, 13, 16, 17, 20, 24, 27, 31, 34, 44, 48, 51, 52, 54, 55, 58, 59, 61, 62, 65, 66, 69, 72, 73, 76, 83, 87, 90,
91, 94, 97, 101, 104, 108, 111, 121, 124, 125, 128, 131, 132, 135, 138, 139, 142, 145, 146, 149, 150, 152, 153]
DHD_132
[5, 6, 9, 12, 13, 16, 19, 20, 23, 26, 27, 30, 33, 34, 37, 39, 43, 46, 47, 50, 54, 57, 64, 68, 71, 74, 75, 85, 86, 89, 92,
93, 95, 99, 100, 103, 105, 106, 107, 110, 114, 120, 123, 127, 130, 134, 137, 141, 144, 148]
DHD_133
[6, 10, 13, 17, 20, 21, 24, 27, 31, 34, 39, 44, 45, 48, 51, 52, 55, 58, 59, 62, 65, 66, 69, 72, 73, 76, 83, 87, 90, 94,
97, 101, 104, 108, 111, 112, 121, 122, 125, 126, 129, 132, 136, 139, 143, 146, 149, 150, 153]
DHD_134
[6, 9, 10, 13, 16, 17, 20, 23, 24, 27, 30, 31, 34, 38, 43, 47, 50, 54, 57, 61, 64, 68, 71, 75, 82, 85, 89, 92, 96, 99, 1
03, 106, 110, 111, 118, 122, 125, 129, 132, 133, 136, 139, 140, 143, 146]
DHD_135
[2, 5, 6, 9, 12, 13, 16, 19, 20, 23, 26, 27, 30, 33, 34, 37, 38, 46, 50, 53, 57, 60, 64, 67, 71, 80, 81, 84, 87, 91, 94,
95, 98, 101, 102, 105, 108, 115, 119, 122, 126, 129, 130, 133, 136, 140, 143]
DHD_136
[5, 6, 8, 9, 12, 15, 16, 19, 22, 23, 26, 29, 30, 33, 36, 37, 41, 45, 48, 52, 55, 59, 62, 66, 69, 70, 72, 73, 79, 82, 83,
86, 89, 90, 93, 97, 100, 104, 107, 117, 120, 121, 124, 127, 128, 131, 134, 135, 138, 141, 142]
DHD_137
[2, 6, 9, 13, 16, 17, 20, 23, 24, 27, 30, 31, 34, 43, 46, 47, 50, 54, 57, 61, 64, 68, 71, 72, 74, 78, 81, 85, 89, 92, 96,
99, 102, 103, 107, 113, 116, 117, 120, 123, 124, 127, 130, 131, 134, 137, 138, 141]
DHD_138
[6, 10, 13, 17, 20, 24, 27, 31, 34, 43, 47, 50, 54, 57, 61, 64, 68, 71, 82, 86, 89, 93, 96, 100, 103, 107, 110, 119, 1
20, 123, 126, 127, 130, 133, 134, 137, 140, 143, 144, 147, 148, 150, 151]
DHD_139
[2, 5, 6, 9, 12, 13, 16, 19, 20, 23, 26, 27, 30, 34, 37, 38, 39, 45, 49, 52, 56, 59, 63, 66, 76, 79, 80, 83, 86, 87, 90,
93, 94, 97, 100, 101, 104, 107, 108, 113, 115, 118, 121, 125, 128, 132, 135, 139, 142, 143, 145, 146]
DHD_140
[5, 6, 9, 12, 13, 16, 19, 20, 23, 26, 27, 30, 33, 36, 38, 43, 47, 50, 51, 54, 57, 61, 64, 68, 71, 78, 81, 85, 88, 89, 92,
95, 96, 99, 102, 103, 120, 124, 127, 131, 134, 135, 138, 141, 145, 148]
DHD_141
[6, 10, 13, 17, 20, 24, 27, 28, 31, 34, 37, 40, 43, 44, 47, 48, 51, 54, 57, 58, 61, 62, 64, 65, 68, 71, 72, 81, 85, 88,
92, 95, 99, 102, 106, 109, 113, 118, 120, 123, 124, 127, 130, 131, 134, 137, 138, 141, 144, 145, 148, 151, 152]
DHD_142
[1, 2, 5, 6, 9, 12, 13, 15, 16, 19, 20, 23, 24, 26, 27, 30, 33, 40, 44, 47, 51, 54, 58, 61, 68, 69, 72, 75, 78, 79, 82, 8
5, 86, 89, 92, 95, 96, 99, 100, 103, 106, 107, 110, 120, 121, 124, 127, 131, 134, 138, 141, 145, 148, 152]
DHD_143
[2, 6, 9, 13, 16, 17, 20, 23, 27, 30, 34, 39, 44, 48, 51, 55, 58, 62, 65, 66, 69, 72, 73, 83, 87, 90, 94, 97, 101, 104,
108, 111, 121, 124, 125, 128, 131, 132, 135, 136, 138, 139, 142, 145, 146, 149, 150, 152, 153]
DHD_144
[1, 5, 6, 9, 12, 13, 16, 19, 20, 23, 26, 27, 30, 33, 42, 46, 49, 53, 56, 60, 63, 67, 70, 73, 74, 76, 77, 80, 81, 83, 84,
87, 88, 90, 91, 94, 95, 98, 99, 101, 102, 105, 108, 109, 117, 120, 121, 124, 128, 131, 135, 138, 142, 145, 149, 15
0]
DHD_145
[5, 6, 9, 12, 13, 16, 19, 20, 23, 26, 30, 33, 34, 47, 50, 54, 57, 61, 64, 68, 71, 75, 78, 85, 86, 88, 89, 92, 95, 96, 99,
102, 103, 106, 109, 110, 113, 123, 127, 130, 134, 137, 141, 144, 145, 148, 151, 155]
DHD_146
[2, 5, 6, 9, 10, 12, 13, 16, 19, 23, 26, 27, 30, 33, 34, 40, 43, 46, 50, 53, 54, 57, 60, 61, 64, 67, 71, 78, 81, 82, 85,
88, 89, 92, 95, 96, 99, 100, 102, 103, 106, 126, 129, 133, 136, 140, 143, 147, 150, 151]
DHD_147
[6, 7, 10, 13, 14, 17, 20, 24, 27, 31, 34, 37, 38, 42, 43, 46, 49, 50, 53, 54, 56, 57, 60, 63, 64, 67, 70, 71, 74, 81, 8
4, 88, 91, 95, 98, 102, 105, 109, 116, 119, 123, 126, 127, 130, 133, 137, 140, 141, 144, 147, 148]
DHD_149
[1, 5, 6, 9, 12, 13, 16, 17, 19, 23, 26, 27, 30, 33, 34, 42, 46, 49, 53, 56, 60, 63, 67, 70, 74, 75, 77, 81, 84, 85, 88,
91, 92, 95, 98, 102, 105, 106, 116, 120, 123, 124, 127, 130, 131, 134, 137, 138, 141, 144, 145, 148]
DHD_150
[1, 2, 5, 6, 9, 12, 13, 16, 19, 20, 23, 26, 27, 30, 33, 34, 42, 46, 49, 50, 53, 56, 60, 63, 67, 70, 74, 75, 77, 81, 88, 9
1, 95, 98, 102, 105, 108, 116, 120, 123, 124, 127, 130, 131, 134, 137, 138, 141, 144, 145, 148]
DHD_151
[5, 9, 12, 13, 16, 19, 20, 23, 26, 27, 30, 33, 34, 36, 43, 46, 47, 50, 54, 57, 61, 64, 68, 71, 74, 78, 81, 82, 85, 88, 8
9, 92, 95, 96, 99, 102, 103, 106, 111, 116, 120, 123, 127, 130, 134, 137, 141, 144, 148]
DHD_152
[5, 6, 9, 12, 13, 19, 20, 23, 26, 27, 30, 33, 34, 36, 40, 43, 46, 47, 50, 54, 57, 61, 64, 68, 71, 74, 78, 81, 82, 85, 88,
89, 92, 95, 96, 99, 102, 103, 106, 111, 116, 120, 123, 127, 134, 137, 141, 144, 148]
DHD_153
[6, 10, 13, 14, 17, 20, 21, 24, 27, 31, 34, 44, 45, 48, 51, 52, 58, 59, 62, 65, 66, 69, 73, 74, 76, 80, 83, 84, 87, 90,
91, 94, 97, 101, 104, 105, 107, 115, 118, 122, 125, 126, 129, 132, 136, 139, 143]
DHD_154
[2, 5, 6, 9, 12, 16, 19, 20, 23, 26, 27, 30, 33, 34, 40, 43, 47, 50, 54, 57, 61, 64, 68, 71, 72, 74, 78, 81, 85, 88, 92,
95, 99, 102, 106, 109, 115, 116, 119, 122, 123, 126, 129, 133, 136, 137, 140, 143, 144, 147]
DHD_155
[5, 6, 9, 13, 16, 19, 20, 23, 27, 30, 34, 43, 47, 50, 53, 54, 57, 61, 64, 68, 75, 78, 79, 81, 82, 85, 88, 89, 92, 95, 96,
99, 102, 109, 113, 116, 120, 123, 127, 130, 134, 137]
DHD_156
[5, 6, 9, 12, 13, 16, 19, 20, 23, 26, 27, 30, 37, 40, 44, 47, 51, 54, 58, 61, 65, 68, 75, 76, 79, 82, 83, 89, 90, 93, 96,
97, 100, 104, 124, 128, 131, 132, 135, 138, 142, 145, 146]
DHD_157
[5, 6, 9, 13, 16, 20, 23, 27, 30, 33, 34, 37, 39, 43, 44, 47, 48, 51, 54, 55, 58, 61, 62, 65, 68, 69, 72, 75, 76, 79, 83,
86, 87, 90, 93, 94, 97, 100, 101, 104, 107, 118, 122, 125, 129, 132, 136, 139, 143, 146, 150]
DHD13_2341
[4, 5, 8, 11, 12, 15, 18, 19, 22, 25, 29, 32, 33, 36, 39, 41, 71, 91, 95, 98, 102, 105, 106, 109, 112, 113, 116, 117,
119, 121, 125, 126, 129, 132, 133, 136, 137, 139, 140, 143, 144, 146, 147, 150, 153, 154]
DHD13_AAAA
[6, 9, 13, 16, 17, 20, 23, 24, 27, 30, 34, 39, 44, 47, 48, 51, 54, 55, 58, 61, 62, 65, 68, 69, 72, 75, 82, 85, 89, 92, 9
6, 99, 100, 103, 106, 107, 109, 110, 111, 115, 123, 124, 127, 128, 130, 134, 137, 138, 141, 144, 145, 148, 149, 1
51]
DHD13_BAAA
[6, 9, 12, 13, 16, 17, 20, 23, 24, 27, 30, 34, 37, 38, 39, 44, 47, 51, 54, 55, 58, 61, 62, 65, 68, 69, 72, 75, 82, 85, 8
9, 92, 95, 96, 99, 100, 103, 106, 110, 115, 123, 127, 130, 134, 137, 138, 141, 144, 145, 148, 149, 151]
DHD13_XAAA
[6, 9, 13, 16, 17, 20, 23, 24, 27, 30, 34, 37, 38, 39, 44, 47, 48, 51, 54, 55, 58, 61, 62, 65, 68, 69, 72, 75, 82, 85, 8
9, 92, 96, 99, 100, 103, 106, 109, 110, 115, 123, 127, 130, 134, 137, 138, 141, 144, 145, 148, 149, 151]
DHD13_XAAX
[6, 9, 12, 13, 16, 17, 20, 23, 24, 27, 30, 34, 37, 38, 39, 44, 47, 48, 51, 54, 55, 58, 61, 62, 65, 68, 69, 72, 75, 82, 8
5, 89, 92, 96, 99, 100, 103, 106, 109, 110, 115, 123, 127, 130, 134, 137, 138, 141, 144, 145, 148, 149, 151]
DHD13_XAXA
[6, 9, 13, 16, 17, 20, 23, 24, 27, 30, 34, 37, 38, 39, 44, 47, 48, 51, 54, 55, 58, 61, 62, 65, 68, 69, 72, 75, 82, 85, 8
9, 92, 96, 99, 100, 103, 106, 109, 110, 115, 123, 127, 130, 134, 137, 138, 141, 144, 145, 148, 149, 151]
DHD15
[5, 6, 9, 12, 13, 16, 19, 20, 23, 26, 27, 30, 33, 34, 37, 39, 40, 44, 47, 48, 51, 52, 55, 58, 59, 62, 65, 69, 72, 78, 82,
83, 86, 89, 90, 93, 96, 97, 100, 103, 104, 107, 110, 111, 114, 116, 117, 121, 125, 128, 129, 132, 135, 136, 139,
142, 143, 146, 149]
DHD17
[6, 9, 10, 13, 16, 17, 20, 23, 24, 27, 30, 31, 34, 38, 42, 43, 46, 50, 53, 57, 60, 64, 67, 71, 75, 81, 84, 88, 91, 95, 9
8, 102, 105, 106, 109, 118, 121, 125, 128, 132, 135, 136, 139, 142, 146]
DHD20
[3, 6, 9, 10, 13, 16, 17, 20, 23, 24, 27, 30, 31, 34, 38, 42, 43, 46, 50, 53, 57, 60, 61, 64, 67, 68, 71, 75, 78, 81, 85,
88, 91, 92, 95, 98, 99, 102, 105, 109, 118, 121, 125, 128, 132, 135, 139, 142, 146, 150]
DHD21
[6, 9, 10, 13, 16, 20, 23, 24, 27, 30, 31, 34, 38, 43, 44, 46, 47, 50, 51, 53, 54, 57, 58, 60, 61, 63, 64, 65, 68, 71, 7
2, 75, 78, 79, 82, 85, 89, 92, 93, 96, 99, 100, 103, 107, 108, 112, 113, 116, 119, 120, 123, 126, 127, 130, 133, 13
4, 137, 141, 142]
DHD25
[2, 5, 6, 9, 12, 13, 16, 19, 23, 26, 27, 30, 44, 47, 48, 51, 54, 55, 58, 61, 62, 65, 68, 69, 72, 75, 76, 83, 86, 87, 90,
93, 94, 97, 100, 101, 104, 107, 114, 115, 117, 118, 121, 124, 128, 131, 132, 135, 138, 142, 145]
DHD27
[5, 6, 9, 12, 15, 16, 19, 22, 23, 26, 29, 30, 33, 37, 41, 42, 45, 49, 52, 56, 59, 63, 66, 70, 73, 74, 77, 81, 84, 88, 91,
95, 98, 102, 105, 108, 115, 116, 119, 122, 123, 126, 129, 130, 133, 136, 137, 140, 141, 143, 144, 147, 148]
DHD30
[5, 6, 8, 9, 12, 15, 16, 19, 22, 23, 26, 29, 30, 33, 34, 37, 39, 42, 45, 49, 52, 56, 59, 63, 66, 70, 75, 78, 82, 85, 89,
92, 96, 99, 103, 106, 113, 116, 117, 120, 124, 127, 131, 134, 138, 141, 144, 145]
DHD33
[1, 5, 6, 9, 12, 13, 16, 19, 20, 23, 26, 27, 30, 33, 34, 39, 40, 43, 46, 47, 50, 57, 60, 61, 64, 68, 71, 75, 78, 82, 85,
86, 89, 92, 93, 96, 99, 100, 103, 112, 113, 116, 119, 120, 123, 126, 127, 130, 133, 134, 137, 140, 141, 144]
DHD34_XAAAA
[1, 5, 8, 9, 12, 16, 19, 23, 26, 29, 30, 33, 37, 45, 48, 49, 52, 55, 56, 59, 62, 63, 66, 69, 70, 73, 76, 77, 86, 90, 93,
97, 100, 104, 105, 107, 111, 114, 124, 125, 128, 131, 132, 135, 138, 142, 145, 149, 152, 153, 156, 157]
DHD34_XAAXA
[1, 5, 8, 9, 12, 16, 19, 23, 26, 29, 30, 33, 37, 45, 48, 49, 52, 55, 56, 59, 62, 63, 66, 69, 70, 73, 76, 77, 86, 90, 93,
97, 100, 104, 105, 107, 108, 111, 114, 124, 125, 128, 131, 132, 135, 138, 142, 145, 149, 152, 153, 156, 157]
DHD34_XAXXA
[1, 5, 8, 9, 12, 16, 19, 22, 23, 26, 29, 30, 33, 37, 45, 48, 49, 52, 55, 56, 59, 62, 63, 66, 69, 70, 73, 76, 77, 86, 90,
93, 97, 100, 104, 105, 107, 108, 111, 114, 117, 124, 125, 128, 131, 132, 135, 138, 142, 145, 149, 152, 153, 156,
157]
DHD36
[2, 6, 9, 13, 16, 20, 23, 27, 30, 33, 34, 39, 40, 42, 43, 46, 47, 50, 51, 53, 54, 57, 58, 61, 64, 75, 79, 82, 86, 89, 93,
96, 97, 100, 103, 110, 114, 117, 118, 121, 124, 125, 128, 131, 132, 135, 138, 139, 142, 143]
DHD37_ABXB
[2, 5, 9, 11, 12, 15, 16, 19, 23, 26, 29, 30, 33, 37, 43, 46, 49, 50, 53, 56, 57, 59, 60, 63, 64, 67, 77, 81, 84, 88, 91,
92, 95, 98, 99, 102, 105, 113, 116, 119, 120, 123, 126, 130, 133, 134, 137, 140, 141, 144]
DHD37_AXBB
[2, 5, 9, 12, 16, 18, 19, 22, 23, 26, 29, 30, 33, 37, 43, 46, 49, 50, 52, 53, 56, 57, 60, 67, 70, 71, 77, 78, 81, 84, 88,
91, 95, 96, 99, 102, 105, 113, 116, 119, 120, 123, 126, 127, 130, 133, 134, 137, 140, 144]
DHD37_AXXB
[2, 5, 9, 11, 12, 15, 16, 19, 22, 23, 26, 29, 30, 33, 37, 43, 46, 49, 50, 53, 56, 57, 59, 60, 63, 64, 67, 77, 81, 84, 88,
89, 92, 95, 98, 99, 102, 105, 113, 116, 119, 120, 123, 126, 130, 133, 134, 137, 140, 141, 144]
DHD37_BBBB
[5, 9, 12, 16, 19, 22, 23, 26, 30, 33, 42, 43, 46, 50, 53, 56, 57, 60, 63, 64, 66, 67, 74, 77, 78, 81, 84, 85, 88, 92, 9
5, 98, 99, 102, 105, 108, 111, 113, 116, 119, 123, 126, 130, 133, 137, 140, 141, 144]
DHD37_XBBA
[2, 5, 8, 9, 12, 16, 19, 23, 25, 26, 29, 30, 33, 37, 43, 45, 46, 49, 50, 53, 60, 63, 64, 67, 70, 71, 77, 78, 81, 84, 85,
88, 91, 95, 98, 102, 103, 105, 113, 116, 119, 120, 123, 126, 127, 130, 133, 137, 140, 141, 144]
DHD37_XBXB
[2, 5, 9, 11, 12, 15, 16, 19, 23, 26, 30, 33, 37, 43, 46, 50, 53, 56, 57, 59, 60, 63, 64, 67, 77, 81, 84, 88, 92, 95, 98,
99, 102, 105, 113, 116, 119, 120, 123, 126, 130, 133, 134, 137, 140, 141, 144]
XAAX
[3, 6, 9, 13, 16, 20, 23, 27, 30, 34, 43, 47, 50, 54, 57, 61, 64, 68, 71, 72, 3, 6, 9, 13, 16, 20, 23, 27, 30, 34, 43, 47,
50, 54, 57, 61, 64, 68, 71, 72]
XAXA
[3, 6, 9, 13, 16, 20, 23, 27, 30, 34, 43, 47, 50, 54, 57, 61, 64, 68, 71, 72, 3, 6, 9, 13, 16, 20, 23, 27, 30, 34, 43, 47,
50, 54, 57, 61, 64, 68, 71, 72]

In one embodiment, the monomer A polypeptide and the monomer B polypeptide have their interaction specificity determined by at least one designed hydrogen bond network at the interface between the monomer A and the monomer B. In some aspects, (i) monomer A comprises 1 helix, and monomer B comprises 1 helix; (ii) monomer A comprises 1 helix and monomer B comprises 2 helices; (iii) monomer A comprises 1 helix and monomer B comprises 3 helices, (iv) monomer A comprises 1 helix and monomer B comprises 4 helices; or (v) monomer A comprises 1 helix and monomer B comprises 5 helices, wherein the monomer A and the monomer B comprise a hydrogen bond network, e.g., hydrogen bonds that are capable of being formed by the interface residues according to Table 2. In some aspects, (i) monomer A comprises 2 helices, and monomer B comprises 1 helix; (ii) monomer A comprises 2 helices and monomer B comprises 2 helices; (iii) monomer A comprises 2 helices and monomer B comprises 3 helices, (iv) monomer A comprises 2 helices and monomer B comprises 4 helices; or (v) monomer A comprises 2 helices and monomer B comprises 5 helices, wherein the monomer A and the monomer B comprise a hydrogen bond network, e.g., hydrogen bonds that are capable of being formed by the interface residues according to Table 2. In some aspects, (i) monomer A comprises 3 helices, and monomer B comprises 1 helix; (ii) monomer A comprises 3 helices and monomer B comprises 2 helices; (iii) monomer A comprises 3 helices and monomer B comprises 3 helices, (iv) monomer A comprises 3 helices and monomer B comprises 4 helices; or (v) monomer A comprises 3 helices and monomer B comprises 5 helices, wherein the monomer A and the monomer B comprise a hydrogen bond network, e.g., hydrogen bonds that are capable of being formed by the interface residues according to Table 2. In some aspects, (i) monomer A comprises 4 helices, and monomer B comprises 1 helix; (ii) monomer A comprises 4 helices and monomer B comprises 2 helices; (iii) monomer A comprises 4 helices and monomer B comprises 3 helices, (iv) monomer A comprises 4 helices and monomer B comprises 4 helices; or (v) monomer A comprises 4 helices and monomer B comprises 5 helices, wherein the monomer A and the monomer B comprise a hydrogen bond network, e.g., hydrogen bonds that are capable of being formed by the interface residues according to Table 2. In some aspects, (i) monomer A comprises 5 helices, and monomer B comprises 1 helix; (ii) monomer A comprises 5 helices and monomer B comprises 2 helices; (iii) monomer A comprises 5 helices and monomer B comprises 3 helices, (iv) monomer A comprises 5 helices and monomer B comprises 4 helices; or (v) monomer A comprises 5 helices and monomer B comprises 5 helices, wherein the monomer A and the monomer B comprise a hydrogen bond network, e.g., hydrogen bonds that are capable of being formed by the interface residues according to Table 2.

In a second aspect, the disclosure provides non-naturally occurring polypeptides comprising a polypeptide having at least 70%, 75%, 80%, 85%, 90%, 91%, 92%, 93%, 94%, 95%, 96%, 97%, 98%, 99%, or 100% sequence identity along the length of the amino acid sequence selected from the group consisting of SEQ ID NOS: 1-290, or the group consisting of SEQ ID NOS:1-290, 331, 332, 334, 336-422, 424, 426, 428, 430, 432, 434, 436, 438, 440, 442, 444, 446, 448, 450, 452, 454, 456, 458-460, 462, 464, 466, 468, 470, 472, 474, 476, 478, 480, 482, 484, 486, 488, 490, 493, and 494. The amino acid sequences of SEQ ID NOS: 1-290 are provided in Table 1A, and the amino acid sequences of SEQ ID NOS: 331, 332, 334, 336-422, 424, 426, 428, 430, 432, 434, 436, 438, 440, 442, 444, 446, 448, 450, 452, 454, 456, 458-460, 462, 464, 466, 468, 470, 472, 474, 476, 478, 480, 482, 484, 486, 488, 490, 493, and 494 are provided in Table 1B, and can be used, for example, to generate the heterodimers of the disclosure.

In some aspects, the disclosure provides non-naturally occurring polypeptides comprising a polypeptide having at least 70%, 75%, 80%, 85%, 90%, 91%, 92%, 93%, 94%, 95%, 96%, 97%, 98%, 99%, or 100% sequence identity along the length of the amino acid sequence selected from the group consisting of SEQ ID NOS: 1-290, wherein GlySer at amino acid positions 1 and 2 of SEQ ID NO: 1, 55, 81, 83, 101, 105, 115, 117, 119, 121, 123, 125, 127, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, or 193 are optional, e.g., not present. The amino acid sequences of SEQ ID NOS: 1-290 are provided in Table 1A, and can be used, for example, to generate the heterodimers of the disclosure.

In some aspects, the disclosure provides non-naturally occurring polypeptides comprising a polypeptide having at least 70%, 75%, 80%, 85%, 90%, 91%, 92%, 93%, 94%, 95%, 96%, 97%, 98%, 99%, or 100% sequence identity along the length of the amino acid sequence selected from the group consisting of SEQ ID NOS: 1-290, wherein GlySer at amino acid positions 1 and 2 of SEQ ID NO: 6, 8, 14, 16, 26, 30, 32, 34, 36, 38, 40, 42, 46, 48, 54, 56, 58, 60, 62, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 128, 130, 132, 134, 136, 138, 140, 142, 144, 146, 148, 150, 152, 154, 156, 158, 160, 162, 164, 166, 168, 170, 172, 176, 178, 180, 182, 184, 186, 188, 190, 192, or 194 are optional, e.g., not present.

In some aspects, the disclosure provides non-naturally occurring polypeptides comprising a polypeptide having at least 70%, 75%, 80%, 85%, 90%, 91%, 92%, 93%, 94%, 95%, 96%, 97%, 98%, 99%, or 100% sequence identity along the length of the amino acid sequence selected from the group consisting of SEQ ID NOS: 331, 332, 334, 336-422, 424, 426, 428, 430, 432, 434, 436, 438, 440, 442, 444, 446, 448, 450, 452, 454, 456, 458-460, 462, 464, 466, 468, 470, 472, 474, 476, 478, 480, 482, 484, 486, 488, 490, 493, and 494. The amino acid sequences of SEQ ID NOS: 331, 332, 334, 336-422, 424, 426, 428, 430, 432, 434, 436, 438, 440, 442, 444, 446, 448, 450, 452, 454, 456, 458-460, 462, 464, 466, 468, 470, 472, 474, 476, 478, 480, 482, 484, 486, 488, 490, 493, and 494 are provided in Table 1B, and can be used, for example, to generate the heterodimers of the disclosure.

In some aspects, the disclosure provides non-naturally occurring polypeptides comprising a polypeptide having at least 70%, 75%, 80%, 85%, 90%, 91%, 92%, 93%, 94%, 95%, 96%, 97%, 98%, 99%, or 100% sequence identity along the length of the amino acid sequence selected from the group consisting of SEQ ID NOS: 331, 332, 334, 336-422, 424, 426, 428, 430, 432, 434, 436, 438, 440, 442, 444, 446, 448, 450, 452, 454, 456, 458-460, 462, 464, 466, 468, 470, 472, 474, 476, 478, 480, 482, 484, 486, 488, 490, 493, and 494, wherein the SEQ ID NOs: 331, 332, 334, 336-422, 424, 426, 428, 430, 432, 434, 436, 438, 440, 442, 444, 446, 448, 450, 452, 454, 456, 458-460, 462, 464, 466, 468, 470, 472, 474, 476, 478, 480, 482, 484, 486, 488, 490, 493, and 494 is not linked to GlySer at the immediate N terminus or the polypeptide does not comprise GlySer at the N terminus.

In some aspects, the disclosure provides non-naturally occurring polypeptides comprising a polypeptide consisting of an amino acid sequence at least 70%, 75%, 80%, 85%, 90%, 91%, 92%, 93%, 94%, 95%, 96%, 97%, 98%, 99%, or 100% sequence identity along the length of the amino acid sequence selected from the group consisting of SEQ ID NOS: 331, 332, 334, 336-422, 424, 426, 428, 430, 432, 434, 436, 438, 440, 442, 444, 446, 448, 450, 452, 454, 456, 458-460, 462, 464, 466, 468, 470, 472, 474, 476, 478, 480, 482, 484, 486, 488, 490, 493, and 494. In some aspects, the disclosure provides non-naturally occurring polypeptides comprising a polypeptide consisting of the sequence of SEQ ID NOs: 331, 332, 334, 336-422, 424, 426, 428, 430, 432, 434, 436, 438, 440, 442, 444, 446, 448, 450, 452, 454, 456, 458-460, 462, 464, 466, 468, 470, 472, 474, 476, 478, 480, 482, 484, 486, 488, 490, 493, and 494.

In one embodiment, the amino acid changes from the reference amino acid sequence are conservative amino acid substitutions. In another embodiment, amino acid residues at 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% of defined interface positions are invariant compared to the reference amino acid sequence. The defined interface residues are as provided in Table 2.

In a second aspect, the disclosure provides proteins comprising 2, 3, 4, or more non-naturally occurring polypeptides having at least 70%, 75%, 80%, 85%, 90%, 91%, 92%, 93%, 94%, 95%, 96%, 97%, 98%, 99%, or 100% sequence identity along the length of the amino acid sequence selected from the group consisting of SEQ ID NOS: 1-290, wherein the 2, 3, 4, or more naturally occurring polypeptides are covalently linked. In some aspects, the disclosure provides proteins comprising 2, 3, 4, or more non-naturally occurring polypeptides having at least 70%, 75%, 80%, 85%, 90%, 91%, 92%, 93%, 94%, 95%, 96%, 97%, 98%, 99%, or 100% sequence identity along the length of the amino acid sequence selected from the group consisting of SEQ ID NOS: 1-290 and 331, 332, 334, 336-422, 424, 426, 428, 430, 432, 434, 436, 438, 440, 442, 444, 446, 448, 450, 452, 454, 456, 458-460, 462, 464, 466, 468, 470, 472, 474, 476, 478, 480, 482, 484, 486, 488, 490, 493, and 494, wherein the 2, 3, 4, or more naturally occurring polypeptides are covalently linked. In some aspects, the sequences of monomer A and monomer B listed herein can be modified (substituted) such that the resulting amino acid sequence maintains a hydrogen bond network of the original amino acid sequence as described in Tables 1A and 1B.

In this aspect, the proteins can be used to generate scaffolds that can be used for any suitable purpose including but not limited to those disclosed herein. In one embodiment, each of the 2, 3, 4, or more non-naturally occurring polypeptides are different. In another embodiment, the 2, 3, 4, or more non-naturally occurring polypeptides may include 2, 3, 4, or more identical polypeptides. In all embodiments, the 2, 3, 4, or more non-naturally occurring polypeptides may, for example, be covalently linked as part of a fusion protein. The 2, 3, 4, or more non-naturally occurring polypeptides may each be separated by an amino acid linker. Any suitable amino acid linker may be used.

In some aspects, the linker is a flexible linker. In some aspects, the linker is a GS linker. In other aspects, the GS linker comprises (GGS)n, (GSEGS)n (SEQ ID NO:423) or (GGGS)n (SEQ ID NO:425), wherein n is an integer between 1 and 100. In some aspects, the linker comprises an amino acid sequence having at least 70%, 75%, 80%, 85%, 90%, 91%, 92%, 93%, 94%, 95%, 96%, 97%, 98%, 99%, or 100% sequence identity along the length of the amino acid sequence as set forth in GSEGSGSEGSGS (SEQ ID NO:427) or GSEGSGSEGSGGS (SEQ ID NO:461). In some aspects, the linker comprises an amino acid sequence having at least 70%, 75%, 80%, 85%, 90%, 91%, 92%, 93%, 94%, 95%, 96%, 97%, 98%, 99%, or 100% sequence identity along the length of the amino acid sequence as set forth in GSEGSGSEGS (SEQ ID NO:429). In some aspects, the linker comprises an amino acid sequence having at least 70%, 75%, 80%, 85%, 90%, 91%, 92%, 93%, 94%, 95%, 96%, 97%, 98%, 99%, or 100% sequence identity along the length of the amino acid sequence as set forth in (GSEGS)n, wherein n is 1 to 10, e.g., 1, 2, 3, 4, 5, 6, 7, 8, 9, or 10 (SEQ ID NO:423).

In one embodiment, each of the 2, 3, 4, or more non-naturally occurring polypeptides have at least 70%, 75%, 80%, 85%, 90%, 91%, 92%, 93%, 94%, 95%, 96%, 97%, 98%, 99%, or 100% sequence identity along the length of the amino acid sequence of an odd-numbered SEQ ID NO: selected from the group consisting of SEQ ID NOS:1-290. In one embodiment, each of the 2, 3, 4, or more non-naturally occurring polypeptides have at least 70%, 75%, 80%, 85%, 90%, 91%, 92%, 93%, 94%, 95%, 96%, 97%, 98%, 99%, or 100% sequence identity along the length of the amino acid sequence of an odd-numbered SEQ ID NO: selected from the group consisting of SEQ ID NOS:1-290, wherein GlySer at amino acid positions 1 and 2 of SEQ ID NO: 1, 55, 81, 83, 101, 105, 115, 117, 119, 121, 123, 125, 127, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, or 193 are optional, e.g., not present. In another embodiment, each of the 2, 3, 4, or more non-naturally occurring polypeptides have at least 70%, 75%, 80%, 85%, 90%, 91%, 92%, 93%, 94%, 95%, 96%, 97%, 98%, 99%, or 100% sequence identity along the length of the amino acid sequence of an even-numbered SEQ ID NO: selected from the group consisting of SEQ ID NOS: 1-290. In another embodiment, each of the 2, 3, 4, or more non-naturally occurring polypeptides have at least 70%, 75%, 80%, 85%, 90%, 91%, 92%, 93%, 94%, 95%, 96%, 97%, 98%, 99%, or 100% sequence identity along the length of the amino acid sequence of an even-numbered SEQ ID NO: selected from the group consisting of SEQ ID NOS:1-290, wherein GlySer at amino acid positions 1 and 2 of SEQ ID NO: 6, 8, 14, 16, 26, 30, 32, 34, 36, 38, 40, 42, 46, 48, 54, 56, 58, 60, 62, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 128, 130, 132, 134, 136, 138, 140, 142, 144, 146, 148, 150, 152, 154, 156, 158, 160, 162, 164, 166, 168, 170, 172, 176, 178, 180, 182, 184, 186, 188, 190, 192, or 194 are optional, e.g., not present. In a further embodiment, the 2, 3, 4, or more non-naturally occurring polypeptides include:
(a) polypeptides having at least 70%, 75%, 80%, 85%, 90%, 91%, 92%, 93%, 94%, 95%, 96%, 97%, 98%, 99%, or 100% sequence identity along the length of the amino acid sequence of an odd-numbered SEQ ID NO: selected from the group consisting of SEQ ID NOS:1-290, or selected from the group consisting of SEQ ID NOS: 1-290, 331, 332, 334, 336-422, 424, 426, 428, 430, 432, 434, 436, 438, 440, 442, 444, 446, 448, 450, 452, 454, 456, 458-460, 462, 464, 466, 468, 470, 472, 474, 476, 478, 480, 482, 484, 486, 488, 490, 493, and 494; and
(b) polypeptides having at least 70%, 75%, 80%, 85%, 90%, 91%, 92%, 93%, 94%, 95%, 96%, 97%, 98%, 99%, or 100% sequence identity along the length of the amino acid sequence of an even-numbered SEQ ID NO: selected from the group consisting of SEQ ID NOS:1-290, or selected from the group consisting of SEQ ID NOS: 1-290, 331, 332, 334, 336-422, 424, 426, 428, 430, 432, 434, 436, 438, 440, 442, 444, 446, 448, 450, 452, 454, 456, 458-460, 462, 464, 466, 468, 470, 472, 474, 476, 478, 480, 482, 484, 486, 488, 490, 493, and 494.

In some aspects, the 2, 3, 4, or more non-naturally occurring polypeptides include:
(a) a polypeptide having at least 70%, 75%, 80%, 85%, 90%, 91%, 92%, 93%, 94%, 95%, 96%, 97%, 98%, 99%, or 100% sequence identity along the length of the amino acid sequence of chain a in Table 1A and/or 1B; and
(b) a polypeptide having at least 70%, 75%, 80%, 85%, 90%, 91%, 92%, 93%, 94%, 95%, 96%, 97%, 98%, 99%, or 100% sequence identity along the length of the amino acid sequence of chain b in Table 1A and/or 1B.

In some aspects, the protein of the present disclosure comprises a heterotrimer. In some aspects, the heterotrimer comprises a monomer having an amino acid sequence at least 70%, 75%, 80%, 85%, 90%, 91%, 92%, 93%, 94%, 95%, 96%, 97%, 98%, 99%, or 100% sequence identity along the length of the amino acid sequence described in Tables 1A and 1B, wherein the amino acid sequence forms a hydrogen bond network, e.g., hydrogen bond network formed by the interface residues according to Table 2. In some aspects, the heterotrimer of the present disclosure comprises at least two monomers, wherein each of the monomers comprises an amino acid sequence at least 70%, 75%, 80%, 85%, 90%, 91%, 92%, 93%, 94%, 95%, 96%, 97%, 98%, 99%, or 100% sequence identity along the length of the amino acid sequence described in Tables 1A and 1B, wherein the amino acid sequence forms a hydrogen bond network, e.g., hydrogen bond network formed by the interface residues according to Table 2.

In some aspects, the heterotrimer of the present disclosure comprises at least three monomers, wherein each of the monomers comprises an amino acid sequence at least 70%, 75%, 80%, 85%, 90%, 91%, 92%, 93%, 94%, 95%, 96%, 97%, 98%, 99%, or 100% sequence identity along the length of the amino acid sequence described in Tables 1A and 1B, wherein the amino acid sequence forms a hydrogen bond network, e.g., hydrogen bond network formed by the interface residues according to Table 2.

In some aspects, the heterotrimer of the present disclosure comprises at least one heterodimer, wherein the heterodimer comprises an amino acid sequence at least 70%, 75%, 80%, 85%, 90%, 91%, 92%, 93%, 94%, 95%, 96%, 97%, 98%, 99%, or 100% sequence identity along the length of the amino acid sequence described in Tables 3 and 4, wherein the amino acid sequence forms a hydrogen bond network, e.g., hydrogen bond network formed by the interface residues according to Table 2.

In some aspects, the heterotrimer of the present disclosure comprises an amino acid sequence at least 70%, 75%, 80%, 85%, 90%, 91%, 92%, 93%, 94%, 95%, 96%, 97%, 98%, 99%, or 100% sequence identity along the length of the amino acid sequence described in Tables 3 and 4, wherein the amino acid sequence forms a hydrogen bond network.

In some aspects, the protein of the present disclosure comprises a heterotetramer. In some aspects, the heterotetramer comprises a monomer having an amino acid sequence at least 70%, 75%, 80%, 85%, 90%, 91%, 92%, 93%, 94%, 95%, 96%, 97%, 98%, 99%, or 100% sequence identity along the length of the amino acid sequence described in Tables 1A and 1B, wherein the amino acid sequence forms a hydrogen bond network, e.g., hydrogen bond network formed by the interface residues according to Table 2. In some aspects, the heterotetramer of the present disclosure comprises at least two monomers, wherein each of the monomers comprises an amino acid sequence at least 70%, 75%, 80%, 85%, 90%, 91%, 92%, 93%, 94%, 95%, 96%, 97%, 98%, 99%, or 100% sequence identity along the length of the amino acid sequence described in Tables 1A and 1B, wherein the amino acid sequence forms a hydrogen bond network, e.g., hydrogen bond network formed by the interface residues according to Table 2.

In some aspects, the heterotetramer of the present disclosure comprises at least three monomers, wherein each of the monomers comprises an amino acid sequence at least 70%, 75%, 80%, 85%, 90%, 91%, 92%, 93%, 94%, 95%, 96%, 97%, 98%, 99%, or 100% sequence identity along the length of the amino acid sequence described in Tables 1A and 1B, wherein the amino acid sequence forms a hydrogen bond network, e.g., hydrogen bond network formed by the interface residues according to Table 2.

In some aspects, the heterotetramer of the present disclosure comprises at least four monomers, wherein each of the monomers comprises an amino acid sequence at least 70%, 75%, 80%, 85%, 90%, 91%, 92%, 93%, 94%, 95%, 96%, 97%, 98%, 99%, or 100% sequence identity along the length of the amino acid sequence described in Tables 1A and 1B, wherein the amino acid sequence forms a hydrogen bond network, e.g., hydrogen bond network formed by the interface residues according to Table 2.

In some aspects, the heterotetramer of the present disclosure comprises at least one heterodimer, wherein the heterodimer comprises an amino acid sequence at least 70%, 75%, 80%, 85%, 90%, 91%, 92%, 93%, 94%, 95%, 96%, 97%, 98%, 99%, or 100% sequence identity along the length of the amino acid sequence described in Tables 1A and 1B, wherein the amino acid sequence forms a hydrogen bond network, e.g., hydrogen bond network formed by the interface residues according to Table 2.

In some aspects, the heterotetramer of the present disclosure comprises at least two heterodimers, wherein each of the two heterodimers comprises an amino acid sequence at least 70%, 75%, 80%, 85%, 90%, 91%, 92%, 93%, 94%, 95%, 96%, 97%, 98%, 99%, or 100% sequence identity along the length of the amino acid sequence described in Tables 1A and 1B, wherein the amino acid sequence forms a hydrogen bond network, e.g., hydrogen bond network formed by the interface residues according to Table 2.

In some aspects, the heterotetramer of the present disclosure comprises at least one heterotrimer, wherein the heterotrimer comprises an amino acid sequence at least 70%, 75%, 80%, 85%, 90%, 91%, 92%, 93%, 94%, 95%, 96%, 97%, 98%, 99%, or 1000 sequence identity along the length of the amino acid sequence described in Tables 3 and 4, wherein the amino acid sequence forms a hydrogen bond network.

In another embodiment, the protein comprises the amino acid sequence having at least 70%, 75%, 80%, 85%, 90%, 91%, 92%, 93%, 94%, 95%, 96%, 97%, 98%, 99%, or 100% sequence identity along the length of the amino acid sequence selected from the group consisting of SEQ ID NOS:291, 294, 296, 299, and 302-305. The amino acid sequence of SEQ ID NOS:291, 294, 296, 299, and 302-305 is provided in Table 3. These are merely exemplary such proteins of this aspect of the disclosure, and those of skill in the art will understand that any suitable combination of the monomers of the disclosure can be used in generating the proteins of this aspect.

TABLE 3

| Design | Oligomerization | | |
|---|---|---|---|
| name | State | Chain | Design Sequence |
| DHDSC_9- | Heterotetramer | 9a-13a- | PKEEARELIRKQKELIKEQKKLIKEAKQKSDSRDAERIWKRSREINRESKK |
| 13-37 | (linker | 37a | INKRIKELIKSGSEGSGSEGSGSTKEDILERQRKIIERAQEIHRRQQEILE |
| | italicized and | | ELERIIRKPGSSEEAMKRMLKLLEESLRLLKELLELSEESAQLLYEQRGSE |
| | underlined) | | GSGSEGSGSDSDEHLKKLKTFLENLRRHLDRLDKHIKQLRDILSENPEDER |
| | | | VKDVIDLSERSVRIVKTVIKIFEDSVRKKE SEQ ID NO: 291 |
| DHD9-13 | Heterotrimer | 9a-13a | PKEEARELIRKQKELIKEQKKLIKEAKQKSDSRDAERIWKRSREINRESKK |
| | | | INKRIKELIKSGSEGSGSEGSGSTKEDILERQRKIIERAQEIHRRQQEILE |
| | | | ELERIIRKPGSSEEAMKRMLKLLEESLRLLKELLELSEESAQLLYEQR |
| | | | SEQ ID NO: 294 |
| DHD15- | Heterotrimer | 15b-37a | TERKLLERSRRLQEESKRLLDEMAEIMRRIKKLLDDPDSEDIAREIKELLR |
| 37 | | | RLKEIIERNQRIAKEHEYIARERSGPGSGSEGSDSDEHLKKLKTFLENLRR |
| | | | HLDRLDKHIKQLRDILSENPEDERVKDVIDLSERSVRIVKTVIKIFEDSVR |
| | | | KKE SEQ ID NO: 296 |
| DHD13- | Heterotrimer | 13b-37b | TEKRLLEEAERAHREQKEIIKKAQELHRRLEEIVRQSGSSEEAKKEAKKIL |
| 37 | | | EEIRELSKRSLELLREILYLSQEQKGSEGSGSEGSGSDDKELDKLLDTLEK |
| | | | ILQTATKIIDDANKLLEKLRRSERKDPKVVETYVELLKRHEKAVKELLEIA |
| | | | KTHAKKVE SEQ ID NO: 299 |
| OPHD_15- | Heterotrimer | 15b-9a | TERKLLERSRRLQEESKRLLDEMAEIMRRIKKLLDDPDSEDIAREIKELLR |
| 9 | | | RLKEIIERNQRIAKEHEYIARERSGSEGSGSEGSGSPKEEARELIRKQKEL |
| | | | IKEQKKLIKEAKQKSDSRDAERIWKRSREINRESKKINKRIKELIKS SEQ |
| | | | ID NO: 302 |
| OPHD_37- | Heterotrimer | 37a-9a | PKKEAEELAEESEELHDRSEKLHERAEQSSNSEEARKILEDIERISERIEE |
| 9 | | | ISDRIERLLRSGSEGSGSEGSGSDDKELDKLLDTLEKILQTATKIIDDANK |
| | | | LLEKLRRSERKDPKVVETYVELLKRHEKAVKELLEIAKTHAKKVE SEQ |
| | | | ID NO: 303 |
| OPHD_13- | Heterotrimer | 13a-9a | TKEDILERQRKIIERAQEIHRRQQEILEELERIIRKPGSSEEAMKRMLKLL |
| 9 | | | EESLRLLKELLELSEESAQLLYEQRGSEGSGSEGSGSPKEEARELIRKQKE |
| | | | LIKEQKKLIKEAKQKSDSRDAERIWKRSREINRESKKINKRIKELIKS |
| | | | SEQ ID NO: 304 |
| OPHD_9- | Heterotrimer | 9b-37a | PKKEAEELAEESEELHDRSEKLHERAEQSSNSEEARKILEDIERISERIEE |
| 37 | | | ISDRIERLLRSGSEGSGSEGSDSDEHLKKLKTFLENLRRHLDRLDKHIKQL |
| | | | RDILSENPEDERVKDVIDLSERSVRIVKTVIKIFEDSVRKKE SEQ ID |
| | | | NO: 305 |

In a third aspect, the disclosure provides protein scaffolds, comprising
a) a first designed component comprised of any number of monomer A polypeptides and/or monomer B polypeptides, each from different heterodimers, connected into a single component by amino acid linkers.
b) a second designed component, comprising corresponding monomers for each monomer A and/or monomer B in the first designed component one;
wherein the first and second designed components interact to form the protein scaffold, and wherein each monomer A only interacts in the scaffold with its monomer B binding partner. In one embodiment, the first designed component may comprise the protein of any embodiment or combination of embodiments disclosed herein, and/or the second designed component may comprise a plurality of individual polypeptides of embodiment or combination of embodiments disclosed herein. In non-limiting embodiments, the first designed component and the second designed component may comprise a set of three (Heterotrimer) or four (Heterotetramer) binding partners as shown in Table 4. As will be understood by those of skill in the art based on the teachings herein, heterotrimers of the disclosure (including but not limited to the exemplary heterotrimers shown in Table 4) include a first component fusion protein of two polypeptides of the disclosure, and the second component comprises two separate polypeptides that are binding partners of the two polypeptides in the fusion protein. For example, the DHD9-13 scaffold comprises a first designed component comprising the DHD9 A monomer covalently linked to the DHD13 A monomer, and the second designed component comprises individual DHD9 B and DHD13 B monomers. Different scaffolds are separated in the Table by a blank row. As will be understood by those of skill I the art, these are merely exemplary; the monomers in the first designed component may be linked in any order, and any monomers may be included in the designed components. As will be further understood by those of skill based on the teachings herein, heterotetramers (including but not limited to the exemplary heterotetramer shown in Table 4) include a first component fusion protein of three polypeptides of the disclosure, and the second component comprises three separate polypeptides that are binding partners of the three polypeptides in the fusion protein.

TABLE 4

| | | | |
|---|---|---|---|
| DHD9-13 | Heterotrimer | 9a-13a | PKEEARELIRKQKELIKEQKKLIKEAKQKSDSRDAERIWKRSREINRESKK |
| | | | INKRIKELIKSGSEGSGSEGSGSTKEDILERQRKIIERAQEIHRRQQEILE |
| | | | ELERIIRKPGSSEEAMKRMLKLLEESLRLLKELLELSEESAQLLYEQR |
| | | | SEQ ID NO: 294 |
| DHD9-13 | Heterotrimer | 9b | PKKEAEELAEESEELHDRSEKLHERAEQSSNSEEARKILEDIERISERIEE |
| | | | ISDRIERLLRS SEQ ID NO: 2 |
| DHD9-13 | Heterotrimer | 13b | GSHHHHHHGSGSENLYFQGSTEKRLLEEAERAHREQKEIIKKAQELHRRLE |
| | | | EIVRQSGSSEEAKKEAKKILEEIRELSKRSLELLREILYLSQEQK SEQ |
| | | | ID NO: 295 |
| DHD9-13 | Heterotrimer | 13b | HHHHHHGSGSENLYFQGSTEKRLLEEAERAHREQKEIIKKAQELHRRLEEI |
| | | | VRQSGSSEEAKKEAKKILEEIRELSKRSLELLREILYLSQEQK SEQ ID |
| | | | NO: 431 |
| DHD15- | Heterotrimer | 15b-37a | TERKLLERSRRLQEESKRLLDEMAEIMRRIKKLLDDPDSEDIAREIKELLR |
| 37 | | | RLKEIIERNQRIAKEHEYIARERSGPGSGSEGSDSDEHLKKLKTFLENLRR |
| | | | HLDRLDKHIKQLRDILSENPEDERVKDVIDLSERSVRIVKTVIKIFEDSVR |
| | | | KKE SEQ ID NO: 296 |
| DHD15- | Heterotrimer | 15a | TREELLRENIELAKEHIEIMREILELLQKMEELLERQSSEDILEELRKIIE |
| 37 | | | RIRELLDRSRKIHERSEEIAYKEE SEQ ID NO: 297 |
| DHD15- | Heterotrimer | 37b | GSHHHHHHGSGSENLYFQGSDDKELDKLLDTLEKILQTATKIIDDANKLLE |
| 37 | | | KLRRSERKDPKVVETYVELLKRHEKAVKELLEIAKTHAKKVE SEQ ID |
| | | | NO: 298 |
| DHD15- | Heterotrimer | 37b | HHHHHHGSGSENLYFQGSDDKELDKLLDTLEKILQTATKIIDDANKLLEKI |
| 37 | | | RRSERKDPKVVETYVELLKRHEKAVKELLEIAKTHAKKVE SEQ ID |
| | | | NO: 433 |
| DHD13- | Heterotrimer | 13b-37b | TEKRLLEEAERAHREQKEIIKKAQELHRRLEEIVRQSGSEEAKKEAKKIL |
| 37 | | | EEIRELSKRSLELLREILYLSQEQKGSEGSGSEGSGSDDKELDKLLDTLEK |
| | | | ILQTATKIIDDANKLLEKLRRSERKDPKVVETYVELLKRHEKAVKELLEIA |
| | | | KTHAKKVE SEQ ID NO: 299 |
| DHD13- | Heterotrimer | 13a | TKEDILERQRKIIERAQEIHRRQQEILEELERIIRKPGSSEEAMKRMLKLL |
| 37 | | | EESLRLLKELLELSEESAQLLYEQR SEQ ID NO: 300 |
| DHD13- | Heterotrimer | 37a | GSSHHHHHHSSGENLYFQGSDSDEHLKKLKTFLENLRRHLDRLDKHIKQLR |
| 37 | | | DILSENPEDERVKDVIDLSERSVRIVKTVIKIFEDSVRKKE SEQ ID |
| | | | NO: 301 |
| DHD13- | Heterotrimer | 37a | SHHHHHHSSGENLYFQGSDSDEHLKKLKTFLENLRRHLDRLDKHIKQLRDI |
| 37 | | | LSENPEDERVKDVIDLSERSVRIVKTVIKIFEDSVRKKE SEQ ID |
| | | | NO: 435 |
| OPHD_15- | Heterotrimer | 15b-9a | TERKLLERSRRLQEESKRLLDEMAEIMRRIKKLLDDPDSEDIAREIKELLR |
| 9 | | | RLKEIIERNQRIAKEHEYIARERSGSEGSGSEGSGSPKEEARELIRKQKEL |
| | | | IKEQKKLIKEAKQKSDSRDAERIWKRSREINRESKKINKRIKELIKS SEQ |
| | | | ID NO: 302 |
| OPHD_15- | Heterotrimer | 15a | TREELLRENIELAKEHIEIMREILELLQKMEELLEKARGADEDVAKTIKEL |
| 9 | | | LRRLKEIIERNQRIAKEHEYIARERS SEQ ID NO: 19 |
| OPHD_15- | Heterotrimer | 9b | PKKEAEELAEESEELHDRSEKLHERAEQSSNSEEARKILEDIERISERIEE |
| 9 | | | ISDRIERLLRS SEQ ID NO: 2 |
| OPHD_37 | Heterotrimer | 37a-9a | PKKEAEELAEESEELHDRSEKLHERAEQSSNSEEARKILEDIERISERIEE |
| 9- | | | ISDRIERLLRSGSEGSGSEGSGSDDKELDKLLDTLEKILQTATKIIDDANK |
| | | | LLEKLRRSERKDPKVVETYVELLKRHEKAVKELLEIAKTHAKKVE SEQ |
| | | | ID NO: 303 |
| OPHD_37- | Heterotrimer | 37b | GSDDKELDKLLDTLEKILQTATKIIDDANKLLEKLRRSERKDPKVVETYVE |
| 9 | | | LLKRHEKAVKELLEIAKTHAKKVE SEQ ID NO: 42 |
| OPHD_37- | Heterotrimer | 37b | DDKELDKLLDTLEKILQTATKIIDDANKLLEKLRRSERKDPKVVETYVELL |
| 9 | | | KRHEKAVKELLEIAKTHAKKVE SEQ ID NO: 352 |
| OPHD_37- | Heterotrimer | 9b | PKKEAEELAEESEELHDRSEKLHERAEQSSNSEEARKILEDIERISERIEE |
| 9 | | | ISDRIERLLRS SEQ ID NO: 2 |
| OPHD_13- | Heterotrimer | 13a-9a | TKEDILERQRKIIERAQEIHRRQQEILEELERIIRKPGSSEEAMKRMLKLL |
| 9 | | | EESLRLLKELLELSEESAQLLYEQRGSEGSGSEGSGSPKEEARELIRKQKE |
| | | | LIKEQKKLIKEAKQKSDSRDAERIWKRSREINRESKKINKRIKELIKS |
| | | | SEQ ID NO: 304 |
| OPHD_13- | Heterotrimer | 13b | GTEKRLLEEAERAHREQKEIIKKAQELHRRLEEIVRQSGSSEEAKKEAKKI |
| 9 | | | LEEIRELSKRSLELLREILYLSQEQKGSLVPR SEQ ID NO: 4 |
| OPHD_13- | Heterotrimer | 9b | PKKEAEELAEESEELHDRSEKLHERAEQSSNSEEARKILEDIERISERIEE |
| 9 | | | ISDRIERLLRS SEQ ID NO: 2 |
| OPHD_9- | Heterotrimer | 9b-37a | PKKEAEELAEESEELHDRSEKLHERAEQSSNSEEARKILEDIERISERIEE |
| 37 | | | ISDRIERLLRSGSEGSGSEGSDSDEHLKKLKTFLENLRRHLDRLDKHIKQL |
| | | | RDILSENPEDERVKDVIDLSERSVRIVKTVIKIFEDSVRKKE SEQ ID |
| | | | NO: 305 |
| OPHD_9- | Heterotrimer | 9a | GSPKEEARELIRKQKELIKEQKKLIKEAKQKSDSRDAERIWKRSREINRES |
| 37 | | | KKINKRIKELIKS SEQ ID NO: 1 |
| OPHD_9- | Heterotrimer | 9a | PKEEARELIRKQKELIKEQKKLIKEAKQKSDSRDAERIWKRSREINRESKK |
| 37 | | | INKRIKELIKS SEQ ID NO: 331 |
| OPHD_9- | Heterotrimer | 37b | GSDDKELDKLLDTLEKILQTATKIIDDANKLLEKLRRSERKDPKVVETYVE |
| 37 | | | LLKRHEKAVKELLEIAKTHAKKVE SEQ ID NO: 42 |
| OPHD_9- | Heterotrimer | 37b | DDKELDKLLDTLEKILQTATKIIDDANKLLEKLRRSERKDPKVVETYVELL |
| 37 | | | KRHEKAVKELLEIAKTHAKKVE SEQ ID NO: 352 |
| DHDSC_9- | Heterotetramer | 9a-13a- | PKEEARELIRKQKELIKEQKKLIKEAKQKSDSRDAERIWKRSREINRESKK |
| 13-37 | | 37a | INKRIKELIKSGSEGSGSEGSGSTKEDILERQRKIIERAQEIHRRQQEILE |
| | | | ELERIIRKPGSSEEAMKRMLKLLEESLRLLKELLELSEESAQLLYEQRGSE |
| | | | GSGSEGSGSDSDEHLKKLKTFLENLRRHLDRLDKHIKQLRDILSENPEDER |
| | | | VKDVIDLSERSVRIVKTVIKIFEDSVRKKE SEQ ID NO: 291 |
| DHDSC_9- | Heterotetramer | 9b | PKKEAEELAEESEELHDRSEKLHERAEQSSNSEEARKILEDIERISERIEE |
| 13-37 | | | ISDRIERLLRS SEQ ID NO: 2 |
| DHDSC_9- | Heterotetramer | 13b | TEKRLLEEAERAHREQKEIIKKAQELHRRLEEIVRQSGSSEEAKKEAKKIL |
| 13-37 | | | EEIRELSKRSLELLREILYLSQEQK SEQ ID NO: 292 |
| DHDSC_9- | Heterotetramer | 37b | GSSHHHHHHSSGENLYFQGSDDKELDKLLDTLEKILQTATKIIDDANKLLE |
| 13-37 | | | KLRRSERKDPKVVETYVELLKRHEKAVKELLEIAKTHAKKVE SEQ ID |
| | | | NO: 293 |
| DHDSC_9- | Heterotetramer | 37b | SHHHHHHSSGENLYFQGSDDKELDKLLDTLEKILQTATKIIDDANKLLEKL |
| 13-37 | | | RRSERKDPKVVETYVELLKRHEKAVKELLEIAKTHAKKVE SEQ ID |
| | | | NO: 437 |

In these embodiments, the scaffold may be stable up to 95° C. and has a guanidine denaturation midpoint of 4 M, as described in the examples that follow.

In some aspects, the heterotrimer or heterotetramer of the present disclosure does not comprise a His tag.

In another aspect, the disclosure provides protein scaffolds, comprising
(a) a fusion protein comprising of 2, 3, 4, or more polypeptides, wherein each polypeptide present in the fusion protein is a non-naturally occurring polypeptide comprises 1-5 alpha helices, wherein adjacent alpha helices may optionally be connected by an amino acid linker;
wherein each polypeptide in the fusion protein is capable of non-covalently interacting with a binding partner, and wherein the fusion protein does not comprise a binding partner for any polypeptide present in the fusion protein; and
(b) a binding partner for at least one of the polypeptides present in the fusion protein;
wherein the fusion protein and the binding partner non-covalently interact to form the protein scaffold, wherein an interaction specificity between the binding partner and the at least polypeptide in the fusion protein are determined by at least one hydrogen bond network at the interface between the binding partner and the at least one polypeptide.

Binding partners are polypeptides capable of forming heterodimers with a polypeptide present in the fusion protein, and are exemplified above with respect to SEQ ID NO: 1-290. The binding partner for at least one polypeptide in the fusion protein may comprise a binding partner for 2, 3, 4, or all polypeptides in the fusion protein. As will be understood, when more than one binding partner is present, they are present as individual binding partner polypeptides, and not linked together.

The fusion protein may comprise 2, 3, 4, or more polypeptides. In certain embodiments, the fusion protein comprises at least 3 or 4 polypeptides in total. Exemplary embodiments of such fusion proteins are provided herein, for example in describing heterotrimer and heterotetramer embodiments in Table 4. The polypeptides in the fusion protein may all be the same, may all be different, or may include both identical and distinct polypeptides. In one specific embodiment, each polypeptide in the fusion protein is a different polypeptide.

In one embodiment,
(i) the fusion protein comprises 2, 3, 4, or more polypeptide having at least 70%, 75%, 80%, 85%, 90%, 91%, 92%, 93%, 94%, 95%, 96%, 97%, 98%, 99%, or 100% sequence identity along the length of the amino acid sequence of SEQ ID NOS: 1-290, or SEQ ID NOS: 1-290, 331, 332, 334, 336-422, 424, 426, 428, 430, 432, 434, 436, 438, 440, 442, 444, 446, 448, 450, 452, 454, 456, 458-460, 462, 464, 466, 468, 470, 472, 474, 476, 478, 480, 482, 484, 486, 488, 490, 493, and 494; and
(ii) the binding partner comprises a binding partner as defined herein for each polypeptide in (i), wherein each binding partner has at least 70%, 75%, 80%, 85%, 90%, 91%, 92%, 93%, 94%, 95%, 96%, 97%, 98%, 99%, or 100% sequence identity along the length of the amino acid sequence selected from the group SEQ ID NOS: 1-290, or selected from the group consisting of SEQ ID NOS: 1-290, 331, 332, 334, 336-422, 424, 426, 428, 430, 432, 434, 436, 438, 440, 442, 444, 446, 448, 450, 452, 454, 456, 458-460, 462, 464, 466, 468, 470, 472, 474, 476, 478, 480, 482, 484, 486, 488, 490, 493, and 494. As described herein, the odd-numbered SEQ ID NOS: between SEQ ID NO:1-290, 331, 332, 334, 336-422, 424, 426, 428, 430, 432, 434, 436, 438, 440, 442, 444, 446, 448, 450, 452, 454, 456, 458-460, 462, 464, 466, 468, 470, 472, 474, 476, 478, 480, 482, 484, 486, 488, 490, 493, and 494 are noted as “A” monomers and the even-numbered SEQ ID NOS between SEQ ID NO:1-290, 331, 332, 334, 336-422, 424, 426, 428, 430, 432, 434, 436, 438, 440, 442, 444, 446, 448, 450, 452, 454, 456, 458-460, 462, 464, 466, 468, 470, 472, 474, 476, 478, 480, 482, 484, 486, 488, 490, 493, and 494 are the “B” monomers, with adjacent A and B monomers in Tables 1A and 1B capable of forming heterodimers as described in detail herein. Thus, for example, if the fusion protein included the polypeptide of SEQ ID NO:1, then binding partner may include SEQ ID NO:2, while if the fusion protein included the polypeptide of SEQ ID NO:2, then binding partner may include SEQ ID NO:1. The numerous combinations of fusion protein polypeptides and binding partners will be clear to those of skill in the art based on the teachings herein.

In one embodiment, amino acid changes in the fusion protein and the binding partner from the reference amino acid sequence are conservative amino acid substitutions. In another embodiment amino acid residues at 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% of defined interface positions in the polypeptides in the fusion protein and the binding partner are invariant compared to the reference amino acid sequence. In a further embodiment, the at least one hydrogen bond network is asymmetric. In a further embodiment, the binding interface comprises at least 25% hydrophobic residues.. In another embodiment, the scaffold is stable up to 95° C. and has a guanidine denaturation midpoint of 4 M.

In another embodiment, the disclosure provides methods of forming the designed heterodimer disclosed herein, comprising:
a) providing two of the monomers as unlinked monomers;
b) providing the other two monomers as linked monomers
whereby the unlinked monomers associate with their respective monomer of the same heterodimer, and not with any of the other monomers. Further details of this aspect are provided in the examples that follow.

In another embodiment, the disclosure provides a designed heterodimer protein comprising:
a) asymmetric buried hydrogen bond networks incorporated into regularly repeating backbone structures; and
b) helix hairpin helix monomers wherein the supercoil phases of the helices are fixed at 0, 90, 180, or 270 degrees and the supercoil twist (ω0) and helical twist (ω1) are held constant for either a two layer left handed super coil (ω0=−2.85 and ω1=102.85), or a 5 layer untwisted bundle (ω0=0 and ω1=100) 27. Further details of this aspect are provided in the examples that follow.

In another embodiment, the disclosure provides uses of the polypeptide, protein, heterodimer protein, protein scaffold, nucleic acid, expression vector, and/or cell of any embodiment or combination of embodiments for any suitable purposed, including but not limited to those disclosed herein such as designing protein logic gates

In a fourth aspect, the disclosure provides fusion proteins comprising a polypeptide of the formula X—B—Z, wherein:
(a) the X domain is a non-naturally occurring polypeptide comprising 1, 2, or 3 alpha helices, wherein the X domain is capable of non-covalently binding to a first target;
(b) the Z domain is a non-naturally occurring polypeptide comprising 1, 2, or 3 alpha helices, wherein the Z domain is capable of non-covalently binding to either (i) a second target that differs from the first target, or (ii) a different non-naturally occurring polypeptide comprising 1, 2, or 3 alpha helices; and
(c) the B domain is an amino acid linker;
wherein a combined number of alpha helices from the X domain and the Z domain is 4, 5, or 6; and
wherein the X domain and the Z domain interact at a binding interface, wherein the binding interface comprises a hydrogen bond network in which at least one side chain in each alpha helix hydrogen of the X domain bonds with a side chain in an alpha helix in the Z domain, and wherein the binding interface comprises a plurality of hydrophobic residues. Each helix in the X domain H-bonds with at least one helix in the Z domain and each helix in the Z domain H-bonds with at least one helix in the X domain.

In a fifth aspect, the disclosure provides kits or compositions, comprising at least two fusion proteins comprising the formula X—B—Z, wherein
the B domain in each fusion protein is independently a polypeptide linker;
the X domain in each fusion protein comprises a first non-naturally occurring polypeptide comprising 1, 2, or 3 alpha helices;
the Z domain in each fusion protein comprises a second non-naturally occurring polypeptide comprising 1, 2, or 3 alpha helices, wherein a combined number of alpha helices from the X domain and the Z domain in each individual fusion protein is 4, 5, or 6; wherein the X domain and the Z domain interact at a binding interface, wherein the binding interface comprises a hydrogen bond network in which at least one side chain in each X domain alpha helix bonds with a side chain in an alpha helix in the Z domain; wherein
the X domain in a first fusion protein is capable of non-covalently binding to a first target;
the Z domain in a second fusion protein is capable of non-covalently binding to a second target; and
the X domains and Z domains in each individual fusion protein that are not capable of non-covalently binding to the first target or the second target are capable of non-covalently binding to an X or a Z domain of a different fusion protein in the plurality of fusion proteins.

The fusion proteins and kits can be used, for example, in the methods disclosed herein such as for logic gate construction, and for any other suitable use as will be appreciated by those of skill in the art based on the teachings herein. Specifically, fusion proteins can be used for designing 2-input AND and OR logic gates built from de novo designed proteins that regulate the association of arbitrary protein units ranging from split enzymes to transcriptional machinery in vitro, and in living cells. Binding interaction cooperativity makes the gates largely insensitive to stoichiometric imbalances in the inputs, and the modularity of the approach enables ready extension to 3-input OR, AND, and disjunctive normal form gates. The modularity and cooperativity of the control elements, coupled with the ability to de novo design an essentially unlimited number of protein components, enables design of sophisticated post-translational control logic over a wide range of biological functions.

In one embodiment, the Z domain is a non-naturally occurring polypeptide comprising 1, 2, or 3 alpha helices, wherein the Z domain is capable of non-covalently binding to a second target that differs from the first target, This embodiment is useful, for example, for generating single component dimerizers for use in AND/NOR gates. In another embodiment, the Z domain is a non-naturally occurring polypeptide comprising 1, 2, or 3 alpha helices, wherein the Z domain is capable of non-covalently binding to a different non-naturally occurring polypeptide comprising 1, 2, or 3 alpha helices. This embodiment is useful, for example, for generating 2 or 3-component dimerizers for use in AND/NOR gates.

The first targets and second targets may be any target suitable for an intended use. In non-limiting embodiments, the first target and/or the second target may comprise polypeptides or nucleic acids.

In one embodiment of the kit or composition,
(i) the first fusion protein has the formula X1-B1-Z1, wherein the X1 domain is capable of non-covalently binding to the first target; and
(ii) the second fusion protein has the formula X2-B2-Z2, wherein the Z2 domain is capable of non-covalently binding to the second target; and wherein the Z1 and X2 domains are capable of non-covalently binding to each other.

In another embodiment of the kit or composition,
(i) the first fusion protein has the formula X1-B1-Z1, wherein the X1 domain is capable of non-covalently binding to the first target; and
(ii) the second fusion protein has the formula X2-B2-Z2,
(iii) the at least two fusion proteins comprise a third fusion protein of formula X3-B3-Z3, wherein the Z3 domain is capable of non-covalently binding to the second target; wherein
   (A) the Z1 and X2 domains are capable of non-covalently binding to each other; and
   (B) the Z2 and X3 domains are capable of non-covalently binding to each other.

In one embodiment of the fusion protein or the kits or compositions, the binding interface comprises at least 25%, 30%, 35%, 40%, 45%, 50%, 55%, 60% or greater hydrophobic residues. The B domain linker may be any suitable amino acid sequence, including but not limited to those described herein. In one embodiment, the B domain for each fusion protein is independently between 6-12, 6-11, 6-10, 7-12, 7-11, 7-10, 8-12, 8-11, 8-10, 9-12, 9-11, 9-10, 10-12, 10-11, 11-12, 6, 7, 8, 9, 10, 11, or 12 amino acids in length.

In another embodiment, the combined number of alpha helices from the X and Z domains in an individual fusion protein is 4. In a further embodiment, the X domain of each fusion protein has 2 alpha helices and the Z domain of each fusion protein has 2 alpha helices. In one embodiment, either the X domain or the Z domain of each fusion protein has 1 alpha helix and the other has 3 alpha helices.

In one embodiment, each X domain and each Z domain comprise a polypeptide that is 50%, 55%, 60%, 65%, 70%, 75%, 80%, 85%, 90%, 91%, 92%, 93%, 94%, 95%, 96%, 97%, 98%, 99%, or 100% identical to the full length of a polypeptide selected from the group including, but not limited to SEQ ID NOS:1-290, or selected from the group consisting of SEQ ID NOS: 1-290, 331, 332, 334, 336-422, 424, 426, 428, 430, 432, 434, 436, 438, 440, 442, 444, 446, 448, 450, 452, 454, 456, 458-460, 462, 464, 466, 468, 470, 472, 474, 476, 478, 480, 482, 484, 486, 488, 490, 493, and 494, with the proviso that the X domain and the Z domain do not form a heterodimer (a-b) pair. In one embodiment, each X domain and each Z domain comprise a polypeptide that is 50%, 55%, 60%, 65%, 70%, 75%, 80%, 85%, 90%, 91%, 92%, 93%, 94%, 95%, 96%, 97%, 98%, 99%, or 100% identical to the full length of a polypeptide selected from the group including, but not limited to SEQ ID NOS:1-290 and 331, 332, 334, 336-422, 424, 426, 428, 430, 432, 434, 436, 438, 440, 442, 444, 446, 448, 450, 452, 454, 456, 458-460, 462, 464, 466, 468, 470, 472, 474, 476, 478, 480, 482, 484, 486, 488, 490, 493, and 494, with the proviso that the X domain and the Z domain do not form a heterodimer (a-b) pair. In one non-limiting embodiment, at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% of amino acid residues at defined interface positions as defined in Table 2 are invariant in the polypeptides relative to the reference polypeptide.

A different nomenclature is used in the examples that follow. Table 5 provides correspondence between the names used in the examples and in Tables TA and 1B. The first column is the numbering used in the examples, while the second column lists the corresponding name in Tables TA and 1B. For example, polypeptide 1 in the examples is DHD37_ABXB (a), 1′ is DHD37 ABXB (b). Polypeptide 2 is DHD15 (a), 2′ is DHD15 (b), and so on.

TABLE 5

| | |
|---|---|
| 1: | DHD 37_ABXB |
| 2: | DHD 15 |
| 3: | DHD 131 |
| 4: | DHD 101 |
| 5: | DHD 9 |
| 6: | DHD 150 |
| 7: | DHD 154 |
| 8: | DHD 17 |
| 9: | DHD 13_XAAA |
| 10: | DHD 39 |
| 11: | DHD 155 |

In one embodiment, each fusion protein independently comprises a polypeptide that is 50%, 55%, 60%, 65%, 70%, 75%, 80%, 85%, 90%, 91%, 92%, 93%, 94%, 95%, 96%, 97%, 98%, 99%, or 100% identical to the full length of a polypeptide selected from the group including, but not limited to a polypeptide having the amino acid sequence of a sequence selected from the group consisting of SEQ ID NO: 302, 303, 306-326, 439, 441, 443, 445, 447, 449, 451, 453, 455, and 457:

2′-1′_2-residue linker
(SEQ ID NO: 306)
GSTERKLLERSRRLQEESKRLLDEMAEIMRRIKKLLDDPDSEDIAREIKELLRRLKEIIERNQRIAKEHEYIARE
RSAADDKELDKLLDTLEKILQTATKIIDDANKLLEKLRRSERKDPKVVETYVELLKRHEKAVKELLEIAKTHAKK
VE
2′-1′_2-residue linker
(SEQ ID NO: 439)
TERKLLERSRRLQEESKRLLDEMAEIMRRIKKLLDDPDSEDIAREIKELLRRLKEIIERNQRIAKEHEYIARERS
AADDKELDKLLDTLEKILQTATKIIDDANKLLEKLRRSERKDPKVVETYVELLKRHEKAVKELLEIAKTHAKKVE
2′-1′_6-residue linker
(SEQ ID NO: 307)
GSTERKLLERSRRLQEESKRLLDEMAEIMRRIKKLLDDPDSEDIAREIKELLRRLKEIIERNQRIAKEHEYIARE
RSGGSGSPDDKELDKLLDTLEKILQTATKIIDDANKLLEKLRRSERKDPKVVETYVELLKRHEKAVKELLEIAKT
HAKKVE
2′-1′_6-residue linker
(SEQ ID NO: 441)
TERKLLERSRRLQEESKRLLDEMAEIMRRIKKLLDDPDSEDIAREIKELLRRLKEIIERNQRIAKEHEYIARERS
GGSGSPDDKELDKLLDTLEKILQTATKIIDDANKLLEKLRRSERKDPKVVETYVELLKRHEKAVKELLEIAKTHA
KKVE
2′-1′_12-residue linker
(SEQ ID NO: 308)
GSTERKLLERSRRLQEESKRLLDEMAEIMRRIKKLLDDPDSEDIAREIKELLRRLKEIIERNQRIAKEHEYIARE
RSGGSGSPGGSGSPDDKELDKLLDTLEKILQTATKIIDDANKLLEKLRRSERKDPKVVETYVELLKRHEKAVKEL
LEIAKTHAKKVE
2′-1′_12-residue linker
(SEQ ID NO: 443)
TERKLLERSRRLQEESKRLLDEMAEIMRRIKKLLDDPDSEDIAREIKELLRRLKEIIERNQRIAKEHEYIARERS
GGSGSPGGSGSPDDKELDKLLDTLEKILQTATKIIDDANKLLEKLRRSERKDPKVVETYVELLKRHEKAVKELLE
IAKTHAKKVE
2′-1′_24-residue linker
(SEQ ID NO: 309)
GSTERKLLERSRRLQEESKRLLDEMAEIMRRIKKLLDDPDSEDIAREIKELLRRLKEIIERNQRIAKEHEYIARE
RSGGSGSPGGSGSPGGSGSPGGSGSPDDKELDKLLDTLEKILQTATKIIDDANKLLEKLRRSERKDPKVVETYVE
LLKRHEKAVKELLEIAKTHAKKVE
2′-1′_24-residue linker
(SEQ ID NO: 445)
TERKLLERSRRLQEESKRLLDEMAEIMRRIKKLLDDPDSEDIAREIKELLRRLKEIIERNQRIAKEHEYIARERS
GGSGSPGGSGSPGGSGSPGGSGSPDDKELDKLLDTLEKILQTATKIIDDANKLLEKLRRSERKDPKVVETYVELL
KRHEKAVKELLEIAKTHAKKVE
11-7′
(SEQ ID NO: 310)
PEDDVVRIIKEDLESNREVLREQKEIHRILELVTRGEVSEEAIDRVLKRQEDLLKKQKESTDKARKVVEERRGSE
GSGSEGSDLEDLLRRLRRLVDEQRRLVEELERVSRRLEKAVRDNEDERELARLSREHSDIQDKHDKLAREILEVL
KRLLERTE
1′-4′
(SEQ ID NO: 311)
GSDAYDLDRIVKEHRRLVEEQRELVEELEKLVRRQEDHRVDKKESHEILERLERIIRRSTRILTELEKLTDEFER
RTRGSEGSGSEGSGSDDKELDKLLDTLEKILQTATKIIDDANKLLEKLRRSERKDPKVVETYVELLKRHEKAVKE
LLEIAKTHAKKVE
1′-4′
(SEQ ID NO: 447)
DAYDLDRIVKEHRRLVEEQRELVEELEKLVRRQEDHRVDKKESHEILERLERIIRRSTRILTELEKLTDEFERRT
RGSEGSGSEGSGSDDKELDKLLDTLEKILQTATKIIDDANKLLEKLRRSERKDPKVVETYVELLKRHEKAVKELL
EIAKTHAKKVE
4-3′
(SEQ ID NO: 312)
GSDEDDELERLLREYHRVLREYEKLLEELRRLYEEYKRGEVSEEESDRILREIKEILDKSERLWDLSEEVWRTLL
YQAEGSEGSGSEGSDEKDYHRRLIEHLEDLVRRHEELIKRQKKVVEELERRGLDERLRRVVDRERRSSERWEEVI
ERFRQVVDKLRKSVE
4-3′
(SEQ ID NO: 449)
DEDDELERLLREYHRVLREYEKLLEELRRLYEEYKRGEVSEEESDRILREIKEILDKSERLWDLSEEVWRTLLYQ
AEGSEGSGSEGSDEKDYHRRLIEHLEDLVRRHEELIKRQKKVVEELERRGLDERLRRVVDRERRSSERWEEVIER
FRQVVDKLRKSVE
3-2′*
(SEQ ID NO: 313)
GSTERKLLERSRRLQEESKRLLDEMAEIMRRIKKLLKKARGADEKVLDELRKIIERIRELLDRSRKIHERSEEIA
YKEEGSEGSGSEGSGSDESDRIRKIVEESDEIVKESRKLAERARELIKESEDKRVSEERNERLLEELLRILDENA
ELLKRNLELLKEVLYRTR
3-2′*
(SEQ ID NO: 451)
TERKLLERSRRLQEESKRLLDEMAEIMRRIKKLLKKARGADEKVLDELRKIIERIRELLDRSRKIHERSEEIAYK
EEGSEGSGSEGSGSDESDRIRKIVEESDEIVKESRKLAERARELIKESEDKRVSEERNERLLEELLRILDENAEL
LKRNLELLKEVLYRTR
1′-3′
(SEQ ID NO: 314)
GSDEDDELERLLREYHRVLREYEKLLEELRRLYEEYKRGEVSEEESDRILREIKEILDKSERLWDLSEEVWRTLL
YQAEGSEGSGSEGSGSDDKELDKLLDTLEKILQTATKIIDDANKLLEKLRRSERKDPKVVETYVELLKRHEKAVK
ELLEIAKTHAKKVE
1′-3′
(SEQ ID NO: 453)
DEDDELERLLREYHRVLREYEKLLEELRRLYEEYKRGEVSEEESDRILREIKEILDKSERLWDLSEEVWRTLLYQ
AEGSEGSGSEGSGSDDKELDKLLDTLEKILQTATKIIDDANKLLEKLRRSERKDPKVVETYVELLKRHEKAVKEL
LEIAKTHAKKVE
1′-5
(SEQ ID NO: 303)
PKKEAEELAEESEELHDRSEKLHERAEQSSNSEEARKILEDIERISERIEEISDRIERLLRSGSEGSGSEGSGSD
DKELDKLLDTLEKILQTATKIIDDANKLLEKLRRSERKDPKVVETYVELLKRHEKAVKELLEIAKTHAKKVE
5′-2′
(SEQ ID NO: 302)
TERKLLERSRRLQEESKRLLDEMAEIMRRIKKLLDDPDSEDIAREIKELLRRLKEIIERNQRIAKEHEYIARERS
GSEGSGSEGSGSPKEEARELIRKQKELIKEQKKLIKEAKQKSDSRDAERIWKRSREINRESKKINKRIKELIKS
1-6
(SEQ ID NO: 315)
DSDEHLKKLKTFLENLRRHLDRLDKHIKQLRDILSENPEDERVKDVIDLSERSVRIVKTVIKIFEDSVRKKEGSE
GSGSEGSGSEGSGSEGSGSEGSGSEGSPTDEVIEVLKELLRIHRENLRVNEEIVEVNERASRVTDREELERLLRR
SNELIKRSRELNEESKKLIEKLERLAT
1′-7
(SEQ ID NO: 316)
DDKELDKLLDTLEKILQTATKIIDDANKLLEKLRRSERKDPKVVETYVELLKRHEKAVKELLEIAKTHAKKVEGS
EGSGSEGSTAEELLEVHKKSDRVTKEHLRVSEEILKVVEVLTRGEVSSEVLKRVLRKLEELTDKLRRVTEEQRRV
VEKLN
6′-7
(SEQ ID NO: 317)
DNEEIIKEARRVVEEYKKAVDRLEELVRRAENAKHASEKELKDIVREILRISKELNKVSERLIELWERSQERARG
SEGSGSEGSTAEELLEVHKKSDRVTKEHLRVSEEILKVVEVLTRGEVSSEVLKRVLRKLEELTDKLRRVTEEQRR
VVEKLN
1′-6-7
(SEQ ID NO: 318)
DDKELDKLLDTLEKILQTATKIIDDANKLLEKLRRSERKDPKVVETYVELLKRHEKAVKELLEIAKTHAKKVEGS
EGSGSEGSGSEGSGSEGSGSEGSGSEGSPTDEVIEVLKELLRIHRENLRVNEEIVEVNERASRVTDREELERLLR
RSNELIKRSRELNEESKKLIEKLERLATGSEGSGSEGSGSEGSGSEGSGSEGSGSEGSTAEELLEVHKKSDRVTK
EHLRVSEEILKVVEVLTRGEVSSEVLKRVLRKLEELTDKLRRVTEEQRRVVEKLN
11-1
(SEQ ID NO: 319)
DSDEHLKKLKTFLENLRRHLDRLDKHIKQLRDILSENPEDERVKDVIDLSERSVRIVKTVIKIFEDSVRKKEGSE
GSGSEGSPEDDVVRIIKEDLESNREVLREQKEIHRILELVTRGEVSEEAIDRVLKRQEDLLKKQKESTDKARKVV
EERR
11-6′
(SEQ ID NO: 320)
DNEEIIKEARRVVEEYKKAVDRLEELVRRAENAKHASEKELKDIVREILRISKELNKVSERLIELWERSQERARG
SEGSGSEGSPEDDVVRIIKEDLESNREVLREQKEIHRILELVTRGEVSEEAIDRVLKRQEDLLKKQKESTDKARK
VVEERR
11-7′
(SEQ ID NO: 321)
DLEDLLRRLRRLVDEQRRLVEELERVSRRLEKAVRDNEDERELARLSREHSDIQDKHDKLAREILEVLKRLLERT
EGSEGSGSEGSGSEGSGSEGSGSEGSGSEGSPEDDVVRIIKEDLESNREVLREQKEIHRILELVTRGEVSEEAID
RVLKRQEDLLKKQKESTDKARKVVEERR
1′-6
(SEQ ID NO: 322)
GSDDKELDKLLDTLEKILQTATKIIDDANKLLEKLRRSERKDPKVVETYVELLKRHEKAVKELLEIAKTHAKKVE
GSEGSGSEGSPTDEVIEVLKELLRIHRENLRVNEEIVEVNERASRVTDREELERLLRRSNELIKRSRELNEESKK
LIEKLERLAT
1′-6
(SEQ ID NO: 455)
DDKELDKLLDTLEKILQTATKIIDDANKLLEKLRRSERKDPKVVETYVELLKRHEKAVKELLEIAKTHAKKVEGS
EGSGSEGSPTDEVIEVLKELLRIHRENLRVNEEIVEVNERASRVTDREELERLLRRSNELIKRSRELNEESKKLI
EKLERLAT
7-1
(SEQ ID NO: 323)
DSDEHLKKLKTFLENLRRHLDRLDKHIKQLRDILSENPEDERVKDVIDLSERSVRIVKTVIKIFEDSVRKKEGSE
GSGSEGSTAEELLEVHKKSDRVTKEHLRVSEEILKVVEVLTRGEVSSEVLKRVLRKLEELTDKLRRVTEEQRRVV
EKLN
4′-2′*
(SEQ ID NO: 324)
GTERKLLERSRRLQEESKRLLDEMAEIMRRIKKLLKKARGADEKVLDELRKIIERIRELLDRSRKIHERSEEIAY
KEEGSEGSGSEGSGSDAYDLDRIVKEHRRLVEEQRELVEELEKLVRRQEDHRVDKKESHEILERLERIIRRSTRI
LTELEKLTDEFERRTR
2*-1′
(SEQ ID NO: 325)
TREELLRENIELAKEHIEIMREILELLQKMEELLEKARGADEDVAKTIKELLRRLKEIIERNQRIAKEHEYIARE
RSGSEGSGSEGSGSDDKELDKLLDTLEKILQTATKIIDDANKLLEKLRRSERKDPKVVETYVELLKRHEKAVKEL
LEIAKTHAKKVE
1′-9
(SEQ ID NO: 326)
GSDDKELDKLLDTLEKILQTATKIIDDANKLLEKLRRSERKDPKVVETYVELLKRHEKAVKELLEIAKTHAKKVE
GSEGSGSEGSGTKEDILERQRKIIERAQEIHRRQQEILEELERIIRKPGSSEEAMKRMLKLLEESLRLLKELLEL
SEESAQLLYEQR
1′-9
(SEQ ID NO: 457)
DDKELDKLLDTLEKILQTATKIIDDANKLLEKLRRSERKDPKVVETYVELLKRHEKAVKELLEIAKTHAKKVEGS
EGSGSEGSGTKEDILERQRKIIERAQEIHRRQQEILEELERIIRKPGSSEEAMKRMLKLLEESLRLLKELLELSE
ESAQLLYEQR

In some aspects, each fusion protein independently comprises a polypeptide that is 50%, 55%, 60%, 65%, 70%, 75%, 80%, 85%, 90%, 91%, 92%, 93%, 94%, 95%, 96%, 97%, 98%, 99%, or 100% identical to the full length of a polypeptide selected from the group including, but not limited to, a polypeptide having the amino acid sequence of SEQ ID NO: 302, 303, 306-326, 439, 441, 443, 445, 447, 449, 451, 453, 455, and 457, wherein GlySer at amino acid residues 1 and 2 of any of 302, 303, 306-326, 439, 441, 443, 445, 447, 449, 451, 453, 455, and 457 are optional, e.g., not present.

In another embodiment, the kits or compositions further comprising the first target and the second target. In one embodiment, the first target and the second target each independently comprise a polypeptide of the formula X10-B10-Z10, wherein
(a) the X10 domain is a non-naturally occurring polypeptide comprising 1, 2, or 3 alpha helices;
(b) the Z10 domain is a non-naturally occurring polypeptide comprising 1, 2, or 3 alpha helices; and
(c) the B10 domain is an amino acid linker;
wherein the X domain and the Z domain interact at a target binding interface, wherein the target binding interface comprises a hydrogen bond network in which at least one side chain in each alpha helix hydrogen of the X domain bonds with a side chain in a different alpha helix in the Z domain, and wherein the target binding interface comprises a plurality of hydrophobic residues. In one embodiment, the target binding interface comprises at least 25% hydrophobic residues. In another embodiment, the B10 domain for the first target and the second target is independently between 6-12, 6-11, 6-10, 7-12, 7-11, 7-10, 8-12, 8-11, 8-10, 9-12, 9-11, 9-10, 10-12, 10-11, 11-12, 6, 7, 8, 9, 10, 11, or 12 amino acids in length. In another embodiment, the combined number of alpha helices from the X and Z domains in the first target and the second target protein is 4. In a further embodiment,
(a) the X10 domain of each of the first target and the second target has 2 alpha helices and the Z10 domain of each of the first target and the second target has 2 alpha helices; or
(b) either the X10 domain or the Z10 domain of each of the first target and the second target has 1 alpha helix and the other has 3 alpha helices. In one embodiment, each X10 domain and each Z10 domain comprise a polypeptide that is 50%, 55%, 60%, 65%, 70%, 75%, 80%, 85%, 90%, 91%, 92%, 93%, 94%, 95%, 96%, 97%, 98%, 99%, or 100% identical to the full length of a polypeptide selected from the group including, but not limited to a polypeptide having the amino acid sequence selected from SEQ ID NOS:1-290, 331, 332, 334, 336-422, 424, 426, 428, 430, 432, 434, 436, 438, 440, 442, 444, 446, 448, 450, 452, 454, 456, 458-460, 462, 464, 466, 468, 470, 472, 474, 476, 478, 480, 482, 484, 486, 488, 490, 493, and 494, with the proviso that the X10 domain forms a heterodimer (a-b) pair with the X domain of the fusion protein, and the Z10 domain forms a heterodimer (a-b) pair with the Z domain of the fusion protein. In one embodiment, at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% of amino acid residues at defined interface positions of the first target and/or the second target are invariant compared to the reference polypeptide amino acid sequence (interface residues shown in Table 2).

In another embodiment, the first target and/or the second target further comprise one or more effector polypeptide domains linked to one or more of the X10 and/or Z10 domains, for example, wherein the one or more effector polypeptide domains may comprise a polypeptide including, but not limited to, nucleic acid binding proteins, transcription factors, receptor binding proteins, split enzymes, effectors of membrane receptors, etc.

In a sixth aspect, the disclosure provides methods, comprising:
(i) contacting the fusion protein of embodiment or combination of embodiments of the fifth or sixth aspects disclosed herein with a biological sample under conditions to promote non-covalent binding of the fusion protein with first target and second target present in the sample, and
(ii) detecting non-covalent binding of the one or more fusion proteins to the first target and/or the second target in the biological sample.

The detecting may comprise any suitable means for detecting binding, including but not limited to mass spectrometry, yeast-2-hybrid detection, functional assays, or any other suitable assay as will be clear to those of skill in the art based on the current disclosure. In one embodiment, the method comprises detecting cooperative non-covalent binding of the one or more fusion proteins to the first target and the second target in the biological sample. This embodiment comprises use of the fusion proteins in AND gate logic, as described in more detail in the examples that follow. As used herein, “cooperative” binding means binding the fusion protein cannot bind to the first target without also binding to the second target, and the fusion protein cannot bind to the second target without binding to the first target.

In another embodiment, the method comprises detecting non-covalent binding of the one or more fusion proteins to the first target or the second target in the biological sample. This embodiment comprises use of the fusion proteins in OR gate logic, as described in more detail in the examples that follow.

In another embodiment, the disclosure provides methods comprising:
(a) contacting a biological sample with at least two fusion proteins, wherein each of the at least two fusion proteins comprises the formula X—B—Z, wherein
each B is independently a polypeptide linker;
each X domain comprises a first non-naturally occurring polypeptide comprising 1, 2, or 3 alpha helices;
each Z domain comprises a second non-naturally occurring polypeptide comprising 1, 2, or 3 alpha helices, wherein a combined number of alpha helices from the X domain and the Z domain in each individual fusion protein is 4, 5, or 6; wherein the X domain and the Z domain interact at a binding interface, wherein the binding interface comprises a hydrogen bond network in which at least one side chain in each X domain alpha helix bonds with a side chain in an alpha helix in the Z domain; wherein
the X domain in a first fusion protein is capable of non-covalently binding to a first target;
the Z domain in a second fusion protein is capable of non-covalently binding to a second target; and
the X domains and Z domains in each individual fusion protein that are not capable of non-covalently binding to the first target or the second target are capable of non-covalently binding to an X or a Z domain of a different fusion protein in the plurality of fusion proteins;
(b) detecting non-covalent binding of the two or more fusion proteins to the first target and/or the second target in the biological sample. This embodiment comprises use of the fusion proteins in 2 component AND or OR gate logic, as described in more detail in the examples that follow.

In one embodiment of the AND or OR gate logic, the detecting comprises detecting cooperative non-covalent binding of the two or more fusion proteins to the first target and the second target in the biological sample. In another embodiment,
(i) the first fusion protein has the formula X1-B1-Z1, wherein the X1 domain is capable of non-covalently binding to the first target; and
(ii) the second fusion protein has the formula X2-B2-Z2, wherein the Z2 domain is capable of non-covalently binding to the first target; and wherein the Z1 and X2 domains are capable of non-covalently binding to each other.

In a further embodiment,
(i) the first fusion protein has the formula X1-B1-Z1, wherein the X1 domain is capable of non-covalently binding to the first target; and
(ii) the second fusion protein has the formula X2-B2-Z2,
(iii) the at least two fusion proteins comprise a third fusion protein of formula X3-B3-Z3, wherein the Z3 domain is capable of non-covalently binding to the second target; wherein
   (A) the Z1 and X2 domains are capable of non-covalently binding to each other; and
   (B) the Z2 and X3 domains are capable of non-covalently binding to each other.

In another embodiment, the X domains, Y domains, B domains, and or fusion proteins are as recited in any embodiment or combination of embodiments disclosed herein, such as in the fourth and fifth aspects. In one embodiment, at least one of the fusion proteins comprises one or more effector polypeptide domains linked to one or more of the X and/or Z domains, and wherein the detecting step comprises detecting an output signal caused by binding the first target and/or the second target. In another embodiment, the detecting step comprises detecting an output signal from the one or more effector polypeptide caused by cooperative non-covalently binding of the first target and the second target. Such detection may be by any suitable means dependent in part on the output signal to be detected, including but not limited to those disclosed herein. The output signal to be detected may be any suitable output signal including but not limited to fluorescence activity, functional activity, etc.

Any suitable effector polypeptide domain may be employed as suitable for an intended use. In one embodiment, the one or more effector polypeptide domains may comprise a polypeptide including, but not limited to, nucleic acid binding proteins, transcription factors, receptor binding proteins, nucleic acid binding proteins, transcription factors, receptor binding proteins, split enzymes, effectors of membrane receptors, etc.

In a seventh aspect, the disclosure provides compositions comprising
(a) a first polypeptide comprising 2 alpha helices, wherein the first polypeptide is capable of non-covalently binding a first target; and
(b) a second polypeptide comprising 2 alpha helices, wherein the first polypeptide is capable of non-covalently binding to the second polypeptide, and wherein the second polypeptide is capable of non-covalently binding a second target that differs from the first target; wherein:
   (i) a binding affinity of the first polypeptide for the first target is approximately equal to a binding affinity of the second polypeptide for the second target; and
   (ii) the binding affinity of the first polypeptide for the first target and the binding affinity of the second polypeptide for the second target are greater than the binding affinity of the first target and the second target for each other.

Compositions of this seventh aspect can be used, for example, as NOR gates as described in detail in the examples that follow.

In one embodiment, the composition further comprises the first target and the second target. The first targets and second targets may be any target suitable for an intended use. In non-limiting embodiments, the first target and/or the second target may comprise polypeptides or nucleic acids. In another embodiment, the first target and/or the second target further comprise one or more effector polypeptide domains. Any effector polypeptide domains may be used as suitable for an intended use. In one embodiment, the one or more effector polypeptide domains may comprise a polypeptide including, but not limited to, nucleic acid binding proteins, transcription factors, receptor binding proteins, split enzymes, effectors of membrane receptors, etc. In another embodiment, the first polypeptide and/or the second polypeptide comprise a polypeptide that is 50%, 55%, 60%, 65%, 70%, 75%, 80%, 85%, 90%, 91%, 92%, 93%, 94%, 95%, 96%, 97%, 98%, 99%, or 100% identical to the full length of a polypeptide selected from the group including, but not limited to a polypeptide having the amino acid sequence selected from the group consisting of SEQ ID NOS:1-290 and 331, 332, 334, 336-422, 424, 426, 428, 430, 432, 434, 436, 438, 440, 442, 444, 446, 448, 450, 452, 454, 456, 458-460, 462, 464, 466, 468, 470, 472, 474, 476, 478, 480, 482, 484, 486, 488, 490, 493, and 494, as listed in Tables 1A and 1B. In one embodiment, at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% of amino acid residues at defined interface positions of the first polypeptide and/or the second polypeptide are invariant compared to the reference polypeptide amino acid sequence (interface residues shown in Table 2).

In one non-limiting and exemplary embodiment,
(a) the first polypeptide comprise a polypeptide that is 50%, 55%, 60%, 65%, 70%, 75%, 80%, 8%, 90%, 91%, 92%, 93%, 94%, 95%, 96%, 97%, 98%, 99%, or 100% identical to the full length of a polypeptide selected from the group including, but not limited to one having the amino acid sequence of SEQ ID NO:3 9 GTKEDILERQRKIIERAQEIHRRQQEILEELERIIRKPGSSEEAMKRMLKLLEESLRLLKELLELSEESAQLLYE QR (SEQ ID NO: 3); and
(b) the second polypeptide comprise a polypeptide that is 50%, 55%, 60%, 65%, 70%, 75%, 80%, 85%, 90%, 91%, 92%, 93%, 94%, 95%, 96%, 97%, 98%, 99%, or 100% identical to the full length of a polypeptide selected from the group including, but not limited to one having the amino acid sequence of SEQ ID NO:58.

10′
(SEQ ID NO: 58)
GSSADDVLEDILKIIRELIEILDQILSLLNQLLKLLRHGVPNAKKVVEKYKEILELYLQLVSLELKIVKTHADAV
SGKIDKKAEEEIKKEEEKIKEKLRQAKDILKKLQEEIDKTR

In one non-limiting and exemplary embodiment,
(a) the first polypeptide comprise a polypeptide that is 50%, 55%, 60%, 65%, 70%, 75%, 80%, 85%, 90%, 91%, 92%, 93%, 94%, 95%, 96%, 97%, 98%, 99%, or 100% identical to the full length of a polypeptide selected from the group including, but not limited to one having the amino acid sequence of SEQ ID NO:3 9 GTKEDILERQRKIIERAQEIHRRQQEILEELERIIRKPGSSEEAMKRMLKLLEESLRLLKELLELSEESAQLLYE QR (SEQ ID NO: 3); and
(b) the second polypeptide comprise a polypeptide that is 50%, 55%, 60%, 65%, 70%, 75%, 80%, 85%, 90%, 91%, 92%, 93%, 94%, 95%, 96%, 97%, 98%, 99%, or 100% identical to the full length of a polypeptide selected from the group including, but not limited to one having the amino acid sequence of SEQ ID NO:362.

10′
(SEQ ID NO: 362)
SADDVLEDILKIIRELIEILDQILSLLNQLLKLLRHGVPNAKKVVEKYKEILELYLQLVSLELKIVKTHADAVSG
KIDKKAEEEIKKEEEKIKEKLRQAKDILKKLQEEIDKTR

In another embodiment, the first target and/or the second target each comprise a polypeptide that is 50%, 55%, 60%, 65%, 70%, 75%, 80%, 85%, 90%, 91%, 92%, 93%, 94%, 95%, 96%, 97%, 98%, 99%, or 100% identical to the full length of a polypeptide selected from the group including, but not limited to a polypeptide having the amino acid sequence selected from the group consisting of SEQ ID NO:1-290, with the proviso that the first target forms a heterodimer (a-b) pair with the first polypeptide, and the second target forms a heterodimer (a-b) pair with the second polypeptide. In another embodiment, the first target and/or the second target each comprises a polypeptide that is 50%, 55%, 60%, 65%, 70%, 75%, 80%, 85%, 90%, 91%, 92%, 93%, 94%, 95%, 96%, 97%, 98%, 99%, or 100% identical to the full length of a polypeptide selected from the group including, but not limited to a polypeptide having the amino acid sequence selected from the group consisting of SEQ ID NO:1-290 and 331, 332, 334, 336-422, 424, 426, 428, 430, 432, 434, 436, 438, 440, 442, 444, 446, 448, 450, 452, 454, 456, 458-460, 462, 464, 466, 468, 470, 472, 474, 476, 478, 480, 482, 484, 486, 488, 490, 493, and 494, with the proviso that the first target forms a heterodimer (a-b) pair with the first polypeptide, and the second target forms a heterodimer (a-b) pair with the second polypeptide. Heterodimer A-B pairs among the polypeptides of SEQ ID NOS:1-290 and 331, 332, 334, 336-422, 424, 426, 428, 430, 432, 434, 436, 438, 440, 442, 444, 446, 448, 450, 452, 454, 456, 458-460, 462, 464, 466, 468, 470, 472, 474, 476, 478, 480, 482, 484, 486, 488, 490, 493, and 494 are described at length above (See also FIG. 16). In one embodiment, at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% of amino acid residues at defined interface positions of the first target and/or the second target are invariant compared to the reference polypeptide amino acid sequence (interface residues shown in Table 2).

The compositions of this seventh aspect can be used for any suitable purpose, including in designing NOR logic gates. In one embodiment, the disclosure provides methods comprising
(a) contacting a biological sample with the composition of any embodiment or combination of embodiments of the seventh aspect of the disclosure; and
(b) detecting binding, of the first polypeptide to the first target and binding of the second polypeptide to the second target in the sample, such as detecting an output signal caused by actions of effector polypeptides upon binding. Additional details of the use of the compositions of the seventh aspect of the disclosure in NOR logic gates re described in detail in the examples that follow.

In an eighth aspect, the disclosure provides compositions comprising:
(a) a first polypeptide comprising 2 alpha helices, wherein the first polypeptide is capable of non-covalently binding a first target; and
(b) a second polypeptide comprising 2 alpha helices, wherein the first polypeptide is capable of non-covalently binding to the second polypeptide, and wherein the second polypeptide is capable of non-covalently binding a second target that differs from the first target; wherein:
   (i) a binding affinity of the first polypeptide for the second polypeptide is greater than a binding affinity of the second polypeptide for the second target;
   (ii) a binding affinity of the first polypeptide for the first target is approximately equal to a binding affinity of the second polypeptide for the second target; and
   (iii) the binding affinity of the first polypeptide for the first target and the binding affinity of the second polypeptide for the second target are greater than the binding affinity of the first target and the second target for each other.

Compositions of this eighth aspect can be used, for example, as XNOR gates as described in detail in the examples that follow. In one embodiment, the composition further comprises the first target and the second target. The first targets and second targets may be any target suitable for an intended use. In non-limiting embodiments, the first target and/or the second target may comprise polypeptides or nucleic acids. In another embodiment, the first target and/or the second target further comprise one or more effector polypeptide domains. Any effector polypeptide domains may be used as suitable for an intended use. In one embodiment, the one or more effector polypeptide domains may comprise a polypeptide including, but not limited to, nucleic acid binding proteins, transcription factors, receptor binding proteins, split enzymes, effectors of membrane receptors, etc. In another embodiment, the first polypeptide and/or the second polypeptide comprise a polypeptide that is 50%, 55%, 60%, 65%, 70%, 75%, 80%, 85%, 90%, 91%, 92%, 93%, 94%, 95%, 96%, 97%, 98%, 99%, or 100% identical to the full length of a polypeptide selected from the group including, but not limited to a polypeptide having the amino acid sequence selected from the group consisting of SEQ ID NOS:1-290 and 331, 332, 334, 336-422, 424, 426, 428, 430, 432, 434, 436, 438, 440, 442, 444, 446, 448, 450, 452, 454, 456, 458-460, 462, 464, 466, 468, 470, 472, 474, 476, 478, 480, 482, 484, 486, 488, 490, 493, and 494, as listed in Tables 1A and 1B. In one embodiment, at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% of amino acid residues at defined interface positions of the first polypeptide and/or the second polypeptide are compared to the reference polypeptide amino acid sequence (interface residues shown in Table 2).

In one non-limiting and exemplary embodiment,
(a) the first polypeptide comprise a polypeptide that is 50%, 55%, 60%, 65%, 70%, 75%, 80%, 85%, 90%, 91%, 92%, 93%, 94%, 95%, 96%, 97%, 98%, 99%, or 100% identical to the full length of a polypeptide selected from the group including, but not limited to one having the amino acid sequence of SEQ ID NO:3 9 GTKEDILERQRKIIERAQEIHRRQQEILEELERIIRKPGSSEEAMKRMLKLLEESLRLLKELLELSEESAQLLYE QR (SEQ ID NO: 3); and
(b) the second polypeptide comprise a polypeptide that is 50%, 55%, 60%, 65%, 70%, 75%, 80%, 85%, 90%, 91%, 92%, 93%, 94%, 95%, 96%, 97%, 98%, 99%, or 100% identical to the full length of a polypeptide selected from the group including, but not limited to one having the amino acid sequence of SEQ ID NO:42.

1′ (b)
(SEQ ID NO: 42)
GSDDKELDKLLDTLEKILQTATKIIDDANKLLEKLRRSERKDPKVVETYVELLKRHEKAVKELLEIAKTHAKKVE

In one non-limiting and exemplary embodiment,
(a) the first polypeptide comprise a polypeptide that is 50%, 55%, 60%, 65%, 70%, 75%, 80%, 85%, 90%, 91%, 92%, 93%, 94%, 95%, 96%, 97%, 98%, 99%, or 100% identical to the full length of a polypeptide selected from the group including, but not limited to one having the amino acid sequence of SEQ ID NO:3 9 GTKEDILERQRKIIERAQEIHRRQQEILEELERIIRKPGSSEEAMKRMLKLLEESLRLLKELLELSEESAQLLYE QR (SEQ ID NO: 3); and
(b) the second polypeptide comprise a polypeptide that is 50%, 55%, 60%, 65%, 70%, 75%, 80%, 85%, 90%, 91%, 92%, 93%, 94%, 95%, 96%, 97%, 98%, 99%, or 100% identical to the full length of a polypeptide selected from the group including, but not limited to one having the amino acid sequence of SEQ ID NO:352.

1′ (b)
(SEQ ID NO: 352)
DDKELDKLLDTLEKILQTATKIIDDANKLLEKLRRSERKDPKVVETYVELLKRHEKAVKELLEIAKTHAKKVE

In another embodiment, the first target and/or the second target each comprise a polypeptide that is 50%, 55%, 60%, 65%, 70%, 75%, 80%, 85%, 90%, 91%, 92%, 93%, 94%, 95%, 96%, 97%, 98%, 99%, or 100% identical to the full length of a polypeptide selected from the group including, but not limited to a polypeptide having the amino acid sequence selected from the group consisting of SEQ ID NO:1-290 and 331, 332, 334, 336-422, 424, 426, 428, 430, 432, 434, 436, 438, 440, 442, 444, 446, 448, 450, 452, 454, 456, 458-460, 462, 464, 466, 468, 470, 472, 474, 476, 478, 480, 482, 484, 486, 488, 490, 493, and 494, with the proviso that the first target forms a heterodimer (a-b) pair with the first polypeptide, and the second target forms a heterodimer (a-b) pair with the second polypeptide. Heterodimer A-B pairs among the polypeptides of SEQ ID NOS:1-290 and 331, 332, 334, 336-422, 424, 426, 428, 430, 432, 434, 436, 438, 440, 442, 444, 446, 448, 450, 452, 454, 456, 458-460, 462, 464, 466, 468, 470, 472, 474, 476, 478, 480, 482, 484, 486, 488, 490, 493, and 494 are described at length above (See also FIG. 16). In one embodiment, at least 20%, 25%, 30%, 35%, 40%, 45%, 50%, 55%, 60%, 6%, 70%, 75% 80%, 85%, 90%, 91%, 92%, 93%, 94%, 95%, 96%, 97%, 98%, 99%, or 100% of amino acid residues at defined interface positions of the first target and/or the second target are invariant compared to the reference polypeptide amino acid sequence (interface residues shown in Table 2).

The compositions of this eighth aspect can be used for any suitable purpose, including in designing XNOR logic gates. In one embodiment, the disclosure provides methods comprising
(a) contacting a biological sample with the composition of any one of claims 50-56; and
(b) detecting binding interactions between the first polypeptide and the first target. the second polypeptide and the second target, the first polypeptide and the second polypeptide, and the first target and the second target in the sample, such as detecting an output signal caused by actions of effector polypeptides upon binding. Additional details of the use of the compositions of the eighth aspect of the disclosure in XNOR logic gates re described in detail in the examples that follow.

In a ninth aspect, the disclosure provides compositions comprising:
(a) a first polypeptide comprising 4 alpha helices, wherein the first polypeptide is capable of non-covalently binding a first target; and
(b) a second polypeptide comprising 4 alpha helices, wherein the second polypeptide is capable of non-covalently binding a second target that differs from the first target; wherein:
   (i) a binding affinity of the first target for the second target is greater than a binding affinity of the first polypeptide for the first target;
   (ii) a binding affinity of the first polypeptide for the first target is approximately equal to a binding affinity of the second polypeptide for the second target; and
   (iii) the sum of the binding affinity of (A) the first polypeptide for the first target and (B) the binding affinity of the second polypeptide for the second target, is greater than the binding affinity of the first target and the second target.

Compositions of this ninth aspect can be used, for example, as NAND gates as described in detail in the examples that follow. In one embodiment, the composition further comprises the first target and the second target. The first targets and second targets may be any target suitable for an intended use. In non-limiting embodiments, the first target and/or the second target may comprise polypeptides or nucleic acids. In another embodiment, the first target and/or the second target further comprise one or more effector polypeptide domains. Any effector polypeptide domains may be used as suitable for an intended use. In one embodiment, the one or more effector polypeptide domains may comprise a polypeptide including, but not limited to, nucleic acid binding proteins, transcription factors, receptor binding proteins, split enzymes, effectors of membrane receptors, etc. In another embodiment, the first polypeptide and/or the second polypeptide comprise a polypeptide that is 50%, 55%, 60%, 65%, 70%, 75%, 80%, 85%, 90%, 91%, 92%, 93%, 94%, 95%, 96%, 97%, 98%, 99%, or 100% identical to the full length of a polypeptide selected from the group including, but not limited to a polypeptide having the amino acid sequence selected from the group consisting of SEQ ID NOS:1-290 and 331, 332, 334, 336-422, 424, 426, 428, 430, 432, 434, 436, 438, 440, 442, 444, 446, 448, 450, 452, 454, 456, 458-460, 462, 464, 466, 468, 470, 472, 474, 476, 478, 480, 482, 484, 486, 488, 490, 493, and 494, as listed in Tables 1A and 1B. In one embodiment, at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% of amino acid residues at defined interface positions of the first polypeptide and/or the second polypeptide are invariant compared to the reference polypeptide amino acid sequence (interface residues shown in Table 2).

In one non-limiting and exemplary embodiment,
(a) the first polypeptide comprise a polypeptide that is 50%, 55%, 60%, 65%, 70%, 75%, 80%, 85%, 90%, 91%, 92%, 93%, 94%, 95%, 96%, 97%, 98%, 99%, or 100% identical to the full length of a polypeptide selected from the group including, but not limited to one having the amino acid sequence of SEQ ID NO:42

1′ (b)
(SEQ ID NO: 42)
GSDDKELDKLLDTLEKILQTATKIIDDANKLLEKLRRSERKDPKVVETYVELLKRHEKAVKELLEIAKTHAKKVE

(b) the second polypeptide comprise a polypeptide that is 50%, 55%, 60%, 65%, 70%, 75%, 80%, 85%, 90%, 91%, 92%, 93%, 94%, 95%, 96%, 97%, 98%, 99%, or 100% identical to the full length of a polypeptide selected from the group including, but not limited to one having the amino acid sequence of SEQ ID NO: 57.

10
(SEQ ID NO: 57)
DHSRKLEEILDRLRKHVKRLLEHLRELLSLVKENPEDKDLVEVLELSLAILRRSLEAVEAFLKSVTKKDPDDEDL
RRKADEIRKEVEEIKKSLAEVEKEIYKLK

In one non-limiting and exemplary embodiment,
(a) the first polypeptide comprise a polypeptide that is 50%, 55%, 60%, 65%, 70%, 75%, 80%, 85%, 90%, 91%, 92%, 93%, 94%, 95%, 96%, 97%, 98%, 99%, or 100% identical to the full length of a polypeptide selected from the group including, but not limited to one having the amino acid sequence of SEQ ID NO:352

1′ (b)
(SEQ ID NO: 352)
DDKELDKLLDTLEKILQTATKIIDDANKLLEKLRRSERKDPKVVETYVELLKRHEKAVKELLEIAKTHAKKVE

(b) the second polypeptide comprise a polypeptide that is 50%, 55%, 60%, 65%, 70%, 75%, 80%, 85%, 90%, 91%, 92%, 93%, 94%, 95%, 96%, 97%, 98%, 99%, or 100% identical to the full length of a polypeptide selected from the group including, but not limited to one having the amino acid sequence of SEQ ID NO: 57.

10
(SEQ ID NO: 57)
DHSRKLEEILDRLRKHVKRLLEHLRELLSLVKENPEDKDLVEVLELSLAILRRSLEAVEAFLKSVTKKDPDDEDL
RRKADEIRKEVEEIKKSLAEVEKEIYKLK

In another embodiment, the first target and/or the second target each comprise a polypeptide that is 50%, 55%, 60%, 65%, 70%, 75%, 80%, 85%, 90%, 91%, 92%, 93%, 94%, 95%, 96%, 97%, 98%, 99%, or 100% identical to the full length of a polypeptide selected from the group including, but not limited to a polypeptide having the amino acid sequence selected from the group consisting of SEQ ID NO:1-290 and 331, 332, 334, 336-422, 424, 426, 428, 430, 432, 434, 436, 438, 440, 442, 444, 446, 448, 450, 452, 454, 456, 458-460, 462, 464, 466, 468, 470, 472, 474, 476, 478, 480, 482, 484, 486, 488, 490, 493, and 494, with the proviso that the first target forms a heterodimer (a-b) pair with the first polypeptide, and the second target forms a heterodimer (a-b) pair with the second polypeptide. Heterodimer A-B pairs among the polypeptides of SEQ ID NOS:1-290 and 331, 332, 334, 336-422, 424, 426, 428, 430, 432, 434, 436, 438, 440, 442, 444, 446, 448, 450, 452, 454, 456, 458-460, 462, 464, 466, 468, 470, 472, 474, 476, 478, 480, 482, 484, 486, 488, 490, 493, and 494 are described at length above (See also FIG. 16). In one embodiment, at least 20%, 25%, 30%, 35%, 40%, 45%, 50%, 55% 60%, 65%, 70%, 75%, 80%, 85%, 90%, 91%, 92%, 93%, 94%, 95%, 96%, 97%, 98%, 99%, or 100% of amino acid residues at defined interface positions of the first target and/or the second target are compared to the reference polypeptide amino acid sequence (interface residues shown in Table 2).

The compositions of this ninth aspect can be used for any suitable purpose, including in designing NAND logic gates. In one embodiment, the disclosure provides methods comprising
(a) contacting a biological sample with the composition of any one of claims 60-66; and
(b) detecting binding interactions between the first polypeptide and the first target. the second polypeptide and the second target, and the first target and the second target in the sample, such as detecting an output signal caused by actions of effector polypeptides upon binding.

As used throughout the present application, the term “polypeptide” is used in its broadest sense to refer to a sequence of subunit amino acids. The polypeptides of the invention may comprise L-amino acids, D-amino acids (which are resistant to L-amino acid-specific proteases in vivo), or a combination of D- and L-amino acids. The polypeptides described herein may be chemically synthesized or recombinantly expressed. The polypeptides may be linked to other compounds to promote an increased half-life in vivo, such as by PEGylation, HESylation, PASylation, glycosylation, or may be produced as an Fc-fusion or in deimmunized variants. Such linkage can be covalent or non-covalent as is understood by those of skill in the art.

As will be understood by those of skill in the art, the polypeptides of the invention may include additional residues at the N-terminus, C-terminus, or both that are not present in the polypeptides of the invention; these additional residues are not included in determining the percent identity of the polypeptides of the invention relative to the reference polypeptide.

As noted above, the polypeptides of the invention may include additional residues at the N-terminus, C-terminus, or both. Such residues may be any residues suitable for an intended use, including but not limited to detection tags (i.e.: fluorescent proteins, antibody epitope tags, etc.), linkers, therapeutic agents, ligands suitable for purposes of purification (His tags, etc.), ligands to drive localization, and peptide domains that add functionality to the polypeptides.

In a tenth aspect, the disclosure provides nucleic acids encoding the polypeptide, protein, fusion protein, scaffold, or design component of any embodiment or combination of embodiments disclosed herein. The nucleic acid sequence may comprise single stranded or double stranded RNA or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded polypeptide, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the disclosure.

In an eleventh aspect, the disclosure provides expression vector comprising one or more nucleic acids of the disclosure operatively linked to a control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In a twelfth aspect, the disclosure provides cells comprising one or more nucleic acid, expression vector, polypeptide, protein, heterodimer protein, and/or protein scaffold of any embodiment or combination of embodiments disclosed herein. Nucleic acids or expression vectors may be episomal or chromosomally integrated. Any suitable cell type may be used, such prokaryotic or eukaryotic cells. The cells can be transiently or stably engineered to incorporate the expression vector of the disclosure, using techniques including but not limited to bacterial transformations, calcium phosphate co-precipitation, electroporation, or liposome mediated-, DEAE dextran mediated-, polycationic mediated-, or viral mediated transfection

In addition, the disclosure provides methods of producing a polypeptide, fusion protein, protein, heterodimer, etc. (collectively referred to as polypeptide) disclosed herein. In one embodiment, the method comprises the steps of (a) culturing a host according to this aspect of the disclosure under conditions conducive to the expression of the polypeptide, and (b) optionally, recovering the expressed polypeptide. The expressed polypeptide can be recovered from the cell free extract or recovered from the culture medium. In another embodiment, the method comprises chemically synthesizing the polypeptides.

### Example 1 Design of Orthogonal Protein Heterodimers

Abstract: Here we demonstrate that heterodimeric interaction specificity can be achieved using extensive and modular buried hydrogen bond networks. We used the Crick generating equations to produce millions of four helix backbones with varying degrees of supercoiling around a central axis, identified those accommodating extensive hydrogen bond networks, and designed connected pairs of helices with short loops and optimize the remainder of the sequence. 65 of 97 such designs expressed in E. coli formed constitutive heterodimers, and crystal structures of four designs were in close agreement with the computational models and confirmed the designed hydrogen bond networks. In cells, a set of six heterodimers were found to be fully orthogonal, and in vitro, following mixing of 32 chains from sixteen heterodimer designs, denaturation in 5M GdnHCl and reannealing, the vast majority of the interactions were between the designed cognate pairs. The ability to design orthogonal protein heterodimers enables sophisticated protein based control logic for synthetic biology, and illustrates that nature has not fully explored the possibilities for programmable biomolecular interaction modalities. Hydrogen bond networks, including modular hydrogen bond networks are described in published patent application number WO2017173356, incorporated by reference herein.

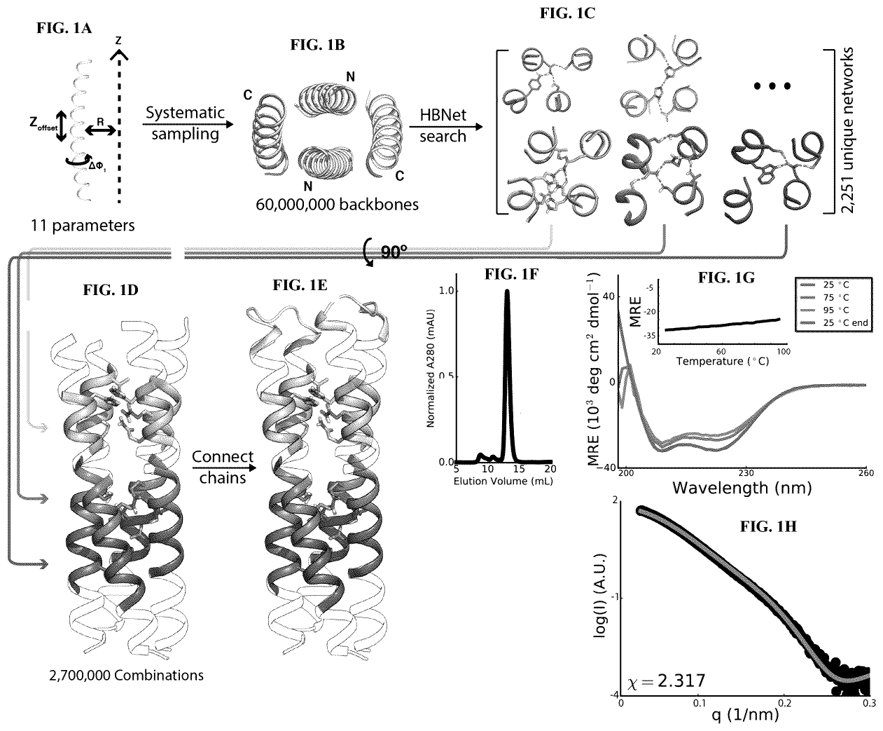

Orthogonal sets of protein-protein and protein-peptide interactions play important roles in biological systems. Creation of new specificities by sequence redesign has been difficult, often resulting in promiscuous binding We hypothesized that large sets of designed heterodimers could be generated by incorporating asymmetric buried hydrogen bond networks into regularly repeating backbone structures. We generated helical bundle heterodimers in which each monomer is a helix-turn-helix starting from four-helix backbones. For each of the four helices, we exhaustively sampled the helical phase (ΔΦ₁), supercoil radius (R) and offset along the Z-axis (Z offset) (FIG.. TA), restricting the supercoil phases of the helices to 0, 90, 180 and 270 degrees, and the supercoil twist (ω₀) and helical twist (ω₁) to the ideal values for either a two layer left handed super coil (ω₀=−2.85 and ω₁=102.85), or a 5 layer untwisted bundle (ω₀=0 and ω₁=100) (FIG. 5A-B). This yielded 27 million untwisted and 60 million left-handed supercoiled backbones for both parallel and antiparallel orientations of opposing helices (FIG. 1B).

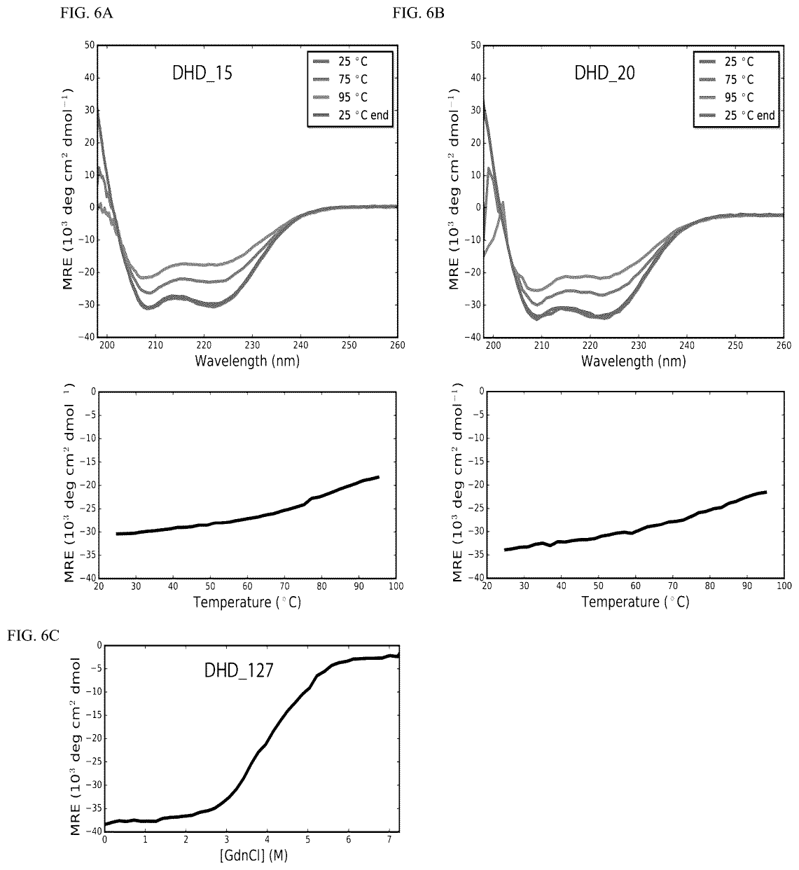

To identify the modular hydrogen bond network equivalents to DNA base pairs, we used ROSETTA™ HBNET²¹ to design buried hydrogen bond networks in the central repeat units of each backbone, and obtained 2251 hydrogen bond networks involving at least 4 side chain residues with all heavy-atom donors and acceptors participating in hydrogen bonds, and connecting all 4 helices (FIG. 1c; FIG. 6, Table 6). We then identified all of the geometrically compatible placements of these hydrogen bond networks in each backbone (FIG. 1d), selected backbones accommodating at least two networks, and connected pairs of helices with short loops (FIG. 1e). Low energy sequences were identified using ROSETTADESIGN™²² calculations in which the hydrogen bond networks were held fixed. Designs with fully satisfied hydrogen bond networks and tight hydrophobic packing were selected for experimental characterization, excluding those with networks with C2 symmetry to disfavor homodimerization of monomers. Designed heterodimers (DHDs) are referred to by numbers with monomers labeled a or b; for example, DHD15_a refers to monomer “a” of design DHD15.

TABLE 6
The frequency of observing each hydrogen bond
networks during the systematic search.

| HBNet | | Percentage |
|---|---|---|
| composition | Frequency | (%) |
| (S/T) 2Q1Y1 | 13954 | 3.87071362 |
| (S/T) 3Q1 | 9959 | 2.76253669 |
| (S/T) 2D1H1 | 8452 | 2.34450849 |
| (S/T) 1Q2Y1 | 7603 | 2.10900356 |
| (S/T) 1D1Q1Y1 | 7359 | 2.04132016 |
| (S/T) 3D1 | 6332 | 1.75643963 |
| (S/T) 3D1Q1 | 5525 | 1.53258512 |
| (S/T) 1D1Q3 | 5071 | 1.40664962 |
| (S/T) 1D1Q2 | 5062 | 1.4041531 |
| (S/T) 2N1Y1 | 5046 | 1.39971484 |
| (S/T) 1N1Q1Y1 | 4921 | 1.36504097 |
| (S/T) 2H2 | 4683 | 1.29902192 |
| (S/T) 2H1Q1 | 4572 | 1.26823152 |
| (S/T) 3H1 | 3955 | 1.09708129 |
| (S/T) 2D1Q2 | 3946 | 1.09458477 |
| (S/T) 1D1N1Q2 | 3862 | 1.07128393 |
| (S/T) 3N1 | 3783 | 1.04937005 |
| (S/T) 2D1Y1 | 3762 | 1.04354484 |
| (S/T) 2D1Q1 | 3669 | 1.01774747 |
| (S/T) 1D1H1Q1 | 3653 | 1.01330922 |
| (S/T) 1D1Q1W1 | 3409 | 0.94562582 |
| (S/T) 1D1Q2Y1 | 3342 | 0.92704063 |
| (S/T) 2Q3 | 3111 | 0.86296331 |
| (S/T) 2D1N1Q1 | 2999 | 0.83189552 |
| (S/T) 2Q2Y1 | 2850 | 0.79056427 |
| (S/T) 1D1W1Y1 | 2849 | 0.79028688 |
| (S/T) 2N1Q2 | 2741 | 0.76032865 |
| (S/T) 2D1Q1Y1 | 2723 | 0.75533562 |
| (S/T) 1D1N1Q1Y1 | 2684 | 0.74451737 |
| (S/T) 2Q1W1 | 2641 | 0.73258956 |
| (S/T) 2H1N1Q1 | 2591 | 0.71872001 |
| (S/T) 2Q2 | 2582 | 0.71622349 |
| (S/T) 2N1Q1 | 2554 | 0.70845654 |
| (S/T) 2D1W1 | 2467 | 0.68432353 |
| (S/T) 2H1N1 | 2377 | 0.65935834 |
| (S/T) 4Q1 | 2305 | 0.63938619 |
| (S/T) 1N1Q2Y1 | 2296 | 0.63688967 |
| (S/T) 1D2Q2 | 2285 | 0.63383837 |
| (S/T) 2D1H1Q1 | 2276 | 0.63134185 |
| (S/T) 2D1Q1W1 | 2267 | 0.62884533 |
| (S/T) 1H1Q1Y1 | 2222 | 0.61636274 |
| (S/T) 1D1N1Q1 | 2207 | 0.61220187 |
| (S/T) 2H1Y1 | 2150 | 0.59639059 |
| (S/T) 1D1N1Y1 | 2109 | 0.58501756 |
| (S/T) 1Q1Y2 | 1962 | 0.54424109 |
| (S/T) 1H1Q2 | 1957 | 0.54285413 |
| (S/T) 1Q1W1Y1 | 1954 | 0.54202196 |
| (S/T) 2N1Q1Y1 | 1935 | 0.53675153 |
| (S/T) 3H1Q1 | 1901 | 0.52732024 |
| (S/T) 1D1H1W1 | 1879 | 0.52121764 |

94 of the 97 selected designs were well-expressed in E. coli with both monomers co-purifying by Ni-affinity chromatography (only one monomer contains a hexahistidine tag). For 85/94, the dominant species observed in size exclusion chromatography (SEC) had the expected size (FIG. 1f). Three designs characterized by CD spectroscopy were found to be all alpha helical and stable at 95° C. (FIG. 1g, FIG. 6). Sequences and other information on the designs are provided in Tables 1A-B (above).

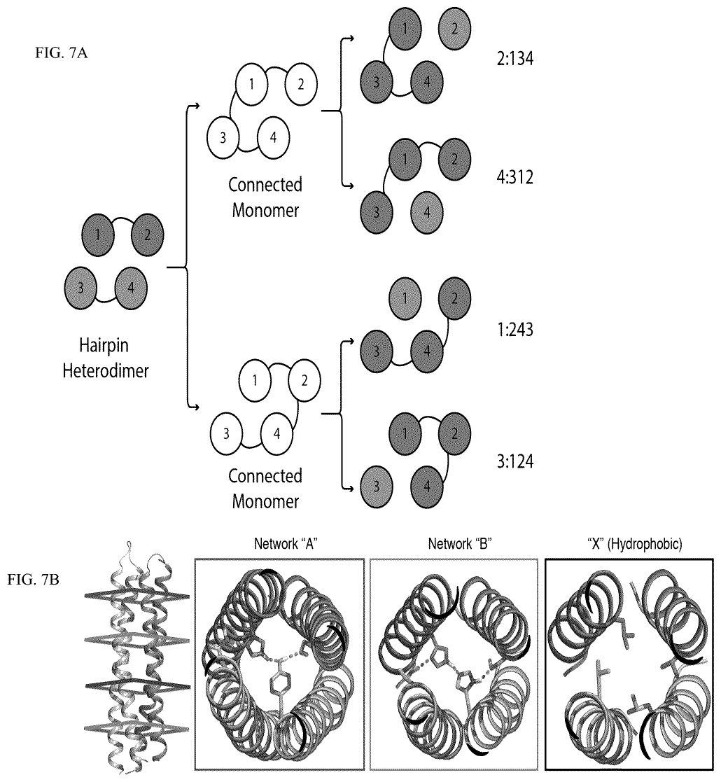

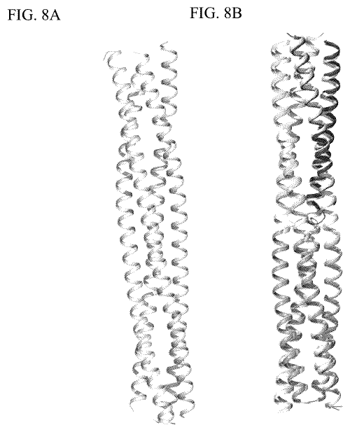

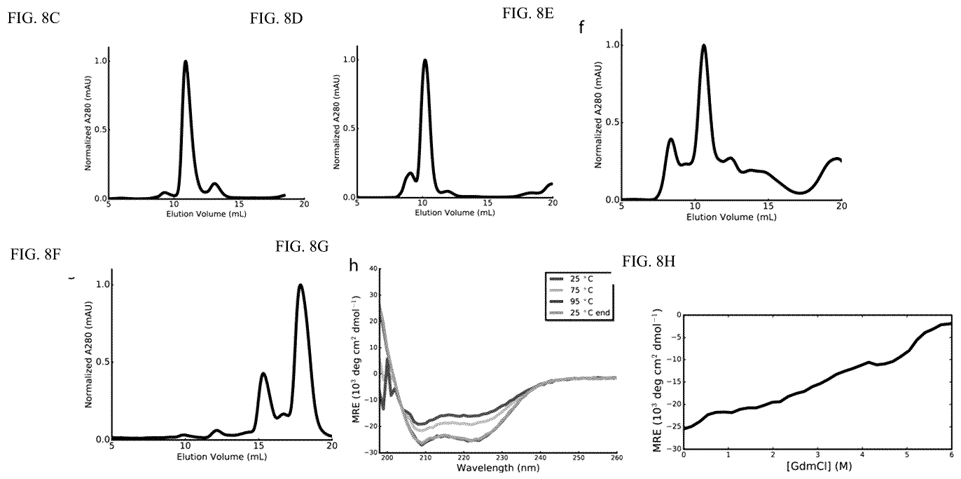

We explored the extent to which the heterodimer set could be expanded by permuting the hydrogen bond networks in the different helical repeat units, and by permuting the backbone connectivity. Assigning each unique network a letter, DHD37_XBBA indicates a variant where the second, third and fourth repeat units have hydrogen bond networks B, B, and A, and the first heptad has exclusively hydrophobic residues in the core, while DHD103_1:423 indicates a heterodimer where one monomer consists of the first helix of DHD103 and the other monomer consists of helices 2 through 4 (FIG. 7). 13 of 14 hydrogen bond network permuted variants and 9 of 10 “3+1” backbone-permuted heterodimers (generated from five starting “2+2” heterodimers) ran as single peaks on SEC.

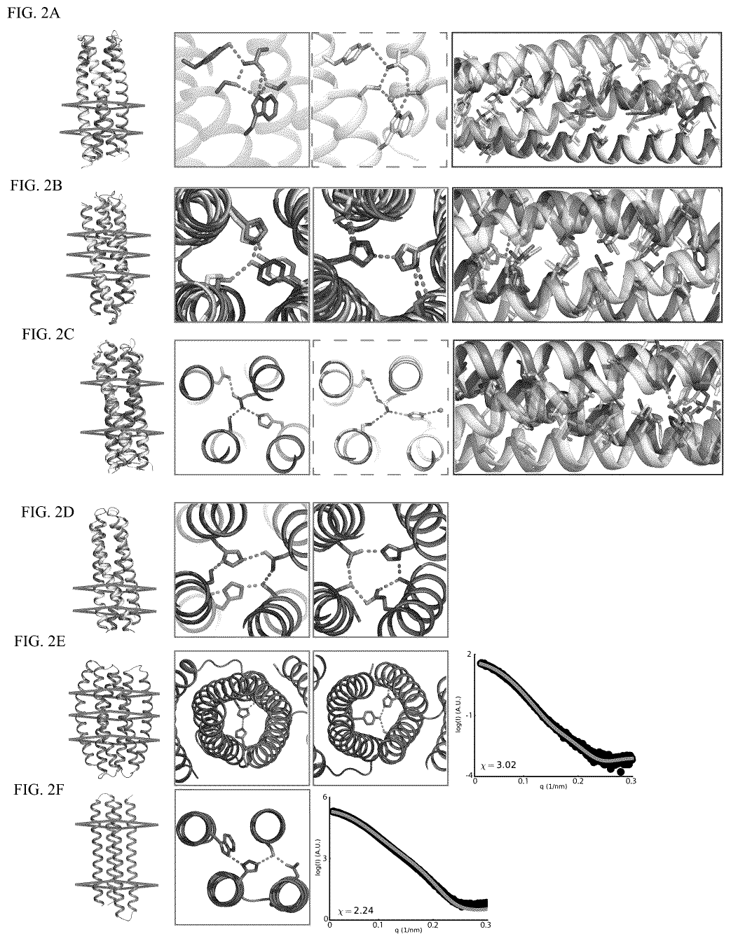

SAXS spectra collected for 44 designs were consistent with the design models (FIG. 1h, FIG. 2f-h,). The X-ray crystal structures of DHD131, DHD37_1:234, DHD127 and DHD15 had backbone Cα atom RMSDs to the design models ranging from 0.95 to 1.7 Å. The extensive five-residue buried hydrogen bond network of DHD131 (involving two serines, an asparagine, a tyrosine, and a tryptophan) is nearly identical in the crystal structure, with an additional water molecule bridging the interactions (FIG. 2a). The two designed hydrogen bond networks in DHD37_1:234, which contain buried histidine and tyrosine aromatic side chains sterically disfavoring homodimers, are in close agreement with the crystal structure (FIG. 2b). In DHD127, the histidines in the two hydrogen bond networks adopt a rotamer different from the design model (FIG. 2c), making a hydrogen bond with a water molecule. A crystal structure of DHD15 at pH 7.0 is similar to the design model (FIG. 2d), while a structure at pH 6.5 is of a domain-swapped, hetero-tetramer conformation.

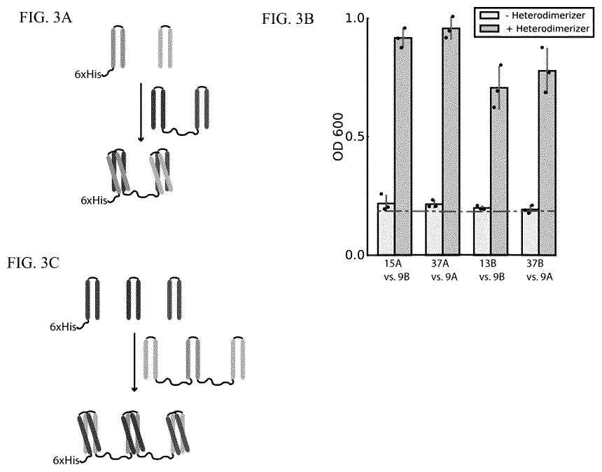

We built three induced dimerization systems by fusing one monomer each from two different heterodimers via a flexible linker, and testing whether the remaining two monomers from each pair could be brought together by the fusion (FIG. 3a). In each case, the three components co-purified by Ni-NTA chromatography (one monomer has a hexahistidine tag); In yeast two-hybrid assays (Y2H) with monomers from two different heterodimers fused to the DNA binding domain (DBD) and transcriptional activation domain (AD), expression of the heterodimerizer fusion as a separate polypeptide chain increased signal significantly over background (FIG. 3b).

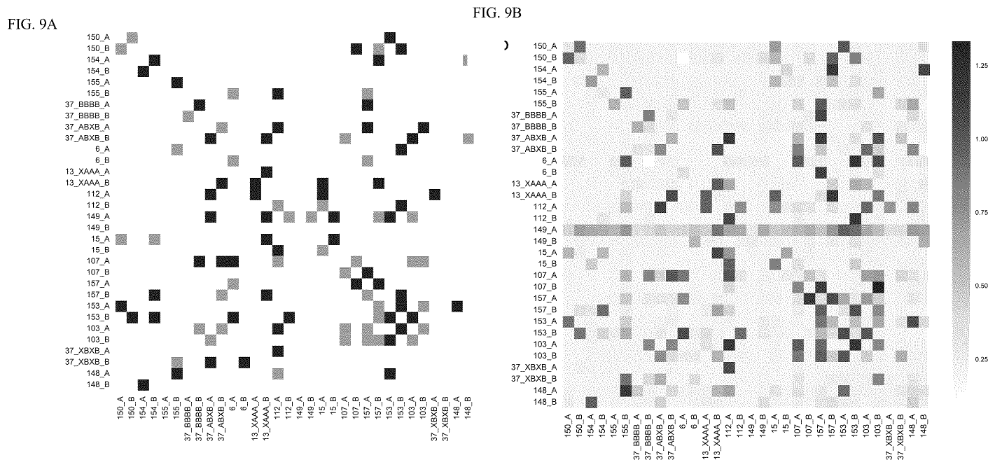

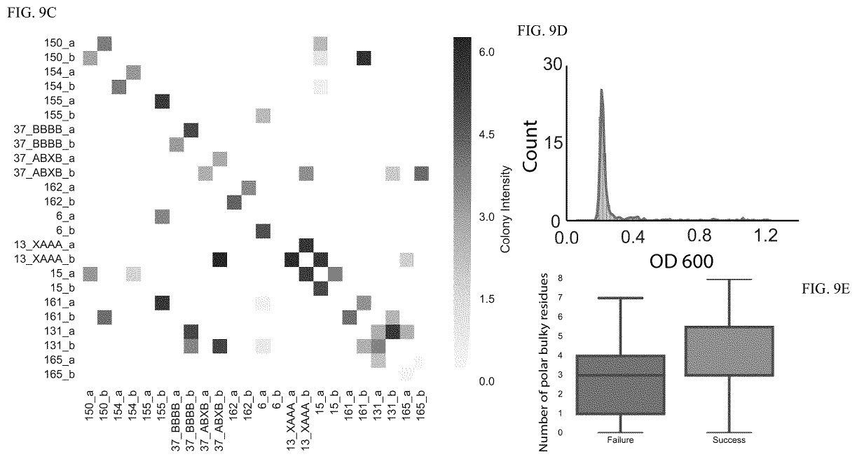

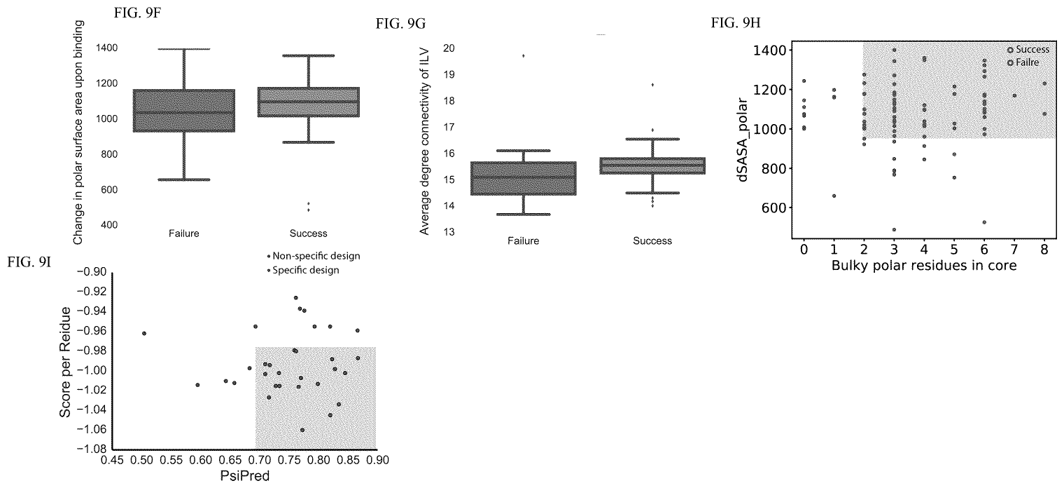

We covalently linked the monomer chain “a” subunits of 3 DHDs via flexible linkers (FIG. 3C), and co-expressed this “scaffold” and the 3 separate chain “b” monomers, one with a hexahistidine tag, in E. coli. The scaffold plus monomer assembly is stable at 95° C. and has a guanidine denaturation midpoint of 4 M (FIG. 9).

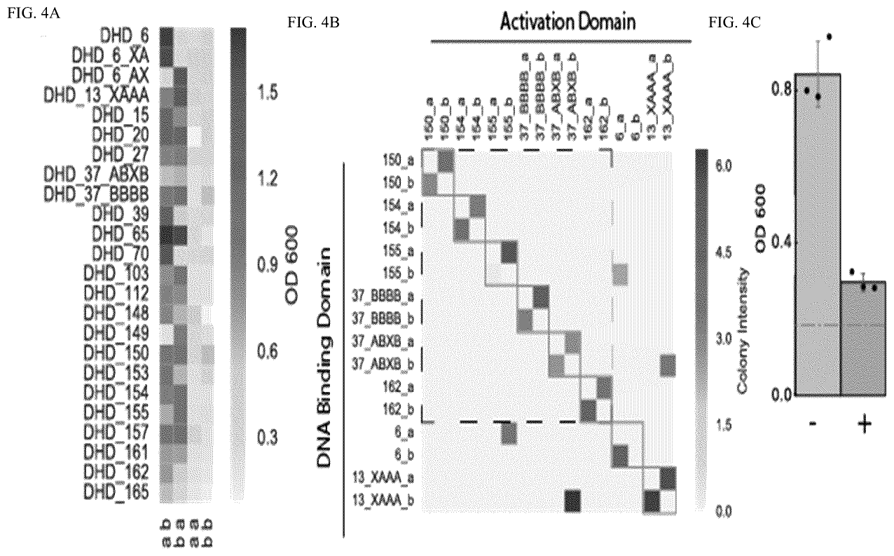

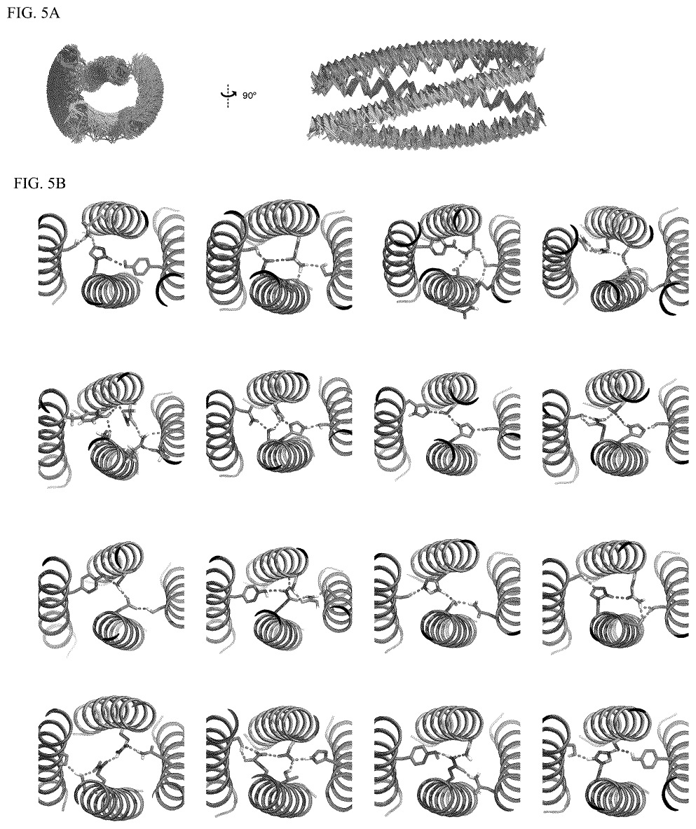

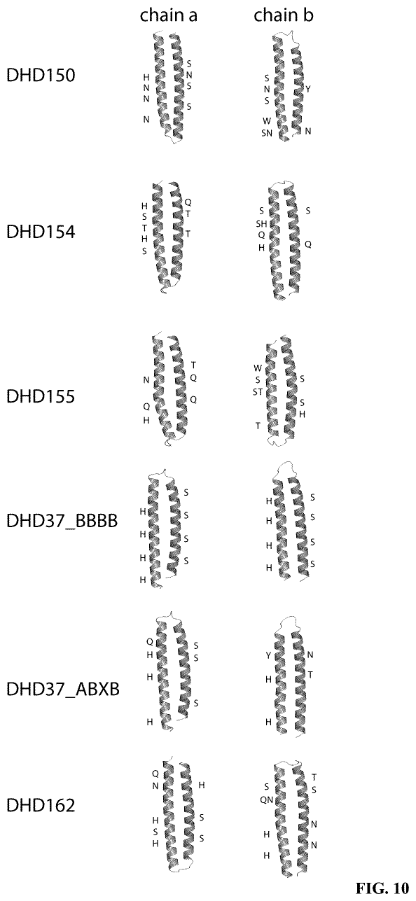

By generating interfaces with many polar groups which are energetically costly to bury without geometrically matched hydrogen bonding interactions, our design protocol implicitly disfavors non-cognate interactions (explicit negative design to disfavor non-cognate interactions is computationally intractable given the very large number of possible off-target binding modes). For 24 designs, strong interactions were observed by Y2H with the two partners fused to DBD and AD, but not when either partner was fused to both domains; the designed heterodimers, but not the homodimers, form in cells (FIG. 4A). The 24 monomers in 12 of these designs were crossed in an all-by-all Y2H experiment; interactions were observed for all cognate pairs, and 27 of the 552 possible non-cognate interactions (FIG. 9). Orthogonality was higher for an 8 DHD subset: of 240 possible non-cognate interactions, only 4 were observed (FIG. 4B; the interacting polar residues are depicted schematically in FIG. 10). Co-expression of unfused monomers eliminated off-target interactions (FIG. 4C); the cognate interactions are evidently stronger than the non-cognate interactions.

Our results demonstrate that the domain of unbounded sets of orthogonal heterodimeric biomolecules constructed from a single repeating backbone is not limited to nucleic acids. Interaction specificity arises from extensive buried hydrogen bond networks such as the fully connected TYR-SER-TRP-ASN-SER (SEQ ID NO:333) crystallographically confirmed network in FIG. 2a, and heterogeneity in the size of the residues at the designed interface (FIG. 9d-i), analogous to the contribution of steric effects to Watson-Crick base pairing specificity. Our large set of orthogonal interactions, together with the retention of specificity in the fused monomer systems (the induced dimerizer and scaffold of FIG. 3), and the interaction strength hierarchy illustrated by the cognate interaction competition experiment (FIG. 4c), can be used, by way of non-limiting example, to prepare protein based cellular control circuits with faster response times and better integration with signaling inputs and outputs than current nucleic acid based circuitry.

### Methods for Example 1

### Computational Design

### 1. Systematic Sampling of Parametric Helical Backbones

We used a generalization of the Crick coiled-coil parameters⁵ to independently sample all four helices of the heterodimers supercoiled around the same axis. The supercoil twist (ω₀) and helical twist (ω₁) were coupled and ideal values were used²⁰ with ω₀ and ω₁ held constant among the helices. A left-handed supercoil results from ω₀=−2.85 and ω₁=102.85, and a straight bundle with no supercoiling from ω₀=0 and ω₁=100. The supercoil phases (ΔΦ₀) for the helices were fixed at 0°, 90°, 1800 and 270°, respectively. The offset along the Z-axis (Z offset) for the first helix was fixed to 0 as a reference point, with the rest of the helices independently sampling from −1.51 Å to 1.51 Å, with a step size of 1.51 Å. All helices sampled helical phases (ΔΦ₁) independently, from 0° to 90°, with a step size of 10°. Two of the helices with a ΔΦ₀ separation of 1800 sampled the radius from Z-axis (R) from 5 Å to 8 Å, while the other two sampled from 7 Å to 10 Å, all with a step size of 1 Å. Each helix is set to have 35 residues to accommodate 5 heptad repeats. After removing redundant sample points from the overlapping regions of radii sampling, the supercoiled helical bundles contained more than 60 million unique backbones, and the straight helical bundles contained more than 27 million unique backbones.

### 2. HBNet Search

For each parametrically generated backbone, HBNet™²¹ was used to search the middle heptad for hydrogen bond networks that connect all four helices, contain at least four side chains contributing hydrogen bonds, have all heavy atom donors and acceptors satisfied, and span the intermolecular interface. Symmetry was not enforced during the HBNet™ search. For buried interface positions, only non-charged polar amino acids were considered; for residues that were at the boundary between protein core and surface, all polar amino acids were considered. A subsequent Rosetta™ design calculation was performed to optimize hydrophobic packing, with atom pair restraints from HBNet™ being put on the newly identified hydrogen bond networks. Finally, a minimization step and side chain repacking step was performed without atom pair restraints on hydrogen bonding residues to evaluate how well the networks remained intact in the absence of the constraints. Designs with at most 5 alanines in the middle heptad and no buried unsatisfied polar heavy atoms were selected for downstream design.

3. Generating Combinations of HBNets™ with Heptad Stacking

The purpose of this step is to identify five-heptad backbones (full backbones) that can accommodate at least 2 HBNets™. Instead of generating one-heptad backbones and full backbones separately, searching for HBNets™ in the one-heptad backbones and aligning them to all full backbones, we reasoned the heptad stacking method remains the same if one simply searches for HBNets™ in the middle heptad on all full backbones, extracts the middle heptads, and aligns them to all full backbones. We therefore extracted the middle heptads containing HBNets™, generated all variants of chain ordering, and did pairwise alignment of middle heptads to full backbones using TMalign³⁰. All alignments with root mean square deviation (RMSD) less than 0.3 were identified and full backbones that can accommodate at least 2 middle heptads were selected for final design.

### 4. Connecting Parametric Helical Backbones

Helical backbones are connected with short 2-5 residue loops such that the RMSD of each loop is less than 0.4 RMSD to a nine residues stretch in a native protein. Distance and directionality between helices limit what loops can connect, as such, our closure extends and shrinks helices by up to 3 residues. We then superimpose all short loops from the PDB onto the first and last two helical residues. The loops with the lowest stub-RMSD are minimized using the Rosetta™ score function onto the helical endpoints to ensure a near perfect closure. Loop quality is assessed by measuring the distance in RMSD to the closest nine stretch in the PDB. The loop with the lowest RMSD is returned as the solution. We repeat this procedure to connect all helices and report the solution with the lowest RMSD.

### 5. Design Calculations

Backbones were regularized using Cartesian space minimization in Rosetta™ to alleviate any torsional strain introduced by heptad stacking. Two consecutive Rosetta™ packing rounds were performed with increasing weight on the repulsive energy to optimize hydrophobic packing, while constraining the hydrogen bond network residues. A FastDesign step was subsequently used within a generic Monte Carlo mover to optimize secondary structure shape complementarity, while allowing at most 8% alanine, 3 methionine and 3 phenylalanine in the protein core. The last step of minimization and side chain repacking to identify the movement of HBNets without atom pair constraints is the same as what was described in Step 2.

### 6. Selection Criteria and Metrics Used to Evaluate Designs

Designs were selected based on the following criteria: change in polar surface area upon binding (dSASA_polar) greater than 800 Å; secondary structure shape complementarity (ss_sc) score greater than 0.65; holes score around HBNets less than −1.4; no buried unsatisfied heavy atoms; at least one buried bulky polar side chains per monomer. Selected designs were then visually inspected for good packing of hydrophobic side chains, especially the interdigitation of isoleucine, leucine and valine. Surface tyrosines were added at non-interfering positions to aid protein concentration measurement by recording OD280. Surface charge residues for a few of the designs were redesigned to shift the theoretical isoelectric point away from buffer pH.

### RMSD Calculations

Crystal structures and the corresponding design models were superimposed with TMalign using all heavy atoms. From this alignment, RMSD was calculated across all alpha-carbon atoms, and also across heavy atoms of the hydrogen bond network residues.

### Logistic Regression

Designs were first scored with various filters in Rosetta™ with the filter values reported. Experimental results and Rosetta™ filter values were used as input to a logistic regression method³¹ to find correlations between computational metrics and experimental observations.

### Visualization and Figures

All structural images for figures were generated using PyMOL³².

### Buffer and Media Recipe

TBM-5052: 1.2% [wt/vol] tryptone, 2.4% [wt/vol] yeast extract, 0.5% [wt/vol]glycerol, 0.05% [wt/vol] D-glucose, 0.2% [wt/vol] D-lactose, 25 mM Na2HPO4, 25 mM KH2PO4, 50 mM NH4Cl, 5 mM Na2SO4, 2 mM MgSO4, 10 μM FeCl3, 4 μM CaCl2, 2 μM MnCl2, 2 μM ZnSO4, 400 nM CoCl2, 400 nM NiCl2, 400 nM CuCl2, 400 nM Na2MoO4, 400 nM Na2SeO3, 400 nM H3B03

Lysis buffer: 20 mM Tris, 300 mM NaCl, 20 mM Imidazole, pH 8.0 at room temperature

Wash buffer: 20 mM Tris, 300 mM NaCl, 30 mM Imidazole, pH 8.0 at room temperature

Elution buffer: 20 mM Tris, 300 mM NaCl, 250 mM Imidazole, pH 8.0 at room temperature

Buffer W: 100 mM Tris-HCl pH 8.0, 150 mM NaCl and 1 mM EDTA

Buffer E: Buffer W containing 2.5 mM D-desthiobiotin

TBS buffer: 20 mM Tris pH 8.0, 100 mM NaCl

### Construction of Synthetic Genes

For the expression of heterodimers, both monomers were encoded in the same plasmid, separated by a ribosome binding sequence (GAAGGAGATATCATC; SEQ ID NO:327). Synthetic genes were ordered from Genscript Inc. (Piscataway, N.J., USA) and delivered in pET21-NESG E. coli expression vector, inserted between the NdeI and XhoI sites. For the pET21-NESG constructs, a hexahistidine tag and a tobacco etch virus (TEV) protease cleavage site (GSSHHHHHHSSGENLYFQGS; SEQ ID NO:328) were added in frame at the N-terminus of the second monomer. A stop codon was introduced at the 3′ end of the second monomer to stop expression of the C-terminal hexahistidine tag in the vector. For purification with Strep-tactin resin, a streptavidin tag (SAWSHPQFEKGGGSGGGSGGSAWSHPQFEKSGENLYFQGS; SEQ ID NO:329) coding sequence was cloned in frame 5′ of the first monomer sequence.

For the co-expression of 3 and 4 proteins from the same plasmid (induced dimerization and synthetic scaffold designs), synthetic genes were cloned in the pRSFDuet-1 expression vector. The first (in the case of 3 proteins) or first two (in the case of 4 proteins) genes were cloned between NcoI and HindIII sites, with a ribosome binding site separating the 2 proteins in the latter case. The last two genes were cloned between NdeI and XhoI sites, separated by a ribosome binding site. A hexahistidine tag and a TEV protease cleavage site coding sequence were cloned in frame 5′ of the last gene.

Genes for yeast-two-hybrid (Y2H) studies were cloned into plasmids bearing the GAL4 transcription activation domain (poAD) and the GAL4 DNA-binding domain (poDBD).

### Protein Expression

Plasmids were transformed into chemically competent E. coli expression strains BL21(DE3)Star (Invitrogen) or Lemo21™ (DE3) (New England Biolabs) for protein expression. Single colonies were picked from agar plates following transformation and growth overnight, and 5 ml starter cultures were grown at 37° C. in Luria-Bertani (LB) medium containing 100 μg/mL carbenicillin (for pET21-NESG vectors) or kanamycin (for pRSFDuet-1 vectors) with shaking at 225 rpm for 18 hours at 37° C. Starter cultures were diluted into 500 ml TBM-5052 containing 100 μg/mL carbenicillin or kanamycin, and incubated with shaking at 225 rpm for 24 hours at 37° C.

For expression of ¹³C1⁵ N- or ¹⁵N-labeled protein, the plasmids were transformed into the Lemo21™ (DE3) E. co/i expression strain and plated on M9/glucose plates containing 50 μg/mL carbenicillin. For the starter culture, a single colony was used for inoculation of 50 mL LB medium with 50 μg/mL carbenicillin in a 250 mL baffled flask, and incubated with shaking at 225 rpm for 18 hours at 37° C. 10 mL of the starter culture was then transferred to a 2 L baffled flask containing 500 mL of Terrific Broth™ (Difco), with 25 mM Na2HPO4, 25 mM KH2PO4, 50 mM NH4Cl, 5 mM Na2SO4, and 100 μg/mL carbenicillin. The culture was grown at 37° C. to an OD600 of approximately 1.0, then centrifuged at 5000 ref for 15 minutes to pellet the cells. The Terrific Broth™ medium was removed, and the cells were washed briefly with 30 mL of phosphate buffered saline (PBS). The cells were then transferred to a fresh 2 L baffled flask containing 500 mL of labeled media (25 mM Na2HPO4, 25 mM KH2PO4, 50 mM 15NH4C1, 5 mM Na2SO4, 0.2% (w/v)¹³C glucose), and 100 μg/mL carbenicillin. The cells were allowed to grow at 37° C. for 2 hours, before IPTG (Carbosynth) was added to 1 mM and the temperature was reduced to 18° C. The labeled glucose and NH4Cl were obtained from Cambridge Isotopes.

### Affinity Purification

Cells were harvested by centrifugation for 15 minutes at 5000 rcf at 4° C. and resuspended in 20 ml lysis buffer. Lysozyme, DNAse, and EDTA-free cocktail protease inhibitor (Roche) were added to the resuspended cell pellet before sonication at 70% power for 5 minutes. For Immobilized metal affinity chromatography (IMAC), lysates were clarified by centrifugation at 4° C. and 18,000 rpm for at least 30 minutes and applied to Ni-NTA (Qiagen) columns pre-equilibrated with lysis buffer. The column was washed two times with 5 column volumes (CV) of wash buffer, followed by 5 CV of elution buffer. For Strep tag purification, elution fractions from IMAC were applied to Strep-Tactin® Superflow resin (IBA) pre-equilibrated in Buffer W. The column was washed with 5 CV Buffer W, before applying 3 CV Buffer E to elute proteins off the column. Mass and purity of eluted proteins were confirmed using electrospray ionization mass spectrometry (ESI-MS) on a Thermo Scientific TSQ Quantum Access mass spectrometer.

### Size-Exclusion Chromatography (SEC)

N-terminal hexahistidine tags and streptavidin tags were cleaved with TEV protease overnight at room temperature, at a ratio of 1 mg TEV for 100 mg of protein. Prior to addition of TEV, buffer was exchanged into lysis buffer. After TEV cleavage, sample was passed over an additional Ni-NTA column and washed with 1.5 CV of lysis buffer, flow through were collected and further purified by SEC using a Superdex™ 75 10/300 increase column (GE Healthcare) in TBS buffer.

### Circular Dichroism (CD) Measurements

CD wavelength scans (260 to 195 nm) and temperature melts (25 to 95° C.) were performed using an AVIV model 420 CD spectrometer. Temperature melts were carried out at a heating rate of 4° C./min and monitored by the change in ellipticity at 222 nm; protein samples were diluted to 0.25 mg/mL in PBS pH 7.4 in a 0.1 cm cuvette. Guanidinium chloride (GdmCl) titrations were performed on the same spectrometer with automated titration apparatus in PBS pH 7.4 at 25° C., with a protein concentration of 0.025 mg/mL in a 1 cm cuvette with stir bar. Each titration consisted of at least 40 evenly distributed GdmCl concentration points with one minute mixing time for each step. Titrant solution consisted of the same concentration of protein in PBS+GdmCl.

### Crystallization of Protein Samples

Purified protein samples were concentrated to approximately 20 mg/ml in 25 mM Tris pH 8.0 and 150 mM NaCl. Samples were screened with a 5-position deck Mosquito™ crystal (ttplabtech) with an active humidity chamber, utilizing the following crystallization screens: JCSG+™ (Qiagen), Crystal Screen™ (Hampton Research), PEG/Ion™ (Hampton Research), PEGRx HT™ (Hampton Research), Index™ (Hampton Research) and Morpheus™ (Molecular Dimensions). The optimal conditions for crystallization of the different designs were found as follows: OPHD_37_N3C1, 0.15 M potassium bromide and 30% w/v polyethylene glycol monomethyl ether 2000; OPHD_127, 0.12 M ethylene glycols, 0.1 M buffer system 3 pH 8,5, and 50% v/v precipitate mix 1 from the Morpheus screen; OPHD_15. 0.2 M Ammonium sulfate, 0.1 M BIS-TRIS pH 6.5, 18% v/v Polyethylene glycol 400; OPHD_15, 0.1 M Imidazole pH 7.0, and 25% v/v Polyethylene glycol monomethyl ether 550; OPHD_131, 0.2 M Ammonium acetate, 0.1 M HEPES pH 7.5, 25% w/v Polyethylene glycol 3,350. Crystals were obtained after 1 to 14 days by the hanging drop vapor diffusion method with the drops consisting of a 1:1, 2:1 and 1:2 mixture of protein solution and reservoir solution.

### X-Ray Data Collection and Structure Determination

The crystals of the designed proteins were looped and placed in the corresponding reservoir solution, containing 20% (v/v) glycerol if the reservoir solution did not contain cryoprotectant, and flash-frozen in liquid nitrogen. The X-ray data sets were collected at the Advanced Light Source at Lawrence Berkeley National Laboratory with beamlines 8.2.1 and 8.2.2. Data sets were indexed and scaled using either XDS³³ or HKL2000³⁴. Initial models were generated by the molecular-replacement method with the program PHASER 35 within the Phenix™ software suite³⁶, using the design models as the initial search models. Efforts were made to reduce model bias through refinement with simulated annealing using Phenix.Refine™³⁷, or, if the resolution was sufficient, by using Phenix.Autobuild™³⁸ with rebuild-in-place set to false, simulated annealing and prime-and-switch phasing. Iterative rounds of manual building in COOT 39 and refinement in Phenix™ were used to produce the final models. Due to the high degree of self-similarity inherit in coiled-coil-like proteins, datasets for the reported structures suffered from a high degree of pseudo translational non-crystallographic symmetry, as report by Phenix.Xtriage™, which complicated structure refinement and may explain the higher than expected R values reported. RMSDs of bond lengths, angles and dihedrals from ideal geometries were calculated with Phenix™³⁶. The overall quality of all final models was assessed using the program MOLPROBITY™⁴⁰.

### Small Angle X-Ray Scattering (SAXS)

Samples were purified by SEC in 25 mM Tris pH 8.0, 150 mM NaCl and 2% glycerol; fractions preceding the void volume of the column were used as blanks for buffer subtraction. Scattering measurements were performed at the SIBYLS™ 12.3.1 beamline at the Advanced Light Source. The X-ray wavelength (k) was 1.27 Å, and the sample-to-detector distance was 1.5 m, corresponding to a scattering vector q (q=4π sin θ/λ, where 2θ is the scattering angle) range of 0.01 to 0.3 Å⁻¹. A series of exposures, in equal sub-second time slices, were taken of each well: 0.3 second exposures for 10 seconds resulting in 32 frames per sample. For each sample, data was collected for two different concentrations to test for concentration-dependent effects; “low” concentration samples ranged from 2-3 mg/mL and “high” concentration samples ranged from 5-7 mg/mL. Data was processed using the SAXS FrameSlice™ online serve and analyzed using the ScAtter™ software package^{41,42} FoXS™^{43,44} was used to compare design models to experimental scattering profiles and calculate quality of fit (x) values.

### Yeast Two-Hybrid Assay

For each pair of binders tested, chemically competent cells of yeast strain PJ69-4a (MATatrpl-901 leu2-3,112 ura3-52 his3-200 gal4(deleted) gal80(deleted) LYS2::GAL1-HIS3 GAL2-ADE2 met2::GAL7-lacZ) were transformed with the appropriate pair of plasmids containing DNA binding domain or activation domains, using the LiAc/SS carrier DNA/PEG method 4. In the case of induced dimerization, the heterodimerizer was cloned downstream of one of the “monomer proteins”, separated by a p2a and nuclear locolization sequence (GSGATNFSLLKQAGDVEENPGPGDKAELIPEPPKKKRKVELGTA; SEQ ID NO:330). The p2a sequence ensures translational cleavage to make the heterodimerizer a separate protein from the “monomer protein”. The selection of transformed yeast cells was performed in synthetic dropout (SDO) media lacking tryptophan and leucine for 48 hours with shaking at 1000 rpm at 30° C. The resulting culture was diluted 1:100 and grown for 16 hours in fresh SDO media lacking tryptophan and leucine, before transferring to a 96 well plate and diluted 1:100 into SDO media containing 100 mM 3-Amino-1,2,4-triazole (3-AT), lacking tryptophan, leucine and histidine (5 mM 3-AT in the case of induced dimerization). The culture was incubated with shaking at 1000 rpm at 30° C. Since bringing the DNA binding domain and the transcription activation domain into proximity is necessary for the growth of yeast cells in media lacking histidine, binding of two proteins was indicated by the growth of yeast cells^{46,47}. The optical density of yeast cells was recorded after 48 hours. For Y2H assay on agar plates, the 1:100 diluted overnight culture was transferred onto Nunc™ OmniTray™ (Thermo Fisher) using a 96 Solid Pin Multi-Blot Replicator (V&P Scientific), with the agar lacking tryptophan, leucine and histidine, and containing 100 mM 3-AT. The plates were imaged daily until Day 5 to monitor the sizes of colonies. Images were analyzed by the ColonyArea⁴⁸ package on ImageJ.

### REFERENCES FOR EXAMPLE 1

1. Jones, S. & Thornton, J. M. Principles of protein-protein interactions. Proc. Natl. Acad. Sci. U.S.A. 93, 13-20 (1996).
2. Harbury, P. B., Zhang, T., Kim, P. S. & Alber, T. A switch between two-, three-, and four-stranded coiled coils in GCN4 leucine zipper mutants. Science 262, 1401-1407 (1993).
3. Diss, M. L. & Kennan, A. J. Orthogonal recognition in dimeric coiled coils via buried polar-group modulation. J. Am. Chem. Soc. 130, 1321-1327 (2008).
4. Thomas, F., Boyle, A. L., Burton, A. J. & Woolfson, D. N. A set of de novo designed parallel heterodimeric coiled coils with quantified dissociation constants in the micromolar to sub-nanomolar regime. J. Am. Chem. Soc. 135, 5161-5166 (2013).
5. Crick, F. H. C. The Fourier transform of a coiled-coil. Acta Cryst (1953). Q6, 685-689 [doi:10.11071S0365110X53001952] 6, 1-5 (1953).
6. Zarrinpar, A., Park, S.-H. & Lim, W. A. Optimization of specificity in a cellular protein interaction network by negative selection. Nature 426, 676-680 (2003).
7. Aakre, C. D. et al. Evolving new protein-protein interaction specificity through promiscuous intermediates. Cell 163, 594-606 (2015).
8. Joachimiak, L. A., Kortemme, T., Stoddard, B. L. & Baker, D. Computational design of a new hydrogen bond network and at least a 300-fold specificity switch at a protein-protein interface. J Mol. Biol. 361, 195-208 (2006).
9. Skerker, J. M. et al. Rewiring the specificity of two-component signal transduction systems. Cell 133, 1043-1054 (2008).
10. Crooks, R. O., Baxter, D., Panek, A. S., Lubben, A. T. & Mason, J. M. Deriving Heterospecific Self-Assembling Protein-Protein Interactions Using a Computational Interactome Screen. J Mol. Biol. 428, 385-398 (2016).
11. Gradisar, H. & Jerala, R. De novo design of orthogonal peptide pairs forming parallel coiled-coil heterodimers. J. Pept. Sci. 17, 100-106 (2011).
12. Thompson, K. E., Bashor, C. J., Lim, W. A. & Keating, A. E. SYNZIP protein interaction toolbox: in vitro and in vivo specifications of heterospecific coiled-coil interaction domains. ACS Synth. Biol. 1, 118-129 (2012).
13. Reinke, A. W., Grant, R. A. & Keating, A. E. A synthetic coiled-coil interactome provides heterospecific modules for molecular engineering. J. Am. Chem. Soc. 132, 6025-6031 (2010).
14. Acharya, A., Rishi, V. & Vinson, C. Stability of 100 homo and heterotypic coiled-coil a-a′ pairs for ten amino acids (A, L, I, V, N, K, S, T, E, and R). Biochemistry 45, 11324-11332 (2006).
15. Grigoryan, G. & Keating, A. E. Structure-based prediction of bZIP partnering specificity. J Mol. Biol. 355, 1125-1142 (2006).
16. Gonzalez, L., Jr, Woolfson, D. N. & Alber, T. Buried polar residues and structural specificity in the GCN4 leucine zipper. Nat. Struct. Biol. 3, 1011-1018 (1996).
17. Lumb, K. J. & Kim, P. S. A buried polar interaction imparts structural uniqueness in a designed heterodimeric coiled coil. Biochemistry 34, 8642-8648 (1995).
18. Tatko, C. D., Nanda, V., Lear, J. D. & DeGrado, W. F. Polar Networks Control Oligomeric Assembly in Membranes. J. Am. Chem. Soc. 128, 4170-4171 (2006).
19. Grigoryan, G. & DeGrado, W. F. Probing Designability via a Generalized Model of Helical Bundle Geometry. J. Mol. Biol. 405, 1079-1100 (2011).
20. Huang, P.-S. et al. High thermodynamic stability of parametrically designed helical bundles. Science 346, 481-485 (2014).
21. Boyken, S. E. et al. De novo design of protein homo-oligomers with modular hydrogen-bond network-mediated specificity. Science 352, 680-687 (2016).
22. Leaver-Fay, A. et al. ROSETTA™3: an object-oriented software suite for the simulation and design of macromolecules. Methods Enzymol. 487, 545-574 (2011).
23. Ruotolo, B. T. & Robinson, C. V. Aspects of native proteins are retained in vacuum. Curr. Opin. Chem. Biol. 10, 402-408 (2006).
24. Sahasrabuddhe, A. et al. Confirmation of intersubunit connectivity and topology of designed protein complexes by native MS. Proc. Natl. Acad. Sci. U.S.A. 115, 1268-1273 (2018).
25. Zhou, M., Huang, C. & Wysocki, V. H. Surface-induced dissociation of ion mobility-separated noncovalent complexes in a quadrupole/time-of-flight mass spectrometer. Anal. Chem. 84, 6016-6023 (2012).
26. Zhou, M. & Wysocki, V. H. Surface induced dissociation: dissecting noncovalent protein complexes in the gas phase. Acc. Chem. Res. 47, 1010-1018 (2014).
27. Anderson, G. P., Shriver-Lake, L. C., Liu, J. L. & Goldman, E. R. Orthogonal Synthetic Zippers as Protein Scaffolds. ACS Omega 3, 4810-4815 (2018).
28. Rothemund, P. W. K. Folding DNA to create nanoscale shapes and patterns. Nature 440, 297-302 (2006).
29. Qian, L. & Winfree, E. Scaling up digital circuit computation with DNA strand displacement cascades. Science 332, 1196-1201 (2011).
30. Zhang, Y. & Skolnick, J. TM-align: a protein structure alignment algorithm based on the TM-score. Nucleic Acids Res. 33, 2302-2309 (2005).
31. Rocklin, G. J. et al. Global analysis of protein folding using massively parallel design, synthesis, and testing. Science 357, 168-175 (2017).
32. Schrödinger, LLC. The PyMOL Molecular Graphics System, Version 1.8. (2015).
33. Kabsch, W. XDS. Acta Crystallogr. D Biol. Crystallogr. 66, 125-132 (2010).
34. Otwinowski, Z. & Minor, W. Processing of X-ray diffraction data collected in oscillation mode. Methods Enzymol. 276, 307-326 (1997).
35. McCoy, A. J. et al. Phaser crystallographic software. J. Appl. Crystallogr. 40, 658-674 (2007).
36. Adams, P. D. et al. PHENIX: a comprehensive Python-based system for macromolecular structure solution. Acta Crystallogr. D Biol. Crystallogr. 66, 213-221 (2010).
37. Afonine, P. V. et al. Joint X-ray and neutron refinement with phenix.refine. Acta Crystallogr. D Biol. Crystallogr. 66, 1153-1163 (2010).
38. Terwilliger, T. C. et al. Iterative model building, structure refinement and density modification with the PHENIX AutoBuild wizard. Acta Crystallogr. D Biol. Crystallogr. 64, 61-69 (2008).
39. Emsley, P. & Cowtan, K. Coot: model-building tools for molecular graphics. Acta Crystallogr. D Biol. Crystallogr. 60, 2126-2132 (2004).
40. Davis, I. W. et al. MolProbity: all-atom contacts and structure validation for proteins and nucleic acids. Nucleic Acids Res. 35, W375-83 (2007).
41. Dyer, K. N. et al. High-throughput SAXS for the characterization of biomolecules in solution: a practical approach. Methods Mol. Biol. 1091, 245-258 (2014).
42. Rambo, R. P. & Tamer, J. A. Characterizing flexible and intrinsically unstructured biological macromolecules by SAS using the Porod-Debye law. Biopolymers 95, 559-571 (2011).
43. Schneidman-Duhovny, D., Hammel, M. & Sali, A. FoXS: a web server for rapid computation and fitting of SAXS profiles. Nucleic Acids Res. 38, W540-4 (2010).
44. Schneidman-Duhovny, D., Hammel, M., Tamer, J. A. & Sali, A. Accurate SAXS profile computation and its assessment by contrast variation experiments. Biophys. J. 105, 962-974 (2013).
45. Schiestl, R. H. & Gietz, R. D. High efficiency transformation of intact yeast cells using single stranded nucleic acids as a carrier. Curr. Genet. 16, 339-346 (1989).
46. Chien, C. T., Bartel, P. L., Stemglanz, R. & Fields, S. The two-hybrid system: a method to identify and clone genes for proteins that interact with a protein of interest. Proc. Natl. Acad. Sci. U.S.A. 88, 9578-9582 (1991).
47. Bartel, P. L., Roecklein, J. A., SenGupta, D. & Fields, S. A protein linkage map of Escherichia coli bacteriophage T7. Nat. Genet. 12, 72-77 (1996).
48. Guzmán, C., Bagga, M., Kaur, A., Westermarck, J. & Abankwa, D. ColonyArea: an ImageJ plugin to automatically quantify colony formation in clonogenic assays. PLoS One 9, e92444 (2014).
49. Dyachenko, A. et al. Tandem Native Mass-Spectrometry on Antibody-Drug Conjugates and Submillion Da Antibody-Antigen Protein Assemblies on an Orbitrap EMR Equipped with a High-Mass Quadrupole Mass Selector. Anal. Chem. 87, 6095-6102 (2015).
50. Waitt, G. M., Xu, R., Wisely, G. B. & Williams, J. D. Automated in-line gel filtration for native state mass spectrometry. J. Am. Soc. Mass Spectrom. 19, 239-245 (2008).
51. Marty, M. T. et al. Bayesian deconvolution of mass and ion mobility spectra: from binary interactions to polydisperse ensembles. Anal. Chem. 87, 4370-4376 (2015).
52. Bern, M. et al. Parsimonious Charge Deconvolution for Native Mass Spectrometry. J. Proteome Res. 17, 1216-1226 (2018).
53. Jones, D. T. Protein secondary structure prediction based on position-specific scoring matrices. J. Mol. Biol. 292, 195-202 (1999).

### Example 2. Orthogonal Protein Heterodimers for Designing Modular Protein Logic Gates

Abstract: The de novo design of modular protein logic for regulating protein function at the post-transcriptional level is a challenge for computational protein design and could have wide ranging applications in synthetic biology. Here we describe the design of 2-input AND, OR, NAND, NOR, XNOR, and NOT gates built from de novo designed proteins that regulate the association of arbitrary protein units ranging from split enzymes to transcriptional machinery in vitro, and in living cells. Binding interaction cooperativity makes the gates largely insensitive to stoichiometric imbalances in the inputs, and the modularity of the approach enables ready extension to 3-input OR, AND, and disjunctive normal form gates. The modularity and cooperativity of the control elements, coupled with the ability to de novo design an essentially unlimited number of protein components, should enable design of sophisticated post-translational control logic over a wide range of biological functions.

### Introduction

The ability to de novo design protein-based logic gates with modular control of arbitrary protein-protein interactions could open the door to the tunable design of novel bio-orthogonal functionalities.

In principle, it should be possible to design a wide range of logic gates de novo using a set of orthogonal heterodimeric molecules. For example, given hypothetical heterodimer pairs A:A′, B:B′, and C:C′, an AND gate modulating the association of A with C′ can be constructed by genetically fusing A′ and B, and B′ and C: association occurs only in the presence of both A′-B, and B′-C (here and below “:” denotes noncovalent interaction, and “-”, genetic fusion via flexible linkers). Several building block properties are desirable for constructing such associative logic gates. First, there should be many mutually orthogonal heterodimeric pairs, so that gate complexity is not limited by the number of individual elements. Second, the building blocks should be modular and similar in structure so that differences in building block shape and other properties do not have to be considered when constructing the gates. Third, single building blocks should be able to bind to multiple partners with different, tunable affinities, allowing inputs to perform negation operations by disrupting pre-existing lower affinity interactions. Fourth, the interactions should be cooperative so gate activation is not sensitive to stoichiometric imbalances in the inputs. In the above AND gate, for example, if the interactions are not cooperative, a large excess of A′-B will pull the equilibrium towards partially assembled complexes (A′-B with either A or B′-C but not both), which will disrupt gate activation.

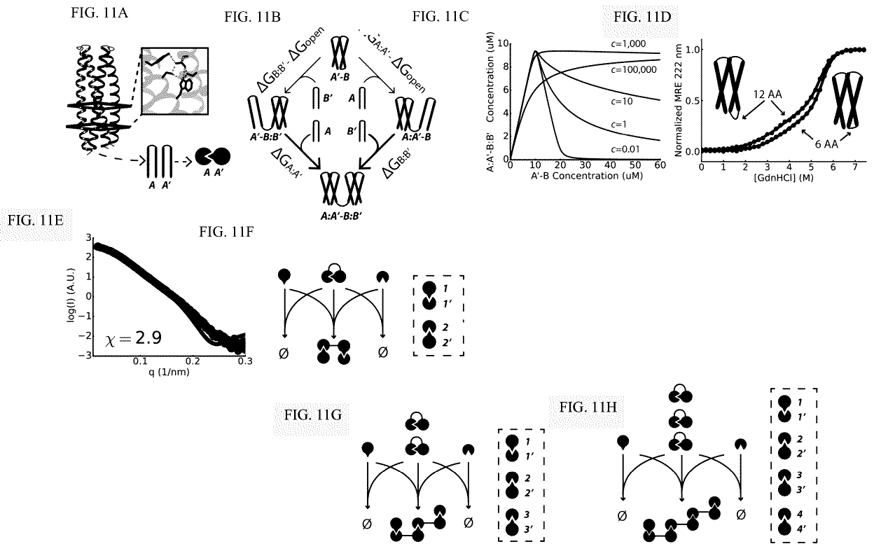

Here, we explored the possibility of designing logic gates satisfying all four of the above criteria using de novo designed protein heterodimers with hydrogen bond network-mediated specificity (34). Sets of 6 (in vivo) and 15 (in vitro) mutually orthogonal designed heterodimers (DHDs, hereafter referred to by numbers, e.g. 1 and 1′ form one cognate pair. with hydrogen bond network (see FIG. 11A inset for example) mediated specificity are available for logic gate construction, satisfying condition 1 (orthogonality). The heterodimeric interfaces all share the same four helix bundle topology (FIG. 11A), satisfying condition 2 (modularity). The shared interaction interface allows a limited amount of cross talk between pairs, leading to a hierarchy of binding affinities, satisfying condition 3 (multiple binding specificities). Inspired by cooperatively activatable systems in nature (35, 36), we sought to achieve condition 4 (cooperativity) by constructing the monomer fusions (A′-B and B′-C in the above example) in such a way that the interaction surfaces (with A and C′) are buried within the fusions. The free energy required to expose these buried interfaces would oppose gate activation, and we reasoned that the system could be tuned so that only the binding energy provided by both interactions would be sufficient to overcome this barrier, thus ensuring cooperative gate activation (FIG. 11B). If condition 2 (modularity) holds, then a single scheme for ensuring cooperativity could in principle work for a wide range of gate configurations.

### Design of Cooperativity

To explore the design of cooperative building blocks, we focused on the simple system A+A′-B+B′ (we refer to this as induced dimerization below, A and B′ as the monomers, and A′-B as the dimerizer). If binding is not cooperative, the amount of the trimeric complex decreases when A′-B is in stoichiometric excess relative to A and B′: the formation of intermediate dimeric species of the linker protein binding to either of the monomers competes with formation of trimeric complexes. On the contrary, if binding is cooperative such that no binding to either monomer occurs in the absence of the other, the amount of trimeric complex formed becomes insensitive to an excess of the dimerizer. A simple thermodynamic model of the effect of binding cooperativity on the stoichiometric response of such induced dimerization systems (FIG. 111B, supplemental materials modeling section) shows that as the binding cooperativity decreases, there is a corresponding decrease in the final concentration of full trimeric complexes at high dimerizer concentrations (FIG. 11C).

We hypothesized that a folded four helix bundle like state of the A′-B dimerizer could oppose binding to either A or B′, as the relatively hydrophobic interacting surfaces would likely be sequestered within the folded structure (FIG. 14A). We tested different flexible linker lengths connecting A′ with B using heterodimers 1:1′ and 2:2′ as a model system. All designs were found to be folded and stable in circular dichroism (CD) guanidine hydrochloride (GdnHCl) denaturation experiments, with unfolding free energies greater than 13 kcal/mol (FIG. 11D, Table 10). Although 1′-2′ dimerizer constructs with short linkers of 0 and 2 residues, or with a very long 24 residue linker could be purified as monomers (FIG. 14B), they were prone to aggregation. In contrast, designs with 6 and 12 residue linkers remained largely monomeric (data not shown). Small angle x-ray scattering (SAXS) experiments (37) indicate their hydrodynamic radii are close to those of folded four-helix bundle DHDs (FIG. 11E). Linkers in this length range likely allow the two monomers (1′ and 2′) to fold back on each other such that the largely hydrophobic interaction surfaces are buried against each other; such a structure would have to partially unfold for 1′-2′ to interact with either 1 or 2 with free energy cost ΔGₒₚₑₙ (FIG. 11B), the magnitude of which determines the extent of cooperativity for the gate. We selected a linker length of 6- or 12-residues for all of the following experiments.

We studied the cooperativity of the induced dimerizer system in vitro using native mass spectrometry (FIG. 11F). 1, 2 and 1′-2′ were separately expressed in E. coli and purified. We first measured the extent to which 1 activates the binding of 2 to 1′-2′. At 10 μM each of 2 and 1′-2′, the fraction of 2 in complex with 1′-2′ increased from 3% to 100% upon addition of 20 μM 1 (data not shown); a fold increase comparable with naturally occurring allosteric systems (35). To assess how this activation of binding influences the sensitivity of binding to stoichiometric imbalance, 10 μM 1 and 2 were titrated with increasing concentrations of 1′-2′ (FIG. 11F), and the species formed determined by nMS. The heterotrimeric 1:1′-2′:2 complex was observed over a wide range of 1′-2′ concentrations (data not shown). Even in the presence of a 6 fold excess of 1′-2′, there was no decrease in the amount of 1:1′-2′:2 formed, and neither 1:1′-2′ or 1′-2′:2 were observed (data not shown). We define cooperativity as the ratio of the affinities in the presence and absence of the other monomer, which in our model directly relates to the free energy of opening of the dimerizer (c=e^{ΔG}^{ₒₚₑₙ}^{/RT}, see supplementary materials). Matching the thermodynamic model to native MS data (data not shown) produces an estimated c value of 991,000, which corresponds to ΔGₒₚₑₙ of 7 kcal/mol. This is about half the measured unfolding free energy of 1′-2′, suggesting that binding may not require complete unfolding of the four helix bundle state of the dimerizer.

With linker units displaying cooperative binding, we reasoned that the lack of dependence on stoichiometric excesses of one of the components should extend to more complex gates. Using nMS, we investigated the cooperativity of a 2-input AND gate constructed from the two inputs 1′-3′ and 3-2′, and monomers 1 and 2 brought together by the two inputs (FIG. 11G). As the concentration of the 2 inputs was increased, the amount of heterotetrameric complex plateaued at a stoichiometry of 2:1, and then remained constant up to a molar ratio of 6:1. Very little partial complexes (heterotrimers and heterodimers) were observed, further indicating high cooperativity (data not shown). We constructed a 3-input AND gate from 1′-4′, 4-3′, and 3-2′, which together should control the association of 1 and 2 (FIG. 11H). Similar to the 2-input AND gate, the amount of full, pentameric complexes only decreased slightly at greater than stoichiometric concentrations of inputs with no detectable competing tetrameric complexes (data not shown).

### Modular Logic Gate Construction

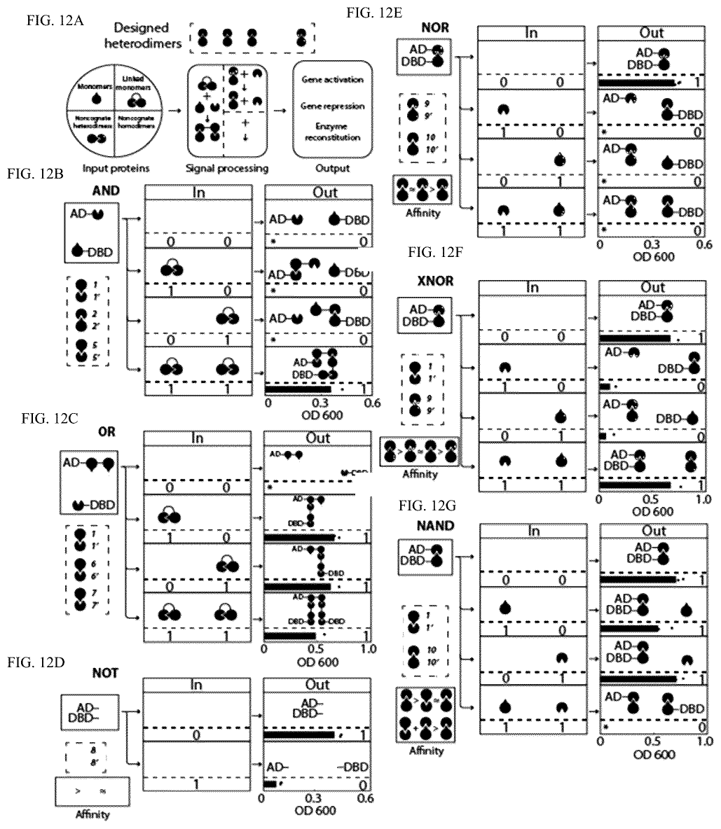

We explored the modular combination of DHDs to generate a range of 2-input Cooperatively Inducible Protein HeterodimeR (CIPHR) logic gates. Monomers from individual DHDs were linked to effector proteins of interest via genetic fusion, whose colocalization or dissociation is dependent upon the inputs. Taking advantage of previously measured all-by-all specificity matrices (34), two modes of interactions were explored: cognate binding between designed protein pairs, or competitive binding involving multispecific interactions. The choice of effector proteins is independent from the input proteins, allowing diverse functional outputs (FIG. 12A).

We used a variant of the yeast-two-hybrid (Y2H) assay to characterize the behavior of the designed logic gates, using a setup similar to previously described yeast-four-hybrid systems (38, 39). To construct an AND gate, we fused 2 to the Gal4 activation domain (AD), and 1 to the Gal4 DNA binding domain (DBD). The colocalization of AD and DBD, and resulting induction of transcription of the His3 gene, is dependent upon the expression of both input proteins (1′-5, 5′-2′). Growth in media lacking histidine required expression of both inputs (FIG. 12B). An OR gate was similarly constructed by linking the 1-6 fusion to the AD and 7′ to the DBD. Expression of either of the inputs 1′-7 or 6′-7 results in growth by driving association of AD with DBD (FIG. 12C).

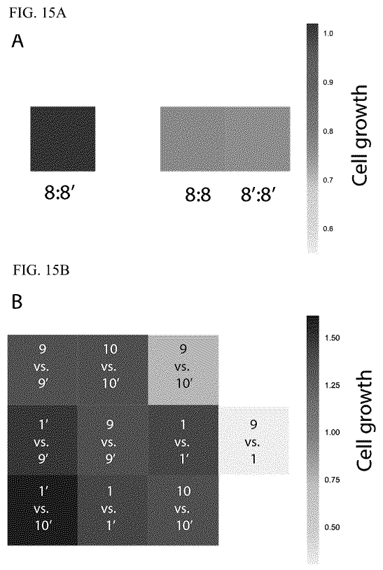

We explored the construction of additional boolean logic gates by exploiting binding affinity hierarchies identified in all by all Y2H experiments (34). 8 not only interacts with 8′ but also forms homodimers (FIG. 15A); hence 8′ must outcompete 8 homodimers to form the heterodimer. We constructed a NOT gate by fusing 8 to both AD or DBD; yeast cells stopped growing in the presence of co-expressed 8′ input protein (FIG. 12D). Based on the affinity hierarchy 9:9′≈10:10′>9:10′ (FIG. 15B), we constructed a NOR gate in which 9 was fused to the AD, 10′ to the DBD, with 9′ and 10 the two inputs. Either or both of the inputs outcompete the 9:10′ interaction and hinder yeast growth (FIG. 12E). Based on the affinity hierarchy 9′:1′>9:9′≈1:1′>9:1 (FIG. 15B), an XNOR gate was constructed by fusing 9 to 10 AD, 1 to DBD, and using 9′ and 1′ as the two inputs: the presence of either outcompetes the 9:1 binding and blocks growth, but when both are expressed they instead interact with each other and growth is observed (FIG. 12F). Similarly, a NAND gate was designed based on the interaction hierarchy 1′:10′>1:1′≈10:10′>1:10 (FIG. 15B). Neither 1 nor 10 alone can outcompete the 1′:10′ binding and hence growth occurs, but when both are expressed, the 15 free energy of formation of both 1:1′ and 10:10′ outweighs that of 1′:10′ and growth is blocked (FIG. 12G).

### 3 Input CIPHR Logic Gates

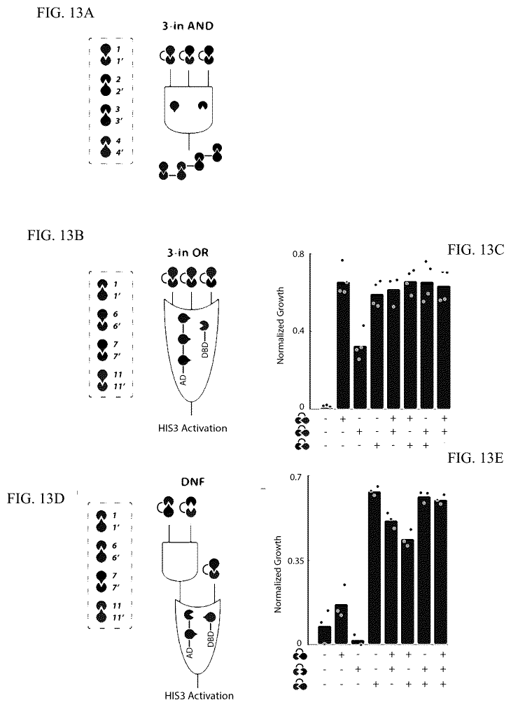

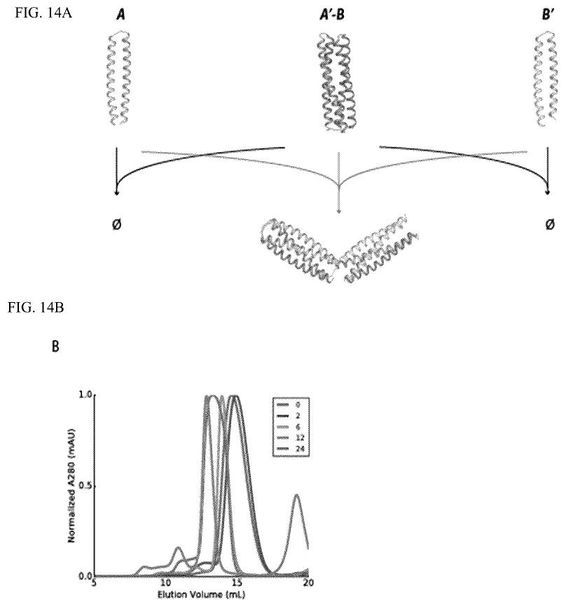

We constructed a 3-input AND gate (FIG. 10M) in which monomers 1 and 2 are 20 brought into proximity by the three inputs 1′-4′, 4-3′, and 3-2′. We experimentally tested all eight possible input combinations (FIG. 13A), quantifying all complexes using nMS with both 1 and 2 present. Consistent with proper function of a 3-input AND gate, 1 and 2 only showed significant co-assembly when all three inputs are present (data not shown).

To test the modularity of CIPHR logic gates, we designed two different 3-input CIPHR logic gates using the same 4 pairs of DHDs and tested them via Y2H. To make a 3-input OR gate, 1′-6-7 was fused to AD, and 11′ to DBD. Either one of the 3 inputs (11-1, 11-6′, 11-7′) is able to bring AD to DBD via their linked proteins (FIG. 13B). Y2H results confirmed the correct behavior of this logic gate in cells: any of the input proteins induces cell growth (FIG. 13C). We constructed a CIPHR disjunctive normal form (DNF, [A AND B]30 OR C) gate by fusing 1′-6 to AD, 11′ to DBD with inputs 11-7′, 7-1, or 11-6′ (FIG. 13D). In Y2H experiments, the DNF gate functioned as designed, with low yeast growth levels when no input or only one of the 11-7′ and 7-1 input proteins are present, and high yeast growth levels otherwise (FIG. 13E).

### REFERENCES FOR EXAMPLE 2

1. R. Nussinov, How do dynamic cellular signals travel long distances? Mol. Biosyst. 8, 22-26 (2012).
2. A. W. Reinke, J. Baek, O. Ashenberg, A. E. Keating, Networks of bZIP protein-protein interactions diversified over a billion years of evolution. Science. 340, 730-734 (2013).
3. Y. E. Antebi, J. M. Linton, H. Klumpe, B. Bintu, M. Gong, C. Su, R. McCardell, M. B. Elowitz, Combinatorial Signal Perception in the BMP Pathway. Cell. 170, 1184-1196.e24 (2017).
4. B. Z. Harris, W. A. Lim, Mechanism and role of PDZ domains in signaling complex assembly. J. Cell Sci. 114, 3219-3231 (2001).
5. G. Seelig, D. Soloveichik, D. Y. Zhang, E. Winfree, Enzyme-free nucleic acid logic circuits. Science. 314, 1585-1588 (2006).
6. L. Qian, E. Winfree, Scaling up digital circuit computation with DNA strand displacement cascades. Science. 332, 1196-1201 (2011).
7. M. B. Elowitz, S. Leibler, A synthetic oscillatory network of transcriptional regulators. Nature. 403, 335-338 (2000).
8. T. S. Gardner, C. R. Cantor, J. J. Collins, Construction of a genetic toggle switch in Escherichia coli. Nature. 403, 339-342 (2000).
9. A. Tamsir, J. J. Tabor, C. A. Voigt, Robust multicellular computing using genetically encoded NOR gates and chemical “wires.” Nature. 469, 212-215 (2011).
10. P. Siuti, J. Yazbek, T. K. Lu, Synthetic circuits integrating logic and memory in living cells. Nat. Biotechnol. 31, 448-452 (2013).
11. J. Bonnet, P. Yin, M. E. Ortiz, P. Subsoontorn, D. Endy, Amplifying genetic logic gates. Science. 340, 599-603 (2013).
12. B. H. Weinberg, N. T. H. Pham, L. D. Caraballo, T. Lozanoski, A. Engel, S. Bhatia, W. W. Wong, Large-scale design of robust genetic circuits with multiple inputs and outputs for mammalian cells. Nat. Biotechnol. 35, 453-462 (2017).
13. S. Auslander, D. Auslander, M. Müller, M. Wieland, M. Fussenegger, Programmable single-cell mammalian biocomputers. Nature. 487, 123-127 (2012).
14. A. S. Khalil, T. K. Lu, C. J. Bashor, C. L. Ramirez, N. C. Pyenson, J. K. Joung, J. J. Collins, A synthetic biology framework for programming eukaryotic transcription functions. Cell. 150, 647-658 (2012).
15. N. Roquet, A. P. Soleimany, A. C. Ferris, S. Aaronson, T. K. Lu, Synthetic recombinase-based state machines in living cells. Science. 353, aad8559 (2016).
16. L. B. Andrews, A. A. K. Nielsen, C. A. Voigt, Cellular checkpoint control using programmable sequential logic. Science. 361, eaap8987 (2018).
17. B. Angelici, E. Mailand, B. Haefliger, Y. Benenson, Synthetic Biology Platform for Sensing and Integrating Endogenous Transcriptional Inputs in Mammalian Cells. Cell Rep. 16, 2525-2537 (2016).
18. J. J. Lohmueller, T. Z. Armel, P. A. Silver, A tunable zinc finger-based framework for Boolean logic computation in mammalian cells. Nucleic Acids Res. 40, 5180-5187 (2012).
19. A. A. Green, P. A. Silver, J. J. Collins, P. Yin, Toehold switches: de-novo-designed regulators of gene expression. Cell. 159, 925-939 (2014).
20. A. A. Green, J. Kim, D. Ma, P. A. Silver, J. J. Collins, P. Yin, Complex cellular logic computation using ribocomputing devices. Nature. 548, 117-121 (2017).
21. K. Rinaudo, L. Bleris, R. Maddamsetti, S. Subramanian, R. Weiss, Y. Benenson, A universal RNAi-based logic evaluator that operates in mammalian cells. Nat. Biotechnol. 25, 795-801 (2007).
22. L. Wroblewska, T. Kitada, K. Endo, V. Siciliano, B. Stillo, H. Saito, R. Weiss, Mammalian synthetic circuits with RNA binding proteins for RNA-only delivery. Nat. Biotechnol. 33, 839-841 (2015).
23. S.-H. Park, A. Zarrinpar, W. A. Lim, Rewiring MAP kinase pathways using alternative scaffold assembly mechanisms. Science. 299, 1061-1064 (2003).
24. P. L. Howard, M. C. Chia, S. Del Rizzo, F.-F. Liu, T. Pawson, Redirecting tyrosine kinase signaling to an apoptotic caspase pathway through chimeric adaptor proteins. Proc. Natl. Acad. Sci. U.S.A. 100, 11267-11272 (2003).
25. B. J. Yeh, R. J. Rutigliano, A. Deb, D. Bar-Sagi, W. A. Lim, Rewiring cellular morphology pathways with synthetic guanine nucleotide exchange factors. Nature. 447, 596-600 (2007).
26. L. Morsut, K. T. Roybal, X. Xiong, R. M. Gordley, S. M. Coyle, M. Thomson, W. A. Lim, Engineering Customized Cell Sensing and Response Behaviors Using Synthetic Notch Receptors. Cell. 164, 780-791 (2016).
27. K. T. Roybal, L. J. Rupp, L. Morsut, W. J. Walker, K. A. McNally, J. S. Park, W. A. Lim, Precision Tumor Recognition by T Cells With Combinatorial Antigen-Sensing Circuits. Cell. 164, 770-779 (2016).
28. J. E. Dueber, B. J. Yeh, K. Chak, W. A. Lim, Reprogramming Control of an Allosteric Signaling Switch Through Modular Recombination. Science. 301, 1904-1908 (2003).
29. R. M. Gordley, R. E. Williams, C. J. Bashor, J. E. Toettcher, S. Yan, W. A. Lim, Engineering dynamical control of cell fate switching using synthetic phospho-regulons. Proc. Natl. Acad. Sci. U.S.A. 113, 13528-13533 (2016).
30. J. E. Dueber, E. A. Mirsky, W. A. Lim, Engineering synthetic signaling proteins with ultrasensitive input/output control. Nat. Biotechnol. 25, 660-662 (2007).
31. X. J. Gao, L. S. Chong, M. S. Kim, M. B. Elowitz, Programmable protein circuits in living cells. Science. 361, 1252-1258 (2018).
32. T. Fink, J. Lonzarid, A. Praznik, T. Plaper, E. Merljak, K. Leben, N. Jerala, T. Lebar, Z. Strmsek, F. Lapenta, M. Bençina, R. Jerala, Design of fast proteolysis-based signaling and logic circuits in mammalian cells. Nat. Chem. Biol. 15, 115-122 (2019).
33. A. J. Smith, F. Thomas, D. Shoemark, D. N. Woolfson, N. J. Savery, Guiding Biomolecular Interactions in Cells Using de Novo Protein-Protein Interfaces. ACS Synth. Biol. 8, 1284-1293 (2019).
34. Z. Chen, S. E. Boyken, M. Jia, F. Busch, D. Flores-Solis, M. J. Bick, P. Lu, Z. L. VanAernum, A. Sahasrabuddhe, R. A. Langan, S. Bermeo, T. J. Brunette, V. K. Mulligan, L. P. Carter, F. DiMaio, N. G. Sgourakis, V. H. Wysocki, D. Baker, Programmable design of orthogonal protein heterodimers. Nature. 565, 106-111 (2019).
35. K. E. Prehoda, J. A. Scott, R. D. Mullins, W. A. Lim, Integration of multiple signals through cooperative regulation of the N-WASP-Arp2/3 complex. Science. 290, 801-806 (2000).
36. B. Yu, I. R. S. Martins, P. Li, G. K. Amarasinghe, J. Umetani, M. E. Fernandez-Zapico, D. D. Billadeau, M. Machius, D. R. Tomchick, M. K. Rosen, Structural and energetic mechanisms of cooperative autoinhibition and activation of Vav1. Cell. 140, 246-256 (2010).
37. K. N. Dyer, M. Hammel, R. P. Rambo, S. E. Tsutakawa, I. Rodic, S. Classen, J. A. Tamer, G. L. Hura, High-throughput SAXS for the characterization of biomolecules in solution: a practical approach. Methods Mol. Biol. 1091, 245-258 (2014).
38. A. Pause, B. Peterson, G. Schaffar, R. Stearman, R. D. Klausner, Studying interactions of four proteins in the yeast two-hybrid system: structural resemblance of the pVHL/elongin BC/hCUL-2 complex with the ubiquitin ligase complex SKP1/cullin/F-box protein. Proc. Natl. Acad. Sci. U.S.A. 96, 9533-9538 (1999).
39. B. Sandrock, J. M. Egly, A yeast four-hybrid system identifies Cdk-activating kinase as a regulator of the XPD helicase, a subunit of transcription factor IIH. J. Biol. Chem. 276, 35328-35333 (2001).
40. A. S. Dixon, M. K. Schwinn, M. P. Hall, K. Zimmerman, P. Otto, T. H. Lubben, B. L. Butler, B. F. Binkowski, T. Machleidt, T. A. Kirkland, M. G. Wood, C. T. Eggers, L. P. Encell, K. V. Wood, NanoLuc Complementation Reporter Optimized for Accurate Measurement of Protein Interactions in Cells. ACS Chem. Biol. 11, 400-408 (2016).
41. Y.-C. Kwon, M. C. Jewett, High-throughput preparation methods of crude extract for robust cell-free protein synthesis. Sci. Rep. 5, 8663 (2015).
42. J. R. Porter, C. I. Stains, B. W. Jester, I. Ghosh, A general and rapid cell-free approach for the interrogation of protein-protein, protein-DNA, and protein-RNA interactions and their antagonists utilizing split-protein reporters. J. Am. Chem. Soc. 130, 6488-6497 (2008).
43. S. L. Maude, T. W. Laetsch, J. Buechner, S. Rives, M. Boyer, H. Bittencourt, P. Bader, M. R. Verneris, H. E. Stefanski, G. D. Myers, M. Qayed, B. De Moerloose, H. Hiramatsu, K. Schlis, K. L. Davis, P. L. Martin, E. R. Nemecek, G. A. Yanik, C. Peters, A. Baruchel, N. Boissel, F. Mechinaud, A. Balduzzi, J. Krueger, C. H. June, B. L. Levine, P. Wood, T. Taran, M. Leung, K. T. Mueller, Y. Zhang, K. Sen, D. Lebwohl, M. A. Pulsipher, S. A. Grupp, Tisagenlecleucel in Children and Young Adults with B-Cell Lymphoblastic Leukemia. N. Engl. J. Med. 378, 439-448 (2018).
44. S. S. Neelapu, F. L. Locke, N. L. Bartlett, L. J. Lekakis, D. B. Miklos, C. A. Jacobson, I. Braunschweig, O. O. Oluwole, T. Siddiqi, Y. Lin, J. M. Timmerman, P. J. Stiff, J. W. Friedberg, I. W. Flinn, A. Goy, B. T. Hill, M. R. Smith, A. Deol, U. Farooq, P. McSweeney, J. Munoz, I. Avivi, J. E. Castro, J. R. Westin, J. C. Chavez, A. Ghobadi, K. V. Komanduri, R. Levy, E. D. Jacobsen, T. E. Witzig, P. Reagan, A. Bot, J. Rossi, L. Navale, Y. Jiang, J. Aycock, M. Elias, D. Chang, J. Wiezorek, W. Y. Go, Axicabtagene Ciloleucel CAR T-Cell Therapy in Refractory Large B-Cell Lymphoma. N. Engl. J. Med. 377, 2531-2544 (2017).
45. J. A. Fraietta, S. F. Lacey, E. J. Orlando, I. Pruteanu-Malinici, M. Gohil, S. Lundh, A. C. Boesteanu, Y. Wang, R. S. O'Connor, W.-T. Hwang, E. Pequignot, D. E. Ambrose, C. Zhang, N. Wilcox, F. Bedoya, C. Dorfmeier, F. Chen, L. Tian, H. Parakandi, M. Gupta, R. M. Young, F. B. Johnson, I. Kulikovskaya, L. Liu, J. Xu, S. H. Kassim, M. M. Davis, B. L. Levine, N. V. Frey, D. L. Siegel, A. C. Huang, E. J. Wherry, H. Bitter, J. L. Brogdon, D. L. Porter, C. H. June, J. J. Melenhorst, Determinants of response and resistance to CD19 chimeric antigen receptor (CAR) T cell therapy of chronic lymphocytic leukemia. Nat. Med. 24, 563-571 (2018).
46. C. H. June, R. S. O'Connor, O. U. Kawalekar, S. Ghassemi, M. C. Milone, CAR T cell immunotherapy for human cancer. Science. 359, 1361-1365 (2018).
47. A. H. Long, W. M. Haso, J. F. Shern, K. M. Wanhainen, M. Murgai, M. Ingaramo, J. P. Smith, A. J. Walker, M. E. Kohler, V. R. Venkateshwara, R. N. Kaplan, G. H. Patterson, T. J. Fry, R. J. Orentas, C. L. Mackall, 4-1BB costimulation ameliorates T cell exhaustion induced by tonic signaling of chimeric antigen receptors. Nat. Med. 21, 581-590 (2015).
48. E. J. Wherry, M. Kurachi, Molecular and cellular insights into T cell exhaustion. Nat. Rev. Immunol. 15, 486-499 (2015).
49. E. John Wherry, S. J. Ha, S. M. Kaech, W. Nicholas Haining, S. Sarkar, V. Kalia, S. Subramaniam, J. Blattman, D. L. Barber, R. Ahmed, Molecular Signature of CD8+ T Cell Exhaustion during Chronic Viral Infection. Immunity. 27 (2007), doi:10.1016/j.immuni.2007.11.006.
50. K. E. Pauken, M. A. Sammons, P. M. Odorizzi, S. Manne, J. Godec, O. Khan, A. M. Drake, Z. Chen, D. R. Sen, M. Kurachi, R. A. Barnitz, C. Bartman, B. Bengsch, A. C. Huang, J. M. Schenkel, G. Vahedi, W. N. Haining, S. L. Berger, E. J. Wherry, Epigenetic stability of exhausted T cells limits durability of reinvigoration by PD-1 blockade. Science. 354, 1160-1165 (2016).
51. K. E. Prehoda, W. A. Lim, How signaling proteins integrate multiple inputs: a comparison of N-WASP and Cdk2. Curr. Opin. Cell Biol. 14, 149-154 (2002).

Referring to Table 7, for an induced dimerization system involving proteins A, A′-B, and B′, a stoichiometric excess (N) of the A′-B protein results in partially assembled dimeric complexes if the binding is non-cooperative, but fully assembled trimeric complexes if the binding is cooperative.

We model the cooperatively induced dimerization system at thermodynamic equilibrium. Shown below (Table 8), assuming a ‘closed’ state for A′-B, where the binding interfaces are buried within the four-helix bundle, the binding of A′-B to either A or B′ helix hairpins needs to overcome an energy barrier of transitioning from the ‘closed’ to ‘open’ state (ΔGₒₚₑₙ). Therefore the free energy of binding between A′-B to A or B′ can be expressed as ΔG_{A:A′}−ΔGₒₚₑₙ and ΔG_{B:B′}−ΔGₒₚₑₙ, respectively, where ΔG_{A:A′} and ΔG_{B:B′} represent the free energy of binding between the cognate pairs in the absence of the fusion. Once the A:A′-B or A-B′:B complexes form, subsequent binding can be simply represented by the binding between cognate heterodimers: ΔG_{A:A′} or ΔG_{B:B′}. We also observed the presence of (A)2 and (B′)₂ homodimers, therefore added free energy terms describing such processes into the model (ΔG_{A:A} or ΔG_{B′:B′}).

ΔG relates to equilibrium constants by ΔG=−RT lnK, and we further consider the system in terms of K. We make the simplifying assumption that the affinity of A′-B to either A or B′ is identical (K₁=[A:A′−B]/([A][A′−B])=[A′−B:B′]/([B][A′−B]). Finally, we define the cooperativity of the system, c, as the ratio between the equilibrium constants in the presence or absence of the other partner (c=K_{B:B′}/K₁=K_{A:A′}/K₁). For an entirely non-cooperative process (c=1), K_{B:B′}=K₁ and K_{A:A′}=K₁ i.e., the first binding event does not affect the affinity of the subsequent binding event.

Since K₁=exp(−(ΔG_{A:A′}−ΔGₒₚₑₙ)/RT), rewriting the equation for c in terms of free energies leads to c=exp(ΔGₒₚₑₙ)/RT. Therefore, the extent of cooperativity is solely determined by the magnitude of the free energy required to partially unfold/expose the buried binding interfaces of the dimerizer A′-B.

We note that explicitly incorporating the equilibrium constants for homodimerization (K_{A:A} and K_{B′:B′}) only affect the absolute position of each equilibrium, but does not affect the magnitude of the cooperativity (see Table 9). Indeed, taking A as an example, the binding to the closed state becomes K₁*K_{A:A}, and the binding to the open state becomes K_{A:A′}*K_{A:A}. Because K_{A:A} is present in both the numerator and the denominator, they cancel out, and c remains purely defined by the relative magnitudes of K₁ and K_{A:A′}.

We solved the following system of equations in Mathematica to simulate the amount of A:A′-B:B′ at equilibrium as a function of the initial concentration of A′-B:

$${K}_{A : A} = \frac{[ {A}_{2} ]}{[ A ] [ A ]} \\ {K}_{B ′ : B ′} = \frac{[ {B}_{2}^{′} ]}{[ {B}^{′} ] [ {B}^{′} ]} \\ {K}_{1} = \frac{[ A : {A}^{′} - B ]}{[ A ] [ {A}^{′} - B ]} \\ {K}_{1} = \frac{[ {B}^{′} : {A}^{′} - B ]}{[ {B}^{′} ] [ {A}^{′} - B ]} \\ {K}_{A : A ′} = \frac{[ A : {A}^{′} - B : {B}^{′} ]}{[ A \} [ {A}^{′} - B : {B}^{′} ]} \\ {K}_{B : B ′} = \frac{[ A : {A}^{′} - B : {B}^{′} ]}{[ {B}^{′} ] [ A : {A}^{′} - B ]} \\ {[ A ]}_{tot} = 2 * [ {A}_{2} ] + [ A ] + [ A : {A}^{′} - B ] + [ A : {A}^{′} - B : {B}^{′} ] \\ {[ {B}^{′} ]}_{tot} = 2 * [ {B}_{2}^{′} ] + [ {B}^{′} ] + [ {A}^{′} - B : {B}^{′} ] + [ A : {A}^{′} - B : {B}^{′} ] \\ {[ {A}^{′} - B ]}_{tot} = [ {A}^{′} - B : {B}^{′} ] + [ A : {A}^{′} - {B}^{′} ] + [ A : {A}^{′} - B : {B}^{′} ]$$

We knew from previous native MS titration experiments that the equilibrium dissociation constants of cognate designed heterodimers (DHDs) is in the ˜10 nM range (1), therefore K_{A:A′}=K_{B:B′}=0.1 nM⁻¹. Varying values of K₁ (and hence the cooperativity factor, c=K_{A:A′}/K₁) showed different responses of the amount of A:A′-B:B′ at equilibrium as a function of the initial concentration of A′-B, as shown in FIG. 12C.

We experimentally estimated K₁ using native MS experiments. Mixing 10 μM of 1 and 1′-2′ resulted in no detectable amount of the 1:1′-2′ complex, suggesting very weak binding. The sensitivity of native MS places a lower-bound on the concentration of species that can be detected (0.0375 ρM). Using this value, a lower-bound for the affinity of 1:1′-2′ can be estimated (1/K₁≥2.65 mM). This is close to the value of 9.91 mM obtained by calculating the affinity based on the c value of 991,000 reported in FIG. 13H.

This thermodynamic modeling demonstrates that binding cooperativity can be achieved for an induced dimerization system through occlusion of the binding interfaces. We achieved this by fusing hairpins via a flexible linker, rationalizing that the spontaneous folding of these constructs would bury the interaction interfaces on the inside of a four helical bundle like topology. Formation of these structures is corroborated by: i) SAXS profiles that are consistent with DHDs structures, ii) m-values from chemical denaturation experiments consistent with ΔSASA for the unfolding of DHD topologies, and iii) ΔGₒₚₑₙ<ΔG_{folding}, suggesting that exposing the binding interfaces requires partial unfolding of these fused constructs, but does not exceed the folding free energy of these proteins (a physically unrealistic scenario).

### Materials and Methods

### Buffer and Media Recipe

TBM-5052: 1.2% [wt/vol] tryptone, 2.4% [wt/vol] yeast extract, 0.5% [wt/vol]glycerol, 0.05% [wt/vol] D-glucose, 0.2% [wt/vol] D-lactose, 25 mM Na2HPO4, 25 mM KH2PO4, 50 mM NH4Cl, 5 mM Na2SO4, 2 mM MgSO4, 10 μM FeCl3, 4 μM CaCl2, 2 μM MnCl2, 2 μM ZnSO4, 400 nM CoCl2, 400 nM NiCl2, 400 nM CuCl2, 400 nM Na2MoO4, 400 nM Na2SeO3, 400 nM H3B03.

Lysis buffer: 20 mM Tris, 300 mM NaCl, 20 mM Imidazole, pH 8.0 at room temperature.

Wash buffer: 20 mM Tris, 300 mM NaCl, 30 mM Imidazole, pH 8.0 at room temperature.

Elution buffer: 20 mM Tris, 300 mM NaCl, 250 mM Imidazole, pH 8.0 at room temperature.

TBS buffer: 20 mM Tris pH 8.0, 100 mM NaCl.

YPAD buffer: Peptone 20 g/L, yeast extract 10 g/L, Adenine hemisulfate 10 μg/L, dextrose (20 g/L).

C-Trp-Ura-Leu-His+Adenine: hemisulfate+Glucose.

Yeast nitrogen base w/o amino acids (6.7 g/L), synthetic DO media (-Leu/-His/-Trp/-Ura) (1.4 g/L), dextrose (20 g/L), adenine hemisulfate (10 μg/L).

### Construction of Synthetic Genes

For the expression of proteins in E. coli, synthetic genes were ordered from Genscript Inc. (Piscataway, N.J., USA) and delivered in pET21-NESG E. coli expression vector, inserted between the NdeI and XhoI sites. For each expression construct, a hexahistidine tag followed by a tobacco etch virus (TEV) protease cleavage site (GSSHHHHHHSSGENLYFQGS) (SEQ ID NO:328) were added in frame at the N-terminus of the protein. A stop codon was introduced at the 3′ end of the protein coding sequence to prevent expression of the C-terminal hexahistidine tag in the vector.

Genes for yeast-two-hybrid (Y2H) studies were cloned into plasmids bearing the GAL4 DNA-binding domain (poDBD) and the GAL4 transcription activation domain (poAD) (2). Input proteins were cloned into plasmids V510 (uracil auxotrophic selection marker) and MX1 (bleomycin selection marker). Genes were expressed under the control of ADH1 promoters.

### Protein Expression

Plasmids were transformed into chemically competent E. coli expression strain Lemo21™ (DE3) (New England Biolabs) for protein expression. Following transformation and overnight growth, single colonies were picked from agar plates into 5 ml Luria-Bertani (LB) medium containing 100 μg/mL carbenicillin (for pET21-NESG vectors) with shaking at 225 rpm for 18 hours at 37° C. Proteins were expressed using the autoinduction method (7): starter cultures were further diluted into 500 ml TBM-5052 containing 100 μg/mL carbenicillin, and incubated with shaking at 225 rpm for 24 hours at 37° C.

### Affinity Purification

E. coli cells were harvested by centrifugation at 5000 rcf for 15 minutes at 4° C. and the pellet resuspended in 18 ml lysis buffer. EDTA-free cocktail protease inhibitor (Roche), lysozyme, and DNAse were added to the resuspended cell pellet, followed by cell lysis via sonication at 70% power for 5 minutes. Lysates were clarified by centrifugation at 4° C. and 18,000 rpm for 45 minutes and applied to columns containing Ni-NTA (Qiagen) resin pre-equilibrated with lysis buffer. The column was washed two times with 5 column volumes (CV) of wash buffer, followed by 5 CV of elution buffer for protein elution.

### Size-Exclusion Chromatography (SEC)

Eluted proteins were buffer exchanged into lysis buffer. N-terminal hexahistidine tags were removed with TEV protease cleavage overnight at room temperature, at a ratio of 1 mg TEV for 100 mg of protein. After TEV cleavage, sample was passed over a fresh Ni-NTA column and washed with 1.5 CV of lysis buffer, collecting flow through. The resulting proteins were purified by SEC using a Superdex™ 75 10/300 increase column (GE Healthcare) in TBS buffer.

### Circular Dichroism (CD) Measurements

Circular dichroism (CD) wavelength scans (260-195 nm) and temperature melts (25-95° C.) were performed using an AVIV™ model 420 CD spectrometer, with protein samples diluted to 0.25 mg/ml in PBS pH 7.4 in a 0.1-cm cuvette. Temperature melts were carried out at a heating rate of 4° C./min and monitored by the change in ellipticity at 222 nm.

GdmCl titrations were performed on a JASCO™ model J-1500 with automated titration apparatus in PBS pH 7.4 at 25° C., with protein concentrations between 0.08 mg/ml to 0.025 mg/ml in a 1-cm cuvette with stir bar. Each titration consisted of at least 34 evenly distributed GdmCl concentration points up to 7.4 M with 30 seconds mixing time for each step. Titrant solution consisted of the same concentration of protein in PBS and GdmCl.

### CD Data Analysis and Model Fitting

Folding free energies were obtained by fitting equilibrium denaturation data. Fused hairpin constructs had biphasic unfolding transitions, indicating the existence of an intermediate on their respective energy landscapes. Since native MS showed that Linker 0, Linker 2, Linker 6, and Linker 12 were almost exclusively monomeric in buffer (data not shown), it was concluded that these intermediates were partially folded monomeric species. Thus, the chemical denaturation data of these proteins was fitted to a unimolecular 3-state model:

N⇔I⇔D

where N represents the fully folded state, I a partially folded intermediate, and D the denatured state. The fraction of each species can be written as a function of K₁=[I]/[N] and K₂=[D]/[I], the equilibrium constants for the first and second transitions respectively:

$${f}_{N} = {( 1 + {K}_{1} + {K}_{1} \cdot {K}_{2} )}^{- 1} \\ {f}_{I} = {( 1 + {K}_{2} + \frac{1}{{K}_{1}} )}^{- 1} \\ {f}_{D} = {( 1 + \frac{1}{{K}_{2}} + \frac{1}{{K}_{1} \cdot {K}_{2}} )}^{- 1}$$

In the context of equilibrium chemical denaturation experiments, the free energy of unfolding is a linear function of denaturant concentration:

ΔG_{[den]}=ΔG_{buffer}−m·[den]

where ΔG_{[den]} represents the free energy of the system at a given concentration of denaturant, ΔG_{buffer} is the corresponding free energy change in the absence of denaturant, and m is a constant of proportionality that relates to the change in solvent-accessible surface area upon unfolding (ΔSASA). Thus, the effect of denaturant on the equilibrium constant relating to each transition can be written as a function of its free energy difference in buffer, and a specific m-value:

$${K}_{1} = exp ( \frac{{m}_{1} \cdot [ den ] - Δ {G}_{1}}{R \cdot T} ) \\ {K}_{2} = exp ( \frac{{m}_{2} \cdot [ den ] - Δ {G}_{2}}{R \cdot T} )$$

By combining these expressions with the definitions for f_{N}, f_{I}, f_{D}, the fractional distribution of each species can be expressed as a function of denaturant concentration, and the free energy change corresponding to each transition (in buffer). Finally, for an ensemble spectroscopic technique such as CD, the observed signal (the dependent variable) as a function of denaturant concentration (the independent variable) can be expressed as a linear combination of the spectroscopic signals corresponding to each species, weighed by their fractional contribution to the ensemble:

MRE_{222 nm}=f_{N}·MRE_{N}+f_{I}·MRE_{I}+f_{D}·MRE_{D}

Where MRE_{N}, MRE_{I}, MRE_{D} represent the spectroscopic signatures (baselines) for the native, intermediate, and denatured states respectively. This equation was used to fit chemical denaturation data for the different linker proteins, and the fitted parameters are reported in Table 10. For Linker 24 in buffer, native MS revealed a significant proportion of dimer (data not shown). Therefore, this model is not entirely appropriate for describing the unfolding, and the fitted values for this construct should be interpreted with care. Nevertheless, denaturation performed at different concentrations of protein revealed that the position of the second transition was concentration-independent, and thus unimolecular. For this event, the model holds.

The total m-values for these linked hairpins were found to be around 3 kcal mol⁻¹ M⁻¹. It has been shown that m-values correlate with ΔSASA of unfolding (8). For the folded state, SASA was estimated from the structures of DHDs (1) using PyMOL™ to be 8800 Å². For the unfolded state, SASA was estimated using ProtSA™ (9, 10), and is about 20,000 Å². Thus, ΔSASA for the unimolecular unfolding of a fused hairpin should be around 11,000 Å², which would have a predicted m-value of 3.3. This number is in close agreement with the fitted parameters reported here, in line with the notion that the folded state for these linker proteins has a four helix bundle topology.

### Small Angle X-Ray Scattering (SAXS)

Protein samples were purified by SEC in 25 mM Tris pH 8.0, 150 mM NaCl and 2% glycerol; elution fractions preceding the void volume of the column were used as blanks for buffer subtraction. Scattering measurements were performed at the SIBYLS™ 12.3.1 beamline at the Advanced Light Source. The sample-to-detector distance was 1.5 m, and the X-ray wavelength (λ) was 1.27 Å, corresponding to a scattering vector q (q=4π sin θ/λ, where 2θ is the scattering angle) range of 0.01 to 0.3 Å⁻¹. A series of exposures were taken of each well, in equal sub-second time slices: 0.3-s exposures for 10 s resulting in 32 frames per sample. For each sample, data were collected for two different concentrations to test for concentration-dependent effects; ‘low’ concentration samples ranged at 2.5 mg/ml and ‘high’ concentration samples at 5 mg/ml. Data were processed using the SAXS FrameSlice™ online serve and analyzed using the ScAtter™ software package (11, 12). The FoXS™ online server (13, 14) was used to compare experimental scattering profiles to design models and calculate quality of fit (χ) values.

### Yeast Two-Hybrid Assay for Logic Gates

Chemically competent cells of yeast strain PJ69-4a (MATa trp1-901 leu2-3,112 ura3-52 his3-200 gal4(deleted) gal80(deleted) LYS2::GAL1-HIS3 GAL2-ADE2 met2::GAL7-lacZ) were transformed with the appropriate pair of plasmids containing DNA binding domains (DBD) or activation domains (AD), using the LiAc/SS carrier DNA/PEG method (15). For two input CIPHR logic gates, genes encoding the input proteins (together with selection markers) were genetically integrated into either or both of the Ura3 locus (uracil auxotrophic selection marker) or the YCR043 locus (bleomycin selection marker). In the case of three input CIPHR logic gates, genes encoding two input proteins were genetically integrated as described, with the additional input cloned downstream of either the AD or DBD plasmid, separated by a p2a and nuclear localization sequence (GSGATNFSLLKQAGDVEENPGPGDKAELIPEPPKKKRKVELGTA; SEQ ID NO:330). The p2a sequence ensures translational cleavage to make the additional input protein a separate protein. The selection of transformed yeast cells was performed in synthetic dropout (SDO) medium lacking tryptophan and leucine for 48 h with shaking at 1,000 r.p.m. at 30° C. The resulting culture was diluted 1:100 and grown for 16 h in fresh SDO medium lacking tryptophan and leucine, before being diluted 1:100 in fresh SDO medium lacking tryptophan, leucine and histidine. The culture was incubated with shaking at 1,000 r.p.m. at 30° C. As it is necessary to bring the DBD and the transcription activation domain into proximity for the growth of yeast cells in medium lacking histidine, successful activation of logic gates was indicated by the growth of yeast cells (16, 17). The optical density of yeast cells was recorded at 24 h, 48 h, and 72 h.

TABLE 10
Fitted parameters for equilibrium chemical denaturation.
Errors represent fitting errors.

| | Linker 0 | Linker 2 | Linker 6 | Linker 12 | Linker 24 |
|---|---|---|---|---|---|
| ΔG₁^{(N⇔I)} | 3.6 | 3.5 | 3.5 | 2.7 | 3.7 |
| (kcal mol⁻¹) | (±0.4) | (±0.2) | (±0.2) | (±0.1) | (±0.3) |
| ΔG₂^{(I⇔D)} | 9.8 | 10.7 | 12.2 | 10.6 | 10.4 |
| (kcal mol⁻¹) | (±0.6) | (±0.4) | (±0.4) | (±0.5) | (±0.8) |
| ΔGₜₒₜ^{(N⇔D)} | 13.5 | 14.1 | 15.7 | 13.3 | 14.1 |
| (kcal mol⁻¹) | (±0.7) | (±0.4) | (±0.5) | (±0.5) | (±0.8) |
| m₁ | 1.1 | 1.0 | 0.9 | 0.75 | 1.1 |
| (kcal mol⁻¹ | (±0.2) | (±0.1) | (±0.1) | (±0.05) | (±0.1) |
| M⁻¹) | | | | | |
| m₂ | 1.8 | 1.97 | 2.22 | 1.96 | 2.0 |
| (kcal mol⁻¹ | (±0.1) | (±0.07) | (±0.08) | (±0.08) | (±0.1) |
| M⁻¹) | | | | | |
| mₜₒₜ | 2.9 | 3.0 | 3.1 | 2.71 | 3.1 |
| (kcal mol⁻¹ | (±0.2) | (±0.1) | (±0.1) | (±0.09) | (±0.2) |
| M⁻¹) | | | | | |
| MRE_{N} | −23,574 | −27,561 | −24,712 | −33,849 | −26,438 |
| (deg cm² | (±114) | (±84) | (±63) | (±131) | (±123) |
| dmol⁻¹) | | | | | |
| MRE_{I} | −16,330 | −18,139 | −14,779 | −17,362 | −15,567 |
| (deg cm² | (±749) | (±540) | (±710) | (±1,158) | (±914) |
| dmol⁻¹) | | | | | |
| MRE_{D} | −525 | −785 | −937 | −1,104 | −1,125 |
| (deg cm² | (±107) | (±82) | (±68) | (±99) | (±133) |
| dmol⁻¹) | | | | | |

### REFERENCES

1. Z. Chen, S. E. Boyken, M. Jia, F. Busch, D. Flores-Solis, M. J. Bick, P. Lu, Z. L. VanAemnum, A. Sahasrabuddhe, R. A. Langan, S. Bermeo, T. J. Brunette, V. K. Mulligan, L. P. Carter, F. DiMaio, N. G. Sgourakis, V. H. Wysocki, D. Baker, Programmable design of orthogonal protein heterodimers. Nature. 565, 106-111 (2019).
2. S. E. Boyken, Z. Chen, B. Groves, R. A. Langan, G. Oberdorfer, A. Ford, J. M. Gilmore, C. Xu, F. DiMaio, J. H. Pereira, B. Sankaran, G. Seelig, P. H. Zwart, D. Baker, De novo design of protein homo-oligomers with modular hydrogen-bond network-mediated specificity. Science. 352, 680-687 (2016).
3. A. S. Dixon, M. K. Schwinn, M. P. Hall, K. Zimmerman, P. Otto, T. H. Lubben, B. L. Butler, B. F. Binkowski, T. Machleidt, T. A. Kirkland, M. G. Wood, C. T. Eggers, L. P. Encell, K. V. Wood, NanoLuc Complementation Reporter Optimized for Accurate Measurement of Protein Interactions in Cells. ACS Chem. Biol. 11, 400-408 (2016).
4. M. E. Lee, W. C. DeLoache, B. Cervantes, J. E. Dueber, A Highly Characterized Yeast Toolkit for Modular, Multipart Assembly. ACS Synth. Biol. 4, 975-986 (2015).
5. A. S. Khalil, T. K. Lu, C. J. Bashor, C. L. Ramirez, N. C. Pyenson, J. K. Joung, J. J. Collins, A synthetic biology framework for programming eukaryotic transcription functions. Cell. 150, 647-658 (2012).
6. A. Aranda-Diaz, K. Mace, I. Zuleta, P. Harrigan, H. El-Samad, Robust Synthetic Circuits for Two-Dimensional Control of Gene Expression in Yeast. ACS Synth. Biol. 6, 545-554 (2017).
7. F. W. Studier, Protein production by auto-induction in high density shaking cultures. Protein Expr. Purif 41, 207-234 (2005).
8. J. K. Myers, C. N. Pace, J. M. Scholtz, Denaturant m values and heat capacity changes: relation to changes in accessible surface areas of protein unfolding. Protein Sci. 4, 2138-2148 (1995).
9. P. Bemadó, M. Blackledge, J. Sancho, Sequence-specific solvent accessibilities of protein residues in unfolded protein ensembles. Biophys. J. 91, 4536-4543 (2006).
10. J. Estrada, P. Bernadó, M. Blackledge, J. Sancho, ProtSA: a web application for calculating sequence specific protein solvent accessibilities in the unfolded ensemble. BMC Bioinformatics. 10, 104 (2009).
11. K. N. Dyer, M. Hammel, R. P. Rambo, S. E. Tsutakawa, I. Rodic, S. Classen, J. A. Tamer, G. L. Hura, High-throughput SAXS for the characterization of biomolecules in solution: a practical approach. Methods Mol. Biol. 1091, 245-258 (2014).
12. R. P. Rambo, J. A. Tamer, Characterizing flexible and intrinsically unstructured biological macromolecules by SAS using the Porod-Debye law. Biopolymers. 95, 559-571 (2011).
13. D. Schneidman-Duhovny, M. Hammel, A. Sali, FoXS: a web server for rapid computation and fitting of SAXS profiles. Nucleic Acids Res. 38, W540-4 (2010).
14. D. Schneidman-Duhovny, M. Hammel, J. A. Tamer, A. Sali, Accurate SAXS profile computation and its assessment by contrast variation experiments. Biophys. J 105, 962-974 (2013).
15. R. H. Schiestl, R. D. Gietz, High efficiency transformation of intact yeast cells using single stranded nucleic acids as a carrier. Curr. Genet. 16, 339-346 (1989).
16. C. T. Chien, P. L. Bartel, R. Sternglanz, S. Fields, The two-hybrid system: a method to identify and clone genes for proteins that interact with a protein of interest. Proc. Natl. Acad. Sci. U.S.A. 88, 9578-9582 (1991).
17. P. L. Bartel, J. A. Roecklein, D. SenGupta, S. Fields, A protein linkage map of Escherichia coli bacteriophage T7. Nat. Genet. 12, 72-77 (1996).
18. Z. L. VanAemum, J. D. Gilbert, M. E. Belov, A. A. Makarov, S. R. Homing, V. H. Wysocki, Surface-Induced Dissociation of Noncovalent Protein Complexes in an Extended Mass Range Orbitrap Mass Spectrometer. Anal. Chem. 91, 3611-3618 (2019).
19. M. T. Marty, A. J. Baldwin, E. G. Marklund, G. K. A. Hochberg, J. L. P. Benesch, C. V. Robinson, Bayesian deconvolution of mass and ion mobility spectra: from binary interactions to polydisperse ensembles. Anal. Chem. 87, 4370-4376 (2015).
20. Y.-C. Kwon, M. C. Jewett, High-throughput preparation methods of crude extract for robust cell-free protein synthesis. Sci. Rep. 5, 8663 (2015).
21. M. C. Jewett, J. R. Swartz, Mimicking the Escherichia coli cytoplasmic environment activates long-lived and efficient cell-free protein synthesis. Biotechnol. Bioeng. 86, 19-26 (2004).
22. A. D. Silverman, N. Kelley-Loughnane, J. B. Lucks, M. C. Jewett, Deconstructing Cell-Free Extract Preparation for in Vitro Activation of Transcriptional Genetic Circuitry. ACS Synth. Biol. 8, 403-414 (2019).
23. J. R. Swartz, M. C. Jewett, K. A. Woodrow, in Recombinant Gene Expression: Reviews and Protocols, P. Balbis, A. Lorence, Eds. (Humana Press, Totowa, N J, 2004), pp. 169-182.
24. V. Muñoz, L. Serrano, Elucidating the folding problem of helical peptides using empirical parameters. III. Temperature and pH dependence. J Mol. Biol. 245, 297-308 (1995).
23. V. Muñoz, L. Serrano, Elucidating the folding problem of helical peptides using empirical parameters. III. Temperature and pH dependence. J. Mol. Biol. 245, 297-308 (1995).

1-116. (canceled)

117. A composition comprising a designed heterodimer protein, wherein the designed heterodimer protein comprises:
(a) a first polypeptide, wherein the first polypeptide is a non-naturally occurring polypeptide comprising 1-5 alpha helices connected by amino acid linkers; and
(b) a second polypeptide, wherein the second polypeptide is a non-naturally occurring polypeptide comprising 1-5 alpha helices connected by amino acid linkers,
wherein the first polypeptide and the second polypeptide non-covalently interact to form the designed heterodimer protein,
wherein the first polypeptide, the second polypeptide, or both are independently linked to one or more: effector polypeptide domains, therapeutic agents, or detection tags at the N-terminus, the C-terminus, or both.

118. The composition of claim 117, wherein the non-covalent interaction that forms the designed heterodimer protein comprises at least one designed hydrogen bond network comprising at least 2 hydrogen bonds between amino acids of the first polypeptide and the second polypeptide, and wherein the at least one designed hydrogen bond network is at the interface between the first polypeptide and the second polypeptide.

119. The composition of claim 117, wherein the first polypeptide, the second polypeptide, or both are independently linked to the one or more: effector polypeptide domains, therapeutic agents, or detection tags by PEGylation, HESylation, PASylation, glycosylation, an amino acid linker, or combinations thereof.

120. The composition of claim 117, wherein the first polypeptide is linked to one or more of: the effector polypeptide domains, the therapeutic agents, or the detection tags is capable of non-covalently binding to a first target.

121. The composition of claim 117, wherein the second polypeptide is linked to one or more of: the effector polypeptide domains, the therapeutic agents, or the detection tags is capable of non-covalently binding to a second target.

122. The composition of claim 117, wherein the first polypeptide consists essentially of 2-5 alpha helices.

123. The composition of claim 117, wherein the first polypeptide consists essentially of 2-4 alpha helices.

124. The composition of claim 117, wherein the second polypeptide consists essentially of 2-5 alpha helices.

125. The composition of claim 117, wherein the second polypeptide consists essentially of 2-4 alpha helices.

126. The composition of claim 117, comprising the first polypeptide, the second polypeptide, or both which are independently linked to the one or more therapeutic agents.

127. The composition of claim 117, comprising the first polypeptide, the second polypeptide, or both which are independently linked to the one or more effector polypeptide domains.

128. The composition of claim 117, comprising the first polypeptide, the second polypeptide, or both which are independently linked to the one or more one or more detection tags.

129. The composition of claim 117, comprising the first polypeptide, the second polypeptide, or both which are independently linked to the one or more: effector polypeptide domains, therapeutic agents, or detection tags at the N-terminus.

130. The composition of claim 117, comprising the first polypeptide, the second polypeptide, or both which are independently linked to the one or more: effector polypeptide domains, therapeutic agents, or detection tags at the C-terminus.

131. The composition of claim 117, wherein the supercoil phases of the alpha helices are fixed at 0, 90, 180, or 270 degrees.

132. The composition of claim 117, wherein the designed heterodimer protein is an Fc-fusion designed heterodimer protein.

133. The composition of claim 117, wherein the composition further comprises an eukaryotic cell.

134. The composition of claim 117, wherein the first polypeptide comprises a sequence with at least 90% sequence identity to SEQ ID NO: 3.

135. The composition of claim 117, wherein the second polypeptide comprises a sequence with at least 90% sequence identity to SEQ ID NO: 4.

136. A method, comprising:
(a) detecting heterodimer formation between:
1) a first polypeptide, wherein the first polypeptide is a non-naturally occurring polypeptide comprising 1-5 alpha helices connected by amino acid linkers linked to a first polypeptide domain capable of binding to a first target; and
2) a second polypeptide, wherein the second polypeptide is a non-naturally occurring polypeptide comprising 1-5 alpha helices connected by amino acid linkers linked to a second polypeptide domain capable of binding to a second target; and
(b) detecting binding between the first polypeptide domain and the first target, or detecting binding between the second polypeptide domain and the second target.

137. The method of claim 136, wherein the detecting comprises detecting an output signal.

138. The method of claim 137, wherein the output signal is fluorescence activity or functional activity.
